(19) United States
(12) Patent Application Publication
(10) Pub. No.: US 2025/0026836 A1
(43) Pub. Date: Jan. 23, 2025

(54) ANTI-HLA-G ANTIBODIES

(71) Applicant: UCB BIOPHARMA SRL, BRUSSELS (BE)

(72) Inventors: CAROLINE BERTEAU, SLOUGH, BERKSHIRE (GB); THOMAS COLLEY, SLOUGH, BERKSHIRE (GB); NEESHA DEDI, SLOUGH, BERKSHIRE (GB); CARL BRENDAN DOYLE, SLOUGH, BERKSHIRE (GB); DAVID PAUL HUMPHREYS, SLOUGH, BERKSHIRE (GB); GAELLE LE FRIEC, SLOUGH, BERKSHIRE (GB); RUTH MCELHONE, SLOUGH, BERKSHIRE (GB); VICTORIA O'DOWD, SLOUGH, BERKSHIRE (GB); CLARE THOMPSON, SLOUGH, BERKSHIRE (GB); KERRY LOUISE TYSON, SLOUGH, BERKSHIRE (GB); ANN LOUISE WHITE, SLOUGH, BERKSHIRE (GB)

(21) Appl. No.: 18/684,711

(22) PCT Filed: Aug. 19, 2022
(86) PCT No.: PCT/EP2022/073195

§ 371 Date: Feb. 19, 2024

(30) Foreign Application Priority Data
Aug. 19, 2021 (GB) 2111905.2

(51) Int. Cl. C07K 16/28

(52) U.S. Cl. CPC C07K 16/2833; C07K 2317/24; C07K 2317/732; C07K 2317/92

(57) ABSTRACT

The present invention relates to antibodies directed against HLA-G and formulations comprising the same. The invention further relates to the use of the HLA-G antibodies and formulations in therapy, notably in the treatment of solid tumors.

### FIELD OF THE INVENTION

The present invention relates to antibodies directed against HLA-G and formulations comprising the same. The invention further relates to the use of the HLA-G antibodies and formulations in therapy, notably in the treatment of solid cancers.

### BACKGROUND OF THE INVENTION

Class I Human Leukocyte Antigens (HLA-Is) comprise classical antigens, HLA-A, HLA-B and HLA-C, as well as non-classical antigens HLA-E, HLA-F, and HLA-G. Human Leukocyte Antigen G (HLA-G) is a non-classical HLA class I molecule expressed in humans and encoded by the HLA-G gene. HLA-G is a heterodimer molecule, comprising a heavy chain which exhibits 3 globular domains (α₁, α₂ and α₃) associated with a light chain, namely beta-2-microglobulin (B2m).

Seven isoforms of HLA-G have been identified, four are membrane-bound (HLA-G1, HLA-G2, HLA-G3, HLA-G4) and three are soluble (HLA-G5, HLA-G6 and HLA-G7), which are the result of alternative splicing of the HLA-G primary transcript.

HLA-G is normally expressed on cytotrophoblasts in the placenta. The expression of HLA-G has been reported to be associated with pathological conditions such as inflammatory diseases and cancers. Notably, it has been reported that HLA-G is a tolerogenic molecule specifically upregulated in solid cancers and associated with poor prognosis.

HLA-G is known to show immune-regulatory activity through binding to at least three receptors expressed on various myeloid and lymphoid cells:
The inhibitory receptor LILRB1 (for leukocyte immunoglobulin-like receptor B1), also referred to as ILT2 or CD85j, expressed on lymphoid (B cells, some T cells and NK cells) and myeloid cells (monocytes, macrophages and dendritic cells)
The inhibitory receptor LILRB2 (for leukocyte immunoglobulin-like receptor B2), also referred to as ILT4 or CD85d, expressed on myeloid cells (monocytes, macrophages and dendritic cells); and
The regulatory receptor KIR2DL4 or CD158d, expressed on NK cells.

HLA-G inhibits the function of immune cells through direct binding of its inhibitory receptors. It has been reported that HLA-G has a tolerogenic function that is mediated mainly by the interaction of its α₃ domain with ILT2 and ILT4. ILT2 only recognizes HLA-G molecules that are associated with B2m, whereas ILT4 recognizes both B2m associated and B2m free HLA-G molecules.

Through this immune inhibitory function, HLA-G expression by tumors can induce an immunosuppressed environment, allowing tumor immune escape and ultimately decreasing patient survival. Antibody mediated blockade of HLA-G may therefore provide an effective strategy to relieve tumor localised immune suppression, promote development of anti-cancer immunity and provide durable therapy.

Whilst in theory the anti-tumor effect of an HLA-G blocking antibody could be augmented by inclusion of an active Fc component capable of engaging FcγR on immune effector cells to allow direct killing of HLA-G+ tumor cells, the reported expression of HLA-G mRNA and protein in a number of normal tissues, including the pancreas and pituitary, suggest that the use of an active Fc may result in unacceptable toxicity, therefore precluding its use in therapy.

HLA-G shares high similarity with other HLA-I molecules (i.e. HLA-A, HLA-B, HLA-C, HLA-E and HLA-F). Of 338 amino acid positions in the HLA-G protein, only 20 have residues that are unique to HLA-G and are not present at that position in any other of the ˜5000 human HLA-I molecules. This makes it very challenging to produce antibodies with high specificity to HLA-G with no cross-reactivity to other HLA-I molecules.

Commercial HLA-G antibodies are available today. However, some have been reported to lack specificity for HLA-G (e.g. cross-reactive with other HLA-Is molecules) and all have epitopes in the α1 and α2 domains of HLA-G which are remote from the HLA-G ILT2/4 binding site in the α3 domain and therefore not predicted to block interaction between HLA-G and ILT2 and/or ILT4. Antibody 87G, that engages an epitope in the HLA-G α1 domain, has been reported to relieve HLA-G inhibition of immune cell function. However, it has not been reported that 87G and other commercially available HLA-G antibodies were able to block HLA-G ILT2/4 interaction. Due to their lack of specificity and/or blocking activity, the commercial antibodies are not suitable for the development of an HLA-G therapeutic antibody.

Other antibodies binding to HLA-G have been reported in WO19202040 and WO2020069133 and whilst such antibodies appear to modulate one or more HLA-G activities, their binding site on HLA-G (i.e. epitope) has not been characterized.

To date, efficacy of an antibody against HLA-G has not been demonstrated in patients, in particular for the treatment of solid cancers.

Therefore, there remains a need to provide antibodies which bind HLA-G and have biological properties useful in therapy, such as improved pharmacokinetic properties and/or improved biological functions (e.g. specificity, binding affinity, neutralisation and/or cell cytotoxicity and phagocytosis) and/or reduced toxicity in humans.

### SUMMARY OF THE INVENTION

The present invention addresses the above-identified need by providing new antibodies against HLA-G useful in therapy, notably in the treatment of solid cancers, with the structural and functional properties as described herein, notably high specificity for HLA-G, the ability to block the interaction of HLA-G with its receptors ILT2 and ILT4. We provide evidence that the epitope recognised by the antibodies of the invention is not present in normal tissues, including pituitary and pancreas, thereby supporting for the first time the use of an antibody format comprising an active Fc, capable of direct tumor cell killing in patients.

In particular, the present invention provides an antibody that specifically binds to human HLA-G, comprising:
a. a light chain variable region comprising:
   a CDR-L1 comprising SEQ ID NO:1,
   a CDR-L2 comprising SEQ ID NO: 2, and
   a CDR-L3 comprising SEQ ID NO: 3; and
b. a heavy chain variable region comprising:
   a CDR-H1 comprising SEQ ID NO: 4,
   a CDR-H2 comprising SEQ ID NO: 5, and
   a CDR-H3 comprising SEQ ID NO: 6.

### BRIEF DESCRIPTION OF THE DRAWINGS

FIG. 1: HLA-G isoforms (FIG. 1B of Carosella et al., Blood, Vol. 111, no 10, 2008)

FIG. 2. Humanization of rabbit variable light chain sequence of antibody 12389. Grafts 12389gL1, gL2, and gL3 are humanized grafts of rabbit variable light chain of antibody 12389 using IGKV1D-13 human germline as the acceptor framework. The CDRs are shown in bold/underlined. Donor residues are shown in bold/italic and are grey shaded: V3 and Q70.

FIG. 3. Humanization of rabbit variable heavy chain sequence of antibody 12389. Grafts 12389gH1, gH4, gH5, gH6, gH8, gH9, gH11, gH12, gH13, gH14, gH15 and gH16 are humanized grafts of rabbit variable heavy chain of antibody 12389 using IGHV3-66 human germline as the acceptor framework. The CDRs are shown in bold/underlined. Donor residues are shown in bold/italic and are grey shaded: V24, 148, G49, K71, S73, V78 and G96.

FIG. 4. Specificity of HLA-G02 in a PBMC assay from 50 different donors. Results are expressed in MFI of each CD4+ cell population for each donor and antibody.

FIG. 5. Specificity of HLA-G02; binding to HLA-G1, HLA-G2, HLA-G3 and HLA-G4 expressed on cells (FIG. 5A.) Binding to HLA-G2 (FIG. 5B).

FIG. 6. Percentage of depleted Epcam+GFP+HCT116 target cells following treatment with different anti-HLA-G antibodies or an IgG1 isotype control antibody. Each antibody was tested at two different concentrations either 1 μg/ml (white bars) or 0.01 μg/ml (striped bars). The E:T ratio was 3.5:1. Each bar represents the mean (and the range) of three data points and each dot/square is an individual replicate. Data is from one representative donor. HLA-G01 to HLA-G08 are represented by their respective ID numbers (01 to 08).

FIG. 7. Percentage of depleted Epcam+GFP+HCT116 target cells following treatment with anti-HLA-G antibodies HLA-G01 and HLA-G02 or an IgG1 isotype control antibody from three separate experiments (3 different donors). Antibodies were tested at 1 μg/ml (FIG. 7A) or 0.01 μg/ml (FIG. 7B). The E:T ratio was between 2.5 and 3:1. Each bar represents the mean (and range) of data from an individual experiment and each dot, square or triangle is an individual replicate.

FIG. 8. Percentage depletion of Epcam+GFP+HCT116 cells (Vertical axis) following treatment with a titration of anti-HLA-G antibodies HLA-G01 and HLA-G02 compared to an isotype control IgG1 (Horizontal axis: antibody concentration in μg/ml). The E:T ratio was 4:1. Each point represents the mean (and range) of 3 replicates. Data shown is from a single representative donor.

FIG. 9A. Percentage depletion of JEG3 cells (Vertical axis) following treatment with conventional HLA-G02 IgG1 (solid line) or afucosylated HLA-G02 IgG1 (“aF HLA-G02”, dotted line). Horizontal axis: antibody concentration in μg/ml. The E:T ratio was 10:1. Each point represents the mean (and range) of 2 replicates. Data is shown from a single representative donor.

FIG. 9B. Percentage depletion of Epcam+GFP+HCT116 cells (Vertical axis) following treatment with conventional HLA-G02 IgG1 (solid line) or afucosylated HLA-G02 IgG1 (“aF HLA-G02”, dotted line) compared to an isotype control IgG1. Horizontal axis: antibody concentration in μg/ml. The E:T ratio was 5:1. Each point represents the mean (and range) of 3 replicates. Data is shown from a single representative donor.

FIG. 10. Titration of the HLA-G-specific phagocytosis activity of HLA-G02 on Mock transfected (FIG. 10A) and HLA-G/B2m transfected K562 target cells (FIG. 10B) compared to anti-CD47 antibody (“aCD47”) and to an isotype control IgG1. Vertical axis: percentage of CTY+CD11b+ double positive cells; Horizontal axis: antibody concentration in μg/ml.

FIG. 11. Titration of the HLA-G-specific phagocytosis activity of conventional and afucosylated (aF) formats of the HLA-G02 on Mock transfected (FIG. 11A) and HLA-G-expressing K562 target cells (FIG. 11B) compared to anti-CD47 antibody and to an isotype control IgG1. Vertical axis: percentage of CTY+CD11b+ double positive cells; Horizontal axis: antibody concentration in μg/ml.

FIG. 12. VR12389 (black surface representation) blocks the interaction of HLA-G with ILT-2 and ILT4. FIG. 12A: Crystal structure (PDB ID 6AEE) of ILT2 (cartoon, white) in complex with HLA-G (cartoon, grey) and B2M (mesh, grey). FIG. 12B: Superposition with the crystal structure of VR12389 in complex with HLA-G and B2M shows that VR12389 (surface, black) blocks the interaction of ILT2 with HLA-G. FIG. 12C: Crystal structure (PDB ID 2DYP) of ILT4 (cartoon, white) in complex with HLA-G (cartoon, grey) and B2M (mesh, grey). FIG. 12D: Superposition with the crystal structure of VR12389 in complex with HLA-G and B2M shows that VR12389 (surface, black) blocks the interaction of ILT4 with HLA-G.

FIG. 13. Tumor cell killing assay. FIG. 13A: data obtained with anti-PDL1 from RCC. FIG. 13B: data obtained with anti-PDL1 from CRC. FIG. 13C: data obtained with HLA-G02 from RCC. FIG. 13D: data obtained with HLA-G02 from CRC. Data is presented as % dead cells for isotype control in light grey, anti-PDL1 or HLA-G02 in dark grey and anti-PDL1 or HLA-G02 treated cultures where a 1.5-fold or greater increase in cell death was observed in black.

FIG. 14. Depletion of HLA-G transfected HCT116 cells at different effector:target ratios after 2.5 to 3 h following treatment with conventional or afucosylated HLA-G02. The number of live HLA-G GFP+HCT116 cells was determined by flow cytometry and percent depletion (vertical axis) was calculated relative to the no antibody controls. Horizontal axis: E:T ratio (effector:target ratio). Data shown is from a single donor (1). Each data point represents the mean of 3 replicates. Error bars represent the 95% confidence interval.

FIG. 15. Specificity of afucosylated HLA-G02 (“aF HLA-G02”) in a PBMC assay from 10 different donors. Results are expressed in MFI of each CD4+ cell population for each donor and antibody.

FIG. 16. CDC mediated by afucosylated HLA-G02. FIG. 16A: Impact of serum activity on HLAG-β2m-Reh cell lysis (Concentration-response curve depicting aF HLA-G02 mediated CDC of HLAG-β2m-Reh in the presence of active or heat-inactivated serum. Vertical axis: lysis (%) normalised against the minimum and maximum controls (N=1, 4-PL fit); horizontal axis: antibody concentration [M]). FIG. 16B: Dependence on HLA-G for aF HLA-G02 mediated lysis (Concentration-response curve depicting aF HLA-G02 mediated CDC of HLAG-β2m-Reh or Reh cells. Vertical axis: lysis (%) normalised against the minimum and maximum controls (N=1, 4-PL fit); horizontal axis: antibody concentration [M]). FIG. 16C: aF HLA-G02 mediated CDC of HLAG-β2m-Reh cells (Concentration-response curve depicting aF HLA-G02 mediated CDC of HLAG-β2m-Reh. Vertical axis: lysis (%) normalised against the minimum and maximum controls (N=3, mean±SEM, 4-PL fit); horizontal axis: antibody concentration [M]).

FIG. 17. Concentration dependent effect of HLA-G02 and afucosylated HLA-G02 on the phagocytosis of target cells expressing HLA-G or mock transfected cells compared to αCD47 antibody. FIGS. 17A and B: representative data (mean±SD) (Vertical axis: percentage of CTY+CD11b+ double positive cells; Horizontal axis: antibody concentration in μg/ml). FIGS. 17 C and D: combined data generated using monocytes from three separate donors, in duplicate, across two independent experiments (Vertical axis: percentage of phagocytosis adjusted to isotype control=(Antibody mean−isotype mean)±SD; Horizontal axis: antibody concentration in μg/ml).

FIG. 18. Target cell killing mediated by afucosylated HLA-G02. Percentage of depletion of HLA-G expressing target cells by ADCP (vertical axis) with different concentrations of afucosylated HLA-G02 or αCD47 (horizontal axis, in μg/ml) after overnight incubation (from four separate donors, in duplicate, across two independent experiments). Mean and standard deviation compared to isotype control.

FIG. 19. Representative data of the concentration dependent effect of afucosylated HLA-G02 and HLA-G02 IgG4P FALA on the phagocytosis of HLA-G expressing cells alone (FIG. 19A) or in combination with anti-CD47 Ab (at 1 μg/mL, FIG. 19B) compared to respective isotype controls. Vertical axis: percentage of CTY+CD11b+ double positive cells; Horizontal axis: antibody concentration in μg/ml.

FIG. 20. Phagocytosis of HLA-G expressing cells treated with afucosylated HLA-G02 alone or in combination with anti-CD47 antibody (at 1 μg/mL). FIG. 20A represents data for afucosylated HLA-G02 (three donors, in duplicate, two independent experiments). FIG. 20B represents data with HLA-G02 IgG4P FALA (data combined from four donors, each analyzed in duplicate, three independent experiments). Phagocytosis values (%, vertical axis) were adjusted to the respective isotype controls (Ab mean−isotype mean) and are presented as mean±SD. Horizontal axis: antibody concentration in μg/ml.

FIG. 21. Levels of cytokines (vertical axis, μg/ml) (FIG. 21A: IFN gamma; FIG. 21B: TNF alpha; FIG. 21C: IL-2; FIG. 21D: IL-6; FIG. 21E: IL-8; FIG. 21F: IL-10), produced in PBMC cultures in the presence and absence of JEG3 cells across a concentration range of afucosylated HLA-G02 (horizontal axis, μg/ml). Values are presented as mean+/−standard deviation for all 16 PBMC donors. Dotted lines indicate the mean level of each cytokine produced in the presence of afucosylated isotype control antibody at 50 μg/ml, anti-CD3 at 50 μg/ml or LPS at 100 ng/ml.

### DETAILED DESCRIPTION OF THE INVENTION

The present disclosure will now be described with respect to particular non-limiting aspects and embodiments thereof and with reference to certain figures and examples.

Technical terms are used by their common sense unless indicated otherwise. If a specific meaning is conveyed to certain terms, definitions of terms will be given in the context of which the terms are used.

Where the term “comprising” is used in the present description and claims, it does not exclude other elements. For the purposes of the present disclosure, the term “consisting of” is considered to be a preferred embodiment of the term “comprising of”.

Where an indefinite or definite article is used when referring to a singular noun, e.g. “a”, “an” or “the”, this includes a plural of that noun unless something else is specifically stated.

The present disclosure provides an antibody against HLA-G. In a first aspect, the present invention provides an antibody that specifically binds to HLA-G, comprising:
a. a light chain variable region comprising:
   a CDR-L1 comprising SEQ ID NO:1,
   a CDR-L2 comprising SEQ ID NO:2, and
   a CDR-L3 comprising SEQ ID NO: 3; and
b. a heavy chain variable region comprising:
   a CDR-H1 comprising SEQ ID NO:4,
   a CDR-H2 comprising SEQ ID NO: 5, and
   a CDR-H3 comprising SEQ ID NO:6.

### HLA-G

The term “HLA-G” or “human HLA-G” refers to Human Leucocyte Antigen G, which is a classical HLA-I molecule also known as human major histocompatibility complex I molecule (MHC). Typically, HLA-G forms an MHC-I complex together with B2m.

Unless otherwise specified, the term “HLA-G” refers to any alternative splicing or natural variants or isoforms of human HLA-G which are naturally expressed by cells. An exemplary sequence of full Human HLA-G comprises the sequence given in SEQ ID NO: 107.

In some aspects, the antibody of the invention selectively binds to the extracellular domain (ECD) of HLA-G (or “HLA-G ECD”). An exemplary sequence of HLA-G ECD comprises the sequence given in SEQ ID NO: 108.

The amino acid and nucleic sequences of HLA-G and its isoforms are also well known in the art.

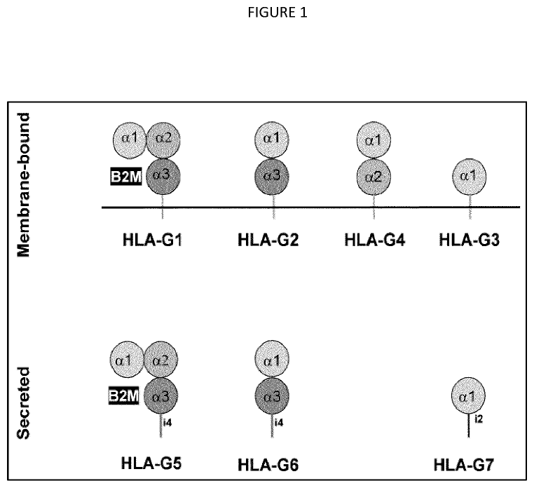

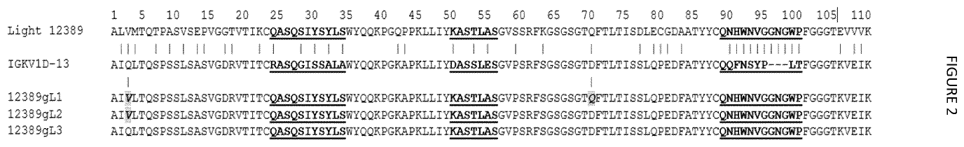

Seven isoforms of HLA-G have been identified, four are membrane-bound (HLA-G1, HLA-G2, HLA-G3, HLA-G4) and three are soluble (HLA-G5, HLA-G6 and HLA-G7), which are the result of alternative splicing of the HLA-G primary transcript (FIG. 1). Additional isoforms, some of which do not have an α₁ domain, are suggested by mRNA analysis of kidney tumors, but these have not been confirmed at the protein level (Tronik-Le Roux et al., Molecular Oncology 11 (2017) 1561-1578).

HLA-G1 and HLA-G5 have a structure similar to that of classical HLA-I molecules, i.e. heterodimer molecules, comprising a heavy chain which exhibits 3 globular domains (α₁, α₂ and α₃) associated with a light chain, namely beta-2-microglobulin (abbreviated as “β2m” or “B2m”). HLA-G1 and HLA-G5 can also exist as free alpha chains, i.e. not in complex with B2m.

HLA-G2 and HLA-G6 comprise only the α1 and α3 domains. HLA-G4 comprises only the α 1 and α2 domains. HLA-G3 and HLA-G7 comprise only the α1 domain.

In one embodiment, the antibody of the invention binds to at least one of HLA-G1, HLA-G2, HLA-G3, HLA-G4, HLA-G5, HLA-G6 and HLA-G7. In one embodiment, the antibody of the invention binds to all the HLA-G isoforms comprising an alpha 3 domain, i.e. to HLA-G1, HLA-G2, HLA-G5 and HLA-G6 including to HLA-G1 and HLA-G5 when complexed with B2m and when expressed as B2m free molecules.

HLA-G1 and HLA-G5 can form homomultimers, such as disulphide bonded dimers and trimers. In one embodiment, the antibody of the invention binds to monomeric HLA-G. In one embodiment, the antibody of the invention binds to dimeric HLA-G. In one embodiment, the antibody of the invention binds to trimeric HLA-G. In one embodiment, the antibody of the invention binds to monomeric, dimeric and trimeric HLA-G.

### Antibodies Binding to HLA-G

Antibodies for use in the context of the present disclosure include whole antibodies and functionally active fragments thereof (i.e., molecules that contain an antigen binding domain that specifically binds an antigen, also termed antigen-binding fragments). Features described herein with respect to antibodies also apply to antibody fragments unless context dictates otherwise. The antibody may be (or derived from) monoclonal, multi-valent, multi-specific, bispecific, fully human, humanized or chimeric.

Whole antibodies, also known as “immunoglobulins (Ig)” generally relate to intact or full-length antibodies i.e. comprising the elements of two heavy chains and two light chains, inter-connected by disulphide bonds, which assemble to define a characteristic Y-shaped three-dimensional structure. Classical natural whole antibodies are monospecific in that they bind one antigen type, and bivalent in that they have two independent antigen binding domains. The terms “intact antibody”, “full-length antibody” and “whole antibody” are used interchangeably to refer to a monospecific bivalent antibody having a structure similar to a native antibody structure, including an Fc region as defined herein.

In whole antibodies, each light chain is comprised of a light chain variable region (abbreviated herein as VL) and a light chain constant region (CL). Each heavy chain is comprised of a heavy chain variable region (abbreviated herein as VH) and a heavy chain constant region (CH) constituted of three constant domains CH1, CH2 and CH3, or four constant domains CH1, CH2, CH3 and CH4, depending on the Ig class. The “class” of an Ig or antibody refers to the type of constant region and includes IgA, IgD, IgE, IgG and IgM and several of them can be further divided into subclasses, e.g. IgG1, IgG2, IgG3, IgG4. The constant regions of the antibodies may mediate the binding of the immunoglobulin to host tissues or factors, including various cells of the immune system (e.g., effector cells) and the first component (Clq) of the classical complement system.

The VH and VL regions of the antibody according to the present invention can be further subdivided into regions of hypervariability (or “hypervariable regions”, or HVR) determining the recognition of the antigen, termed complementarity determining regions (CDR), interspersed with regions that are more structurally conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The CDRs and the FR together form a variable region. By convention, the CDRs in the heavy chain variable region of an antibody or antigen-binding fragment thereof are referred as CDR-H1, CDR-H2 and CDR-H3 and in the light chain variable regions as CDR-L1, CDR-L2 and CDR-L3. They are numbered sequentially in the direction from the N-terminus to the C-terminus of each chain.

CDRs are conventionally numbered according to a system devised by Kabat et al. This system is set forth in Kabat et al., 1991, in Sequences of Proteins of Immunological Interest, US Department of Health and Human Services, NIH, USA (hereafter “Kabat et al. (supra)”). This numbering system is used in the present specification except where otherwise indicated.

The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence.

The CDRs of the heavy chain variable domain are located at residues 31-35 (CDR-H1), residues 50-65 (CDR-H2) and residues 95-102 (CDR-H3) according to the Kabat numbering system. However, according to Chothia (Chothia, C. and Lesk, A. M. J. Mol. Biol., 196, 901-917 (1987)), the loop equivalent to CDR-H1 extends from residue 26 to residue 32. Thus, unless indicated otherwise ‘CDR-H1’ as employed herein is intended to refer to residues 26 to 35, as described by a combination of the Kabat numbering system and Chothia's topological loop definition.

The CDRs of the light chain variable domain are located at residues 24-34 (CDR-L1), residues 50-56 (CDR-L2) and residues 89-97 (CDR-L3) according to the Kabat numbering system.

In addition to the CDR loops, a fourth loop exists between CDR-2 (CDR-L2 or CDR-H2) and CDR-3 (CDR-L3 or CDR-H3) which is formed by framework 3 (FR3). The Kabat numbering system defines framework 3 as positions 66-94 in a heavy chain and positions 57-88 in a light chain.

Based on the alignment of sequences of different members of the immunoglobulin family, numbering schemes have been proposed and are for example described in Kabat et al., 1991, and Dondelinger et al., 2018, Frontiers in Immunology, Vol 9, article 2278.

The antibody of the invention comprises a light chain variable region comprising a CDR-L1 comprising SEQ ID NO:1, a CDR-L2 comprising SEQ ID NO:2 and a CDR-L3 comprising SEQ ID NO:3, and a heavy chain variable region comprising a CDR-H1 comprising SEQ ID NO:4, a CDR-H2 comprising SEQ ID NO: 5 and a CDR-H3 comprising SEQ ID NO:6.

In one embodiment, the antibody of the invention comprises a light chain variable region comprising the CDRs of a light chain variable region of SEQ ID NO: 19 and a heavy chain variable region comprising the CDRs of a heavy chain variable region of SEQ ID NO 93.

The antibodies comprising such CDR sequences are particularly inventive because they provide for an antibody with high affinity for HLA-G, high specificity for HLA-G (in particular no cross-reactivity with other HLA-I molecules despite of their very high homology), high inhibition for HLA-G biological functions and high stability which is essential for manufacturability. Furthermore, when combined with an active Fc, they provide for an antibody having the ability to directly kill HLA-G+ tumor cells.

The term “constant domain(s)”, “constant region”, as used herein are used interchangeably to refer to the domain(s) of an antibody which is outside the variable regions. The constant domains are identical in all antibodies of the same isotype but are different from one isotype to another. Typically, the constant region of a heavy chain is formed, from N to C terminal, by CH1-hinge —CH2-CH3-optionally CH4, comprising three or four constant domains.

The constant region domains of the antibody molecule of the present invention, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required. It will be appreciated that sequence variants of these constant region domains may also be used. For example, IgG4 molecules in which the serine at position 241 (numbered according to the Kabat numbering system) has been changed to proline as described in Angal et al. (Angal et al., 1993. A single amino acid substitution abolishes the heterogeneity of chimeric mouse/human (IgG4) antibody as observed during SDS-PAGE analysis Mol Immunol 30, 105-108) and termed IgG4P herein, may be used.

“Fc”, “Fc fragment”, and “Fc region” are used interchangeably to refer to the C-terminal region of an antibody comprising the constant region of an antibody excluding the first constant region immunoglobulin domain. Thus, Fc refers to the last two constant domains, CH2 and CH3, of IgA, IgD, and IgG, or the last three constant domains of IgE and IgM, and the flexible hinge N-terminal to these domains. The human IgG1 heavy chain Fc region is defined herein to comprise residues C226 to its carboxyl-terminus, wherein the numbering is according to the EU index as in Kabat. In the context of human IgG1, the lower hinge refers to positions 226-236, the CH2 domain refers to positions 237-340 and the CH3 domain refers to positions 341-447 according to the EU index as in Kabat. The corresponding Fc region of other immunoglobulins can be identified by sequence alignments.

In the context of the present disclosure, when present, the constant region or Fc region may be natural, as defined above, or else may be modified in various ways, provided that it comprises a functional FcR binding domain, and preferably a functional FcRn binding domain. Preferably, the modified constant region or Fc region leads to improve functionalities and/or pharmacokinetics. The modifications may include deletion of certain portions of the Fc fragment. The modifications may further include various amino acid substitutions able to affect the biological properties of the antibody. Mutations for increasing FcRn binding and thus in vivo half-life may also be present. The modifications may further include modification in the glycosylation profile of the antibody. The natural Fc fragment is glycosylated in the CH2 domain with the presence, on each of the two heavy chains, of an N-glycan bound to the asparagine residue at position 297 (Asn297). In the context of the present disclosure, the antibody may be glyco-modified, i-e engineered to have a particular glycosylation profile, which, for example, lead to improved properties, e.g. improved effector function, and/or improved serum half-life.

The antibodies described herein are isolated. An “isolated” antibody is one which has been separated (e.g. by purification means) from a component of its natural environment.

The term “antibody” encompasses monovalent, i-e antibodies comprising only one antigen binding domain (e.g. one-armed antibodies comprising a full-length heavy chain and a full-length light chain interconnected, also termed “half-antibody”), and multivalent antibodies, i-e antibodies comprising more than one antigen binding domain.

The term “antibody” according to the invention also encompasses antigen-binding fragments of antibodies.

Antigen-binding fragments of antibodies include single chain antibodies (e.g. scFv, and dsscfv), Fab, Fab′, F(ab′)₂, Fv, single domain antibodies or nanobodies (e.g. VH or VL, or VHH or VNAR). Other antibody fragments for use in the present invention include the Fab and Fab′ fragments described in International patent applications WO2011/117648, WO2005/003169, WO2005/003170 and WO2005/003171.

The methods for creating and manufacturing these antibody fragments are well known in the art (see for example Verma et al., 1998, Journal of Immunological Methods, 216, 165-181).

The term “Fab fragment” as used herein refers to an antibody fragment comprising a light chain fragment comprising a VL (variable light) domain and a constant domain of a light chain (CL), and a VH (variable heavy) domain and a first constant domain (CH1) of a heavy chain.

A typical “Fab′ fragment” comprises a heavy and a light chain pair in which the heavy chain comprises a variable region VH, a constant domain CH1 and a natural or modified hinge region and the light chain comprises a variable region VL and a constant domain CL. Dimers of a Fab′ according to the present disclosure create a F(ab′)₂ where, for example, dimerization may be through the hinge.

The term “single domain antibody” as used herein refers to an antibody fragment consisting of a single monomeric variable antibody domain. Examples of single domain antibodies include VH or VL or VHH or VNAR.

The “Fv” refers to two variable domains, for example co-operative variable domains, such as a cognate pair or affinity matured variable domains, i.e. a VH and VL pair.

“Single chain variable fragment” or “scFv” as employed herein refers to a single chain variable fragment which is stabilised by a peptide linker between the VH and VL variable domains.

“Disulphide-stabilised single chain variable fragment” or “dsscFv” as employed herein refer to a single chain variable fragment which is stabilised by a peptide linker between the VH and VL variable domain and also includes an inter-domain disulphide bond between VH and VL. (see for example, Weatherill et al., Protein Engineering, Design & Selection, 25 (321-329), 2012, WO2007109254.

In one embodiment, disulfide bond between the variable domains VH and VL is between two of the residues listed below (unless the context indicates otherwise Kabat numbering is employed in the list below). Wherever reference is made to Kabat numbering the relevant reference is Kabat et al., 1991 (5^{th} edition, Bethesda, Md.), in Sequences of Proteins of Immunological Interest, US Department of Health and Human Services, NIH, USA. In one embodiment the disulfide bond is in a position selected from the group comprising:
VH37+VL95C see for example Protein Science 6, 781-788 Zhu et al (1997);
VH44+VL100 see for example Weatherill et al., Protein Engineering, Design & Selection, 25 (321-329), 2012;
VH44+VL105 see for example J Biochem. 118, 825-831 Luo et al (1995);
VH45+VL87 see for example Protein Science 6, 781-788 Zhu et al (1997);
VH55+VL101 see for example FEBS Letters 377 135-139 Young et al (1995);
VH100+VL50 see for example Biochemistry 29 1362-1367 Glockshuber et al (1990);
VH100b+VL49; see for example Biochemistry 29 1362-1367 Glockshuber et al (1990);
VH98+VL46 see for example Protein Science 6, 781-788 Zhu et al (1997);
VH101+VL46; see for example Protein Science 6, 781-788 Zhu et al (1997);
VH105+VL43 see for example Proc. Natl. Acad. Sci. USA Vol. 90 pp. 7538-7542 Brinkmann et al (1993); or Proteins 19, 35-47 Jung et al (1994),
VH106+VL57 see for example FEBS Letters 377 135-139 Young et al (1995)
and a position or positions corresponding thereto in variable region pair located in the molecule.

In one embodiment, the disulphide bond is formed between positions VH44 and VL100.

### Multispecific Antibodies

An antibody of the invention may be a multispecific antibody. “Multispecific or Multi-specific antibody” as employed herein refers to an antibody as described herein which has at least two binding domains, i.e. two or more binding domains, for example two or three binding domains, wherein the at least two binding domains independently bind two different antigens or two different epitopes on the same antigen. Multi-specific antibodies are generally monovalent for each specificity (antigen). Multi-specific antibodies described herein encompass monovalent and multivalent, e.g. bivalent, trivalent, tetravalent multi-specific antibodies.

In one embodiment the construct is a bi-specific antibody. “Bispecific or Bi-specific antibody” as employed herein refers to an antibody with two antigen binding specificities. In one embodiment, the antibody comprises two antigen binding domains wherein one binding domain binds ANTIGEN 1 and the other binding domain binds ANTIGEN 2, i.e. each binding domain is monovalent for each antigen. In one embodiment, the antibody is a tetravalent bispecific antibody, i.e. the antibody comprises four antigen binding domains, wherein for example two binding domains bind ANTIGEN 1 and the other two binding domains bind ANTIGEN 2. In one embodiment, the antibody is a trivalent bispecific antibody.

In one embodiment the antibody construct is a tri-specific antibody. “Trispecific or Tri-specific antibody” as employed herein refers to an antibody with three antigen binding specificities. For example, the antibody is an antibody with three antigen binding domains (trivalent), which independently bind three different antigens or three different epitopes on the same antigen, i.e. each binding domain is monovalent for each antigen.

A paratope is a region of an antibody which recognises and binds to an antigen. An antibody of the invention may be a multi-paratopic antibody. “Multi-paratopic antibody” as employed herein refers to an antibody as described herein which comprises two or more distinct paratopes, which interact with different epitopes either from the same antigen or from two different antigens. Multi-paratopic antibodies described herein may be biparatopic, triparatopic, tetraparatopic.

“Antigen binding domain” as employed herein refers to a portion of the antibody, which comprises a part or the whole of one or more variable domains, for example a part or the whole of a pair of variable domains VH and VL, that interact specifically with the target antigen. A binding domain may comprise a single domain antibody. In one embodiment, each binding domain is monovalent. Preferably each binding domain comprises no more than one VH and one VL.

A variety of multi-specific antibody formats have been generated. Different classifications have been proposed, but multispecific IgG antibody formats generally include bispecific IgG, appended IgG, multispecific (e.g. bispecific) antibody fragments, multispecific (e.g. bispecific) fusion proteins, and multispecific (e.g. bispecific) antibody conjugates, as described for example in Spiess et al., Alternative molecular formats and therapeutic applications for bispecific antibodies. Mol Immunol. 67(2015):95-106.

Techniques for making bispecific antibodies include, but are not limited to, CrossMab technology (Klein et al. Engineering therapeutic bispecific antibodies using CrossMab technology, Methods 154 (2019) 21-31), Knobs-in-holes engineering (e.g. WO1996027011, WO1998050431), DuoBody technology (e.g. WO2011131746), Azymetric technology (e.g. WO2012058768). Further technologies for making bispecific antibodies have been described for example in Godar et al., 2018, Therapeutic bispecific antibody formats: a patent applications review (1994-2017), Expert Opinion on Therapeutic Patents, 28:3, 251-276.

Bispecific antibodies include in particular CrossMab antibodies, DAF (two-in-one), DAF (four-in-one), DutaMab, DT-IgG, Knobs-in-holes common LC, Knobs-in-holes assembly, Charge pair, Fab-arm exchange, SEEDbody, Triomab, LUZ-Y, Fcab, κλ-body and orthogonal Fab.

Appended IgG classically comprise full-length IgG engineered by appending additional antigen-binding domain or antigen-binding fragment to the N- and/or C-terminus of the heavy and/or light chain of the IgG. Examples of such additional antigen-binding fragments include sdAb antibodies (e.g. VH or VL), Fv, scFv, dsscFv, Fab, scFav. Appended IgG antibody formats include in particular DVD-IgG, IgG(H)-scFv, scFv-(H)IgG, IgG(L)-scFv, scFv-(L)IgG, IgG(L,H)-Fv, IgG(H)—V, V(H)-IgG, IgC(L)-V, V(L)-IgG, KIH IgG-scFab, 2scFv-IgG, IgG-2scFv, scFv4-Ig, Zybody and DVI-IgG (four-in-one), for example as described in Spiess et al., Alternative molecular formats and therapeutic applications for bispecific antibodies. Mol Immunol. 67(2015):95-106.

Multispecific antibody fragments include nanobody, nanobody-HAS, BiTEs, diabody, DART, TandAb, scDiabody, sc-Diabody-CH3, Diabody-CH3, Triple Body, Miniantibody; Minibody, Tri Bi minibody, scFv-CH3 KIH, Fab-scFv, scFv-CH-CL-scFv, F(ab′)2, F(ab′)2-scFv2, scFv-KIH, Fab-scFv-Fc, Tetravalent HCAb, scDiabody-Fc, Diabody-Fc, Tandem scFv-Fc; and intrabody, as described, for example, Spiess et al., Alternative molecular formats and therapeutic applications for bispecific antibodies. Mol Immunol. 67(2015):95-106.

Multispecific fusion proteins include Dock and Lock, ImmTAC, HSAbody, scDiabody-HAS, and Tandem scFv-Toxin.

Multispecific antibody conjugates include IgG-IgG; Cov-X-Body; and scFv1-PEG-scFv2.

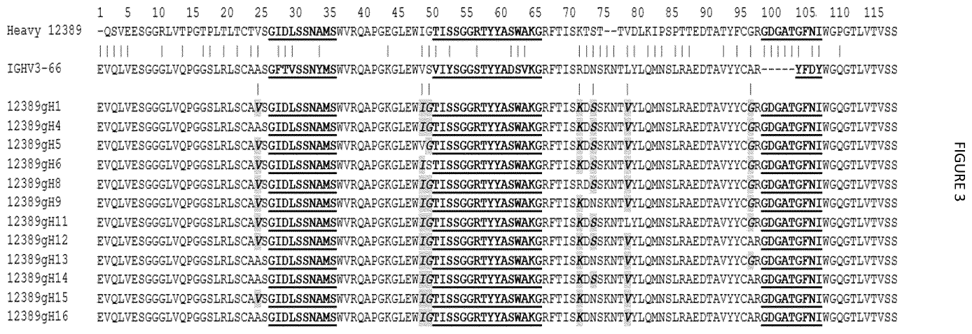

Additional multispecific antibody formats have been described for example in Brinkmann and Kontermann, The making of bispecific antibodies, mAbs, 9:2, 182-212 (2017), in particular in FIG. 2, for example tandem scFv, triplebody, Fab-VHH, taFv-Fc, scFv4-Ig, scFv2-Fcab, scFv4-IgG. Bibodies, tribodies and methods for producing the same are disclosed for example in WO99/37791.

Examples of antibodies for use in the present invention include appended IgG and appended Fab, wherein a whole IgG or a Fab fragment, respectively, is engineered by appending at least one additional antigen-binding domain (e.g. two, three or four additional antigen-binding domains), for example a single domain antibody (such as VH or VL, or VHH), a scFv, a dsscFv, a dsFv to the N- and/or C-terminus of the heavy and/or light chain of said IgG or Fab, for example as described in WO2009/040562, WO2010/035012, WO2011/030107, WO2011/061492, WO2011/061246 and WO2011/086091. In particular, the Fab-Fv format is described in WO2009/040562 and the disulphide stabilized version thereof, the Fab-dsFv, is described in WO2010/035012. A single linker Fab-dsFv, wherein the dsFv is connected to the Fab via a single linker between either the VL or VH domain of the Fv, and the C terminal of the LC or HC of the Fab, is decsribed in WO2014/096390. An appended IgG comprising a full-length IgG1 engineered by appending a dsFv to the C-terminus of the heavy or light chain of the IgG, is described in WO2015/197789.

Another example antibody for use in the present invention comprises a Fab linked to two scFvs or dsscFvs, each scFv or dsscFv binding the same or a different target (e.g., one scFv or dsscFv binding a therapeutic target and one scFv or dsscFv that increases half-life by binding, for instance, albumin). Such antibodies are described in WO2015/197772. Another example antibody for use in the present invention fragment comprises a Fab linked to only one scFv or dsscFv, as described for example in WO2013/068571 and Dave et al., Mabs, 8(7) 1319-1335 (2016).

Other well-known formats of multispecific antibodies comprise:

Diabody as employed herein refers to two Fv pairs, a first VH/VL pair and a further VH/VL pair which have two inter-Fv linkers, such that the VH of a first Fv is linked to the VL of the second Fv and the VL of the first Fv is linked to the VH of the second Fv.

Triabody as employed herein refers to a format similar to the diabody comprising three Fvs and three inter-Fv linkers.

Tetrabody as employed herein refers to a format similar to the diabody comprising fours Fvs and four inter-Fv linkers.

Tandem scFv as employed herein refers to at least two scFvs linked via a single linker such that there is a single inter-Fv linker.

Tandem scFv-Fc as employed herein refers to at least two tandem scFvs, wherein each one is appended to the N-terminus of a CH2 domain, for example via a hinge, of constant region fragment —CH2CH3.

Fab-Fv as employed herein refers to a Fv fragment with a variable region appended to the C-terminal of each of the following, the CH1 of the heavy chain and CL of the light chain. The format may be provided as a PEGylated version thereof.

Fab′-Fv as employed herein is similar to FabFv, wherein the Fab portion is replaced by a Fab′. The format may be provided as a PEGylated version thereof.

Fab-dsFv as employed herein refers to a FabFv wherein an intra-Fv disulfide bond stabilizes the appended C-terminal variable regions. The format may be provided as a PEGylated version thereof.

Fab-scFv as employed herein is a Fab molecule with a scFv appended on the C-terminal of the light or heavy chain.

Fab′-scFv as employed herein is a Fab′ molecule with a scFv appended on the C-terminal of the light or heavy chain.

DiFab as employed herein refers to two Fab molecules linked via their C-terminus of the heavy chains.

DiFab′ as employed herein refers to two Fab′ molecules linked via one or more disulfide bonds in the hinge region thereof.

As employed herein scdiabody is a diabody comprising an intra-Fv linker, such that the molecule comprises three linkers and forms a normal scFv whose VH and VL terminals are each linked to a one of the variable regions of a further Fv pair.

Scdiabody-Fc as employed herein is two scdiabodies, wherein each one is appended to the N-terminus of a CH2 domain, for example via a hinge, of constant region fragment —CH2CH3.

ScFv-Fc-scFv as employed herein refers to four scFvs, wherein one of each is appended to the N-terminus and the C-terminus of both the heavy and light chain of a —CH2CH3 fragment.

Scdiabody-CH3 as employed herein refers to two scdiabody molecules each linked, for example via a hinge to a CH3 domain.

IgG-scFv as employed herein is a full-length antibody with a scFv on the C-terminal of each of the heavy chains or each of the light chains.

scFv-IgG as employed herein is a full-length antibody with a scFv on the N-terminal of each of the heavy chains or each of the light chains.

V-IgG as employed herein is a full-length antibody with a variable domain on the N-terminal of each of the heavy chains or each of the light chains.

IgG-V as employed herein is a full-length antibody with a variable domain on the C-terminal of each of the heavy chains or each of the light chains

DVD-Ig (also known as dual V domain IgG) is a full-length antibody with 4 additional variable domains, one on the N-terminus of each heavy and each light chain.

The present disclosure provides a multispecific antibody comprising one binding domain that specifically binds to HLA-G, said binding domain comprising:
a. a light chain variable region comprising:
   a CDR-L1 comprising SEQ ID NO:1,
   a CDR-L2 comprising SEQ ID NO:2, and
   a CDR-L3 comprising SEQ ID NO: 3; and
b. a heavy chain variable region comprising:
   a CDR-H1 comprising SEQ ID NO:4,
   a CDR-H2 comprising SEQ ID NO:5, and
   a CDR-H3 comprising SEQ ID NO:6.

The antibodies of the invention specifically (or selectively) bind HLA-G. An antibody “specifically binds” a protein when it binds with preferential or high affinity to the protein of interest (e.g. HLA-G) but does not substantially bind to other proteins. In other words, the antibody binds to the protein of interest with no significant cross-reactivity to any other molecule. The specificity of an antibody may be further studied by determining whether or not the antibody binds to other related proteins as discussed above or whether it discriminates between them.

In particular, an antibody that “specifically binds” HLA-G is not cross-reactive with another human protein, notably another HLA-I molecule. In one embodiment, the antibody of the invention does not substantially bind to any of HLA-A, HLA-B, HLA-C, HLA-E, and HLA-F. In one embodiment, the antibody of the invention does not bind to any of HLA-A, HLA-B, HLA-C, HLA-E, and HLA-F. Exemplary sequences of HLA-A, HLA-B, HLA-C, HLA-E, and HLA-F are SEQ ID NO: 132, 134, 136, 138 and 140 respectively. In one embodiment, the antibody of the invention does not bind to B2m.

The antibody of the invention may specifically bind the alpha 3 domain of HLA-G. By “specifically bind the alpha 3 domain of HLA-G”, it will be understood that the antibody binds the alpha 3 domain of HLA-G with no cross-reactivity with another human protein and no cross-reactivity with another domain of HLA-G (e.g. alpha 1 or alpha 2 domain).

Cross-reactivity may for example be assessed by any suitable method described herein. Cross-reactivity of an antibody may be considered significant if the antibody binds to the other molecule at least about 5%, 10%, 15%, 20%, 25%, 30%, 35%, 40%, 45%, 50%, 55%, 60%, 65%, 70%, 75%, 80%, 85%, 90% or 100% as strongly as it binds to the protein of interest. An antibody that is specific (or selective) may bind to another molecule at less than about 90%, 85%, 80%, 75%, 70%, 65%, 60%, 55%, 50%, 45%, 40%, 35%, 30%, 25% or 20% the strength that it binds to the protein of interest. The antibody may bind to the other molecule at less than about 20%, less than about 15%, less than about 10% or less than about 5%, less than about 2% or less than about 1% the strength that it binds to the protein of interest.

In one embodiment, according to the present invention, the binding of the antibody to HLA-G is characterized by a dissociation constant (K_{D}) of less than 20 nM, in particular less than 15 nM, in particular less than 10 nM, in particular less than 9 nM, in particular less than 8 nM, in particular less than 7 nM, in particular less than 6 nM, in particular less than 5 nM.

The term “K_{D}” as used herein refers to the equilibrium dissociation constant which is obtained from the ratio of K_{d} to Kₐ (i.e. K_{d}/Kₐ) and is expressed as a molar concentration (M). K_{d} and Kₐ refers to the dissociation rate and association rate, respectively, of a particular antigen-antibody (or antigen-binding fragment thereof) interaction. K_{D} values for antibodies can be determined using methods well established in the art. A method for determining the K_{D} of an antibody is by using surface plasmon resonance (SPR), such as Biacore® system for example as described in the Examples herein, using recombinant HLA-G or a suitable fusion protein/polypeptide thereof. Typically, K_{D} values are determined by SPR at a temperature of 25° C. In one example, affinity is measured using recombinant HLA-G Extra Cellular Domain (ECD) which has been expressed in complex with B2m as described in the Examples herein. For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al, Cancer Res. 53:2560-65 (1993)).

The term “affinity” refers to the strength of an interaction between the antibody and HLA-G.

Binding affinity to HLA-G may be measured against HLA-G or HLA-G ECD, associated or not with B2m. For example, binding to HLA-G may be assessed by measuring binding affinity to soluble HLA-G ECD comprising the sequence SEQ ID NO: 108 or 110. In one example, binding to HLA-G is assessed by measuring binding affinity to soluble HLA-G ECD e.g. comprising the sequence SEQ ID NO: 108 or 110, associated with B2m. In one embodiment, the antibody of the invention binds to the ECD of HLA-G with a dissociation constant (K_{D}) of less than 20 nM, in particular less than 15 nM, in particular less than 10 nM, in particular less than 9 nM, in particular less than 8 nM, in particular less than 7 nM, in particular less than 6 nM, in particular less than 5 nM. In one embodiment, the constant of dissociation is determined by SPR at a temperature of 25° C., between an antibody of the invention expressed as a full-length antibody and a monomeric form of HLA-G (e.g. HLA-G ECD associated with B2m). In one embodiment, the dissociation constant is determined by SPR as described in Example 7.1.

In another example, binding to HLA-G may be assessed by measuring binding affinity to cell membrane expressed HLA-G comprising the sequence SEQ ID NO: 107. Binding to cell membrane expressed HLA-G may be analysed by FACS. Typically, HLA-G expressed at the surface of cells is expressed in a dimeric form. In one embodiment, the antibody of the invention binds to HLA-G on cells, as determined by FACS, with a dissociation constant (K_{D}) of less than 2 nM, preferably less than 1 nM. In one embodiment, the antibody of the invention binds to JEG3 cells, as determined by FACS, with a dissociation constant (K_{D}) of less than 1 nM.

In one embodiment, the present invention provides an antibody that specifically binds to HLA-G, comprising a light chain variable region and a heavy chain variable region, wherein the light chain variable region comprises a CDR-L1 comprising SEQ ID NO:1, a CDR-L2 comprising SEQ ID NO:2, and a CDR-L3 comprising SEQ ID NO:3; and wherein the heavy chain variable region comprises a CDR-H1 comprising SEQ ID NO:4, a CDR-H2 comprising SEQ ID NO:5, and a CDR-H3 comprising SEQ ID NO:6; and wherein the antibody has a dissociation constant (K_{D}) of less than 20 nM, in particular less than 15 nM, in particular less than 10 nM, in particular less than 9 nM, in particular less than 8 nM, in particular less than 7 nM, in particular less than 6 nM, or in particular less than 5 nM. In one embodiment, the constant of dissociation is determined by SPR at a temperature of 25° C., between an antibody of the invention expressed as a full-length antibody and a monomeric form of HLA-G.

In one embodiment, the present invention provides an antibody that specifically binds to HLA-G, comprising a light chain variable region and a heavy chain variable region, wherein the light chain variable region comprises a CDR-L1 comprising SEQ ID NO:1, a CDR-L2 comprising SEQ ID NO:2, and a CDR-L3 comprising SEQ ID NO:3; and wherein the heavy chain variable region comprises a CDR-H1 comprising SEQ ID NO:4, a CDR-H2 comprising SEQ ID NO:5, and a CDR-H3 comprising SEQ ID NO:6; and wherein the antibody, expressed as a full-length antibody, binds to JEG3 cells with a dissociation constant (K_{D}) of less than 2 nM, preferably less than 1 nM, as determined by FACS.

In one embodiment, the antibody of the present invention is a blocking antibody. The term “blocking” (or “blocks”) in the context of antibodies describes an antibody that is capable of inhibiting or attenuating the binding of its target (HLA-G) to its receptors. In one embodiment, the antibody of the present invention blocks the interaction between HLA-G and ILT2. In one embodiment, the antibody of the present invention blocks the interaction between HLA-G and ILT4. In one embodiment, the antibody of the present invention blocks the interaction between HLA-G and ILT2 and between HLA-G and ILT4. In one embodiment, the antibody of the present invention blocks the interaction between HLA-G and ILT2 and/or between HLA-G and ILT4, when HLA-G is expressed as a monomer and/or a dimer and/or a trimer.

Blocking HLA-G binding to ILT2 and/or ILT4 may be assessed by measuring blocking of the interaction between the extracellular domain (ECD) of HLA-G, associated or not with B2m, expressed at the surface of cells, with ILT2 and/or ILT4, for example expressed as fusion proteins, such as Fc fusion proteins (ITT2-Fc. ILT4-Fc). An ILT2-rabbitFc fusion protein may be used, which comprises for example SEQ ID NO: 142. An ILT4-rabbitFc fusion protein may be used, which comprises for example SEQ ID NO: 144. Blocking HLA-G binding to ILT2 and/or ILT4 may be assessed as described in Example 8.

In some embodiments, the antibody of the invention does not block the association between HLA-G and B2m. In some embodiment, the antibody of the invention does not block the association between HLA-G and its cognate peptides, naturally expressed in complex with HLA-G.

In some embodiments, the antibody of the invention inhibits the multimerization of HLA-G. In some embodiments, the antibody of the invention inhibits the dimerization of HLA-G. In some embodiments, the antibody of the invention inhibits the trimerization of HLA-G.

In one embodiment, an antibody according to the present invention has an IC₅₀ of less than 50 pM for blocking the binding of ILT2 to HLA-G, preferably, the antibody according to the present invention has an IC₅₀ of less than 40 pM, or less than 30 pM, or less than 20 pM, for blocking the binding of ILT2 to HLA-G as naturally expressed at the surface of JEG3 cells, as determined for example using the in-vitro assay using large volume of reaction as described in Example 8. In a preferred embodiment, an antibody according to the present invention has an IC₅₀ of less than 20 pM for blocking the binding of ILT2 to HLA-G as naturally expressed at the surface of JEG3 cells. In one embodiment, ILT2 is expressed as a ILT2-rabbitFc fusion protein, comprising for example SEQ ID NO: 142. In one embodiment, the antibody according to the present invention has an IC₅₀ of less than 1800 pM for blocking the binding of ILT4 to HLA-G, preferably, the antibody according to the present invention has an IC₅₀ of less than 1500 pM, or less than 1400 pM, for blocking the binding of ILT4 to HLA-G in the in-vitro assay as described herein. In one embodiment, ILT4 is expressed as a ILT4-rabbitFc fusion protein, comprising for example SEQ ID NO: 144. Blocking HLA-G binding to ILT4 may be assessed as described in Example 8.

In one embodiment, the present invention provides an antibody that specifically binds to HLA-G, comprising a light chain variable region and a heavy chain variable region, wherein the light chain variable region comprises a CDR-L1 comprising SEQ ID NO:1, a CDR-L2 comprising SEQ ID NO:2, and a CDR-L3 comprising SEQ ID NO:3; and wherein the heavy chain variable region comprises a CDR-H1 comprising SEQ ID NO:4, a CDR-H2 comprising SEQ ID NO:5, and a CDR-H3 comprising SEQ ID NO:6; and wherein the antibody has:
a. an IC₅₀ of less than 20 pM for blocking the binding of ILT2 to HLA-G as naturally expressed at the surface of JEG3 cells as determined for example using the in-vitro assay using large volume of reaction as described in Example 8; and/or
b. an IC₅₀ of less than 1400 pM for blocking the binding of ILT4 to HLA-G as determined for example as described in Example 8.

The term IC₅₀ as used herein refers to the half maximal inhibitory concentration which is a measure of the effectiveness of a substance, such as an antibody, in inhibiting a specific biological or biochemical function, which in the present invention is the binding activity of ILT2 or ILT4 to HLA-G. The IC₅₀ is a quantitative measure which indicates how much of a particular substance is needed to inhibit a given biological process or function or activity by half.

In some embodiments, the antibody of the invention inhibits HLA-G mediated immune suppressive function. In some embodiments, the antibody of the invention inhibits HLA-G mediated immune suppressive function by blocking the interaction between HLA-G and ILT2 and/or the interaction between HLA-G and ILT4. In some embodiments, the antibody of the invention inhibits HLA-G mediated suppressive function of NK cells. In some embodiments, the antibody of the invention inhibits HLA-G mediated suppressive function of cytotoxic T lymphocytes, such as CD8+T lymphocytes and/or CD4+T lymphocytes. In some embodiments, the antibody of the invention inhibits HLA-G mediated suppressive function of regulatory T lymphocytes. In some embodiments, the antibody of the invention inhibits HLA-G mediated suppressive function of B cells. In some embodiments, the antibody of the invention inhibits HLA-G mediated suppressive function of monocytes. In some embodiments, the antibody of the invention inhibits HLA-G mediated suppressive function of macrophages. In some embodiments, the antibody of the invention inhibits HLA-G mediated suppressive function of dendritic cells. In some embodiments, the antibody of the invention inhibits HLA-G mediated suppression of neutrophils. In some embodiments, the antibody of the invention inhibits HLA-G mediated suppression of phagocytosis.

In some embodiments, the antibody of the invention induces the production of proinflammatory cytokines, such as TNF alpha, IL-1, IL-10, IL-2, IL-4, IL-6, IL-8, IL-10, IL-12 (also known as IL-12p70), IL-13, IL-15, IL-18, IFN gamma, GM-CSF, CCL2, CCL3, CCL4, CCL5, TNF alpha. In some embodiments, the antibody of the invention promotes the recruitment of immune cells (monocytes, macrophages, dendritic cells, B cells, T cells or NK cells) into the tumor microenvironment. In some embodiments, the antibody of the invention inhibits HLA-G function on tumor cells expressing HLA-G. In some embodiments, the antibody of the invention induces activation of myeloid cells. In some embodiments, the antibody of the invention induces tumor cell killing, e.g. by ADCC or CDC. In some embodiments, the antibody of the invention induces tumor cell phagocytosis, e.g. ADCP. In some embodiments, the antibody of the invention inhibits angiogenesis. In some embodiments, the antibody of the invention inhibits tumor cell metastasis. In some embodiments, the antibody of the invention inhibits tumor cell proliferation.

In one embodiment, the invention provides an antibody that specifically binds to HLA-G, comprising:
a. a light chain variable region comprising:
   a CDR-L1 comprising SEQ ID NO:1,
   a CDR-L2 comprising SEQ ID NO:2, and
   a CDR-L3 comprising SEQ ID NO: 3; and
b. a heavy chain variable region comprising:
   a CDR-H1 comprising SEQ ID NO:4,
   a CDR-H2 comprising SEQ ID NO: 5, and
   a CDR-H3 comprising SEQ ID NO:6,
      wherein the antibody blocks HLA-G binding to ILT2 and/or ILT4, preferably to ILT2 and ILT4, and wherein the antibody has a dissociation constant (KD) of less than 10 nM for HLA-G. In one embodiment, the KD value is determined by SPR at a temperature of 25° C., using a full-length antibody of the invention. In one embodiment, the constant of dissociation is determined against a monomeric form of HLA-G.

In one embodiment, the invention provides an antibody that specifically binds to HLA-G, comprising:
a. a light chain variable region comprising:
   a CDR-L1 comprising SEQ ID NO:1,
   a CDR-L2 comprising SEQ ID NO:2, and
   a CDR-L3 comprising SEQ ID NO: 3; and
b. a heavy chain variable region comprising:
   a CDR-H1 comprising SEQ ID NO:4,
   a CDR-H2 comprising SEQ ID NO: 5, and
   a CDR-H3 comprising SEQ ID NO:6,
      wherein the antibody inhibits at least one of HLA-G mediated immune suppressive function as described above.

Antibodies for use in the present invention may be chimeric antibodies, humanised, or fully human.

In one embodiment the antibody is chimeric. The term “chimeric” antibody refers to an antibody in which the variable domain (or at least a portion thereof) of the heavy and/or light chain is derived from a particular source or species, for example a mouse, rat, rabbit or similar while the remainder of the heavy and/or light chain (i.e. the constant region) is derived from another species such as a human. (Morrison; PNAS 81, 6851 (1984)). Chimeric antibodies are composed of elements derived from two different species such that the element retains the characteristics of the species from which it is derived. A subcategory of “chimeric antibodies” is “humanized antibodies”.

Chimeric antibodies are typically produced using recombinant DNA methods. The DNA may be modified by substituting the coding sequence for human L and H chain constant regions for the corresponding non-human (e.g. murine or rabbit) H and L constant regions.

Humanised antibodies (which include CDR-grafted antibodies) are antibody molecules having one or more complementarity determining regions (CDRs) from a non-human species and a framework region from a human immunoglobulin molecule (see, e.g. U.S. Pat. No. 5,585,089; WO91/09967). It will be appreciated that it may only be necessary to transfer the specificity determining residues of the CDRs rather than the entire CDR (see for example, Kashmiri et al., 2005, Methods, 36, 25-34). Humanised antibodies may optionally further comprise one or more framework residues derived from the non-human species from which the CDRs were derived.

Fully human antibodies are those antibodies in which the variable regions and the constant regions (where present) of both the heavy and the light chains are all of human origin, or substantially identical to sequences of human origin, but not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP 0546073, U.S. Pat. Nos. 5,545,806, 5,569,825, 5,625,126, 5,633,425, 5,661,016, 5,770,429, EP 0438474 and EP 0463151.

In one embodiment the antibody is human. Human antibodies comprise heavy or light chain variable regions or full length heavy or light chains that are “the product of” or “derived from” a particular germline sequence if the variable regions or full-length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody or fragment thereof that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally occurring somatic mutations or intentional introduction of site-directed mutation. However, a selected human antibody typically is at least 90% identical in amino acid sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

Human antibodies may be produced by a number of methods known to those of skill in the art. Human antibodies can be made by the hybridoma method using human myeloma or mouse-human heteromyeloma cells lines (Kozbor, J Immunol; (1984) 133:3001; Brodeur, Monoclonal Isolated Antibody Production Techniques and Applications, pp 51-63, Marcel Dekker Inc, 1987). Alternative methods include the use of phage libraries or transgenic mice both of which utilize human variable region repertories (Winter G; (1994) Annu Rev Immunol 12:433-455, Green L L, (1999) J Immunol Methods 231:1 1-23).

Antibodies according to the present invention may be obtained using any suitable method known in the art. HLA-G including fusion proteins thereof, cells (recombinantly or naturally) expressing HLA-G can be used to produce antibodies which specifically recognize HLA-G. Various forms of HLA-G as described herein may be used.

In another embodiment, the antigen used is HLA-G, preferably expressed at the surface of rabbit fibroblast cells, preferably produced as described in the Examples below. In one embodiment, the antigen used is HLA-G complexed with B2m, preferably expressed at the surface of rabbit fibroblast cells, preferably produced as described in the Examples below.

HLA-G or fragments thereof, for use to immunize a host, may be prepared by processes well known in the art from genetically engineered host cells comprising expression systems. HLA-G or a fragment thereof may in some instances be part of a larger protein such as a fusion protein for example fused to an affinity tag or similar.

Antibodies generated against HLA-G may be obtained, where immunization of an animal is necessary, by administering HLA-G or a portion thereof to an animal, preferably a non-human animal, using well-known and routine protocols, see for example Handbook of Experimental Immunology, D. M. Weir (ed.), Vol 4, Blackwell Scientific Publishers, Oxford, England, 1986). Many animals, such as rabbits, mice, rats, sheep, cows, camels or pigs may be immunized. However, mice, rabbits, pigs and rats are generally used. In one embodiment, the antibody of the invention is obtained by administering a rat fibroblast cell expressing HLA-G at its surface.

Monoclonal antibodies may be made by a variety of techniques, including but not limited to, the hybridoma method, recombinant DNA methods, phage-display methods, and methods utilizing transgenic animals containing all or a part of the human immunoglobulin loci. Some exemplary methods for making monoclonal antibodies are described herein.

For example, monoclonal antibodies may be prepared using the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp 77-96, Alan R Liss, Inc., 1985).

Antibodies for use in the invention may also be generated using single lymphocyte antibody methods by cloning and expressing immunoglobulin variable region cDNAs generated from single lymphocytes selected for the production of specific antibodies by for example the methods described by Babcook, J. et al., 1996, Proc. Natl. Acad. Sci. USA 93(15):7843-78481; WO92/02551; WO2004/051268 and International Patent Application number WO2004/106377.

Monoclonal antibodies can also be generated using various phage display methods known in the art and include those disclosed by Brinkman et al. (in J. Immunol. Methods, 1995, 182: 41-50), Ames et al. (J. Immunol. Methods, 1995, 184:177-186), Kettleborough et al. (Eur. J. Immunol. 1994, 24:952-958), Persic et al. (Gene, 1997 187 9-18), Burton et al. (Advances in Immunology, 1994, 57:191-280). In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol, 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol, 227: 381-388 (1992). Patent publications describing human antibody phage libraries include, for example: U.S. Pat. No. 5,750,373, and US 2005/0079574, US2005/0119455, US2005/0266000, US2007/0117126, US2007/0160598, US2007/0237764, US2007/0292936, and US2009/0002360.

Screening for antibodies can be performed using assays to measure binding to HLA-G and/or assays to measure the ability to block the binding of HLA-G to one or more of its receptors. An example of a binding assay is an ELISA, for example, using a fusion protein of the target polypeptide, which is immobilized on plates, and employing a conjugated secondary antibody to detect the antibody bound to the target. An example of a blocking assay is a flow cytometry based assay measuring the blocking of a ligand protein binding to the target polypeptide. A fluorescently labelled secondary antibody is used to detect the amount of such ligand protein binding to the target polypeptide.

Antibodies may be isolated by screening combinatorial libraries for antibodies with the desired activity or activities. For example, a variety of methods are known in the art for screening such libraries for antibodies possessing the desired binding characteristics.

The antibody according to the present invention may comprise the framework regions of the animal in which the antibody was raised. For example, it will comprise the CDRs as defined above and the framework regions of the rabbit antibody such as an antibody comprising a light chain variable region according to SEQ ID NO: 7 (which nucleotide sequence is shown in SEQ ID NO: 8) and a heavy chain variable region according to SEQ ID NO: 11 (which nucleotide sequence is shown in SEQ ID NO: 12).

In one preferred embodiment, the antibody according to the present invention is humanized.

In one preferred embodiment, the antibody which binds to HLA-G, wherein the antibody is a humanized antibody, comprises a variable light chain and a variable heavy chain, wherein:
a. the variable light chain comprises a CDR-L1 comprising SEQ ID NO: 1, a CDR-L2 comprising SEQ ID NO: 2 and a CDR-L3 comprising SEQ ID NO: 3; and
b. the variable heavy chain comprises a CDR-H1 comprising SEQ ID NO: 4, a CDR-H2 comprising SEQ ID NO: 5 and a CDR-H3 comprising SEQ ID NO: 6.

As used herein, the term “humanized” antibody refers to an antibody wherein the heavy and/or light chain contains one or more CDRs (including, if desired, one or more modified CDRs) from a donor antibody (e.g. a non-human antibody such as a murine or rabbit monoclonal antibody) grafted into a heavy and/or light chain variable region framework of an acceptor antibody (e.g. a human antibody). For a review, see Vaughan et al, Nature Biotechnology, 16, 535-539, 1998. In one embodiment, rather than the entire CDR being transferred, only one or more of the specificities determining residues from any one of the CDRs described herein above are transferred to the human antibody framework (see for example, Kashmiri et al., 2005, Methods, 36, 25-34). In one embodiment, only the specificity determining residues from one or more of the CDRs described herein above are transferred to the human antibody framework. In another embodiment, only the specificity determining residues from each of the CDRs described herein above are transferred to the human antibody framework.

When the CDRs are grafted, any appropriate acceptor variable region framework sequence may be used having regard to the class/type of the donor antibody from which the CDRs are derived, including mouse, primate and human framework regions.

Preferably, the humanized antibody according to the present invention has a variable domain comprising human acceptor framework regions as well as one or more of the CDRs provided specifically herein. Thus, in one embodiment there is provided a humanized antibody which binds HLA-G, wherein the variable domain comprises human acceptor framework regions and non-human donor CDRs.

Examples of human frameworks which can be used in the present invention are KOL, NEWM, REI, EU, TUR, TEI, LAY and POM (Kabat et al., supra). For example, KOL and NEWM can be used for the heavy chain, REI can be used for the light chain and EU, LAY and POM can be used for both the heavy chain and the light chain. Alternatively, human germline sequences may be used; these are available at: http://www.imgt.org/

In a humanized antibody according to the present invention, the acceptor heavy and light chains do not necessarily need to be derived from the same antibody and may, if desired, comprise composite chains having framework regions derived from different chains.

A suitable framework region for the light chain of the humanized antibody according to the present invention is derived from the human germline IGKV1D-13 IGKJ4 having SEQ ID NO: 103 and which nucleotide sequence is shown in SEQ ID NO: 104.

A suitable framework region for the heavy chain of the humanized antibody according to the present invention is derived from the human germline IGHV3-66 IGHJ4 having the sequence as shown in SEQ ID NO: 105 and which nucleotide sequence is shown in SEQ ID NO: 106.

Accordingly, in one embodiment there is provided a humanized antibody which binds to HLA-G, wherein the antibody comprises a light chain variable region and a heavy chain variable region and wherein:
a. the light chain variable region comprises:
   i. a CDR-L1 comprising SEQ ID NO: 1; and
   ii. a CDR-L2 comprising SEQ ID NO: 2; and
   iii. a CDR-L3 comprising SEQ ID NO: 3; and
b. the heavy chain variable region comprising:
   i. a CDR-H1 comprising SEQ ID NO: 4; and
   ii. a CDR-H2 comprising SEQ ID NO: 5 and
   iii. a CDR-H3 comprising SEQ ID NO: 6; and
      wherein the light chain framework region is derived from the human germline IGKV1D-13 IGKJ4 comprising SEQ ID NO: 103; and the heavy chain framework region is derived from the human germline IGHV3-66 IGHJ4 comprising SEQ ID NO: 105.

In one embodiment, the antibody of the invention comprises:
a. a light chain variable region comprising SEQ ID NO: 19 or 15 or 23; and/or
b. a heavy chain variable region comprising SEQ ID NO: 93, 27, 33, 57, 69, 75, 81 or 87.

In one embodiment, the antibody of the invention comprises a light chain variable region comprising SEQ ID NO: 19 and a heavy chain variable region comprising SEQ ID NO: 93.

In one embodiment, the antibody of the invention comprises a light chain variable region comprising SEQ ID NO: 15, and a heavy chain variable region comprising SEQ ID NO: 27. In one embodiment, the antibody of the invention comprises a light chain variable region comprising SEQ ID NO: 19, and a heavy chain variable region comprising SEQ ID NO: 27. In one embodiment, the antibody of the invention comprises a light chain variable region comprising SEQ ID NO: 23, and a heavy chain variable region comprising SEQ ID NO: 27. In one embodiment, the antibody of the invention comprises a light chain variable region comprising SEQ ID NO: 19, and a heavy chain variable region comprising SEQ ID NO: 33. In one embodiment, the antibody of the invention comprises a light chain variable region comprising SEQ ID NO: 19, and a heavy chain variable region comprising SEQ ID NO: 57. In one embodiment, the antibody of the invention comprises a light chain variable region comprising SEQ ID NO: 19, and a heavy chain variable region comprising SEQ ID NO: 69. In one embodiment, the antibody of the invention comprises a light chain variable region comprising SEQ ID NO: 19, and a heavy chain variable region comprising SEQ ID NO: 75. In one embodiment, the antibody of the invention comprises a light chain variable region comprising SEQ ID NO: 19, and a heavy chain variable region comprising SEQ ID NO: 81. In one embodiment, the antibody of the invention comprises a light chain variable region comprising SEQ ID NO: 19, and a heavy chain variable region comprising SEQ ID NO: 87. In one embodiment, the antibody of the invention comprises a light chain variable region comprising SEQ ID NO: 23, and a heavy chain variable region comprising SEQ ID NO: 93.

In one embodiment, the antibody of the invention is an IgG1. In one embodiment, the antibody of the invention is an IgG1 and comprises:
a. a light chain comprising SEQ ID NO: 21 or 17, or 25; and/or
b. a heavy chain comprising SEQ ID NO: 95, 29, 35, 59, 71, 77, 83, or 89.

In one embodiment, the antibody of the invention comprises a light chain comprising SEQ ID NO: 21, and a heavy chain comprising SEQ ID NO: 95.

In one embodiment, the antibody of the invention comprises a light chain comprising SEQ ID NO: 17, and a heavy chain comprising SEQ ID NO: 29. In one embodiment, the antibody of the invention comprises a light chain comprising SEQ ID NO: 21, and a heavy chain comprising SEQ ID NO: 29. In one embodiment, the antibody of the invention comprises a light chain comprising SEQ ID NO: 25, and a heavy chain comprising SEQ ID NO: 29. In one embodiment, the antibody of the invention comprises a light chain comprising SEQ ID NO: 21, and a heavy chain comprising SEQ ID NO: 35. In one embodiment, the antibody of the invention comprises a light chain comprising SEQ ID NO: 21, and a heavy chain comprising SEQ ID NO: 59. In one embodiment, the antibody of the invention comprises a light chain comprising SEQ ID NO: 21, and a heavy chain comprising SEQ ID NO: 71. In one embodiment, the antibody of the invention comprises a light chain comprising SEQ ID NO: 21, and a heavy chain comprising SEQ ID NO: 77. In one embodiment, the antibody of the invention comprises a light chain comprising SEQ ID NO: 21, and a heavy chain comprising SEQ ID NO: 83. In one embodiment, the antibody of the invention comprises a light chain comprising SEQ ID NO: 21, and a heavy chain comprising SEQ ID NO: 89. In one embodiment, the antibody of the invention comprises a light chain comprising SEQ ID NO: 25, and a heavy chain comprising SEQ ID NO: 95.

Advantageously, an IgG1 comprises an active Fc fragment, i.e. has Fc-mediated effector functions. Therefore, in one embodiment, the antibody of the invention comprises Fc-mediated effector functions.

The term “effector functions” refer to those biological activities attributable to the Fc region of an antibody, which vary with the antibody isotype. Examples of antibody effector functions include: Clq binding and complement dependent cytotoxicity (CDC), Fc receptor binding, antibody-dependent cell-mediated cytotoxicity (ADCC), antibody-dependent cell-mediated phagocytosis (ADCP).

The term “complement-dependent cytotoxicity”, or “CDC” refers to a mechanism for inducing cell death in which an Fc effector domain of a target-bound antibody binds and activates complement component Clq which in turn activates the complement cascade leading to target cell death.

The term “Antibody-dependent cellular cytotoxicity” or “ADCC” is a mechanism for inducing cell death that depends upon the interaction of antibody-coated target cells with effector cells possessing lytic activity, such as natural killer cells, monocytes, macrophages and neutrophils via Fc gamma receptors (FcγR) expressed on effector cells.

The term “Antibody-dependent cellular phagocytosis” or “ADCP” is a mechanism for inducing phagocytosis that depends upon the interaction of antibody-coated target cells or antibody-coated soluble targets with effector cells possessing phagocytic activity, such as macrophages and neutrophils via Fc gamma receptors (FcγR) expressed on effector cells.

As described in the Examples, the expression pattern of HLA-G in normal, non-tumoral tissues was investigated and advantageously, it was found that the forms of HLA-G comprising the epitope bound by the antibody of the invention are not expressed in healthy tissues, notably in pancreas and pituitary tissues.

This is in contrast to what has been previously reported in the literature where the expression of HLA-G protein in pancreatic islets was reported by Cirulli et al. (Cirulli et al, DIABETES, Vol. 55, May 2006); they observed a significant upregulation of HLA-G in islet cells cultured on an extracellular matrix supporting cell replication. Also, for example, the gene expression of HLA-G in pituitary glands, as well as in pancreatic islets and testis has been reported by Boegel et al. (Boegel et al, BMC Medical Genomics (2018) 11:36).

Therefore, the results described in the Examples herein are surprising, and show that contrary to what would have been expected from the teaching of the prior art, an antibody against HLA-G which is capable of killing cells expressing HLA-G, for example through Fc mediated effector functions, represents a potential candidate for the treatment of solid tumors, with no expectation of toxicity for the patients through binding to normal tissues. In addition, an antibody comprising an active Fc may promote effector cell recruitment and activation in the tumor microenvironment (TME), in addition, the direct killing of HLA-G+ tumor cells may induce the release of tumor antigens into the local environment that further stimulate the immune response.

The dual mechanism of such an antibody as described herein, capable of blocking the interaction between HLA-G and its inhibitory receptors, and capable of cell killing, represents a considerable advantage for the treatment of patients with upregulation of HLA-G, such as in solid cancers.

In addition, tumor heterogeneity suggests that the importance of each mechanism (HLA-G blockade to promote immune cell activation, and direct tumor cell killing via active Fc-dependent mechanisms) may differ between patients. Therefore, multiple mechanisms of tumor cell killing may allow benefit to a broader range of patients through the ability to engage different mechanisms in tumors with diverse characteristics, e.g. diverse HLA-G expression patterns.

### Methods for Selecting HLA-G Antibodies of the Invention

Because of the specific challenges associated with the production of antibodies against HLA-G (such as the high homology with other HLA-I, the identification of antibodies able to block the interaction between HLA-G and its inhibitory receptors) and in order to identify antibodies that would be useful in therapy, a special discovery, screening and testing strategy had to be developed, that involves measurement of binding to HLA-G, assessment of the affinity and specificity of the binding (no cross-reactivity to other HLA-Is), and assessment of functional properties of the test antibodies, as well as high-throughput measurement of the structural aspects of the binding (the target epitope residues).

Hence, a method of identifying an antibody according to the invention is provided, said method comprising:
a) immunizing a non-human mammal with an HLA-G immunogenic composition;
b) recovering B cells from said non-human mammal;
c) selecting the antibodies produced by said B cells that have the following properties:
   i. bind to HLA-G with an affinity represented by a dissociation constant K_{D} of less than 20 nM; and
   ii. do not bind to a HLA-I other than HLA-G; and
   iii. block the binding between HLA-G and ILT2 and/or between HLA-G and ILT4

### Step a)

An “immunogenic composition” refers to a composition which is able to generate an immune response in a non-human mammal administered with said composition. An immunogenic composition typically allows the expression of an immunogenic antigen of interest in the administered mammal, against which antibodies may be raised as part of the immune response. An “HLA-G immunogenic composition” refers to a composition which is able to generate an immune response against HLA-G in a mammal administered with said composition.

“Protein immunisation” refers to the technique of administration of an immunogenic protein comprising an antigen of interest, or immunogenic portion of said protein, comprising said antigen of interest or immunogenic portion thereof.

In one embodiment, the immunogenic composition comprises a full-length protein. In another embodiment, the immunogenic composition comprises an immunogenic portion of a protein. For example, in one embodiment, the immunogenic composition comprises a full-length HLA-G in complex with B2m. In another embodiment, the immunogenic composition comprises a full-length HLA-G in the absence of B2m. In another embodiment, the immunogenic composition comprises an immunogenic portion of HLA-G, associated or not with B2m. In another embodiment, the immunogenic composition comprises the extracellular domain of HLA-G in complex with B2m. In another embodiment, the immunogenic composition comprises the extracellular domain of HLA-G in the absence of B2m.

“DNA immunisation” refers to the technique of direct administration into the cells of the mammal of a genetically engineered nucleic acid molecule encoding a full-length protein or an immunogenic portion thereof comprising an antigen of interest (also referred to as nucleic acid vaccine or DNA vaccine herein) to produce an immunological response in said cells, against said antigen of interest. DNA immunisation uses the host cellular machinery for expressing peptide(s) corresponding to the administered nucleic acid molecule and/or achieving the expected effect, in particular antigen expression at the cellular level, and furthermore immunotherapeutic effect(s) at the cellular level or within the host organism.

“Cell immunisation” refers to the technique of administration of cells naturally expressing or transfected with an immunogenic protein comprising an antigen of interest, or immunogenic portion of said protein, comprising said antigen of interest or immunogenic portion thereof. In one embodiment, the immunisation at step a) is performed using cell immunisation with fibroblasts transfected with an immunogenic protein comprising an antigen of interest, or immunogenic portion of said protein, comprising said antigen of interest or immunogenic portion thereof.

By “Immunogenic portion”, it is meant a portion of the protein or antigen of interest which retains the capacity of inducing an immune response in the non-human mammal administered with said portion of the protein or antigen of interest or DNA encoding the same, in order to enable the production of antibodies of the invention.

HLA-G including fusion proteins thereof, cells (recombinantly or naturally) expressing the HLA-G can be used to produce antibodies which specifically recognize HLA-G. Various forms of HLA-G as described herein may be used.

HLA-G or fragments thereof, for use to immunize a host, may be prepared by processes well known in the art from genetically engineered host cells comprising expression systems or they may be recovered from natural biological sources. HLA-G or a fragment thereof may in some instances be part of a larger protein such as a fusion protein for example fused to an affinity tag or similar.

In one embodiment, the immunisation step may be performed using protein immunisation, DNA immunisation, or cell immunisation or any combination thereof.

In one embodiment, the non-human mammal is a mouse. In one embodiment, the non-human mammal is a rat. In one embodiment, the non-human mammal is a rabbit. In one embodiment, the rabbit is a New Zealand White rabbit. In one embodiment, the mammal is immunised by subcutaneous injection of the immunogenic composition. In one embodiment, the immunogenic composition comprises rabbit fibroblast cells transiently expressing HLA-G on the cell surface. In one example, the rabbit fibroblast cells have been transfected with a DNA sequence coding HLA-G comprising SEQ ID NO: 111. In another embodiment, the immunogenic composition comprises rabbit fibroblast cells transiently co-expressing HLA-G and B2m on the cell surface. In one embodiment, the rabbit fibroblast cells are Rab9 rabbit fibroblast cells.

The immunisation step may be performed using a prime-boost immunisation protocol implying a first administration (prime immunisation or prime administration) of the immunogenic composition, and then at least one further administration (boost immunisation or boost administration) that is separated in time from the first administration within the course of the immunisation protocol. Boost immunisations encompass one, two, three or more administrations. In one embodiment, boost immunisations comprise two administrations of the immunogenic composition at 14-day intervals. In one embodiment, the immunisation step comprises a first administration of rabbit fibroblast cells transiently expressing HLA-G on the cell surface, followed by two boost immunisations at 14-day intervals. In one embodiment, the rabbit fibroblast cells are Rab9 rabbit fibroblast cells.

In one embodiment, the prime immunisation comprises the administration of an adjuvant. In one embodiment, the adjuvant is administered at a site which is different from the site of injection of the immunogenic composition. In one embodiment, the adjuvant is a Freund's adjuvant. In one embodiment, both the prime immunisation and the boost immunisations comprise the administration of an adjuvant, such as a Freund's adjuvant.

In one embodiment, the immunisation step comprises a prime immunisation in presence of a first adjuvant then at least one boost immunisation in presence of a second adjuvant.

In one embodiment, the immunogenic composition is administered by sub-cutaneous injection, for example into the shoulder.

“Adjuvant” refers to an immune stimulator. Adjuvants are substances well known in the art. Traditional adjuvants, which act as immune stimulators or antigen delivery systems, or both, encompass, for example, Alum, polysaccharides, liposomes, nanoparticles based on biodegradable polymers, lipopolysaccharides. For example, the adjuvant may be a Freund's adjuvant, a Montanide adjuvant, or a Fama adjuvant.

### Step b)

Methods for isolating B-cells are well known and generally comprise isolating B-cells from PBMC (Peripheral Blood Mononuclear Cells), bone marrow or from secondary lymphoid organs, i.e. from lymphoid node, or the spleen. In one embodiment, isolating antigen-specific memory B-cells is performed 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after the immunisation step a). In one embodiment, isolating B-cells is performed 14 days after the immunisation step a). In one embodiment, step b) comprises sorting of the antigen-specific B cells by flow cytometry.

### Step c)

The screening steps in step c) may be performed according to the methods for measuring binding and blocking activity for example as described in the present disclosure.

i. Bind to HLA-G with an Affinity Represented by a Dissociation Constant (K_{D}) of Less than 20 nM

Binding may be determined against soluble and/or membrane-bound HLA-G. Binding to HLA-G may be determined by using surface plasmon resonance, such as Biacore® system for example as described in the Examples. In one example, affinity is measured by Biacore, against recombinant HLA-G ECD in complex with B2m as described in the Examples herein, and antibodies binding with a K_{D} of less than 20 nM may be selected for further analysis. In one example, the dissociation constant K_{D} is determined by SPR at a temperature of 25° C., between an antibody expressed as a full-length antibody and a monomeric form of HLA-G.

Alternatively, or in addition, binding to HLA-G may be determined by FACS against cells expressing HLA-G, such as HEK293 transfected with HLA-G and B2m, or JEG3 cells naturally expressing HLA-G. Methods for measuring the affinity of the antibodies to HLA-G by FACS are provided in the Examples described herein.

Advantageously, the antibodies may be screened using binding assays to both soluble HLA-G and cell expressed HLA-G.

ii. Do not Bind to a HLA-I Other than HLA-G

A special screening strategy has been developed to assess specificity of the binding, i.e. the absence of cross-reactivity to other HLA-Is, that involved the generation of HLA-G constructs variants, wherein amino-acid specific to HLA-G have been substituted with consensus amino-acids found in other HLA-Is (“HLA-G null constructs”). HLA-G null constructs as described in Example 1 may be particularly useful for the screening of antibodies specific to HLA-G. “HLA-G Null 1,2,3” corresponds to a HLA-G variant wherein the amino acids specifically expressed on HLA-G α1, α2 and α3 are substituted with consensus amino acids expressed on other HLA-Is (20 amino acids mutated).

In one embodiment, the method of identifying an antibody according to the invention comprises screening the antibodies recovered after step b) against “HLA-G Null 1,2,3” and selecting antibodies for which no binding is detected. Screening may be performed against “HLA-G Null 1,2,3” expressed at the cell surface (e.g. comprising SEQ ID NO: 115), or against soluble “HLA-G Null 1,2,3” (ECD) (e.g. comprising SEQ ID NO: 113).

Binding to other HLA-Is may be further assessed according to the methods described in the Examples. HEK293 cells may be transfected with DNA sequences coding either HLA-A, B, C, E or F (e.g. DNA sequences comprising SEQ ID NO: 131, 133, 135, 137 and 139 respectively) and B2m (e.g. DNA sequence comprising SEQ ID NO: 130). Binding to the cell expressed HLA-Is may be assessed by FACS.

iii. Block the Binding Between HLA-G and ILT2 and/or Between HLA-G and ILT4

Blocking HLA-G binding to ILT2 and/or ILT4 may be assessed by measuring blocking of the interaction between HLA-G, associated with B2m, expressed at the surface of cells (e.g. as naturally expressed at the surface of JEG3 cells, or as transiently expressed at the surface of HCT116 cells, as described in the Examples), with ILT2 and/or ILT4, for example expressed as fusion proteins, such as Fc fusion proteins (ITT2-Fc. ILT4-Fc).

The antibodies having the required properties and selected after step c) may be further characterized and differentiated based on additional assays, which include for example, specificity assays, ADCC, ADCP, CDC, biophysical and stability assays, and ability to modulate the immune environment and induce tumor cell killing in ex vivo culture human primary tumors, such as the methods described in the Examples herein.

### Epitope

Within the present invention, the term “epitope” is used interchangeably for both conformational and linear epitopes. A conformational epitope is composed of discontinued sections of the antigen's amino acid primary sequence and a linear epitope is formed by a sequence formed by continuous amino acids.

In one embodiment, the antibody of the invention specifically binds to HLA-G alpha 3 domain. In one embodiment, the antibody of the invention binds to an epitope of HLA-G comprising residues F195 and Y197 with reference to SEQ ID NO: 107.

In some embodiments, the antibody of the invention binds to an epitope on HLA-G, said epitope comprising residues V194, F195, Y197, E198, Q224, Q226, D227, V248, V249, P250 and Y257 (where the numbering is according to SEQ ID NO: 107).

In one embodiment, the antibody of the invention does not bind B2m. Therefore, advantageously, the antibody of the invention binds to HLA-G in complex with B2m or in the absence of B2m. In one embodiment, the antibody of the invention does not bind to the same binding site on HLA-G as HLA-G cognate peptides, naturally expressed in complex with HLA-G. Therefore, in one embodiment, the antibody of the invention does not block the association between HLA-G and its cognate peptides, naturally expressed in complex with HLA-G.

In one embodiment, the present invention provides an anti-HLA-G antibody which binds to an epitope on HLA-G, said epitope comprising at least 3, at least 4, at least 5, at least 6, at least 7, at least 8, at least 9, at least 10, or all of residues selected from the list consisting of V194, F195, Y197, E198, Q224, Q226, D227, V248, V249, P250 and Y257 of HLA-G (SEQ ID NO: 107). In one embodiment, the present invention provides a humanised IgG1 antibody that binds to an epitope of HLA-G, the epitope comprising residues V194, F195, Y197, E198, Q224, Q226, D227, V248, V249, P250 and Y257 of human HLA-G (SEQ ID NO: 107). In one embodiment, the antibody is an afucosylated IgG1.

In one embodiment, the present invention provides an anti-HLA-G antibody which binds to an epitope on HLA-G, said epitope comprising at least 3, at least 4, at least 5, at least 6, at least 7, at least 8, at least 9, at least 10, or all of residues selected from the list consisting of V194, F195, Y197, E198, Q224, Q226, D227, V248, V249, P250 and Y257 of HLA-G (SEQ ID NO: 107) as determined at less than 4 Å contact distance. In one embodiment, the antibody is an afucosylated IgG1.

In one embodiment, the present invention provides a humanised IgG1 antibody that binds to an epitope of HLA-G, said epitope comprising at least 3, at least 4, at least 5, at least 6, at least 7, at least 8, at least 9, at least 10, or all of residues selected from the list consisting of V194, F195, Y197, E198, Q224, Q226, D227, V248, V249, P250 and Y257 of HLA-G (SEQ ID NO: 107) as determined at less than 4 Å contact distance. In one embodiment, the antibody is an afucosylated IgG1.

In one embodiment, the present invention provides a humanised IgG1 antibody that binds to an epitope on HLA-G, said epitope comprising at least 3, at least 4, at least 5, at least 6, at least 7, at least 8, at least 9, at least 10, or all of residues selected from the list consisting of V194, F195, Y197, E198, R219, Q224, Q226, D227, V248, V249, P250, E253, and Y257 of HLA-G (SEQ ID NO: 107) as determined at less than 5 Å contact distance. In one embodiment, the antibody is an afucosylated IgG1.

The epitope can be identified by any suitable epitope mapping method known in the art in combination with any one of the antibodies provided by the present invention. Examples of such methods include screening peptides of varying lengths derived from full length HLA-G for binding to the antibody or fragment thereof of the present invention and identifying the smallest fragment that can specifically bind to the antibody containing the sequence of the epitope recognized by the antibody. HLA-G peptides may be produced synthetically or by proteolytic digestion of the HLA-G. Peptides that bind the antibody can be identified by, for example, mass spectrometric analysis. Methodologies such as X-ray crystallography, Nuclear magnetic resonance (NMR) spectroscopy or Hydrogen deuterium exchange mass spectrometry (HDX-MS) can be used to identify the epitope bound by an antibody. Typically, when the epitope determination is performed by X-ray crystallography, amino acid residues of the antigen within 4 Å from CDRs are considered to be amino acid residues part of the epitope. Once identified, the epitope may serve for preparing fragments which bind an antibody of the present invention and, if required, used as an immunogen to obtain additional antibodies which bind the same epitope.

The epitope as indicated in the aspects and embodiments describing the present invention is preferably an epitope characterized by X-ray crystallography. In one embodiment, the present invention provides an anti-HLA-G antibody which binds to an epitope on HLA-G, said epitope comprising at least 3, at least 4, at least 5, at least 6, at least 7, at least 8, at least 9, at least 10, or all of residues selected from the list consisting of V194, F195, Y197, E198, Q224, Q226, D227, V248, V249, P250 and Y257 of HLA-G (SEQ ID NO: 107) as determined at less than 4 Å contact distance, wherein the epitope is characterized by X-ray crystallography.

In one embodiment, the present invention provides a humanised IgG1 antibody that binds to an epitope of HLA-G, said epitope comprising at least 3, at least 4, at least 5, at least 6, at least 7, at least 8, at least 9, at least 10, or all of residues selected from the list consisting of V194, F195, Y197, E198, Q224, Q226, D227, V248, V249, P250 and Y257 of HLA-G (SEQ ID NO: 107) as determined at less than 4 Å contact distance, wherein the epitope is characterized by X-ray crystallography. In one embodiment, the antibody is an afucosylated IgG1.

In one embodiment, the present invention provides a humanised IgG1 antibody that binds to an epitope of HLA-G, said epitope comprising at least 3, at least 4, at least 5, at least 6, at least 7, at least 8, at least 9, at least 10, or all of residues selected from the list consisting of V194, F195, Y197, E198, R219, Q224, Q226, D227, V248, V249, P250, E253, and Y257 of HLA-G (SEQ ID NO: 107) as determined at less than 5 Å contact distance by X-ray crystallography. In one embodiment, the antibody is an afucosylated IgG1.

In addition, HDX-MS and NMR may be used to analyse interactions in solution and allow to show allosteric or conformational changes that are not always apparent by crystallography. For example, from the HDX-MS at 30 seconds of deuterium incubation, potential binding areas identified were 178-MLQRADPPKTHVTHHPVFD-196 and 214-ILTWQRDGEDQTQDVEL-230.

In one embodiment, the epitope determined by NMR as defined with increasing stringency as exceeding the mean of all calculated shifts (>0.0764) comprises residues T200, L201, L215, W217, R219, D220, E229, A245, A246, V247, V249, S251, E253, Q255, T258, H260, V261 and W274.

In one embodiment, the epitope determined by NMR as defined with increasing stringency as exceeding the mean plus one standard deviation of all calculated shifts (>0.1597) comprises residues H191, Y197, E198, R202, L230, V248, G252, C259 and K275.

Antibodies may compete for binding to HLA-G with, or bind to the same epitope as, those defined above in terms of light-chain, heavy-chain, light chain variable region, heavy chain variable region or CDR sequences.

In particular, the present invention provides an antibody that competes for binding to HLA-G with, or bind to the same epitope as, an antibody which comprises a CDR-L1/CDR-L2/CDR-L3/CDR-H1/CDR-H2/CDR-H3 sequence combination of SEQ ID NOs: 1/2/3/4/5/6. An antibody may compete for binding to HLA-G with, or bind to the same epitope as, an antibody which comprises a VL and VH sequence pair of SEQ ID NOs: 19 and 93 respectively. An antibody may compete for binding to HLA-G with or bind to the same epitope as an IgG1 comprising a CDR-L1/CDR-L2/CDR-L3/CDR-H1/CDR-H2/CDR-H3 sequence combination of SEQ ID NOs: 1/2/3/4/5/6. An antibody may compete for binding to HLA-G with or bind to the same epitope as an IgG1 comprising a VL and VH sequence pair of SEQ ID NOs: 19 and 93 respectively.

In one embodiment, the invention provides an antibody that cross-competes with an antibody comprising a CDR-L1/CDR-L2/CDR-L3/CDR-H1/CDR-H2/CDR-H3 sequence combination of SEQ ID NOs: 1/2/3/4/5/6, for binding to HLA-G.

In the context of the invention, the antibodies provided herein that compete for binding to HLA-G with, or bind to the same epitope as an antibody of reference according to the invention retain advantageous properties of the reference antibody as described in the above sections, for example specificity to HLA-G, high affinity, ILT2 and/or ILT4 blocking activity. In one example, an antibody that competes for binding to HLA-G with, or bind to the same epitope as an antibody of reference according to the invention, has:
a. a dissociation constant (K_{D}) of less than 20 nM, in particular less than 15 nM, in particular less than 10 nM, in particular less than 9 nM, in particular less than 8 nM, in particular less than 7 nM, in particular less than 6 nM, or in particular less than 5 nM, as determined for example by SPR at a temperature of 25° C., between said antibody expressed as a full-length antibody and a monomeric form of HLA-G; and/or
b. an IC₅₀ of less than 20 pM for blocking the binding of ILT2 to HLA-G as naturally expressed at the surface of JEG3 cells as determined for example using the in-vitro assay using large volume of reaction as described in Example 8; and/or
c. an IC₅₀ of less than 1400 pM for blocking the binding of ILT4 to HLA-G as determined for example as described in Example 8.

To determine if an antibody competes for binding with a reference antibody, the above-described binding methodology is performed in two different experimental setups. In a first setup, the reference antibody is allowed to bind to the antigen under saturating conditions followed by assessment of binding of the test antibody to the antigen. In a second setup, the test antibody is allowed to bind to the antigen under saturating conditions followed by assessment of binding of the reference antibody to the protein/peptide. If, in both experimental setups, only the first (saturating) antibody is capable of binding to the protein/peptide, then it is concluded that the test antibody and the reference antibody compete for binding to the antigen. As will be appreciated by the skilled person, an antibody that competes for binding with a reference antibody may not necessarily bind to the identical epitope as the reference antibody, but may sterically block binding of the reference antibody by binding an overlapping or adjacent epitope or cause a conformational change leading to the lack of binding.

Two antibodies bind to the same or overlapping epitope if each competitively inhibits (blocks) binding of the other to the antigen. Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same part of the antigen as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

### Antibody Variants

It will also be understood by one skilled in the art that antibodies may undergo a variety of posttranslational modifications. The type and extent of these modifications often depends on the host cell line used to express the antibody as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, RJ. Journal of Chromatography 705:129-134, 1995). Accordingly, the C-terminal lysine of the antibody heavy chain may be absent.

In one embodiment, a C-terminal amino acid from the antibody is cleaved during post-translation modifications.

In one embodiment, an N-terminal amino acid from the antibody is cleaved during post-translation modifications.

In certain embodiments, antibody variants having one or more amino acid substitutions, insertions, and/or deletions are provided. Sites of interest for substitutional mutagenesis include the CDRs and FRs. Amino acid substitutions may be introduced into an antibody of interest and the products screened for a desired activity, e.g., retained/improved antigen binding, decreased immunogenicity, or improved ADCC, CDC and/or ADCP.

In certain embodiments, amino acid sequence variants of the antibodies described herein are contemplated. For example, it may be desirable to improve the binding affinity and/or other biological properties of the antibody. Amino acid sequence variants of the anti-HLA-G antibody may be prepared by introducing appropriate modifications into the nucleotide sequence encoding the protein, or by peptide synthesis. Such modifications include, for example, deletions from, and/or insertions into and/or substitutions of residues within the amino acid sequences (such as in one or more CDRs and/or framework sequences or in a VH and/or a VL domain) of the anti-HLA-G antibody. Any combination of deletion, insertion, and substitution can be made to arrive at the final construct, provided that the final construct possesses the desired characteristics.

In certain embodiments of the variant VH and VL sequences provided herein, each HVR either is unaltered, or contains no more than one, two or three amino acid substitutions.

It will be appreciated that one or more amino acid substitutions, additions and/or deletions may be made to the CDRs provided by the present invention without significantly altering the ability of the antibody to bind to HLA-G and to neutralize HLA-G activity. The effect of any amino acid substitutions, additions and/or deletions can be readily tested by one skilled in the art, for example by using the methods described herein, particularly those illustrated in the Examples, to determine HLA-G binding and inhibition of the HLA-G interactions with its natural ligands.

Consequently, in certain embodiments of the variant VH and VL sequences, each CDR either contains no more than one, two or three amino acid substitutions, wherein such amino-acid substitutions are conservative, and wherein the antibody retains its binding properties to HLA-G.

Accordingly, the present invention provides an anti-HLA-G antibody comprising one or more CDRs selected from CDR-L1 (comprising SEQ ID NO: 1), CDR-L2 (comprising SEQ ID NO: 2), CDR-L3 (comprising SEQ ID NO: 3), CDR-H1 (comprising SEQ ID NO: 4), CDR-H2 (comprising SEQ ID NO: 5) and CDR-H3 (comprising SEQ ID NO: 6) in which one or more amino acids in one or more of the CDRs has been substituted with another amino acid, for example a similar amino acid as defined herein below.

In one embodiment, the present invention provides an anti-HLA-G antibody comprising CDR-L1 (comprising SEQ ID NO: 1), CDR-L2 (comprising SEQ ID NO:2), CDR-L3 (comprising SEQ ID NO: 3), CDR-H1 (comprising SEQ ID NO: 4), CDR-H2 (comprising SEQ ID NO: 5) and CDR-H3 (comprising SEQ ID NO: 6), for example in which one or more amino acids in one or more of the CDRs has been substituted with another amino acid, such as a similar amino acid as defined herein below.

In one embodiment, an anti-HLA-G antibody of the present invention comprises a light chain variable region which comprises three CDRs wherein the sequence of CDR-L1 comprises a sequence that has at least 70%, 80%, 90%, 91%, 92%, 93%, 94%, 95%, 96%, 97%, 98%, or 99% identity or similarity to the sequence given in SEQ ID NO: 1, and/or CDR-L2 comprises a sequence that has at least 70%, 80%, 90%, 91%, 92%, 93%, 94%, 95%, 96%, 97%, 98%, or 99% identity or similarity to the sequence given in SEQ ID NO: 2 and/or CDR-L3 comprises a sequence that has at least 70%, 80%, 90%, 91%, 92%, 93%, 94%, 95%, 96%, 97%, 98%, or 99% identity or similarity to the sequence given in SEQ ID NO: 3.

In one embodiment, an anti-HLA-G antibody of the present invention comprises a heavy chain variable region which comprises three CDRs wherein the sequence of CDR-H1 comprises a sequence that has at least 70%, 80%, 90%, 91%, 92%, 93%, 94%, 95%, 96%, 97%, 98%, or 99% identity or similarity to the sequence given in SEQ ID NO: 4, and/or CDR-H2 comprises a sequence that has at least 70%, 80%, 90%, 91%, 92%, 93%, 94%, 95%, 96%, 97%, 98%, or 99% identity or similarity to the sequence given in SEQ ID NO: 5 and/or CDR-H3 comprises a sequence that has at least 70%, 80%, 90%, 91%, 92%, 93%, 94%, 95%, 96%, 97%, 98%, or 99% identity or similarity to the sequence given in SEQ ID NO: 6.

In one embodiment, an anti-HLA-G antibody of the present invention comprises a light chain variable region comprising a sequence having at least 70%, 80%, 90%, 91%, 92%, 93%, 94%, 95%, 96%, 97%, 98%, or 99% identity or similarity to the sequence given in SEQ ID NO: 19, or SEQ ID NO: 15 or SEQ ID NO: 23.

In one embodiment, an anti-HLA-G antibody of the present invention comprises a heavy chain variable region comprising a sequence having at least 70%, 80%, 90%, 91%, 92%, 93%, 94%, 95%, 96%, 97%, 98%, or 99% identity or similarity to the sequence given in SEQ ID NO: 93, 27, 33, 57, 69, 75, 81 or 87.

In one embodiment, an anti-HLA-G antibody of the present invention comprises a light chain variable region and a heavy chain variable region, wherein the light chain variable region comprises a sequence having at least 70%, 80%, 90%, 91%, 92%, 93%, 94%, 95%, 96%, 97%, 98%, or 99% identity or similarity to the sequence given in SEQ ID NO: 19 and/or the heavy chain variable region comprises a sequence having at least 70%, 80%, 90%, 91%, 92%, 93%, 94%, 95%, 96%, 97%, 98%, or 99% identity or similarity to the sequence given in SEQ ID NO: 93.

In one embodiment, an anti-HLA-G antibody of the present invention comprises CDR-L1/CDR-L2/CDR-L3/CDR-H1/CDR-H2/CDR-H3 sequences comprising SEQ ID NOs: 1, 2, 3, 4, 5 and 6 respectively, and the remainder of the light chain and heavy chain variable regions have at least 70%, 80%, 90%, 91%, 92%, 93%, 94%, 95%, 96%, 97%, 98%, or 99% identity or similarity to SEQ ID NO: 19 and SEQ ID NO: 93 respectively.

In one embodiment, an anti-HLA-G antibody of the present invention comprises a light chain comprising a sequence having at least 70%, 80%, 90%, 91%, 92%, 93%, 94%, 95%, 96%, 97%, 98%, or 99% identity or similarity to the sequence given in SEQ ID NO: 21, or SEQ ID NO: 17 or SEQ ID NO: 25.

In one embodiment, an anti-HLA-G antibody of the present invention comprises a heavy chain comprising a sequence having at least 70%, 80%, 90%, 91%, 92%, 93%, 94%, 95%, 96%, 97%, 98%, or 99% identity or similarity to the sequence given in SEQ ID NO: 95, 29, 35, 59, 71, 77, 83 or 89.

In one embodiment, the anti-HLA-G antibody of the present invention is a IgG1 comprising a light chain comprising a sequence having at least 70%, 80%, 90%, 91%, 92%, 93%, 94%, 95%, 96%, 97%, 98%, or 99% identity or similarity to the sequence given in SEQ ID NO: 21 and a heavy chain comprising a sequence having at least 70%, 80%, 90%, 91%, 92%, 93%, 94%, 95%, 96%, 97%, 98%, or 99% identity or similarity to the sequence given in SEQ ID NO: 95.

In one embodiment, an anti-HLA-G antibody of the present invention is a IgG1 comprising CDR-L1/CDR-L2/CDR-L3/CDR-H1/CDR-H2/CDR-H3 sequences given in SEQ ID NOs: 1, 2, 3, 4, 5 and 6 respectively, and the remainder of the light chain and heavy chain has at least 70%, 80%, 90%, 91%, 92%, 93%, 94%, 95%, 96%, 97%, 98%, or 99% identity or similarity to SEQ ID NO: 21 and SEQ ID NO: 95 respectively.

The anti-HLA-G antibody variants provided herein by the invention retain the advantageous properties of the parental antibody (i.e. unmodified antibody), i.e. the functional properties described above, for example high specificity, high affinity, ILT2 and/or ILT4 blocking activity. In one example, an anti-HLA-G antibody variant provided by the invention has:
a. a dissociation constant (K_{D}) of less than 20 nM, in particular less than 15 nM, in particular less than 10 nM, in particular less than 9 nM, in particular less than 8 nM, in particular less than 7 nM, in particular less than 6 nM, or in particular less than 5 nM, as determined for example by SPR at a temperature of 25° C., between said antibody expressed as a full-length antibody and a monomeric form of HLA-G; and/or
b. an IC₅₀ of less than 20 pM for blocking the binding of ILT2 to HLA-G as naturally expressed at the surface of JEG3 cells as determined for example using the in-vitro assay using large volume of reaction as described in Example 8; and/or
c. an IC₅₀ of less than 1400 pM for blocking the binding of ILT4 to HLA-G as determined for example as described in Example 8.

### Sequence Identity and Similarity

Degrees of identity and similarity between sequences can be readily calculated. The “% sequence identity” (or “% sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) amino-acids (e.g., identical amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to obtain the % sequence identity or percent sequence similarity.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). Polypeptide sequences also can be compared using FASTA using default or recommended parameters. FASTA (e.g., FASTA2 and FASTA3) provides alignments and percent sequence identity of the regions of the best overlap between the query and search sequences.

In certain embodiments, substitutions, insertions, or deletions may occur within one or more CDR so long as such alterations do not substantially reduce the ability of the antibody to bind the target.

For example, conservative alterations that do not substantially reduce binding affinity may be made in CDRs. Such alterations may be made outside of antigen contacting residues in the CDRs.

Conservative substitutions are shown in Table 1 together with more substantial “exemplary substitutions”.

TABLE 1
Examples of amino-acid substitutions

| Original | Exemplary | Conservative |
|---|---|---|
| Residue | Substitutions | Substitution |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys(C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |

Substantial modifications in the biological properties of an antibody variant can be accomplished by selecting substitutions that differ significantly in their effect on maintaining the structure of the polypeptide backbone in the area of the substitution, the charge or hydrophobicity of the molecule at the target site, or the bulk of the side chain. Amino acids may be grouped according to similarities in the properties of their side chains (in A. L. Lehninger, Biochemistry second ed., pp. 73-75, Worth Publishers, New York (1975))

One type of substitutional variant involves substituting one or more CDR region residues of a parent antibody (humanized or human antibody). Generally, the resulting variant(s) selected for further study will have changes in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207: 179-196 (2008)), and/or residues that contact antigen, with the resulting variant VH or VL being tested for binding affinity. Affinity maturation by constructing and reselecting from secondary libraries has been described, e.g., in Hoogenboom et al. Methods in Molecular Biology 178: 1-37 (O'Brien et al., ed., Human Press, Totowa, NJ, (2001). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity.

One of the methods that can be used for identification of residues or regions of an antibody that may be targeted for mutagenesis is alanine scanning mutagenesis (Cunningham and Wells (1989) Science, 244: 1081-1085). In this method, a residue or a number of target residues are identified and replaced by alanine to determine whether the interaction of the antibody with antigen is affected. Alternatively, or additionally, an X-ray structure of an antigen-antibody complex can be used to identify contact points between the antibody and its antigen. Variants may be screened to determine whether they contain the desired properties.

### Constant Region Variants

In some embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).

Antibodies with increased half-lives and improved binding to the neonatal Fc receptor (FcRn) are described for example in US2005/0014934A1. Those antibodies comprise an Fc region with one or more substitutions therein which improve binding of the Fc region to FcRn.

In certain embodiments, an antibody variant comprises an Fc region with one or more amino acid substitutions which improve ADCC, e.g., substitutions at positions 298, 333, and/or 334 of the Fc region (EU numbering of residues).

Antibodies with reduced effector function include those with substitution of one or more of Fc region residues 234, 235, 237, 238, 265, 269, 270, 297, 327 and 329 (see, e.g., U.S. Pat. No. 6,737,056). Such Fc mutants include Fc mutants with substitutions at two or more of amino acid positions 265, 269, 270, 297 and 327 wherein the amino acid residue is numbered according to the EU numbering system.

In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcRI, FcγRII and FcγRIII. FcR expression on hematopoietic cells is summarized in Ravetch and Kinet, Annu. Rev. Immunol. 9:457-492 (1991). Non-limiting examples of in vitro assays to assess ADCC activity of a molecule of interest is described in U.S. Pat. Nos. 5,500,362; 5,821,337. Alternatively, or additionally, ADCC activity of the molecule of interest may be assessed in vivo, e.g., in an animal model such as that disclosed in Clynes et al. Proc. Nat 1 Acad. Sci. USA 95:652-656 (1998). Clq binding assays may also be carried out to confirm that the antibody is unable to bind Clq and hence lacks CDC activity. See, e.g., Clq and C3c binding ELISA in WO 2006/029879 and WO 2005/100402. To assess complement activation, a CDC assay may be performed (see, for example, Gazzano-Santoro et al, J. Immunol. Methods 202: 163 (1996); Cragg, M. S. et al, Blood 101: 1045-1052 (2003); and Cragg, M. S. and M. I Glennie, Blood 103:2738-2743 (2004)). FcRn binding and in vivo clearance/half-life determinations can also be performed using methods known in the art (see, e.g., Petkova, S. B. et al, Int l. Immunol. 18(12): 1759-1769 (2006)).

The constant region domains of the antibody molecule of the present invention, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required. It will be appreciated that sequence variants of these constant region domains may also be used.

In some embodiments, the antibody of the invention is an wild-type human IgG1 (referred to as IgG1).

In some embodiments, the antibody of the invention is an IgG1 LALA, a mutant of the wild-type human IgG1 isoform in which amino acid substitutions L234A/L235A (according to EU numbering) in the constant region of IgG1 have been introduced. In one embodiment, the antibody of the invention comprises a light chain comprising the sequence SEQ ID NO: 21 and a heavy chain comprising the sequence SEQ ID NO: 97.

In some embodiments, the antibody of the invention is an IgG1 LALAGA, a mutant of the wild-type human IgG1 isoform in which amino acid substitutions L234A/L235A/G237A (according to EU numbering) in the constant region of IgG1 have been introduced.

In some embodiments, the antibody of the invention is an IgG4P, a mutant of the wild-type human IgG4 isoform in which amino acid 228 (according to EU numbering) is replaced by proline, as described for example in Angal et al., Molecular Immunology, 1993, 30 (1), 105-108. In one embodiment, the antibody of the invention comprises a light chain comprising the sequence SEQ ID NO: 21 and a heavy chain comprising the sequence SEQ ID NO: 99.

In some embodiments, the antibody of the invention is an IgG4 FALA, a mutant of the wild-type human IgG4 isoform in which substitutions F234A/L235A (according to EU numbering) in the constant region of IgG4 have been introduced.

In some embodiments, the antibody of the invention is an IgG4P FALA, a mutant of the wild-type human IgG4 isoform in which amino acid 228 (according to EU numbering) is replaced by proline and acid substitutions F234A/L235A (according to EU numbering) in the constant region of IgG4 have been introduced. In one embodiment, the antibody of the invention comprises a light chain comprising the sequence SEQ ID NO: 21 and a heavy chain comprising the sequence SEQ ID NO: 101.

### Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

In one embodiment, the antibody of the invention is glyco-modified. In one embodiment, the antibody of the invention has a low or no fucose content. By “fucose content” it is meant the percentage of fucosylated forms within N-glycans attached to the Asn297 residue of the Fc fragment of each heavy chain of each antibody. By “low fucose content” is meant a fucose content of less than or equal to 65 percent. Indeed, it is known today that a low fucose content of an antibody composition plays a crucial role in the capacity of said composition to induce a strong ADCC response via FcgammaRIII. Advantageously, the fucose content is less than or equal to 65 percent, preferably less than or equal to 60 percent, 55 percent or 50 percent, even less than or equal to 45 percent, 40 percent, 35 percent, 30 percent, 25 percent or 20 percent. However, it is not necessary that the fucose content be null, and it may for example be less than or equal to 5 percent, 10 percent, 15 percent or 20 percent.

In one embodiment, the antibody of the invention is an afucosylated IgG1. Methods for the production of afucosylated IgG1 are well known and include the production of cells genetically modified for the production of afucosylated antibodies. In one embodiment, the afucosylated IgG1 of the invention is produced in CHO cells wherein the gene coding for alpha1,6 fucosyltransferase (FUT8) was genetically knocked-out by methods well known in the art. For example, KO FUT8 CHOSXE/DG44 cells may be used as described in the Examples provided herein.

In one embodiment, the antibody of the invention has an improved ADCC and/or ADCP and/or has an improved ability to deplete tumor cells expressing HLA-G. In one embodiment, the ADCC and/or ADCP function, and/or the ability to deplete tumor cells expressing HLA-G of an afucosylated antibody according to the invention is improved as compared to the corresponding conventional (i.e. fucosylated) antibody (i.e. antibody comprising the same amino-acid sequence, but that comprises fucose, e.g. produced in CHO cells that have not been modified and that express the FUT8). In the context of the invention, by “improved” activity (e.g. ADCC and/or ADCP and/or tumor cell depletion), it is meant that the activity (e.g. ADCC and/or ADCP and/or tumor cell depletion) of the afucosylated antibody is at least 1%, 5%, 10%, 15%, 20%, 25%, 30%, 35%, 40%, 45%, 50% higher than the same activity of the antibody of reference (i.e. the corresponding conventional, i.e. fucosylated, antibody).

In vitro and/or in vivo cytotoxicity assays that are well known in the art can be conducted to confirm the increase of ADCC and/or ADCP activities, for example as described herein.

In one embodiment, the antibody of the invention is an afucosylated IgG1 which comprises:
a. a light chain comprising SEQ ID NO: 21 or 17, or 25; and/or
b. a heavy chain comprising SEQ ID NO: 95, 29, 35, 59, 71, 77, 83, or 89.

In one embodiment, the antibody of the invention is an afucosylated IgG1 which comprises:
a. a light chain comprising at least 90% identity or similarity to SEQ ID NO: 21 or 17, or 25; and/or
b. a heavy chain comprising at least 90% identity or similarity to SEQ ID NO: 95, 29, 35, 59, 71, 77, 83, or 89.

In one embodiment, the anti-HLA-G antibody of the present invention is an afucosylated IgG1 comprising a light chain comprising a sequence having at least 70%, 80%, 90%, 91%, 92%, 93%, 94%, 95%, 96%, 97%, 98%, or 99% identity or similarity to the sequence given in SEQ ID NO: 21 and a heavy chain comprising a sequence having at least 70%, 80%, 90%, 91%, 92%, 93%, 94%, 95%, 96%, 97%, 98%, or 99% identity or similarity to the sequence given in SEQ ID NO: 95.

In one embodiment, the anti-HLA-G antibody of the present invention is an afucosylated IgG1 comprising CDR-L1/CDR-L2/CDR-L3/CDR-H1/CDR-H2/CDR-H3 sequences given in SEQ ID NOs: 1, 2, 3, 4, 5 and 6 respectively, and the remainder of the light chain and heavy chain has at least 70%, 80%, 90%, 91%, 92%, 93%, 94%, 95%, 96%, 97%, 98%, or 99% identity or similarity to SEQ ID NO: 21 and SEQ ID NO: 95 respectively.

In one embodiment, the anti-HLA-G antibody of the present invention is an afucosylated IgG1 which comprises a light chain variable region comprising SEQ ID NO: 19 and a heavy chain variable region comprising SEQ ID NO: 93, and wherein the reminder of the light chain and heavy chain has at least 90% identity or similarity to SEQ ID NOs: 21 and 95 respectively.

In one embodiment, the antibody of the invention comprises a light chain comprising SEQ ID NO: 21, and a heavy chain comprising SEQ ID NO: 95, and has an improved ADCC and/or ADCP function, and/or has an improved ability to deplete tumor cells expressing HLA-G. In one embodiment, the ADCC and/or ADCP function, and/or the ability to deplete tumor cells expressing HLA-G is improved as compared to the corresponding conventional antibody (i.e. to a fucosylated antibody which comprises a light chain comprising SEQ ID NO: 21, and a heavy chain comprising SEQ ID NO: 95).

In one embodiment, the antibody of the invention comprises a light chain comprising SEQ ID NO: 21, and a heavy chain comprising SEQ ID NO: 95, and has an improved CDC function, and/or has an improved ability to deplete tumor cells expressing HLA-G. In one embodiment, the CDC function, and/or the ability to deplete tumor cells expressing HLA-G is improved as compared to the corresponding conventional antibody (i.e. to a fucosylated antibody which comprises a light chain comprising SEQ ID NO: 21, and a heavy chain comprising SEQ ID NO: 95).

In one embodiment, the anti-HLA-G antibody of the present invention is an afucosylated IgG1 which comprises a light chain variable region comprising SEQ ID NO: 19 and a heavy chain variable region comprising SEQ ID NO: 93, or a light chain comprising SEQ ID NO: 21 and a heavy chain comprising SEQ ID NO: 95, and has an improved ADCC and/or ADCP and/or CDC function and has an improved ability to deplete tumor cells expressing HLA-G.

Biological molecules, such as antibodies, contain acidic and/or basic functional groups, thereby giving the molecule a net positive or negative charge. The amount of overall “observed” charge will depend on the absolute amino acid sequence of the entity, the local environment of the charged groups in the 3D structure and the environmental conditions of the molecule. The isoelectric point (pI) is the pH at which a particular molecule or solvent accessible surface thereof carries no net electrical charge. In one example, the antibody binding HLA-G may be engineered to have an appropriate isoelectric point. This may lead to antibodies with more robust properties, in particular suitable solubility and/or stability profiles and/or improved purification characteristics.

The antibody may, for example be engineered by replacing an amino acid residue such as replacing an acidic amino acid residue with one or more basic amino acid residues. Alternatively, basic amino acid residues may be introduced or acidic amino acid residues can be removed. Alternatively, if the molecule has an unacceptably high pI value, acidic residues may be introduced to lower the pI, as required. It is important that when manipulating the pI care must be taken to retain the desirable activity of the antibody or fragment. Thus, in one embodiment the engineered antibody has the same or substantially the same activity as the “unmodified” antibody or fragment.

Programs such as ** ExPASY http://www.expasy.ch/tools/pi_tool.html, and http://www.iut-arles.up.univ-mrs.fr/w3bb/d_abim/compo-p.html, may be used to predict the isoelectric point of the antibody.

### Effector Molecules

If desired, an antibody for use in the present invention may be conjugated to one or more effector molecule(s).

It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO 03031581. Alternatively, where the effector molecule is a protein or polypeptide the linkage may be achieved using recombinant DNA procedures, for example as described in WO 86/01533 and EP 0392745.

The term effector molecule as used herein includes, for example, antineoplastic agents, drugs, toxins, biologically active proteins, for example enzymes, other antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Examples of effector molecules may include cytotoxins or cytotoxic agents including any agent that is detrimental to (e.g. kills) cells. Examples include combrestatins, dolastatins, epothilones, staurosporin, maytansinoids, spongistatins, rhizoxin, halichondrins, roridins, hemiasterlins, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Effector molecules also include, but are not limited to, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, anthramycin (AMC), calicheamicins or duocarmycins), and anti-mitotic agents (e.g. vincristine and vinblastine).

Other effector molecules may include chelated radionuclides such as ¹¹¹In and ⁹⁰Y, Lu¹⁷⁷, Bismuth²¹³, Californium²⁵², Iridium¹⁹² and Tungsten¹⁸⁸/Rhenium¹⁸⁸; or drugs such as but not limited to, alkylphosphocholines, topoisomerase I inhibitors, taxoids and suramin.

Other effector molecules include proteins, peptides and enzymes. Enzymes of interest include, but are not limited to, proteolytic enzymes, hydrolases, lyases, isomerases, transferases. Proteins, polypeptides and peptides of interest include, but are not limited to, immunoglobulins, toxins such as abrin, ricin A, pseudomonas exotoxin, or diphtheria toxin, a protein such as insulin, tumor necrosis factor, α-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), granulocyte macrophage colony stimulating factor (GM-CSF), granulocyte colony stimulating factor (G-CSF), nerve growth factor (NGF) or other growth factor and immunoglobulins.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO 05/117984.

Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Specific naturally occurring polymers include lactose, amylose, dextran, glycogen or derivatives thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

The size of the polymer may be varied as desired, but will generally be in an average molecular weight range from 500 Da to 50000 Da, for example from 5000 to 40000 Da such as from 20000 to 40000 Da. The polymer size may in particular be selected on the basis of the intended use of the product for example ability to localize to certain tissues such as tumors or extend circulating half-life (for review see Chapman, 2002, Advanced Drug Delivery Reviews, 54, 531-545). Thus, for example, where the product is intended to leave the circulation and penetrate tissue it may be advantageous to use a small molecular weight polymer, for example with a molecular weight of around 5000 Da. For applications where the product remains in the circulation, it may be advantageous to use a higher molecular weight polymer, for example having a molecular weight in the range from 20000 Da to 40000 Da.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example antibodies for use in the present invention are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO 98/25971). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

Suitably PEG molecules may be covalently linked through a thiol group of at least one cysteine residue located in the antibody fragment. Each polymer molecule attached to the modified antibody fragment may be covalently linked to the sulphur atom of a cysteine residue located in the fragment. The covalent linkage will generally be a disulphide bond or, in particular, a sulphur-carbon bond. Where a thiol group is used as the point of attachment appropriately activated effector molecules, for example thiol selective derivatives such as maleimides and cysteine derivatives may be used. An activated polymer may be used as the starting material in the preparation of polymer-modified antibody fragments as described above. The activated polymer may be any polymer containing a thiol reactive group such as an α-halocarboxylic acid or ester, e.g. iodoacetamide, an imide, e.g. maleimide, a vinyl sulphone or a disulphide. Such starting materials may be obtained commercially (for example from Nektar, formerly Shearwater Polymers Inc., Huntsville, AL, USA) or may be prepared from commercially available starting materials using conventional chemical procedures. Particular PEG molecules include 20K methoxy-PEG-amine (obtainable from Nektar, formerly Shearwater; Rapp Polymere; and SunBio) and M-PEG-SPA (obtainable from Nektar, formerly Shearwater).

In one embodiment, the antibody is a modified Fab fragment or diFab which is PEGylated, i.e. has PEG (poly(ethyleneglycol)) covalently attached thereto, e.g. according to the method disclosed in EP 0948544 or EP 1090037 [see also “Poly(ethyleneglycol) Chemistry, Biotechnical and Biomedical Applications”, 1992, J. Milton Harris (ed), Plenum Press, New York, “Poly(ethyleneglycol) Chemistry and Biological Applications”, 1997, J. Milton Harris and S. Zalipsky (eds), American Chemical Society, Washington DC and “Bioconjugation Protein Coupling Techniques for the Biomedical Sciences”, 1998, M. Aslam and A. Dent, Grove Publishers, New York; Chapman, A. 2002, Advanced Drug Delivery Reviews 2002, 54:531-545]. In one example PEG is attached to a cysteine in the hinge region. In one example, a PEG modified Fab fragment has a maleimide group covalently linked to a single thiol group in a modified hinge region. A lysine residue may be covalently linked to the maleimide group and to each of the amine groups on the lysine residue may be attached a methoxypoly(ethyleneglycol) polymer having a molecular weight of approximately 20,000 Da. The total molecular weight of the PEG attached to the Fab fragment may therefore be approximately 40,000 Da.

In one embodiment, the antibody is a modified Fab′ fragment having at the C-terminal end of its heavy chain a modified hinge region containing at least one cysteine residue to which an effector molecule is attached. Suitably the effector molecule is PEG and is attached using the methods described in WO 98/25971 and WO 2004072116 or in WO 2007/003898. Effector molecules may be attached to antibody fragments using the methods described in International patent applications WO 2005/003169, WO 2005/003170 and WO 2005/003171.

In one embodiment the antibody is not attached to an effector molecule.

### Polynucleotides and Vectors

The present invention also provides an isolated polynucleotide encoding the antibody or a part thereof according to the present invention (such as Amino-acid SEQ IDs listed in Table 2). The term “isolated” means, throughout this specification, that the polynucleotide exists in a physical milieu distinct from that in which it may occur in nature.

The isolated polynucleotide according to the present invention may comprise synthetic DNA, for instance produced by chemical processing, cDNA, genomic DNA or any combination thereof.

TABLE 2
Amino-acid sequences of the 12389 anti-HLA-G antibodies
and their corresponding nucleic acid sequences.

| | Amino-acid | Nucleic acid |
|---|---|---|
| Antibody sequence | SEQ ID NO | SEQ ID NO |
| 12389gL2gH16 VL | 19 | 20 |
| 12389gL2gH16 light chain | 21 | 22 |
| 12389gL2gH16 VH | 93 | 94 |
| 12389gL2gH16 heavy chain IgG1 | 95 | 96 |
| 12389gL2gH16 heavy chain IgG1 | 97 | 98 |
| LALA | | |
| 12389gL2gH16 heavy chain IgG4P | 99 | 100 |
| 12389gL2gH16 heavy chain IgG4P | 101 | 102 |
| FALA | | |

Examples of suitable sequences are provided herein. Thus, in one embodiment the present invention provides an isolated polynucleotide encoding an antibody, comprising a sequence given in SEQ ID Nos 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102.

In one embodiment, the present invention provides an isolated polynucleotide encoding an antibody of the invention, wherein the polynucleotide encodes a light chain variable region, wherein the polynucleotide:
i. is at least 90% identical to SEQ ID NO: 20, 16 or 24; or
ii. comprises or consists of SEQ ID NO: 20, 16 or 24.

In one embodiment, the present invention provides an isolated polynucleotide encoding an antibody of the invention, wherein the polynucleotide encodes a heavy chain variable region, wherein the polynucleotide:
i. is at least 90% identical to SEQ ID NO: 94, 28, 34, 58, 70, 76, 82 or 88; or
ii. comprises or consists of SEQ ID NO: 94, 28, 34, 58, 70, 76, 82 or 88.

In one embodiment, the present invention provides an isolated polynucleotide encoding an antibody of the invention, wherein the polynucleotide encodes a light chain, wherein the polynucleotide:
i. is at least 90% identical to SEQ ID NO: 22, 18, or 26; or
ii. comprises or consists of SEQ ID NO: 22, 18, or 26.

In one embodiment, the present invention provides an isolated polynucleotide encoding an antibody of the invention, wherein the polynucleotide encodes a heavy chain, wherein the polynucleotide:
i. is at least 90% identical to SEQ ID NO: 96, 30, 36, 60, 72, 78, 84, or 90; or
ii. comprises or consists of SEQ ID NO: 96, 30, 36, 60, 72, 78, 84, or 90.

In one embodiment, the present invention provides an isolated polynucleotide encoding the heavy chain of an IgG1 antibody of the present invention which comprises the sequence given in SEQ ID NO: 96, 30, 36, 60, 72, 78, 84, or 90.

Also provided is an isolated polynucleotide encoding the light chain of an IgG1 antibody of the present invention which comprises the sequence given in SEQ ID NO: 22, 18, or 26.

In a preferred embodiment, the present invention provides an isolated polynucleotide encoding the heavy chain and the light chain of an IgG1 antibody of the present invention in which the polynucleotide encoding the heavy chain comprises the sequence given in SEQ ID NO: 96 and the polynucleotide encoding the light chain comprises the sequence given in SEQ ID NO: 22.

The present invention also provides for a cloning or expression vector comprising one or more polynucleotides described herein. In one example, the cloning or expression vector according to the present invention comprises one or more isolated polynucleotides as described above.

Standard techniques of molecular biology may be used to prepare DNA sequences coding for the antibody of the present invention. Desired DNA sequences may be synthesized completely or in part using oligonucleotide synthesis techniques. Site-directed mutagenesis and polymerase chain reaction (PCR) techniques may be used as appropriate.

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

### Host Cells for Production of the Antibodies

Also provided is a host cell comprising one or more isolated polynucleotide sequences according to the invention or one or more cloning or expression vectors comprising one or more isolated polynucleotide sequences encoding an antibody of the present invention. Any suitable host cell/vector system may be used for expression of the polynucleotide sequences encoding the antibody of the present invention. Bacterial, for example E. coli, and other microbial systems may be used or eukaryotic, for example mammalian, host cell expression systems may also be used. Suitable mammalian host cells include CHO, myeloma or hybridoma cells.

In a further embodiment, a host cell comprising such nucleic acid(s) or vector(s) is provided. In one such embodiment, a host cell comprises (e.g., has been transformed with): (1) a vector comprising a nucleic acid that encodes an amino acid sequence comprising the VL of the anti-HLA-G antibody and an amino acid sequence comprising the VH of the anti-HLA-G antibody, or (2) a first vector comprising a nucleic acid that encodes an amino acid sequence comprising the VL of the anti-HLA-G antibody and a second vector comprising a nucleic acid that encodes an amino acid sequence comprising the VH of the anti-HLA-G antibody. In one embodiment, the host cell is eukaryotic, e.g. a Chinese Hamster Ovary (CHO) cell or lymphoid cell (e.g., Y0, NS0, Sp20 cell). In one embodiment, the host cell is prokaryotic, e.g. an E. coli cell. In one embodiment, a method of making an anti-HLA-G antibody is provided, wherein the method comprises culturing a host cell comprising a nucleic acid encoding the antibody, as provided above, under conditions suitable for expression of the antibody, and optionally recovering the antibody from the host cell (or host cell culture medium).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.). After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22: 1409-1414 (2004), and Li et al., Nat. Biotech. 24:210-215 (2006).

Suitable types of Chinese Hamster Ovary (CHO cells) for use in the present invention may include CHO and CHO-K1 cells including dhfr− CHO cells, such as CHO-DG44 cells and CHO-DXB11 cells and which may be used with a DHFR selectable marker or CHOK1-SV cells which may be used with a glutamine synthetase selectable marker. Other cell types of use in expressing antibodies include lymphocytic cell lines, e.g., NS0 myeloma cells and SP2 cells, COS cells. The host cell may be stably transformed or transfected with the isolated polynucleotide sequences or the expression vectors according to the present invention.

In one embodiment, the antibody of the invention is produced in a host cell that has been genetically modified to decrease or abolish the function of the alpha1,6 fucosyltransferase (FUT8). In one embodiment, the genetically modified cell is a CHO cell and the FUT8 gene has been knock-out (KO FUT8). In one embodiment, a host cell for the production of the antibody of the invention is a CHO-DG44. In one embodiment, a host cell for the production of the antibody of the invention is a KO FUT8 CHOSXE/DG44 cells, which may be produced according to the methods described in the Examples provided herein.

### Process for the Production of the Antibodies

The present invention also provides a process for the production of an antibody according to the present invention comprising culturing a host cell according to the present invention under conditions suitable for producing the antibody according to the invention and isolating the antibody.

The present invention also provides a process for the production of a pharmaceutcial composition comprising an antibody according to the present invention comprising culturing a host cell according to the present invention under conditions suitable for producing the antibody according to the invention, isolating the antibody, and formulating the antibody into a pharmaceutical composition.

The antibody may comprise only a heavy or light chain polypeptide, in which case only a heavy chain or light chain polypeptide coding sequence needs to be used to transfect the host cells. For production of antibodies comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

Thus, there is provided a process for culturing a host cell and expressing an antibody, isolating the antibody and optionally purifying the antibody to provide an isolated antibody. In one embodiment, the process further comprises the step of conjugating an effector molecule to the isolated antibody.

The present invention also provides a process for the production of an antibody according to the present invention comprising culturing a host cell containing a vector of the present invention under conditions suitable for leading to expression of protein from DNA encoding the antibody molecule of the present invention and isolating the antibody molecule.

The antibodies according to the present invention are expressed at good levels from host cells. Thus the properties of the antibodies appear to be optimized for commercial processing.

In one embodiment there is provided a purified antibody, for example a humanized antibody, in particular an antibody according to the invention, in substantially purified form, in particular free or substantially free of endotoxin and/or host cell protein or DNA.

Substantially free of endotoxin is generally intended to refer to an endotoxin content of 1 EU per mg antibody product or less such as 0.5 or 0.1 EU per mg product.

Substantially free of host cell protein or DNA is generally intended to refer to host cell protein and/or DNA content 400 μg per mg of antibody product or less such as 100 μg per mg or less, in particular 20 μg per mg, as appropriate.

### Pharmaceutical Compositions, Dosages and Dosage Regimes

An antibody of the invention may be provided in a pharmaceutical composition or diagnostic composition. Hence, the present invention also provides for a pharmaceutical or diagnostic composition comprising the antibody according to the present invention in combination with one or more of a pharmaceutically acceptable carrier, excipient or diluents.

Preferably, the pharmaceutical or diagnostic composition comprises an antibody which specifically binds HLA-G, wherein the antibody comprises:
a. a light chain variable region comprising:
   i. a CDR-L1 comprising SEQ ID NO: 1;
   ii. a CDR-L2 comprising SEQ ID NO: 2 and
   iii. a CDR-L3 comprising SEQ ID NO: 3; and
b. a heavy chain variable region comprising:
   i. a CDR-H1 comprising SEQ ID NO: 4;
   ii. a CDR-H2 comprising SEQ ID NO: 5 and
   iii. a CDR-H3 comprising SEQ ID NO: 6.

In one embodiment, the antibody according to the present invention is the sole active ingredient. In another embodiment, the antibody according to the present invention is in combination with one or more additional active ingredients. In one embodiment, the antibody according to the present invention is in combination with an antibody directed against CD47. Therefore, in one embodiment, there is provided an antibody which specifically binds HLA-G, wherein the antibody comprises:
a. a light chain variable region comprising:
   i. a CDR-L1 comprising SEQ ID NO: 1;
   ii. a CDR-L2 comprising SEQ ID NO: 2 and
   iii. a CDR-L3 comprising SEQ ID NO: 3; and
b. a heavy chain variable region comprising:
   i. a CDR-H1 comprising SEQ ID NO: 4;
   ii. a CDR-H2 comprising SEQ ID NO: 5 and
   iii. a CDR-H3 comprising SEQ ID NO: 6,
      and wherein the antibody is in combination with a second antibody which binds to CD47.

Alternatively, the pharmaceutical compositions comprise the antibody according to the present invention which is the sole active ingredient and it may be administered individually to a patient in combination (e.g. simultaneously, sequentially or separately) with other therapeutic, diagnostic or palliative agents.

The pharmaceutical compositions according to the invention may be administered suitably to a patient to identify the therapeutically effective amount required. The term “therapeutically effective amount” as used herein refers to an amount of a therapeutic agent needed to treat, ameliorate or prevent a targeted disease or condition, or to exhibit a detectable therapeutic or preventative effect. For any antibody, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. Generally, a therapeutically effective amount will be from 0.01 mg/kg to 500 mg/kg, for example 0.1 mg/kg to 200 mg/kg, such as 100 mg/kg. Pharmaceutical compositions may be conveniently presented in unit dose forms containing a predetermined amount of an active agent of the invention per dose.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions.

Suitable forms for administration include forms suitable for parenteral administration, e.g. by injection or infusion, for example by bolus injection or continuous infusion, in intravenous, inhalable or sub-cutaneous form. Where the product is for injection or infusion, it may take the form of a suspension, solution or emulsion in an oily or aqueous vehicle and it may contain formulatory agents, such as suspending, preservative, stabilizing and/or dispersing agents. Alternatively, the antibody according to the invention may be in dry form, for reconstitution before use with an appropriate sterile liquid. Solid forms suitable for solution in, or suspension in, liquid vehicles prior to injection may also be prepared.

Once formulated, the compositions of the invention can be administered directly to the subject. Accordingly, provided herein is the use of an antibody according to the invention for the manufacture of a medicament.

Preferably, the pharmaceutical composition according to the present invention is adapted for administration to human subjects.

Hence, in another aspect the present invention provides for an antibody which specifically binds HLA-G wherein the antibody or a pharmaceutical composition comprising the antibody, for use in therapy, wherein the antibody comprises:
a. a light chain variable region comprising:
   i. a CDR-L1 comprising SEQ ID NO: 1;
   ii. a CDR-L2 comprising SEQ ID NO: 2; and
   iii. a CDR-L3 comprising SEQ ID NO: 3; and
b. a heavy chain variable region comprising:
   i. a CDR-H1 comprising SEQ ID NO: 4;
   ii. a CDR-H2 comprising SEQ ID NO: 5 and
   iii. a CDR-H3 comprising SEQ ID NO: 6.

### Therapeutic Indications

As used herein, the terms “treatment”, “treating” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. Treatment thus covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

A “therapeutically effective amount” refers to the amount of a HLA-G antibody that, when administered to a mammal or other subject for treating a disease, is sufficient to produce such treatment for the disease. The therapeutically effective amount will vary depending on the anti-HLA-G antibody, the disease and its severity and the age, weight, etc., of the subject to be treated.

The antibodies of the invention, formulations, or pharmaceutical compositions thereof may be administered for prophylactic and/or therapeutic treatments. In prophylactic applications, antibodies, formulations, or compositions are administered to a subject at risk of a disorder or condition as described herein, in an amount sufficient to prevent or reduce the subsequent effects of the condition or one or more of its symptoms. In therapeutic applications, the antibodies are administered to a subject already suffering from a disorder or condition as described herein, in an amount sufficient to cure, alleviate or partially arrest the condition or one or more of its symptoms. Such therapeutic treatment may result in a decrease in severity of disease symptoms, or an increase in frequency or duration of symptom-free periods.

The present invention provides a method of treating a disorder or condition as described herein in a subject in need thereof, the method comprising administering to the subject an antibody or a pharmaceutical composition according to the present invention. Such antibody is administered in a therapeutically effective amount.

The present invention also provides an antibody or a pharmaceutical composition of the invention for use in therapy, in particular for use in the treatment of a disorder or condition as described herein.

The present invention also provides the use of an antibody or a pharmaceutical composition of the invention for the manufacture of a medicament, in particular for use in the treatment of a disorder or condition as described herein.

Antibodies of the present invention may be used in treating, preventing or ameliorating any condition that is associated with HLA-G activity; for example, any condition which results in whole or in part from signalling through an HLA-G receptor.

HLA-G associated diseases or disorders include especially cancers (or tumors), infections and autoimmune disorders.

In one embodiment, the invention provides an antibody or pharmaceutical composition of the invention for use in the treatment of a disease characterized by over expression of HLA-G.

Antibodies of the present invention may be especially useful for treating or preventing cancer, including cancer characterized by over expression of HLA-G. Therefore, in one embodiment, the invention provides an antibody or pharmaceutical composition of the invention for use in the treatment of cancer. In one embodiment, the invention provides an antibody or pharmaceutical composition of the invention for use in the treatment of a cancer characterized by over expression of HLA-G.

Cancers in the context of the present invention include, for example Renal clear cell carcinoma (RCC), Colorectal carcinoma (CRC), Pancreatic cancer, Ovarian cancer, Breast cancer, Head and neck carcinoma, Stomach cancer, Hepatocellular carcinoma, lung cancer, neuroblastoma, and haematological cancers. Antibodies of the present invention may be useful for treating or preventing a liquid cancer such as a haematological cancer.

Antibodies of the present invention may be especially useful for treating or preventing a solid tumor. Therefore, in one embodiment, the invention provides an antibody or pharmaceutical composition of the invention for use in the treatment of a solid tumor. In one embodiment, the invention provides an antibody or pharmaceutical composition of the invention for use in the treatment of a solid tumor characterized by over expression of HLA-G. In one embodiment, the invention provides an antibody or pharmaceutical composition of the invention for use in the treatment of Renal clear cell carcinoma (RCC), Colorectal carcinoma (CRC), Pancreatic cancer, Ovarian cancer, Head and neck carcinoma, Stomach cancer or Hepatocellular carcinoma. In one particular embodiment, the solid tumor is Renal clear cell carcinoma (RCC). In another particular embodiment, the solid tumor is Colorectal carcinoma (CRC).

In one embodiment, the present invention provides the use of an antibody or a pharmaceutical composition of the invention for the manufacture of a medicament for use in the treatment of a solid tumor. In one embodiment, the present invention provides the use of an antibody or a pharmaceutical composition of the invention for the manufacture of a medicament for use in the treatment of a solid tumor characterized by over expression of HLA-G. In one embodiment, the present invention provides the use of an antibody or a pharmaceutical composition of the invention for the manufacture of a medicament for use in the treatment of Renal clear cell carcinoma (RCC), Colorectal carcinoma (CRC), Pancreatic cancer, Ovarian cancer, Head and neck carcinoma, Stomach cancer or Hepatocellular carcinoma. In one particular embodiment, the solid tumor is Renal clear cell carcinoma (RCC). In another particular embodiment, the solid tumor is Colorectal carcinoma (CRC).

In one embodiment, the invention provides a method of treating a solid tumor in a patient comprising administering to said patient a therapeutically effective amount of an antibody or pharmaceutical composition of the invention. In one embodiment, the invention provides a method of treating a solid tumor characterized by over expression of HLA-G in a patient comprising administering to said patient a therapeutically effective amount of an antibody or pharmaceutical composition of the invention.

In one embodiment, the solid tumor is selected from Renal clear cell carcinoma (RCC), Colorectal carcinoma (CRC), Pancreatic cancer, Ovarian cancer, Head and neck carcinoma, Stomach cancer and Hepatocellular carcinoma. In one particular embodiment, the solid tumor is Renal clear cell carcinoma (RCC). In another particular embodiment, the solid tumor is Colorectal carcinoma (CRC).

The present invention also provides the use of the antibodies of the present invention as diagnostically active agents or in diagnostic assays, for example, for diagnosing a disease or its severity.

In one embodiment, the invention provides a method for diagnosing a solid tumor by using an antibody or a pharmaceutical composition according to the invention. In one embodiment, the invention provides a method for diagnosing a solid tumor characterized by over expression of HLA-G by using an antibody or a pharmaceutical composition according to the invention.

In one embodiment, the invention provides a method for diagnosing Renal clear cell carcinoma (RCC), Colorectal carcinoma (CRC), Pancreatic cancer, Ovarian cancer, Head and neck carcinoma, Stomach cancer or Hepatocellular carcinoma by using an antibody or a pharmaceutical composition according to the invention. In one particular embodiment, the solid tumor is Renal clear cell carcinoma (RCC). In another particular embodiment, the solid tumor is Colorectal carcinoma (CRC).

The diagnosis may preferably be performed on biological samples. A “biological sample” encompasses a variety of sample types obtained from an individual and can be used in a diagnostic or monitoring assay. The definition encompasses cerebrospinal fluid, blood such as plasma and serum, and other liquid samples of biological origin such as urine and saliva, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides.

Diagnostic testing may preferably be performed on biological samples which are not in contact with the human or animal body. Such diagnostic testing is also referred to as in vitro testing. In vitro diagnostic testing may rely on an in vitro method of detecting of HLA-G in a biological sample, which has been obtained from a subject.

In one embodiment, the invention provides a method for diagnosing a solid tumor expressing HLA-G in a biological sample by using an antibody or a pharmaceutical composition according to the invention. In one embodiment, the invention provides a method for diagnosing Renal clear cell carcinoma (RCC), Colorectal carcinoma (CRC), Pancreatic cancer, Ovarian cancer, Head and neck carcinoma, Stomach cancer or Hepatocellular carcinoma in a biological sample by using an antibody or a pharmaceutical composition according to the invention.

### EXAMPLES

### Example 1: Generation of HLA-G, HLA-Is, and ILT2, ILT4 Proteins for Use in the Screening Assays

### 1.1. HLA-G Proteins: Sequences

The sequence of the most abundant isoforms of HLA-G, HLA-G1 (membrane bound) or HLA-G5 (soluble), comprising alpha 1, 2 and 3 domains of HLA-G, was used to generate HLA-G constructs for screening antibodies against HLA-G.

The sequence of the HLA-G Extracellular domain or ECD, was defined based on crystal structure analysis (Alpha 1 domain in italic-alpha 2 domain-alpha 3 domain underlined-final residues KQ as determined by crystallography). The 20 residues specific to HLA-G are in bold:

(SEQ ID NO: 108)
GSHSMRYFSAAVSRPGRGEPRFIAMGYVDDTQFVRF
DSDSACPRMEPRAPWVEQEGPEYWEEETRNTKAHAQ
TDRMNLQTLRGYYNQSEASSHTLQWMIGCDLGSDGR
LLRGYEQYAYDGKDYLALNEDLRSWTAADTAAQISK
RKCEAANVAEQRRAYLEGTCVEWLHRYLENGKEMLQ
RADPPKTHVTHHPVFDYEATLRCWALGFYPAEIILT
WQRDGEDQTQDVELVETRPAGDGTFQKWAAVVVPSG
EEQRYTCHVQHEGLPEPLMLRWKQ

### Soluble HLA-G (HLA-G ECD):

The protein was expressed with an AVItev10HisTag (signal peptide in bold, avidin affinity tag or AVI tag for biotinylation underlined, Tev protease site underlined and in italic, 10His Tag in italic):

(SEQ ID NO: 109)
MVVMAPRTLFLLLSGALTLTETWAGSHSMRYFSAAVSRPGRGEPRF
IAMGYVDDTQFVRFDSDSACPRMEPRAPWVEQEGPEYWEEETRNTK
AHAQTDRMNLQTLRGYYNQSEASSHTLQWMIGCDLGSDGRLLRGYE
QYAYDGKDYLALNEDLRSWTAADTAAQISKRKCEAANVAEQRRAYL
EGTCVEWLHRYLENGKEMLQRADPPKTHVTHHPVFDYEATLRCWAL
GFYPAEIILTWQRDGEDQTQDVELVETRPAGDGTFQKWAAVVVPSG
EEQRYTCHVQHEGLPEPLMLRWKQGLNDIFEAQKIEWHELEENLYF
QGSGGSHHHHHHHHH

The purified final protein sequence used for screening assays comprises the following sequence (comprising the AVItev10His Tag):

(SEQ ID NO: 110)
GSHSMRYFSAAVSRPGRGEPRFIAMGYVDDTQFVRFDSDSACPRME
PRAPWVEQEGPEYWEEETRNTKAHAQTDRMNLQTLRGYYNQSEASS
HTLQWMIGCDLGSDGRLLRGYEQYAYDGKDYLALNEDLRSWTAADT
AAQISKRKCEAANVAEQRRAYLEGTCVEWLHRYLENGKEMLQRADP
PKTHVTHHPVFDYEATLRCWALGFYPAEIILTWQRDGEDQTQDVEL
VETRPAGDGTFQKWAAVVVPSGEEQRYTCHVQHEGLPEPLMLRWKQ
GLNDIFEAQKIEWHELEENLYFQGSGGSHHHHHHHHH

### HLA-G DNA Sequence for Cell Membrane Bound Expression:

(SEQ ID NO: 111)
atggtcgtcatggcgcccaggactctgtttctgcttctgtccggcg
ccttgaccttgaccgagacttgggccggaagccactcgatgcggta
cttctccgcggccgtgtctagaccgggtcggggagaaccccggttc
atcgccatgggctacgtggatgacacccagttcgtgcggttcgaca
gcgattcagcctgccctcgcatggagccgagagccccttgggtgga
acaggaagggccggagtactgggaagaggaaacacgcaacaccaag
gcccacgctcaaaccgaccggatgaacttgcagacgctgcggggat
actataaccagtccgaggcgtcgagccatacccttcaatggatgat
tggctgtgacctgggttccgacgggagactgctgcggggctacgag
cagtacgcctatgacggaaaggactacctggccctcaacgaagatc
tccgctcctggaccgctgccgatactgcggcccagatctccaagcg
caaatgcgaagcggctaatgtcgccgaacagcgaagggcctacctg
gaaggcacttgcgtggagtggctgcaccgctacctggagaacggaa
aggaaatgctgcagagggcagacccccctaagacccatgtcaccca
ccatcccgtgttcgactacgaagccaccctgagatgctgggcgctg
ggcttttaccctgccgagatcatcctgacctggcaacgcgacgggg
aagatcagacccaagacgtggaacttgtggagactagaccagccgg
cgatgggactttccagaaatgggcagccgtggtcgtgccgtcggga
gaggaacaacgctacacctgtcacgtgcagcacgagggtctgccag
agcccctgatgctgcggtggaagcagagctccctccccaccattcc
gatcatgggaattgtggcgggactcgtggtgctcgccgctgtcgtg
actggagccgcagtggcagctgtgctctggcggaagaagtcctcag
ac

The corresponding membrane-bound protein comprises the following sequence (signal peptide MVVMAPRTLFLLLSGALTLTETWA cleaved after expression, transmembrane and cytoplasmic domain in italic):

(SEQ ID NO: 107)
GSHSMRYFSAAVSRPGRGEPRFIAMGYVDDTQFVRFDSDSACPRME
PRAPWVEQEGPEYWEEETRNTKAHAQTDRMNLQTLRGYYNQSEASS
HTLQWMIGCDLGSDGRLLRGYEQYAYDGKDYLALNEDLRSWTAADT
AAQISKRKCEAANVAEQRRAYLEGTCVEWLHRYLENGKEMLQRADP
PKTHVTHHPVFDYEATLRCWALGFYPAEIILTWQRDGEDQTQDVEL
VETRPAGDGTFQKWAAVVVPSGEEQRYTCHVQHEGLPEPLMLRWKQ
SSLPTIPIMGIVAGLVVLAAVVTGAAVAAVLWRKKSSD

### “HLA-G Null 1,2,3”

“HLA-GNull 1,2,3” corresponds to HLA-G wherein the amino acids specifically expressed on HLA-G α1, α2 and α3 were substituted with consensus amino acids expressed on other HLA-Is (20 amino acids mutated, in bold in the sequences below)

HLA-Is consensus amino acids were derived from sequence information obtained from the Immuno Polymorphism Database at the EBI. HLA-Is full length proteins were analysed, and residue profile plots were generated for each domain (alpha 1-3) across the full HLA-I set which allowed HLA-G specific residues to be identified (20 in total).

The sequence information was also used to generate an allelic consensus sequence for each HLA protein that substituted positions in the canonical sequence with the most common residue found across all alleles.

To obtain the HLA-G Null 1,2,3 sequence, HLA-G specific residues were changed to the consensus residues found in the other HLA molecules at the specific 20 positions.

The soluble protein (HLA-G Null 1,2,3 ECD) was expressed with an AVItev10HisTag (signal peptide in bold, AVI tag underlined, Tev protease site underlined and in italic, 10His Tag in italic):

(SEQ ID NO: 112)
MVVMAPRTLFLLLSGALTLTETWAGSHSMRYFSTAVSRPGRGEPRF
IAVGYVDDTQFVRFDSDAASPRMEPRAPWVEQEGPEYWERETRNAK
ANAQTDRVNLRTLRGYYNQSEAGSHTLQWMYGCDLGSDGRLLRGYE
QYAYDGKDYLALNEDLRSWTAADTAAQISKRKCEAAREAEQLRAYL
EGTCVEWLHRYLENGKETLQRADPPKTHVTHHPVSDHEATLRCWAL
GFYPAEITLTWQRDGEDQTQDTELVETRPAGDGTFQKWAAVVVPSG
EEQRYTCHVQHEGLPEPLTLRWKQGLNDIFEAQKIEWHELEENLYF
QGSGGSHHHHHHHHHH

The peptide signal was cleaved after expression and the purified final protein sequence used for screening assays comprises the following sequence:

(SEQ ID NO: 113)
GSHSMRYFSTAVSRPGRGEPRFIAVGYVDDTQFVRFDSDAASPRME
PRAPWVEQEGPEYWERETRNAKANAQTDRVNLRTLRGYYNQSEAGS
HTLQWMYGCDLGSDGRLLRGYEQYAYDGKDYLALNEDLRSWTAADT
AAQISKRKCEAAREAEQLRAYLEGTCVEWLHRYLENGKETLQRADP
PKTHVTHHPVSDHEATLRCWALGFYPAEITLTWQRDGEDQTQDTEL
VETRPAGDGTFQKWAAVVVPSGEEQRYTCHVQHEGLPEPLTLRWKQ
GLNDIFEAQKIEWHELEENLYFQGSGGSHHHHHHHHHH

“HLA-G Null 1,2,3” DNA sequence for cell membrane bound expression:

(SEQ ID NO: 114)
atggtcgtcatggcgcccaggactctgtttctgcttctgtccggcg
ccttgaccttgaccgagacttgggccggaagccactcgatgcggta
cttctccaccgccgtgtctagaccgggtcggggagaaccccggttc
atcgccgtgggctacgtggatgacacccagttcgtgcggttcgaca
gcgatgccgcctcacctcgcatggagccgagagccccttgggtgga
acaggaagggccggagtactgggaacgcgaaacacgcaacgccaag
gccaacgctcaaaccgaccgggtcaacttgagaacgctgcggggat
actataaccagtccgaggcgggaagccatacccttcaatggatgta
cggctgtgacctgggttccgacgggagactgctgcggggctacgag
cagtacgcctatgacggaaaggactacctggccctcaacgaagatc
tccgctcctggaccgctgccgatactgcggcccagatctccaagcg
caaatgcgaagcggctagagaagccgaacagctgagggcctacctg
gaaggcacttgcgtggagtggctgcaccgctacctggagaacggaa
aggaaacgctgcagagggcagacccccctaagacccatgtcactca
ccacccggtgtccgatcacgaggccaccctgaggtgctgggcactg
ggattctacccggcggagatcaccctgacctggcaacgggacggcg
aagatcagacccaagacaccgagctcgtggaaaccaggcctgcggg
tgatggaaccttccagaagtgggctgccgtggtggtgccatccggg
gaggagcaacggtacacttgtcacgtgcagcacgagggactgcctg
aacccctgactctgcggtggaagcagagctccctccccaccattcc
gatcatgggaattgtgggggactcgtggtgctcgccgctgtcgtga
ctggagccgcagtggcagctgtgctctggcggaagaagtcctcaga
c

The corresponding membrane-bound protein comprises the following sequence (signal peptide MVVMAMPRTLFLLLSGALTLTETWA cleaved after expression, transmembrane and cytoplasmic domain in italic):

(SEQ ID NO: 115)
GSHSMRYFSTAVSRPGRGEPRFIAVGYVDDTQFVRFDSDAASPRME
PRAPWVEQEGPEYWERETRNAKANAQTDRVNLRTLRGYYNQSEAGS
HTLQWMYGCDLGSDGRLLRGYEQYAYDGKDYLALNEDLRSWTAADT
AAQISKRKCEAAREAEQLRAYLEGTCVEWLHRYLENGKETLQRADP
PKTHVTHHPVSDHEATLRCWALGFYPAEITLTWQRDGEDQTQDTEL
VETRPAGDGTFQKWAAVVVPSGEEQRYTCHVQHEGLPEPLTLRWKQ
SSLPTIPIMGIVAGLVVLAAVVTGAAVAAVLWRKKSSD

### “HLA-G Null 1,3”

“HLA-G Null 1,3” corresponds to HLA-G wherein amino acids specifically expressed on HLA-G α1 and α3 were substituted with consensus amino acids expressed on other HLA-Is (mutated amino acids in bold in the sequence SEQ ID NO: 117 below).

The protein was expressed at the surface of cells. The DNA sequence for cell membrane bound expression comprises:

(SEQ ID NO: 116)
atggtcgtcatggcgcccaggactctgtttctgcttctgtccggcg
ccttgaccttgaccgagacttgggccggaagccactcgatgcggta
cttctccaccgccgtgtctagaccgggtcggggagaaccccggttc
atcgccgtgggctacgtggatgacacccagttcgtgcggttcgaca
gcgatgccgcctcacctcgcatggagccgagagccccttgggtgga
acaggaagggccggagtactgggaacgcgaaacacgcaacgccaag
gccaacgctcaaaccgaccgggtcaacttgagaacgctgcggggat
actataaccagtccgaggcgtcgagccatacccttcaatggatgat
tggctgtgacctgggttccgacgggagactgctgcggggctacgag
cagtacgcctatgacggaaaggactacctggccctcaacgaagatc
tccgctcctggaccgctgccgatactgcggcccagatctccaagcg
caaatgcgaagcggctaatgtcgccgaacagcgaagggcctacctg
gaaggcacttgcgtggagtggctgcaccgctacctggagaacggaa
aggaaatgctgcagagggcagacccccctaagacccatgtcactca
ccacccggtgtccgatcacgaggccaccctgaggtgctgggcactg
ggattctacccggcggagatcaccctgacctggcaacgggacggcg
aagatcagacccaagacaccgagctcgtggaaaccaggcctgcggg
tgatggaaccttccagaagtgggctgccgtggtggtgccatccggg
gaggagcaacggtacacttgtcacgtgcagcacgagggactgcctg
aacccctgactctgcggtggaagcagagctccctccccaccattcc
gatcatgggaattgtgggggactcgtggtgctcgccgctgtcgtga
ctggagccgcagtggcagctgtgctctggcggaagaagtcctca

The corresponding membrane-bound protein comprises the following sequence (signal peptide MVVMAPRTLFLLLSGALTLTETWA cleaved after expression, transmembrane and cytoplasmic domain in italic):

(SEQ ID NO: 117)
GSHSMRYFSTAVSRPGRGEPRFIAVGYVDDTQFVRFDSDAASPRME
PRAPWVEQEGPEYWERETRNAKANAQTDRVNLRTLRGYYNQSEASS
HTLQWMIGCDLGSDGRLLRGYEQYAYDGKDYLALNEDLRSWTAADT
AAQISKRKCEAANVAEQRRAYLEGTCVEWLHRYLENGKEMLQRADP
PKTHVTHHPVSDHEATLRCWALGFYPAEITLTWQRDGEDQTQDTEL
VETRPAGDGTFQKWAAVVVPSGEEQRYTCHVQHEGLPEPLTLRWKQ
SSLPTIPIMGIVAGLVVLAAVVTGAAVAAVLWRKKSSD

### “HLA-G Null3”

“HLA-G Null3” corresponds to HLA-G wherein amino acids specifically expressed on HLA-G α3 were substituted with consensus amino acids expressed on other HLA-Is (5 amino acids mutated, in bold in the sequence SEQ ID NO:119 below).

The protein was expressed at the surface of cells. The DNA sequence for cell membrane bound expression comprises:

(SEQ ID NO: 118)
atggtcgtcatggcgcccaggactctgtttctgcttctgtccggcg
ccttgaccttgaccgagacttgggccggaagccactcgatgcggta
cttctccgcggccgtgtctagaccgggtcggggagaaccccggttc
atcgccatgggctacgtggatgacacccagttcgtgcggttcgaca
gcgattcagcctgccctcgcatggagccgagagccccttgggtgga
acaggaagggccggagtactgggaagaggaaacacgcaacaccaag
gcccacgctcaaaccgaccggatgaacttgcagacgctgcggggat
actataaccagtccgaggcgtcgagccatacccttcaatggatgat
tggctgtgacctgggttccgacgggagactgctgcggggctacgag
cagtacgcctatgacggaaaggactacctggccctcaacgaagatc
tccgctcctggaccgctgccgatactgcggcccagatctccaagcg
caaatgcgaagcggctaatgtcgccgaacagcgaagggcctacctg
gaaggcacttgcgtggagtggctgcaccgctacctggagaacggaa
aggaaatgctgcagagggcagacccccctaagacccatgtcactca
ccacccggtgtccgatcacgaggccaccctgaggtgctgggcactg
ggattctacccggcggagatcaccctgacctggcaacgggacggcg
aagatcagacccaagacaccgagctcgtggaaaccaggcctgcggg
tgatggaaccttccagaagtgggctgccgtggtggtgccatccggg
gaggagcaacggtacacttgtcacgtgcagcacgagggactgcctg
aacccctgactctgcggtggaagcagagctccctccccaccattcc
gatcatgggaattgtggcgggactcgtggtgctcgccgctgtcgtg
actggagccgcagtggcagctgtgctctggcggaagaagtcctcag
ac

The corresponding membrane-bound protein comprises the following sequence (signal peptide MVVMAPRTLFLLLSGALTLTETWA cleaved after expression, transmembrane and cytoplasmic domain in italic):

(SEQ ID NO: 119)
GSHSMRYFSAAVSRPGRGEPRFIAMGYVDDTQFVRFDSDSACPRME
PRAPWVEQEGPEYWEEETRNTKAHAQTDRMNLQTLRGYYNQSEASS
HTLQWMIGCDLGSDGRLLRGYEQYAYDGKDYLALNEDLRSWTAADT
AAQISKRKCEAANVAEQRRAYLEGTCVEWLHRYLENGKEMLQRADP
PKTHVTHHPVSDHEATLRCWALGFYPAEITLTWQRDGEDQTQDTEL
VETRPAGDGTFQKWAAVVVPSGEEQRYTCHVQHEGLPEPLTLRWKQ
SSLPTIPIMGIVAGLVVLAAVVTGAAVAAVLWRKKSSD

### “HLA-G Null3 2AA”

“HLA-G Null3 2AA” corresponds to HLA-G wherein only 2 amino acids specifically expressed on HLA-G α3 and reported to interact with ILT2/ILT4 (F195, Y197 of HLA-G) are substituted with consensus amino acids expressed on other HLA-Is (amino acids mutated in bold).

The protein was expressed at the surface of cells. The DNA sequence for cell membrane bound expression comprises:

(SEQ ID NO: 120)
atggtcgtcatggcgcccaggactctgtttctgcttctgtccggcg
ccttgaccttgaccgagacttgggccggaagccactcgatgcggta
cttctccgcggccgtgtctagaccgggtcggggagaaccccggttc
atcgccatgggctacgtggatgacacccagttcgtgcggttcgaca
gcgattcagcctgccctcgcatggagccgagagccccttgggtgga
acaggaagggccggagtactgggaagaggaaacacgcaacaccaag
gcccacgctcaaaccgaccggatgaacttgcagacgctgcggggat
actataaccagtccgaggcgtcgagccatacccttcaatggatgat
tggctgtgacctgggttccgacgggagactgctgcggggctacgag
cagtacgcctatgacggaaaggactacctggccctcaacgaagatc
tccgctcctggaccgctgccgatactgcggcccagatctccaagcg
caaatgcgaagcggctaatgtcgccgaacagcgaagggcctacctg
gaaggcacttgcgtggagtggctgcaccgctacctggagaacggaa
aggaaatgctgcagagggcagacccccctaagacccatgtcaccca
ccatcccgtgtcagaccacgaagccaccctgagatgctgggcgctg
ggcttttaccctgccgagatcatcctgacctggcaacgcgacgggg
aagatcagacccaagacgtggaacttgtggagactagaccagccgg
cgatgggactttccagaaatgggcagccgtggtcgtgccgtcggga
gaggaacaacgctacacctgtcacgtgcagcacgagggtctgccag
agcccctgatgctgcggtggaagcagagctccctccccaccattcc
gatcatgggaattgtggcgggactcgtggtgctcgccgctgtcgtg
actggagccgcagtggcagctgtgctctggcggaagaagtcctcag
ac

The corresponding membrane-bound protein comprises the following sequence (signal peptide MVVMAPRTLFLLLSGALTLTETWA cleaved after expression, transmembrane and cytoplasmic domain in italic):

(SEQ ID NO: 121)
GSHSMRYFSAAVSRPGRGEPRFIAMGYVDDTQFVRFDSDSACPRME
PRAPWVEQEGPEYWEEETRNTKAHAQTDRMNLQTLRGYYNQSEASS
HTLQWMIGCDLGSDGRLLRGYEQYAYDGKDYLALNEDLRSWTAADT
AAQISKRKCEAANVAEQRRAYLEGTCVEWLHRYLENGKEMLQRADP
PKTHVTHHPVSDHEATLRCWALGFYPAEIILTWQRDGEDQTQDVEL
VETRPAGDGTFQKWAAVVVPSGEEQRYTCHVQHEGLPEPLMLRWKQ
SSLPTIPIMGIVAGLVVLAAVVTGAAVAAVLWRKKSSD

### Isolated HLA-G Alpha 3 Domain (Wild-Type)

The soluble protein was expressed as Tev-humanFc fusion protein (signal peptide in bold, Tev underlined, Fc fragment in italic) and was not co-expressed with B2m (“B2m free isolated HLA-G alpha 3 domain”):

(SEQ ID NO: 122)
MSVPTQVLGLLLLWLTDARCDPPKTHVTHHPVFDYEATLRCWALGF
YPAEIILTWQRDGEDQTQDVELVETRPAGDGTFQKWAAVVVPSGEE
QRYTCHVQHEGLPEPLMLRWKQSSLPTIPILEENLYFQGVDKKVEP
KSCDKTHTCPPCPAPELLGGPSVFLFPPKPKDTLMISRTPEVTCVV
VDVSHEDPEVKFNWYVDGVEVHNAKTKPREEQYNSTYRVVSVLTVL
HQDWLNGKEYKCKVSNKALPAPIEKTISKAKGQPREPQVYTLPPSR
DELTKNQVSLTCLVKGFYPSDIAVEWESNGQPENNYKTTPPVLDSD
GSFFLYSKLTVDKSRWQQGNVFSCSVMHEALHNHYTQKSLSLSPGK

The peptide signal was cleaved after expression and the purified final protein sequence used for screening assays comprises the following sequence (the Fc Tag was cleaved off):

(SEQ ID NO: 123)
DPPKTHVTHHPVFDYEATLRCWALGFYPAEIILTWQRDGEDQTQDVEL
VETRPAGDGTFQKWAAVVVPSGEEQRYTCHVQHEGLPEPLMLRWKQSS
LPTIPILEENLYFQ

DNA sequence for cell membrane bound expression:

(SEQ ID NO: 124)
atgtccgtgccgacccaagtgctgggactgctcctgctctggctgactga
cgctcgctgtgacccccctaagacccacgtcactcatcaccctgtgtccg
accatgaagctaccctgagatgctgggccctgggtttctaccccgccgag
attaccttgacctggcaaagggacggcgaagatcagacgcaagacaccga
gctcgtggagactcggccagcgggggatggaacattccagaaatgggccg
cagtggtcgtgccgtccggagaagaacagcggtacacttgccacgtgcag
cacgaaggcctgccggagcctctgacccttcgctggaagcagtcgagcct
ccccaccatcccgatcatggggattgtggccggccttgtggtgctggccg
cagtcgtgaccggagcagctgtggcggctgtcctgtggcggaagaagtca
agcgat

The corresponding membrane-bound protein comprises the following sequence (signal peptide cleaved after expression, transmembrane and cytoplasmic domain in italic):

(SEQ ID NO: 125)
DPPKTHVTHHPVFDYEATLRCWALGFYPAEIILTWQRDGEDQTQDVEL
VETRPAGDGTFQKWAAVVVPSGEEQRYTCHVQHEGLPEPLMLRWKQSS
LPTIPIMGIVAGLVVLAAVVTGAAVAAVLWRKKSSD

### Isolated HLA-G Alpha 3 Domain, Null (5 Amino Acids Mutated in Bold)

The protein was expressed as 10HistevAVI tag protein (Tag and GS linkers in italic, signal peptide in bold)

(SEQ ID NO: 126)
MSVPTQVLGLLLLWLTDARCGGSHHHHHHHHHHGSGSENLYFQGLNDI
FEAQKIEWHGGGSGSDPPKTHVTHHPVSDHEATLRCWALGFYPAEITL
TWQRDGEDQTQDTELVETRPAGDGTFQKWAAVVVPSGEEQRYTCHVQH
EGLPEPLTLRWKQSSLPTIPI

The peptide signal was cleaved after expression and the 10His Tag was removed during purification. The purified final protein sequence used for screening assays comprises the following sequence (N-terminal AVI Tag and GS linker in italic):

(SEQ ID NO: 127)
GLNDIFEAQKIEWHGGGSGSDPPKTHVTHHPVSDHEATLRCWALGFYP
AEITLTWQRDGEDQTQDTELVETRPAGDGTFQKWAAVVVPSGEEQRYT
CHVQHEGLPEPLTLRWKQSSLPTIPI

### B2m

All the HLA-G constructs used in the screening assays and described above (except the B2m free isolated HLA-G alpha 3 domain, wild-type) were co-expressed with B2m. For expression of soluble HLA-G constructs, B2m was co-expressed, with the following sequence (signal peptide in bold):

(SEQ ID NO: 128)
MSRSVALAVLALLSLSGLEAIQRTPKIQVYSRHPAENGKSNFLNCYVS
GFHPSDIEVDLLKNGERIEKVEHSDLSFSKDWSFYLLYYTEFTPTEKD
EYACRVNHVTLSQPKIVKWDRDM

The signal peptide was cleaved after expression and the final purified protein used for the screening assays comprises the following sequence:

(SEQ ID NO: 129)
IQRTPKIQVYSRHPAENGKSNFLNCYVSGFHPSDIEVDLLKNGERIEK
VEHSDLSFSKDWSFYLLYYTEFTPTEKDEYACRVNHVTLSQPKIVKWD
RDM

All the membrane-bound HLA-G constructs (including cell expressed isolated alpha 3 domains) were co-expressed with B2m. The DNA sequence used for transfection is as follows:

(SEQ ID NO: 130)
atgtcacgctccgtggcactggctgtgctggccctgctctccctgtcg
ggtcttgaggccatccagaggactccgaagattcaagtctactcccgc
catcctgccgaaaacggaaagtccaattttctgaactgctatgtgtcg
ggcttccacccctccgacatcgaagtggacctcctgaagaacggggag
cggattgaaaaggtcgagcacagcgacctgagcttctcgaaggactgg
tcattctacctcctgtactacactgaattcaccccaaccgaaaaggat
gagtacgcgtgcagagtgaaccacgtgaccttgagccagccgaagatc
gtgaaatgggaccgggatatg

The corresponding B2m complexed with membrane bound HLA-G constructs comprises the sequence SEQ ID NO: 129 (signal peptide MSRSVALAVLALLSLSGLEA cleaved after expression).

### Methods for the Production and Purification of the HLA-G Constructs Expressed as Soluble Proteins

### Protein Expression and Purification of Isolated HLA-G α3 Domain, Wild-Type (TevHumanFc or TevHFc)

HLA-G α3 TevHFc was co-expressed with β2m using the Expi293™ Expression System (Life Technologies™) following manufacturers protocol. Cells were harvested 5 days post transfection and supernatants used immediately for purification.

Supernatants comprising HLA-G α3 TevHFc+β2m protein was applied to Hitrap Protein A column. Unbound protein and contaminants were washed with PBS and HLA-G α3 TevHFc+β2m protein eluted with 0.1M citric acid buffer, pH 2 and the peak fractions were neutralised with 0.5 ml 2M Tris PH 8. Fractions containing purified HLA-G α3 TevHFc+β2m protein were pooled and the HFc tag removed by incubation of the protein with tev protease at a ratio of 1:100 for 2 hours at room temperature and 2 hours at 4 C. Protein was concentrated and purified further by size exclusion chromatography on S75 26/60 which had been equilibrated with PBS buffer. Fractions containing purified HLA-G α3 protein were pooled, concentrated and aliquots stored at −80 C until needed.

### Protein Expression and Purification of Isolated HLA-G Apha 3 Domain, Null

10histevAVI HLA-G α3 null was co-expressed with β2m using the Expi293™ Expression System (Life Technologies™) following manufacturers protocol. Cells were harvested 5 days post transfection and supernatants used immediately for purification. Supernatants comprising 10histevAVI HLAG α3 null+B2m protein was applied to HisTrap Excel column (GE Healthcare) using an Akta Purifier (GE Healthcare). Unbound protein and contaminants were washed with Cytiva HyClone™ Phosphate Buffered Saline (PBS), 500 mM NaCl (pH 7.5). 10 mM Imidazole and protein was eluted with Cytiva HyClone™ Phosphate Buffered Saline (PBS), 500 mM NaCl (pH 7.5), 500 mM imidazole. Fractions containing purified 10histevAVI HLAG α3 null+B2m protein were pooled and the 10his tag removed by incubation of the protein with tev protease at a ratio of 1:100 for 2 hours at room temperature and 2 hours at 4 C. Protein was concentrated and purified further by size exclusion chromatography on S75 26/60 which had been equilibrated with Cytiva HyClone™ Phosphate Buffered Saline (PBS). Fractions containing purified AVI HLAG α3 null+B2m protein were pooled, concentrated and aliquots stored at −80 C until needed.

Protein Expression and Purification of HLA-G ECD (Wild Type or Null Variants) Co-Expressed with B2m

HLA-G ECD (WT or null mutants) were co-expressed with β2m using the CHO-SXE expression system following the manufacturer's protocol. In brief, CHO-SXE cells were grown in a shaking incubator at 37° C. with 8% CO₂ in serum-free CD CHO medium (Gibco) supplemented with Gibco® GlutaMAX™ (1:1000), to a cell density of 6×10⁶/mL. The cells were then spun down at 1500 rpm and resuspended in fresh ExpiCHO™ Expression Medium (Gibco). The cells were transfected using 1 mg/L of DNA at 1:1 ratio of HLA-G ECD and β2m. Transfections were carried out using ExpiFectamine™ CHO Transfection Kit and OptiPRO™ SFM. The conditioned media containing the secreted proteins were collected 96 hr after transfection. The filtered cell culture supernatant was loaded onto a 5-ml HisTrap Excel column (GE Healthcare) using an Akta Purifier (GE Healthcare). The column was washed with Cytiva HyClone™ Phosphate Buffered Saline (PBS), 500 mM NaCl (pH 7.5) and the protein was eluted with the same buffer containing 500 mM imidazole. Fractions containing protein were analysed by SDS-PAGE using NuPAGE 4-20% Tris-Glycine (Thermo) and NuPAGE MES SDS Running Buffer (Thermo) stained with Quick Coomassie Stain (VWR). Pure fractions were pooled before being concentrated using an using an Amicon® Ultra-15 Centrifugal Filter Unit (Millipore). The proteins were then further purified using a Superdex 200 16/600 column (GE Healthcare) using Cytiva HyClone™ Phosphate Buffered Saline (PBS) as the running buffer.

Protein purity was assessed by analytical size exclusion HPLC and sodium dodecyl sulfate polyacrylamide gel electrophoresis (SDS-PAGE). Proteins were greater than 97% pure (generally at least 99%). Also, the proteins were analysed by liquid chromatography mass spectrometry (LC-MS) to confirm that the sequence molecular weight (MW) was as expected.

### Methods for the Production of Cells Expressing the HLA-G Constructs at the Cell Surface

Transient Expression of HLA-G (Including Null Constructs) with B2m at the Surface of ExpiHEK293

Co-transfection with 1:1 ratio HLA-G and B2m expression vectors into Expi293™ suspension cells was achieved using ExpiFectamine™ 293 transfection reagent (ThermoFisher Scientific) and gave expression of the cell surface protein from 24 hrs.

Transient Expression of HLA-G (Including Null Constructs) with B2m at the Surface of CHO (PHAGE Panning)

HLA-G or HLA-G Null 1,2,3 was co-transfected at 1:1 ratio with B2m expression vector into proprietary CHO-SXE cells with ExpiFectamine™ CHO Transfection Kit (Gibco) following the manufacturer's recommendations. The cells expressing HLA-G at their surface were harvested after 48 hours.

### HCT116 Cells Expressing HLA-G

The day before transfection, HCT116 cells (ATCC CCL-247) were seeded at 4×10⁶ cells per T 75 cm² flask in 20 ml of complete RPMI growth medium and incubate for 24 h, 37° C., 5% CO₂. On the day of transfection, growth medium was removed replace with 16 ml of complete growth medium. For each flask of cells to be transfected, 20 μg HLA-G and β2m plasmids (1:1 ratio) were diluted in 4 ml Opti-MEM® I Reduced Serum Media without serum. 80 μl of Lipofectamine LTX® Reagent were added into the above diluted Opti-MEM® DNA solution and incubated at room temperature for 30 min. After incubation, the DNA-Lipofectamine LTX® Reagent complexes were directly added to each flask containing cells and flasks were placed in a CO₂ incubator at 37° C. in for 22±2 h.

### 1.2. HLA-Is Constructs

HLA-Is constructs were co-expressed with β2m using the Expi293™ Expression System (Life technologies) following manufacturers protocol.

HLA-Is consensus sequences were derived from the amino acid sequences of the HLA-Is alleles that have been publicly reported. The relevant sequence information taken forward for analysis was obtained from the Immuno Polymorphism Database at the EBI. This information was used to generate an allelic consensus sequence for each HLA protein that substituted positions in the canonical sequence with the most common residue found across all alleles. The DNA sequences coding for the consensus amino acid sequences and used for transfection/cell membrane expression are listed below. The DNA sequence coding for B2m was as described above. The signal peptide in the secreted HLA-Is proteins was removed after expression.

### HLA-A

DNA sequence used for transfection/cell membrane expression:

(SEQ ID NO: 131)
atggccgtgatggccccaaggacccttctgctcctcctgtcgggagcg
ctcgcactgactcagacctgggctggctcacactccatgagatacttc
ttcacttctgtgtcccggcctggaagaggggagcccaggttcatcgcg
gtcggctacgtggacgacacccagttcgtgcgcttcgactccgatgcc
gcctcgcaacgcatggagccgagagctccgtggatcgaacaagagggc
ccggagtactgggaccaggaaactagaaacgtgaaggcccacagccag
accgaccgcgtggatctgggaaccctccgcggttactacaatcagtcg
gaagctggatcccacacgattcagatgatgtacggttgcgacgtgggc
tccgatggacggtttctgcgggggtatcggcaggatgcctatgacggg
aaggactacatcgccttgaacgaggacctccggtcatggactgccgca
gacatggcggcccaaatcaccaagcgcaaatgggaagccgcgcatgtg
gcagagcagctgcgggcctaccttgagggcacttgcgtggaatggctg
cgccgatacctggaaaacgggaaggaaaccctgcagcggactgaccca
cctaagacccacatgacccaccacgccgtgtccgaccatgaggccaca
ctgcggtgctgggccttgtccttctaccctgccgaaatcaccctcact
tggcaacgcgacggagaggatcagacccaagacaccgaactggtcgaa
actcggcctgcgggagatggaaccttccagaaatgggccgctgtcgtg
gtgccgagcggacaggaacagaggtacacctgtcatgtgcagcacgag
ggtctgccgaagcccctgacgctgagatgggagctgtcaagccagccc
actattcccattgtgggcattatcgccggactggtgctgcttggcgcc
gtcatcaccggtgctgtggtggcagccgtcatgtggcgccggaagtcc
agcgacaggaaggggggctcctacacccaagcggcgtcgagcgatagc
gcccagggatccgacgtgtccctcaccgcctgcaaggtc

The corresponding membrane-bound protein comprises the following sequence:

(SEQ ID NO: 132)
GSHSMRYFFTSVSRPGRGEPRFIAVGYVDDTQFVRFDSDAASQRMEPRAP
WIEQEGPEYWDQETRNVKAHSQTDRVDLGTLRGYYNQSEAGSHTIQMMYG
CDVGSDGRFLRGYRQDAYDGKDYIALNEDLRSWTAADMAAQITKRKWEAA
HVAEQLRAYLEGTCVEWLRRYLENGKETLQRTDPPKTHMTHHAVSDHEAT
LRCWALSFYPAEITLTWQRDGEDQTQDTELVETRPAGDGTFQKWAAVVVP
SGQEQRYTCHVQHEGLPKPLTLRWELSSQPTIPIVGIIAGLVLLGAVITG
AVVAAVMWRRKSSDRKGGSYTQAASSDSAQGSDVSLTACKV

### HLA-B

DNA sequence used for transfection/cell membrane expression:

(SEQ ID NO: 133)
atgcgcgtgactgcccctcgaaccgtgctcctgctgctctggggagctgt
ggcactcaccgaaacttgggccggatcccacagcatgcggtacttttaca
ctgcgatgtcgcgccctggaagaggggagccacgcttcattgccgtgggc
tacgtcgacgatacccagttcgtgcggttcgacagcgatgcagcctcgcc
tagagaggaacccagagccccgtggatcgaacaggaaggcccggagtact
gggatcggaacacccagatctccaagaccaacacgcagacctatcgggaa
tccctgaggaacctcaggggttactacaaccagtccgaggccggaagcca
caccctgcaaaggatgtacggatgcgacgtgggacccgatgggagactcc
tgcgcggtcacaaccagtacgcctacgacgggaaggactacatcgccctg
aatgaggacctgtcatcctggaccgcggctgatacagcagcccagatcac
ccagcggaaatgggaggccgccagagtggcagaacagctgcgcgcgtatc
tggagggtttgtgcgtggaatggctgcggcggtacctcgaaaacggaaag
gaaaccctgcagagagcagacccccccaagactcacgtcacccatcaccc
gatctctgaccatgaagccaccctgcggtgttgggccctcggcttctacc
cggcggaaattactctgacatggcagcgggacggagaggaccagacccag
gacaccgagctggtcgaaactcgccctgccggagacaggactttccagaa
atgggctgccgtggtggtgccgagcggagaggaacagcggtacacctgtc
acgtgcaacatgagggccttccgaagcccctgactctgcgctgggagcct
tcctcccaatcgacgatcccaattgtcggcatcgtggccggtctggctgt
gcttgcggtggtggtcattggcgcggtggtggctactgtgatgtgccgcc
gcaagagctcaggagggaagggcggctcctactcgcaagccgcctcctcg
gactccgcccaaggatccgatgtctcattgaccgcc

The corresponding membrane-bound protein comprises the following sequence:

(SEQ ID NO: 134)
GSHSMRYFYTAMSRPGRGEPRFIAVGYVDDTQFVRFDSDAASPREEPRAP
WIEQEGPEYWDRNTQISKTNTQTYRESLRNLRGYYNQSEAGSHTLQRMYG
CDVGPDGRLLRGHNQYAYDGKDYIALNEDLSSWTAADTAAQITQRKWEAA
RVAEQLRAYLEGLCVEWLRRYLENGKETLQRADPPKTHVTHHPISDHEAT
LRCWALGFYPAEITLTWQRDGEDQTQDTELVETRPAGDRTFQKWAAVVVP
SGEEQRYTCHVQHEGLPKPLTLRWEPSSQSTIPIVGIVAGLAVLAVVVIG
AVVATVMCRRKSSGGKGGSYSQAASSDSAQGSDVSLTA

### HLA-C

DNA sequence used for transfection/cell membrane expression:

(SEQ ID NO: 135)
atgcgcgtgatggcccctcgcaccttgattctgctcctgtccggtgccct
tgctctcaccgagacttgggcctgctcgcactccatgcggtacttctaca
ccgccgtgtctaggccgggaaggggagagccgcggttcatcgccgtgggc
tacgtcgacgatacccagtttgtgcggttcgacagcgatgccgcaagccc
cagaggagagcctagagctccgtgggtcgaacaggagggccccgagtact
gggaccgggaaacccagaagtacaagcgccaggcacaagcggatagagtg
tccctgcggaacctccggggttactacaaccagtcagaagccggttccca
tactctgcaacggatgtacggttgcgaccttggacccgacggcagactgc
tccggggctacgatcagtccgcctacgacgggaaggactacattgccctg
aacgaggacctccggtcctggactgcggcagataccgcggcgcagatcac
ccagcgcaaatgggaagcggcccgagaagcagagcagctgagagcctatc
tggagggcacctgtgtcgagtggctgaggcgctatttggaaaacgggaag
gagactctgcagagagcggaacaccccaagactcacgtgacccaccaccc
tgtgtccgaccatgaagccaccctgcgctgttgggccctggggttctacc
ctgccgaaatcaccctgacctggcaaagggacggagaggaccagacccag
gacaccgaactggtggagactcggcccgctggagatgggaccttccaaaa
atgggccgctgtggtcgtgccatccggggaggaacaacggtacacttgcc
acgtgcagcatgagggactcccggaaccgctgacacttcgctgggaaccg
agcagccagccaacgatccccattgtgggcatcgtggccggactcgcagt
gctggccgtgctggctgtcctgggagccgtgatggccgtcgtgatgtgcc
ggcgcaagtcgtccggcggaaagggaggcagctgctcccaagccgcgtcc
tcgaattcggcccagggctcagacgaatcactgatcgcctgcaaggcg

The corresponding membrane-bound protein comprises the following sequence:

(SEQ ID NO: 136)
CSHSMRYFYTAVSRPGRGEPRFIAVGYVDDTQFVRFDSDAASPRGEPRAP
WVEQEGPEYWDRETQKYKRQAQADRVSLRNLRGYYNQSEAGSHTLQRMYG
CDLGPDGRLLRGYDQSAYDGKDYIALNEDLRSWTAADTAAQITQRKWEAA
REAEQLRAYLEGTCVEWLRRYLENGKETLQRAEHPKTHVTHHPVSDHEAT
LRCWALGFYPAEITLTWQRDGEDQTQDTELVETRPAGDGTFQKWAAVVVP
SGEEQRYTCHVQHEGLPEPLTLRWEPSSQPTIPIVGIVAGLAVLAVLAVL
GAVMAVVMCRRKSSGGKGGSCSQAASSNSAQGSDESLIACKA

### HLA-E

DNA sequence used for transfection/cell membrane expression:

(SEQ ID NO: 137)
atggtcgacgggacgttgctcctcctgttgtccgaagccctggcgcttac
tcaaacctgggccggatcgcactccctgaagtatttccacacctccgtgt
caagacccggtagaggcgaacctcggttcatctccgtgggctacgtggac
gacactcagttcgtgcgcttcgacaatgatgccgcatcgcctcggatggt
gcccagagccccttggatggaacaggagggatcggagtactgggaccgcg
agactcggtcagcccgcgatactgcccagattttccgcgtcaacctgagg
accctgaggggctactataaccagagcgaagcgggttcccataccctcca
atggatgcatggctgcgaactgggtccagacaggcgcttcttgcggggct
acgaacagtttgcctacgatggaaaggattacctgaccctcaacgaggac
ctccgctcttggaccgccgtggacactgcggcccaaatctccgaacagaa
gtcgaacgacgcctcagaggcggagcaccagcgagcttaccttgaggaca
cttgcgtggagtggctgcacaagtacctggaaaagggaaaggaaaccctc
ctgcacctggagcctcccaagacccatgtcacccaccacccgatctccga
ccacgaagctaccctccggtgttgggccctgggcttttacccggcggaaa
tcaccctgacgtggcagcaggacggggagggacacacccaggacaccgaa
ctggtggagactagaccagccggcgatgggactttccagaaatgggccgc
tgtggtagtgcccagcggcgaagaacagcggtacacatgccatgtgcaac
acgagggactgcccgagcctgtgacccttcggtggaagccggcctcacaa
ccgaccatcccgattgtcggaattatcgccggactggtgctgctcgggtc
cgtcgtgtccggagcagtggtggccgctgtgatctggcggaagaagtcca
gcggaggaaaggggggttcctactcgaaagcggagtggagcgatagcgca
cagggatccgagagccactcgctg

The corresponding membrane-bound protein comprises the following sequence:

(SEQ ID NO: 138)
GSHSLKYFHTSVSRPGRGEPRFISVGYVDDTQFVRFDNDAASPRMVPRAP
WMEQEGSEYWDRETRSARDTAQIFRVNLRTLRGYYNQSEAGSHTLQWMHG
CELGPDRRFLRGYEQFAYDGKDYLTLNEDLRSWTAVDTAAQISEQKSNDA
SEAEHQRAYLEDTCVEWLHKYLEKGKETLLHLEPPKTHVTHHPISDHEAT
LRCWALGFYPAEITLTWQQDGEGHTQDTELVETRPAGDGTFQKWAAVVVP
SGEEQRYTCHVQHEGLPEPVTLRWKPASQPTIPIVGIIAGLVLLGSVVSG
AVVAAVIWRKKSSGGKGGSYSKAEWSDSAQGSESHSL

### HLA-F

DNA sequence used for transfection/cell membrane expression:

(SEQ ID NO: 139)
atggcaccacggtcgttgctcctgctcctgtcgggcgctcttgccctcac
cgacacttgggccggcagccattctcttcggtacttctccaccgccgtca
gcagaccgggaaggggagaaccgcggtatatcgcggtggaatatgtggac
gatacccagttcctgcggttcgactccgatgccgcgattccaaggatgga
gcccagagaaccctgggtggagcaggaaggcccgcagtactgggaatgga
ccaccggctacgccaaggccaacgctcagaccgatagggggcgctgcgca
acctcttgcggcggtacaatcagtcagaagcgggttcccacacgctgcaa
gggatgaacggctgcgacatgggacctgacggtagactgctccgaggcta
ccaccaacacgcgtacgatggaaaggactacattagcctgaacgaggatc
tgcggtcctggactgcggccgacactgtggcccaaatcacccagcgcttt
tacgaggcagaagaatacgccgaagaattccgcacctacctggagggcga
atgcttggagcttctgcggcgctacctggaaaacgggaaggagactctgc
agagagccgaccctcccaaggcccatgtggcccaccaccctatctcggac
catgaggcgaccctgcggtgttgggccctggggttctacccggctgagat
taccctgacctggcaacgcgacggagaggagcagacccaggacaccgaac
tcgtggaaaccagaccggccggagatggaacattccagaaatgggccgct
gtcgtggtgccctccggagaggaacagcgctacacttgccacgtgcagca
cgagggactgccacagcccctgatcctgcgctgggagcagtcccctcaac
cgactatccctatcgtcggtatcgtggctggtctggtggtgctcggagcc
gtcgtgactggggcagtggtggcagccgtgatgtggcgcaagaagtcctc
agaccggaacaggggcagctactcccaagccgccgtc

The corresponding membrane-bound protein comprises the following sequence:

(SEQ ID NO: 140)
GSHSLRYFSTAVSRPGRGEPRYIAVEYVDDTQFLRFDSDAAIPRMEPREP
WVEQEGPQYWEWTTGYAKANAQTDRVALRNLLRRYNQSEAGSHTLQGMNG
CDMGPDGRLLRGYHQHAYDGKDYISLNEDLRSWTAADTVAQITQRFYEAE
EYAEEFRTYLEGECLELLRRYLENGKETLQRADPPKAHVAHHPISDHEAT
LRCWALGFYPAEITLTWQRDGEEQTQDTELVETRPAGDGTFQKWAAVVVP
SGEEQRYTCHVQHEGLPQPLILRWEQSPQPTIPIVGIVAGLVVLGAVVTG
AVVAAVMWRKKSSDRNRGSYSQAAV

### 1.3. ILT2/ILT4 Constructs (Fc Fusions):

ILT2 was expressed as a soluble ILT2 ECD-rabbit Fc fusion protein (signal peptide in bold, rabbit Fc underlined):

(SEQ ID NO: 141)
MTPILTVLICLGLSLGPRTHVQAGHLPKPTLWAEPGSVITQGSPVTLR
CQGGQETQEYRLYREKKTALWITRIPQELVKKGQFPIPSITWEHAGRY
RCYYGSDTAGRSESSDPLELVVTGAYIKPTLSAQPSPVVNSGGNVILQ
CDSQVAFDGFSLCKEGEDEHPQCLNSQPHARGSSRAIFSVGPVSPSRR
WWYRCYAYDSNSPYEWSLPSDLLELLVLGVSKKPSLSVQPGPIVAPEE
TLTLQCGSDAGYNRFVLYKDGERDFLQLAGAQPQAGLSQANFTLGPVS
RSYGGQYRCYGAHNLSSEWSAPSDPLDILIAGQFYDRVSLSVQPGPTV
ASGENVTLLCQSQGWMQTFLLTKEGAADDPWRLRSTYQSQKYQAEFPM
GPVTSAHAGTYRCYGSQSSKPYLLTHPSDPLELVVSGPSGGPSSPTTG
PTSTSGPEDQPLTPTGSDPQSGLGRHLEKTVAPSTCSKPTCPPPELLG
GPSVFIFPPKPKDTLMISRTPEVTCVVVDVSQDDPEVQFTWYINNEQV
RTARPPLREQQFNSTIRVVSTLPIAHQDWLRGKEFKCKVHNKALPAPI
EKTISKARGOPLEPKVYTMGPPREELSSRSVSLTCMINGFYPSDISVE
WEKNGKAEDNYKTTPAVLDSDGSYFLYSKLSVPTSEWQRGDVFTCSVM
HEALHNHYTQKSISRSPGK

The purified final protein sequence used for screening assays comprises the following sequence:

(SEQ ID NO: 142)
GHLPKPTLWAEPGSVITQGSPVTLRCQGGQETQEYRLYREKKTALWIT
RIPQELVKKGQFPIPSITWEHAGRYRCYYGSDTAGRSESSDPLELVVT
GAYIKPTLSAQPSPVVNSGGNVILQCDSQVAFDGFSLCKEGEDEHPQC
LNSQPHARGSSRAIFSVGPVSPSRRWWYRCYAYDSNSPYEWSLPSDLL
ELLVLGVSKKPSLSVQPGPIVAPEETLTLQCGSDAGYNRFVLYKDGER
DFLQLAGAQPQAGLSQANFTLGPVSRSYGGQYRCYGAHNLSSEWSAPS
DPLDILIAGQFYDRVSLSVQPGPTVASGENVTLLCQSQGWMQTFLLTK
EGAADDPWRLRSTYQSQKYQAEFPMGPVTSAHAGTYRCYGSQSSKPYL
LTHPSDPLELVVSGPSGGPSSPTTGPTSTSGPEDQPLTPTGSDPQSGL
GRHLEKTVAPSTCSKPTCPPPELLGGPSVFIFPPKPKDTLMISRTPEV
TCVVVDVSQDDPEVQFTWYINNEQVRTARPPLREQQFNSTIRVVSTLP
IAHQDWLRGKEFKCKVHNKALPAPIEKTISKARGQPLEPKVYTMGPPR
EELSSRSVSLTCMINGFYPSDISVEWEKNGKAEDNYKTTPAVLDSDGS
YFLYSKLSVPTSEWQRGDVFTCSVMHEALHNHYTQKSISRSPGK

ILT4 was expressed as a soluble ILT4 ECD-rabbit Fc fusion protein (signal peptide in bold, rabbit Fc underlined):

(SEQ ID NO: 143)
MTPIVTVLICLGLSLGPRTHVQTGTIPKPTLWAEPDSVITQGSPVTLS
CQGSLEAQEYRLYREKKSASWITRIRPELVKNGQFHIPSITWEHTGRY
GCQYYSRARWSELSDPLVLVMTGAYPKPTLSAQPSPVVTSGGRVTLQC
ESQVAFGGFILCKEGEEEHPQCLNSQPHARGSSRAIFSVGPVSPNRRW
SHRCYGYDLNSPYVWSSPSDLLELLVPGVSKKPSLSVQPGPVVAPGES
LTLQCVSDVGYDRFVLYKEGERDLRQLPGRQPQAGLSQANFTLGPVSR
SYGGQYRCYGAHNLSSECSAPSDPLDILITGQIRGTPFISVQPGPTVA
SGENVTLLCQSWRQFHTFLLTKAGAADAPLRLRSIHEYPKYQAEFPMS
PVTSAHAGTYRCYGSLNSDPYLLSHPSEPLELVVSGPSMGSSPPPTGP
ISTPAGPEDQPLTPTGSDPQSGLGRHLELEKTVAPSTCSKPTCPPPEL
LGGPSVFIFPPKPKDTLMISRTPEVTCVVVDVSQDDPEVQFTWYINNE
QVRTARPPLREQQFNSTIRVVSTLPIAHQDWLRGKEFKCKVHNKALPA
PIEKTISKARGQPLEPKVYTMGPPREELSSRSVSLTCMINGFYPSDIS
VEWEKNGKAEDNYKTTPAVLDSDGSYFLYSKLSVPTSEWQRGDVFTCS
VMHEALHNHYTQKSISRSPGK

The purified final protein sequence used for screening assays comprises the following sequence:

(SEQ ID NO: 144)
QTGTIPKPTLWAEPDSVITQGSPVTLSCQGSLEAQEYRLYREKKSASW
ITRIRPELVKNGQFHIPSITWEHTGRYGCQYYSRARWSELSDPLVLVM
TGAYPKPTLSAQPSPVVTSGGRVTLQCESQVAFGGFILCKEGEEEHPQ
CLNSQPHARGSSRAIFSVGPVSPNRRWSHRCYGYDLNSPYVWSSPSDL
LELLVPGVSKKPSLSVQPGPVVAPGESLTLQCVSDVGYDRFVLYKEGE
RDLRQLPGRQPQAGLSQANFTLGPVSRSYGGQYRCYGAHNLSSECSAP
SDPLDILITGQIRGTPFISVQPGPTVASGENVTLLCQSWRQFHTFLLT
KAGAADAPLRLRSIHEYPKYQAEFPMSPVTSAHAGTYRCYGSLNSDPY
LLSHPSEPLELVVSGPSMGSSPPPTGPISTPAGPEDQPLTPTGSDPQS
GLGRHLELEKTVAPSTCSKPTCPPPELLGGPSVFIFPPKPKDTLMISR
TPEVTCVVVDVSQDDPEVQFTWYINNEQVRTARPPLREQQFNSTIRVV
STLPIAHQDWLRGKEFKCKVHNKALPAPIEKTISKARGQPLEPKVYTM
GPPREELSSRSVSLTCMINGFYPSDISVEWEKNGKAEDNYKTTPAVLD
SDGSYFLYSKLSVPTSEWQRGDVFTCSVMHEALHNHYTQKSISRSPGK

### Expression and Purification of ILT2rbFc and ILT4rbFc

The proteins were expressed by transient transfection using the Expi293™ HEK Expression System (Life Technologies™) following manufacturers protocol. Cells were harvested 5 days post transfection and supernatants used immediately for purification.

Supernatants comprising ILT2rbFc or ILT4rbFc protein was applied to Hitrap Protein A column. Unbound protein and contaminants were washed with PBS and ILT2rbFc or ILT4rbFc protein eluted with endo free 0.1M citric acid buffer, pH 2 and the peak fractions were neutralised with 0.5 ml 2M Tris PH 8. Fractions containing purified protein were pooled and concentrated and purified further by size exclusion chromatography on S200 26/60 using Cytiva HyClone™ Phosphate Buffered Saline (PBS) as the running buffer. Fractions containing purified ILT2rbFc or ILT4rbFc protein were pooled, concentrated and aliquoted before storing at −80° C.

### Example 2: Generation of Antibodies by Immunization with HLA-G

Because of the specific challenges associated with the production of antibodies against HLA-G (such as the high homology with other HLA-I molecules, the identification of antibodies able to block the interaction between HLA-G and its inhibitory receptors) and in order to identify antibodies that would be useful in therapy, a special discovery strategy including a special screening and testing strategy had to be developed and is described below.

### Immunization and Screening Strategy

A number of animals across different species (including mice and rabbits) were immunized with syngeneic cells expressing different forms of HLA-G with or without B2m co-expression.

Following 3-5 shots, the animals were sacrificed and PBMC, spleen, bone marrow and lymph nodes harvested. Sera was monitored for binding to the immunogen used.

Memory B cell cultures were set up and supernatants were first screened for their ability to bind HLA-G above an irrelevant control in a multiplexed no-wash assay either on the TTP Labtech Mirrorball system (plate reader) or the Intellicyt iQue (flow cytometry). Cultures were screened on an irrelevant control protein, in-house generated HLA-G proteins and/or using EXPI293 HEKs transiently expressing the constructs of interest on the cell surface (HLA-G, HLA-G Null 1,2,3, HLA-G Null3). Protein reagents were biotinylated to enable streptavidin capture to beads. A fluorescently labelled species-specific anti-Fc secondary antibody was used for the assays to detect the test antibody.

Approximately 3800 HLA-G-specific positive hits were identified in the primary screens from a total of 18 B cell culture experiments each containing 100-300 plates. Positive supernatants from the primary screens were then progressed for further characterization in binding assays (soluble isolated alpha 3 domain null, and cell-expressed isolated alpha 3 domains, HLA-G Null 1,3, HLA-G Null3 2AA).

Wells with desirable profiles were progressed for variable V region recovery using the fluorescent foci method and binding to the HLA-G ECD protein.

In parallel, plasma cells from the bone marrow and Lymph node were also directly screened for their ability to bind human HLA-G or specifically the 3 domain using the fluorescent foci method. Here, B cells secreting HLA-G specific antibodies were picked on biotinylated human HLA-G ECD or isolated wild-type HLA-G α3 domain immobilized on streptavidin beads. A goat anti-species Fc-FITC conjugate reveal reagent was used. Approximately 1700 direct foci were picked.

Following reverse transcription (RT) and PCR of the picked cells, ‘transcriptionally active PCR’ (TAP) products encoding the antibodies' V regions were generated and used to transiently transfect EXPI293 HEK cells. The resultant TAP supernatants, containing recombinant antibody, were tested in the following assays:
Cell binding to HLA-G and null mutants as described above to help establish domain binding (multiplexed iQue)
ILT2 Blocking assay
Measurement of affinity by Biacore

Heavy and light chain variable region gene pairs from interesting TAP products were then cloned as species matched full length IgG antibodies and re-expressed in a transient expression system. Recombinant cloned antibodies were then retested in the assays above.

In total 109 V (variable) regions were cloned and registered, only 30 of these were specific to HLA-G and did not bind other HLA-Is amongst which only 9 showed blocking of the HLA-G ILT2 interaction. All of these antibodies bound the alpha 3 domain of HLA-G and 5 of the 9 specific and blocking antibodies had diverse sequences and were taken forward for further testing (listed in Table 3 below).

TABLE 3
Antibodies generated by immunisation selected for further analysis

| Antibody ID | Derivation/Immunogen |
|---|---|
| HLA-G01 | Direct foci, bone marrow/ |
| | Rab9 cells + hHLA-G |
| HLA-G02 | B cell culture, spleen/ |
| (VR12389 gL2gH16) | Rab9 cells + hHLA-G |
| HLA-G03 | B cell culture, lymphoid node/ |
| | Rab9 cells + hHLA-G |
| HLA-G04 | B cell culture, lymphoid node/ |
| | Rab9 cells + hHLA-G + B2m |
| HLA-G05 | Direct foci, bone marrow/ |
| | Rab9 cells + hHLA-G |

Humanization campaigns were conducted for some antibodies based on their properties, and further assessed in the characterization assays, including HLA-G02 and HLA-G01 that appeared to be the best antibodies. Humanization of VR12389 is described in Example 4. HLA-G03 was also humanized, nevertheless, the high affinity of the humanized antibody did not translate into improved functional activity as will be described in further Examples.

Additional assays performed on the purified antibodies included Cell based specificity assays, ILT4 blocking assay, ADCC. The data generated for the purified IgG1 antibodies are described in further Examples below.

In the present disclosure, antibody ID HLA-G02 refers to the humanised VR12389gL2gH16 IgG1 antibody.

Method of Immunisation that LED to the Discovery of the Antibody 12389

### Rab9 Cells Transiently Expressing HLA-G

Rab9 fibroblast cells were cultured in RPMI media+10% FBS and 1% glutamine in a 5-stack cell culture flask. When cells were 90-100% confluent, media was removed, cells washed with 100 ml PBS and cells were removed from the cell culture flask using 100 ml Accutase and 10-15-minute incubation at room temperature. Harvested cells were spun down and resuspended at 5×10⁷ cells/ml in Earles Balanced Salt buffer. HLA-G DNA was added to the cells at 250 μg DNA per ml cells. Rab9+HLA-G DNA mix was then transferred to electroporation cuvettes at 3×10⁷ cells/cuvette. Cuvettes were then pulsed with 150-170V electricity (20 ms 5.5 Amps) using our in-house electroporator device (Zapper) and a Gene Pulser Xcell ShockPod Cuvette Chamber (BIORAD.) Following electric pulse, cells were transferred quickly to warm Rab9 media and placed back into a fresh 5-stack cell culture flask.

Following electroporation of all cells and transfer into a new culture flask, they were then incubated for 24 hours at 37° C., 5% CO2 before cells were harvested using Accutase (as described before), counted, and frozen down in a −80 freezer into cryovials at 2×10⁷ cells per cryovial. After 24 hours, frozen cells were transferred to a liquid nitrogen dewar for longer term storage.

Prior to freezing, 5×10⁵ transfected cells were tested for expression of HLA-G by staining for 1 hour at 4° C. with Sigma APC conjugated anti-HLA-G antibody (clone MEM/G9) and running through samples on a FACS Calibre.

On day of immunisation, for each shot, 1 vial of transfected cells was defrosted rapidly at 37° C. and washed twice in 50 ml PBS prior to resuspension in 500 μl for administration into the rabbits.

The DNA sequence coding for the full-length HLA-G (SEQ ID NO: 111) was used for electroporation (nucleic sequence of HLA-G optimized for the expression in mammalian cells).

### Immunisation

One female New Zealand White rabbit was immunised sub-cutaneously with 2×10⁷ Rab9 rabbit fibroblast cells transiently expressing HLA-G on the cell surface prepared as described above. An equal volume of complete Freunds adjuvant was injected sub-cutaneous into the rabbit at a separate site but at the same time as immunisation with cells.

The rabbit was given two booster injections at 14-day intervals with the Rab9 rabbit fibroblast cells transiently expressing HLA-G on the cell surface. Heparinised bleeds (200 μl) were taken from the ear vein prior to each immunisation. Sera was collected from the bleeds after spinning 10,000 rpm for 5 minutes in a bench top centrifuge and frozen down at −20° C. Termination occurred 14 days after the final boost with single cell suspensions of spleen, lymph node, bone marrow and peripheral blood mononuclear cells prepared and frozen in 10% DMSO/FCS at −80° C. until required for B cell discovery purposes. A bleed was also taken at termination and sera prepared as previously described.

### B Cell Recovery and Screening

VR12389 was discovered from memory B cell culture of spleen. Spleen cells were cultured with a feeder cell line and supplements at 37 C for 5 days in 96 well plates. This culture was then screened via a no-wash multiplexed flowcytometry assay (Intellicyt iQue). Culture supernatant containing secreted antibody was mixed with the screening reagents as described above (in-house generated HLA-G proteins and cell-expressed HLA-G constructs: HLA-G, HLA-G Null 1,2,3, HLA-G Null3). The screening cells used in the screen were differentially stained to allow gating of the different populations and a Dylight 405 labelled goat anti-Rabbit antibody was used as the secondary antibody to identify antibody binding.

Hits were defined as HLA-G binders which were specific i.e. did not bind to the HLA-G Null 1,2,3 mutant or the irrelevant control transfection. B cells responsible for the hits were recovered going back to the original culture wells then using the fluorescent foci method as previously described.

The picked cells for both leads followed the same workflow after the foci step. Reverse transcription (RT) and PCR of the picked cells led to ‘transcriptionally active PCR’ (TAP) products encoding the antibodies' V regions which were used to transiently transfect EXPI293 HEK cells. The resultant TAP supernatants, containing recombinant antibody, were characterized for cell binding, ILT2 blocking and affinity, before progressing to clone the antibodies and express at a larger scale.

As mentioned above, a number of animals across different species (including rats, mice and rabbits) were immunized with syngeneic cells expressing different forms of HLA-G with or without B2m co-expression, but not all the immunisation strategies were successful for the production of antibodies against HLA-G, or, when the production of antibodies against HLA-G was confirmed, the antibodies were not specific to HLA-G and/or not blocking, or were unable to bind cell surface expressed HLA-G protein. Of note, the immunisation of rabbits with Rab9 cells expressing the isolated alpha 3 domain, or expressing a rabbit-human HLA-G chimera, did not raise any antibodies against HLA-G. Therefore, the present disclosure provides a method of immunisation that is particularly useful for the discovery of anti-HLA-G antibodies useful in therapy, said method comprising the immunisation of a rabbit with Rab9 rabbit fibroblast cells transiently expressing the full-length sequence of HLA-G on the cell surface.

### Example 3: Generation of Antibodies by Phage Display

In order to identify antibodies that would be useful in therapy, a second approach was developed in parallel of the immunization campaigns to try to identify antibodies specifically binding to HLA-G that would be useful in therapy, from phage display libraries. Again, because of the specific challenges associated with the production of HLA-G antibodies useful in therapy, a special screening and testing strategy was developed.

### Phage Display Libraries

Three human naïve combinatorial scFv phage libraries were utilised to obtain antibodies that bound HLA-G using different constructs with the aim to obtain selective binders to HLA-G with non or minimal binding to other HLA-Is. The libraries were bio-panned using three or four rounds of selection using recombinant HLA-G expressed on cell-surface only or followed by recombinant HLA-G extracellular domain protein in the final round. An optional subtraction step on HLA-G Null 1,2,3 (soluble protein or cell-expressed) was included in the final round to enrich for HLA-G-specific binders.

Briefly, the bio-panning on cells consisted in co-transfecting DNA constructs encoding human HLA-G and β2m in ExpiCHOs for the first round or Expi293 HEKs for subsequent rounds and incubating these cells with blocked phage virions previously depleted on non-transfected or HLA-G Null 1,2,3-transfected cells. The bio-panning on protein was performed by incubating blocked phage particles with either plate-coated HLA-G, or with biotinylated HLA-G in solution followed by a capture on streptavidin or neutravidin magnetic beads. After several washes using PBS Tween, the target-bound phages were eluted and re-amplified by infecting E. coli TG1s.

### Phage Screening

After the last round of selection, 1692 monoclonal rescued phages were screened by ELISA on biotinylated HLA-G ECD captured on streptavidin-coated plates. Binding was detected by an HRP-conjugated anti-M13 pVIII coat protein antibody. Biotinylated HLA-G Null 1,2,3 was used to assess the specificity of these monoclonal phage clones. The 359 binders of interest were sequenced, and the diversity was analysed based on their variable heavy chain CDR3 sequence motif. 81 unique clones were then reformatted as scFv-rabbit IgG Fc fusions in a mammalian expression vector for further characterisation.

### Further Characterisation of HLA-G-Selective Binders

The scFv-Fcs were expressed in Expi293 HEKs. Their binding and specificity were tested by flow cytometry using an IntelliCyt iQue Screener Plus. Diluted antibody-containing supernatants were added to ExpiHEKs co-transfected with human HLA-G or HLA-G and β2 microglobulin. Binding was detected with an Fc-fragment-specific fluorescent antibody.

The 21 HLA-G cell binders that didn't bind HLA-G Null 1,2,3+B2m were further characterised as scFv-Fcs and/or after reformatting into full length human IgG1s by SPR, ILT2 blocking assay, binding to HLA-A, -B, -C, -E, -F expressed on HEKs and binding to JEG3 cells by flow cytometry.

14 antibodies were confirmed to be highly specific to HLA-G with no or minimal binding to other HLA-I molecules, amongst which, only 6 antibodies were found to block the interaction between HLA-G and ILT2.

Amongst the 6 specific and blocking antibodies, 3 had diverse sequences and were taken forward for further testing (HLA-G06, HLA-G07, HLA-G08).

Additional assays performed on the purified antibodies included Cell based specificity assays, ILT4 blocking assay, ADCC. The data generated for the purified antibodies are described in further Examples below.

### Example 4: Antibody 12389 Humanization

Antibody 12389 was humanized by grafting the CDRs from the rabbit V-region onto human germline antibody V-region frameworks. In order to recover the activity of the antibody, a number of framework residues from the rabbit V-region were also retained in the humanized sequence. These residues were selected using the protocol outlined by Adair et al. (1991) (WO91/09967). Alignments of the rabbit antibody (donor) V-region sequences with the human germline (acceptor) V-region sequences are shown in FIGS. 2 and 3, together with the designed humanized sequences. The CDRs grafted from the donor to the acceptor sequence are as defined by Kabat (Kabat et al., 1987), with the exception of CDR-H1 where the combined Chothia/Kabat definition is used (see Adair et al., WO91/09967).

For antibody 12389, the human V-region IGKV1D-13 plus IGKJ4 J-region (IMGT, http://www.imgt.org/) was chosen as the acceptor for the light chain CDRs. The light chain framework residues in the humanized graft variants are all from the human germline gene, with the exception of none, one or two residues from the group comprising residues 3 and 70, where the donor residues Valine (V3) and Glutamine (Q70) with reference to SEQ ID NO: 7 (rabbit VL) were retained, respectively.

Human V-region IGHV3-66 plus IGHJ4 J-region (IMGT, http://www.imgt.org/) was chosen as the acceptor for the heavy chain CDRs of antibody 12389. In common with many rabbit antibodies, the VH gene of antibody 12389 is shorter than the selected human acceptor. When aligned with the human acceptor sequence, framework 1 of the VH region of antibody 12389 lacks the N-terminal residue, which is retained in the humanized antibody (FIG. 3). Framework 3 of the 12389 rabbit VH region also lacks two residues (75 and 76, with reference to SEQ ID NO:11, rabbit VH) in the loop between beta sheet strands D and E: in the humanized graft variants the gap is filled with the corresponding residues (Lysine 75, K75; Asparagine 76, N76) from the selected human acceptor sequence. The heavy chain framework residues in the humanized graft variants are all from the human germline gene, with the exception of one or more residues from the group comprising residues 24, 48, 49, 71, 73, 78 and 96, where the donor residues Valine (V24), Isoleucine (I48), Glycine (G49), Lysine (K71), Serine (S73), Valine (V78) and Glycine (G96) with reference to SEQ ID NO: 11 were retained, respectively.

The variant humanized antibody chains, and combinations thereof, were expressed and assessed for their binding affinity for human HLA-G relative to the parent antibody.

### Expression in Expi293 Cells

Genes encoding variant heavy and light chain V-region sequences were designed and constructed by an automated synthesis approach by ATUM (Newark, CA). For transient expression in mammalian cells, the humanized light chain V-region genes were cloned into a light chain expression vector pMhCK, which contains DNA encoding the human Kappa chain constant region (Km3 allotype). The humanized heavy chain V-region genes were cloned into a human gamma-4 heavy chain expression vector pMhg4PFL, which contains DNA encoding the human gamma-4 heavy chain constant region with the hinge stabilising mutation S228P (Angal S., King D. J., Bodmer M. W., Turner A., Lawson A. D. G., Roberts G., Pedley B. and Adair J. R. A single amino acid substitution abolishes the heterogeneity of chimeric mouse/human (IgG4) antibody. Mol. Immunol. 1993, 30 (1):105-8), or into a gamma-1 heavy chain expression vector pMhg1FL, which contains DNA encoding the human gamma-1 heavy chain constant region (G1m17, 1 allotype). Co-transfection of the resulting heavy and light chain vectors into Expi293™ suspension cells was achieved using ExpiFectamine™ 293 transfection reagent (A14525, ThermoFisher Scientific), and gave expression of the humanized, recombinant IgG4P and IgG1 antibodies.

### Affinity Measurement by SPR

As described below, the assay format was capture of the anti-HLA-G IgG by an immobilised anti-human IgG Fc-specific antibody, followed by titration of HLA-G over the captured surface.

The affinity of anti-HLA-G IgG binding HLA-G was determined by Surface Plasmon Resonance using a Biacore T200 (GE Healthcare Biosciences AB). Assays were performed at 25° C. Affinipure F(ab′)₂ fragment goat anti-human IgG, Fc specific (Jackson ImmunoResearch) was immobilised on a Series S CM5 Sensor Chip (GE Healthcare Bio-Sciences AB) via amine coupling chemistry to a level of approximately 6000 response units (RU). HBS-EP⁺ buffer (10 mM HEPES pH7.4, 0.15M NaCl, 3 mM EDTA, 0.05% Surfactant P20, GE Healthcare Bio-Sciences AB) was used as the running buffer with a flow rate of 10 μL/min. A reference surface was prepared by activating and deactivating the appropriate flow cell.

A 10 μL injection of anti-HLA-G IgG at concentrations from 0.15-0.7 μg/mL was used for capture by the immobilised anti-human IgG, Fc. Human HLA-G ECD+B2m was titrated over the captured anti-HLA-G IgG at 50 nM at a flow rate of 30 μL/min for 60 s followed by dissociation for 150 s. The surface was regenerated at a flow rate of 10 μL/min by a 10 μL injection of 40 mM HCl followed by a 5 μL injection of 5 mM NaOH.

Background subtraction binding curves were analysed using the Biacore T200 Evaluation Software (Version 3.0) using 1:1 binding fitted with local Rmax.

The antibodies were analysed at the start and end of the assay and showed good precision. High quality data was generated for all samples as summarized in Tables 4 and 5.

TABLE 4
Affinity data for the 12389 (chimeric rabbit V regions/human
Fc) and humanized grafts expressed as hIgG4P

| | Light | Heavy | | | |
|---|---|---|---|---|---|
| | chain | chain | | | Affinity |
| Antibody | Donor | Donor | kₐ | k_{d} | (KD) |
| 12389 | residues | residues | (M⁻¹s⁻¹) | (s⁻¹) | nM |
| 12389 | — | — | 9.49E+05 | 9.20E−03 | 9.69 |
| (chimeric | | | | | |
| rabbit V | | | | | |
| regions/human | | | | | |
| Fc) | | | | | |
| 12389gL1gH1 | V3, Q70 | V24, I48, | 8.48E+05 | 6.78E−03 | 7.99 |
| | | G49, K71, | | | |
| | | S73, V78, | | | |
| | | G93 | | | |
| 12389gL2gH1 | V3 | V24, I48, | 9.28E+05 | 7.08E−03 | 7.63 |
| | | G49, K71, | | | |
| | | S73, V78, | | | |
| | | G93 | | | |
| 12389gL3gH1 | — | V24, I48, | 8.45E+05 | 6.97E−03 | 8.25 |
| | | G49, K71, | | | |
| | | S73, V78, | | | |
| | | G93 | | | |
| 12389gL2gH4 | V3 | I48, G49, | 8.88E+05 | 7.18E−03 | 8.09 |
| | | K71, S73, | | | |
| | | V78, G93 | | | |
| 12389gL2gH5 | V3 | V24, G49, | 8.17E+05 | 1.01E−02 | 12.4 |
| | | K71, S73, | | | |
| | | V78, G93 | | | |
| 12389gL2gH6 | V3 | V24, I48, | 8.39E+05 | 1.04E−02 | 12.5 |
| | | K71, S73, | | | |
| | | V78, G93 | | | |
| 12389gL2gH8 | V3 | V24, I48, | 7.87E+05 | 1.21E−02 | 15.4 |
| | | G49, S73, | | | |
| | | V78, G93 | | | |
| 12389gL2gH9 | V3 | V24, I48, | 8.50E+05 | 6.03E−03 | 7.09 |
| | | G49, K71, | | | |
| | | V78, G93 | | | |
| 12389gL2gH11 | V3 | V24, I48, | 8.50E+05 | 9.07E−03 | 10.7 |
| | | G49, K71, | | | |
| | | S73, G93 | | | |
| 12389gL2gH12 | V3 | V24, I48, | 8.44E+05 | 6.24E−03 | 7.39 |
| | | G49, K71, | | | |
| | | S73, V78 | | | |
| 12389gL2gH13 | V3 | I48, G49, | 9.56E+05 | 6.32E−03 | 6.61 |
| | | K71, V78, | | | |
| | | G93 | | | |
| 12389gL2gH14 | V3 | I48, G49, | 8.75E+05 | 6.64E−03 | 7.58 |
| | | K71, S73, | | | |
| | | V78 | | | |
| 12389gL2gH15 | V3 | V24, I48, | 8.96E+05 | 5.67E−03 | 6.33 |
| | | G49, K71, | | | |
| | | V78 | | | |
| 12389gL2gH16 | V3 | I48, G49, | 9.02E+05 | 5.46E−03 | 6.06 |
| | | K71, V78 | | | |
| 12389gL3gH16 | — | I48, G49, | 8.33E+05 | 5.60E−03 | 6.72 |
| | | K71, V78 | | | |

TABLE 5
Affinity data for the 12389 (chimeric rabbit V regions/human
Fc) and 12389gL2gH16 expressed as hIgG1, in two experiments

| | Light | Heavy | | | |
|---|---|---|---|---|---|
| | chain | chain | | | Affinity |
| Antibody | Donor | Donor | kₐ | k_{d} | (KD) |
| 12389 | residues | residues | (M⁻¹s⁻¹) | (s⁻¹) | nM |
| 12389(chimeric | — | — | 8.41E+05 | 9.10E−03 | 10.80 |
| rabbit V | | | | | |
| regions/human | | | | | |
| Fc) | | | | | |
| 12389gL2gH16 | V3 | I48, G49, | 7.29E+05 | 3.48E−03 | 4.78 |
| IgG1 | | K71, V78 | | | |
| 12389(chimeric | — | — | 7.82E+05 | 9.18E−03 | 11.70 |
| rabbit V | | | | | |
| regions/human | | | | | |
| Fc) | | | | | |
| 12389gL2gH16 | V3 | I48, G49, | 6.72E+05 | 3.64E−03 | 5.42 |
| IgG1 | | K71, V78 | | | |

As shown in Table 4, all the grafts, except 12389gL2gH5, 12389gL2gH6, 12389gL2gH8, 12389gL2gH11, had a KD of less than 10 nM, and less than the KD measured for the parent 12389 (chimeric rabbit V regions/human Fc).

Graft 12389gL2gH16, which retained VH framework donor residues 148, G49, K71 and V78, and VL framework donor residue V3, had the highest affinity binding to human HLA-G, as measured by surface plasmon resonance, and retained functionality when expressed in different formats, such as IgG4P and IgG1 (Tables 4-5), and was selected for further characterization.

### Example 5. Expression and Purification of HLA-G01-HLA-G08

The antibodies were transiently expressed as IgG1 in CHO cells transfected with a DNA vector coding for the LC and HC of the HLA-G01-HLA-G08 antibodies (1:1 ratio of LC:HC) and purified on a protein A affinity chromatography according to well-known methods for further testing.

Protein concentration was determined by reading absorbance at 280 nm using a nanodrop and purity was determined by analytical size exclusion HPLC. Monomer content was determined using analytical size exclusion chromatography and SDS Page electrophoresis. Endotoxin level was determined using Charles River Endosafe® LAL Reagent Cartridge Technology and Endosafe® nexgen-PTS reader, with a level of <1 EU/ml being of acceptable quality.

Final Purified sample was highly pure and contained >98% monomer content. Final Purified sample were analysed by intact Mass Spectrometry to confirm heavy and light chain masses, expected modifications and identity.

### Example 6: Production of Afucosylated HLA-G02 Antibody

### Method of Producing KO FUT8 CHOSXE/DG44 Cells

RNA guides (gRNAs) were designed to knockout 2 exons comprising the sequence coding for alpha1,6 fucosyltransferase (FUT8) active site. gRNAs 2-8 (7 in total) were designed using Benchling software to make multiple deletions in both forward and reverse strands, the largest possible deletion being 4 kb, guides were used as a pool to maximise possible knockouts. The sequence of the gRNAs is provided in the Table 6 below.

TABLE 6
gRNA sequences

| Name | Sequence |
|---|---|
| FUT8-gRNA-2 | GAUGGAGGCUGUCUACAAUG |
| FUT8-gRNA-3 | GUCAGGGCUGUAGCACACUG |
| FUT8-gRNA-4 | GAAGUGGUAGUAACUUUACA |
| FUT8-gRNA-5 | AUUAGUAUCCCUAGUCAUGG |
| FUT8-gRNA-6 | UGGUACACCUAGUACUACUG |
| FUT8-gRNA-7 | UGACUAUACAAAUUUCUGGG |
| FUT8-gRNA-8 | AGUCAACAAUGUCUUAGACA |

gRNA pools were prepared at final pmol concentrations, as provided in Table 7. Cas09 (ThermoFisher) was prepared to the required concentration as provided in Table 7.

3×10⁶ CHO SXE/DG44 cells were prepared for nucleofection, after centrifugation at ×100 g for 8 minutes, the cells were washed in PBS, centrifuged again at ×100 g for 8 minutes and resuspend in 100 μl nucleofection solution to give 3×10⁴/μl of cells. Pre-transfection mixes were prepared as shown in the Table 7 below, left without cells for 10-60 minutes at room temperature to allow Cas09/gRNA complexes to form.

TABLE 7
Pre-transfection mixes

| | 9:1 ratio | | 6:1 ratio | | 3:1 ratio | | negative | | Mock |
|---|---|---|---|---|---|---|---|---|---|
| Nucleofection sol | 60 | μl | 60 | μl | 60 | μl | 60 | μl | 60 μl |
| gRNA | 20 μl | | 20 μl | | 20 μl | | — | | — |
| | (90 pmol) | | (60 pmol) | | (30 pmol) | | | | |
| Cas09 | 3.33 | μl | 3.33 | μl | 3.33 | μl | 3.33 | μl | — |
| (3.22 mg/ml) | | | | | | | | | |

16.67 μl cells (3×10⁴ cells/μl) were added to the complexes, before proceeding with nucleofection (nucleofector 4D, Lonza), as manufacturers' instructions. The cells were recovered with addition of 900 μl pre-warmed CD CHO media, placed in small T25 flasks stood vertically. Left for 24 hours 37° C., 5% CO₂, the media was replace with fresh prewarmed CD CHO (antibiotics penicillin, streptomycin, and Amphotericin B were added to reduce the risk of contamination) once the cells had recovered and were dividing they were moved to a 125 ml shake flask (96-120 hours later).

10 days after nucleofection the cells were ready to be sorted by FACS. Cells were stained with LCA stain (Lens Culinaris Agglutinin conjugated to Fluorescein). The cells were prepared for FACS, by centrifugation at ×100 g for 8 minutes, the cells were washed with PBS, centrifuged again at ×100 g for 8 minutes and resuspend in prewarmed CD CHO media, 20 μg/ml LCA stain was added, left 45 mins, the cells were washed ×2 PBS (centrifugation at ×100 g for 8 minutes, resuspension in PBS) to remove any unbound stain. LCA bound Fucose on the cell surface of FUT8 positive cells, FUT8 negative (knock out cells) were not stained and collected into pre-warmed CD CHO media. The cells were place at 37° C., 5% CO2 to recover and passage on until the cells reached the required cell density.

### Production of Afucosylated HLA-G Antibodies

HLA-G02 antibody constructs were expressed in an engineered CHO-SXE cell line (Cain et al 2012) that has been further modified to have the α-1,6-fucosyltransferase enzyme (FUT8) knocked down as described above. The ExpiCHO transfection system (Thermo Fisher Scientific) was used to transiently generate afucosylated antibodies from these cells. Following the high yield protocol, cells were seeded at a cell density of 6×10\hat{ }6 cells/ml in ExpiCHO expression media. For a 200 ml culture, 200 μg DNA was diluted into 8 ml Opti-PRO serum free media (SFM) and mixed with 7.4 ml Opti-PRO SFM containing 640 μl ExpiFectamine transfection reagent before adding to cells. Cells were transferred to an incubator set to 8% CO₂ and 37° C., on a shaking platform set to 190 RPM, On day 1 post-transfection, 48 ml feed and 1200 μl enhancer was added to the cells, and returned to the incubator, reducing the temperature to 32° C. Cultures were harvested on day 10 by centrifugation for 1 hour at 4 000 RPM. Cell culture supernatants were clarified by application to a 0.22 μM Stericup Filter.

Product titre was determined by loading 100 μl of supernatant onto a protein G column attached to an Infinity high-performance liquid chromatography (HPLC) system. Product was eluted off the column with 150 mM sodium chloride, pH 2.1 and the A280 signal was compared to a purified Fab standard. Clarified supernatant was loaded onto a MabSelect Sure column (GE Healthcare) at 5 ml/min and washed with 3 column volumes (CVs) PBS pH7.4. Captured protein was eluted off the column with a low pH buffer, 0.1 M sodium Citrate buffer pH 3.6. Eluate was neutralised with 2 M Tris-HCl pH 8.5. In order to remove high molecular weight species, the affinity purified protein was loaded onto Superdex 16/60 gel filtration chromatography column equilibrated in 10 mM phosphate buffered saline, pH 7.4 at 1 ml/min and eluted fractions were analysed by analytical SE-UPLC prior to pooling appropriate fraction. Pooled fractions were subjected to SDS-PAGE and SE-UPLC to determine protein quality and purity.

In the present disclosure, unless otherwise specified, “HLA-G02” refers to the unmodified, conventional (i.e. fucosylated) VR12389gL2gH16 IgG1. “Afucosylated HLA-02” refers to the corresponding afucosylated IgG1 antibody as produced according to the methods described herein.

### Example 7: Binding, Affinity and Specificity of the HLA-G Antibodies

### 7.1. Affinity for HLA-G WT Measured by SPR

The binding affinity of anti-HLA-G antibodies (hIgG1 format) for HLA-G was determined by Surface Plasmon Resonance using a Biacore T200 (GE Healthcare Biosciences AB). Assays were performed at 25° C. Affinipure F(ab′)2 fragment goat anti-human IgG, Fc specific (Jackson ImmunoResearch) was immobilised on a Series S CM5 Sensor Chip (GE Healthcare Bio-Sciences AB) via amine coupling chemistry to a level of approximately 6000 response units (RU). HBS-EP⁺ buffer (10 mM HEPES pH7.4, 0.15M NaCl, 3 mM EDTA, 0.05% Surfactant P20, GE Healthcare Bio-Sciences AB) was used as the running buffer with a flow rate of 10 μL/min. A reference surface was prepared by activating and deactivating the appropriate flow cell.

A 10 μL injection of anti-HLA-G antibodies at concentrations from 0.6-0.9 μg/mL was used for capture by the immobilised anti-human IgG, Fc. Human HLA-G ECD+B2m was titrated over the captured anti-HLA-G hIgG1 from various top concentrations 4000 nM, 400 nM, 100 nM and 50 nM at a flow rate of 30 μL/min for 60 s, 90 s or 120 s followed by dissociation for 120 s, 180 s or 240 s (Table 8).

TABLE 8

| | | Concentration | Contact | Dissociation |
|---|---|---|---|---|
| Capture | Sample | (nM) | (s) | (s) |
| Both conventional | HLA-G | 50 | 90 | 180 |
| HLA-G02, and | | | | |
| afucosylated | | | | |
| HLA-G02 | | | | |
| HLA-G01, HLA-G03, | HLA-G | 100 | 90 | 180 |
| HLA-G05 | | | | |
| HLA-G04 | HLA-G | 400 | 120 | 240 |
| HLA-G06, HLA-G07, | HLA-G | 4000 | 60 | 120 |
| HLA-G08 | | | | |

The results are presented in Table 9.

TABLE 9
Affinity of the HLA-G antibodies as determined by SPR

| | kₐ | k_{d} | KD |
|---|---|---|---|
| Antibody ID | (M⁻¹s⁻¹) | (s⁻¹) | (nM) |
| HLA-G01 | 5.85E+05 | 3.35E−03 | 5.72 |
| HLA-G02 | 6.18E+05 | 3.15E−03 | 5.09 |
| (VR12389gL2gH16) | | | |
| HLA-G06 | 3.09E+05 | 7.91E−02 | 256 |
| HLA-G03 | 4.04E+05 | 2.34E−04 | 0.58 |
| HLA-G07 | 4.39E+05 | 2.00E−01 | 456 |
| HLA-G04 | 5.61E+04 | 4.33E−04 | 7.72 |
| HLA-G05 | 2.00E+05 | 1.40E−03 | 6.98 |
| HLA-G08 | 2.19E+05 | 7.66E−02 | 349 |

HLA-G06, HLA-G07 and HLA-G08 had the lowest affinity (higher KD value) for HLA-G as determined by SPR. HLA-G01, HLA-G02, HLA-G03 had the highest affinity for HLA-G.

In another assay, the affinity of the afucosylated HLA-G02 was also assessed and found similar to the conventional (fucosylated) HLA-G02 counterpart. The results are presented in Table 10 below.

TABLE 10
Affinity of the conventional and afucosylated
HLA-G02 antibody by SPR

| | kₐ | k_{d} | KD |
|---|---|---|---|
| Antibody ID | (M⁻¹s⁻¹) | (s⁻¹) | (nM) |
| HLA-G02 | 8.24E+05 | 3.54E−03 | 4.30 |
| (conventional, i.e. | | | |
| fucosylated) | | | |
| Afucosylated HLA-G02 | 8.07E+05 | 3.55E−03 | 4.40 |

### 7.2. Affinity for HLA-G Null 1,2,3 Measured by SPR to Assess Specificity

The binding affinity of anti-HLA-G IgG1 antibodies for HLA-G Null 1,2,3 was determined by Surface Plasmon Resonance using a Biacore T200 (GE Healthcare Biosciences AB). Assays were performed at 25° C. Affinipure F(ab′)2 fragment goat anti-human IgG, Fc specific (Jackson ImmunoResearch) was immobilised on a Series S CM5 Sensor Chip (GE Healthcare Bio-Sciences AB) via amine coupling chemistry to a level of approximately 6000 response units (RU). HBS-EP⁺ buffer (10 mM HEPES pH7.4, 0.15M NaCl, 3 mM EDTA, 0.05% Surfactant P20, GE Healthcare Bio-Sciences AB) was used as the running buffer with a flow rate of 10 μL/min. A reference surface was prepared by activating and deactivating the appropriate flow cell.

A 10 μL injection of anti-HLA-G antibodies at concentrations from 0.6-0.9 μg/mL was used for capture by the immobilised anti-human IgG, Fc. Human “HLA-G Null 1,2,3” mutant AVI tev 10HisTag+B2m was titrated over the captured anti-HLA-G IgG from 20 μM at a flow rate of 30 μL/min for 60 s followed by a 150 s dissociation. The surface was regenerated at a flow rate of 10 μL/min by a 10 μL injection of 40 mM HCl followed by a 5 μL injection of 5 mM NaOH.

Background subtraction binding curves were analysed using the Biacore T200 Evaluation Software (Version 3.0) using steady state analysis. The results are presented in Table 11.

TABLE 11
Affinity for HLA-G Null 1,2,3 determined by SPR

| Antibody ID | KD (μM) |
|---|---|
| HLA-G01 | no binding |
| HLA-G02 | no binding |
| (Conventional, i.e. fucosylated) | |
| Afucosylated HLA-G02 | no binding |
| HLA-G06 | no binding |
| HLA-G03 | no binding |
| HLA-G07 | no binding |
| HLA-G04 | no binding |
| HLA-G05 | no binding |
| HLA-G08 | 7.8 |

No binding was detected for HLA-G01-HLA-G07. HLA-G08 showed some binding to HLA-G Null 1,2,3 and therefore was less specific to HLA-G.

### 7.3. Binding to HEK-Expressed HLA-G Wild Type Versus HLA-G Null 1,2,3 to Assess Specificity of the Antibodies

Binding to HLA-G wild type expressed on Human embryonic kidney (HEK293) cells was measured and compared to “HLA-G Null 1,2,3” to determine binding specificity. Advantageously, using cell-based assays, binding to dimeric HLA-G may be assessed, while SPR assay described above only measures binding to monomeric HLA-G.

HEK293 cells transfected with either HLA-G/β2m or HLA-G Null 1,2,3/β2m were incubated with the anti-HLA-G IgG1 for two hours at 4 degrees in 384-well V-bottom plates (Greiner). IgG concentrations ranging from 100 nM to 0.05 nM—diluted in PBS, 1% FBS, 0.1% Sodium azide. After incubation period, cells were washed three times in assay buffer, then incubated with 20 μl of staining solution for 20 min at 4 degrees (R-Phycoerythrin AffiniPure F(ab′)₂ Fragment Goat Anti-Human IgG (H+L) (Jackson ImmunoResearch)—3.75 μg/ml and Viability Dye e780 (Life Technologies)). After washing step, cells were incubated for 10 min in 10% neutral buffered formalin solution (Sigma-Aldrich) at room temperature, protected from light. Cells were then washed and re-suspended in 40 μl PBS. Samples were run on a FACS Canto II instrument in HTS mode to determine the percentage of PE positive cells. EC50 and Emax were calculated from the median fluorescence intensity using FlowJo analysis software. The results are presented in Table 12.

TABLE 12
Affinity for HEK-expressed HLA-G and
HLA-G Null 1,2,3 determined by FACS

| | Binding on HEK cells | | | |
|---|---|---|---|---|
| | | | | HLA-G |
| | | | HLA-G | null 1,2,3 |
| | HLA-G WT | HLA-G WT | null 1,2,3 | Emax |
| Antibody ID | EC50 (nM) | Emax (MFI) | EC50 (nM) | (MFI) |
| HLA-G01 | 1.54 | 129298 | ND | 61 |
| HLA-G02 | 1.65 | 141300 | ND | 67 |
| (VR12389 | | | | |
| gL2gH16) | | | | |
| Afucosylated | 0.92 | 89198 | ND | 63 |
| HLA-02 | | | | |
| HLA-G06 | 1.26 | 115689 | ND | 53 |
| HLA-G03 | 2.02 | 189443 | ND | 101 |
| HLA-G07 | 1.03 | 104296 | ND | 37 |
| HLA-G04 | 2.73 | 178887 | ND | 30 |
| HLA-G05 | 2.96 | 165437 | ND | 143 |
| HLA-G08 | 1.57 | 137718 | 35.90 | 2858 |

HLA-G03 had a lowest affinity for HLA-G expressed on HEK293, as compared to HLA-G01 and HLA-G02. Therefore, HLA-G01 and HLA-G02 were preferred over HLA-G03. The affinity of afucosylated HLA-G02 for HEK-expressed HLA-G was similar to that of its conventional (fucosylated) counterpart. Binding of HLA-G08 to HLA-G Null 1,2,3 was detected, which confirmed that this antibody was less specific.

### 7.4. Binding Affinity to JEG3 Cells as Determined by FACS Assay

Binding affinity of anti-HLA-G IgG was measured in a flow cytometry cell-based assay using Human choriocarcinoma trophoblastic (JEG3) naturally expressing HLA-G. This assay may advantageously be used to measure binding to cells naturally expressing HLA-G, including binding to dimeric HLA-G on cells, while the SPR assay described above only measures binding to monomeric HLA-G.

JEG3 cells were incubated with 1.5 ml anti-HLA-G IgG1 solution for two hours at 4 degrees in microcentrifuge tubes (Eppendorf). IgG concentrations ranging from 10 nM to 0.0005 nM—diluted in PBS, 1% FBS, 0.1% Sodium azide. Cells were transferred into 384-well V-bottom plates (Greiner) and washed three times in assay buffer, incubated with 20 μl of staining solution for 20 min at 4 degrees (R-Phycoerythrin AffiniPure F(ab′)₂ Fragment Goat Anti-Human IgG (H+L) (Jackson ImmunoResearch)—7.5 μg/ml and Viability Dye e780 (Life Technologies)). After washing step, cells were incubated for 10 min in 10% neutral buffered formalin solution (Sigma-Aldrich) at room temperature, protected from light. Cells were then washed and re-suspended in 20 μl PBS. Samples were run on a FACS Canto II instrument in HTS mode to determine the percentage of PE positive cells. KD were calculated from the median fluorescence intensity using FlowJo analysis software. The results are presented in Table 13.

TABLE 13
Affinity for JEG3 cells as determined by FACS

| | FACS |
|---|---|
| | Affinity |
| Antibody ID | KD (nM) |
| HLA-G01 | 0.045 |
| HLA-G02 | 0.020 |
| (VR12389 gL2gH16) | |
| HLA-G06 | — |
| HLA-G03 | 0.068 |
| HLA-G07 | — |
| HLA-G04 | — |
| HLA-G05 | 0.174 |
| HLA-G08 | — |

HLA-G01 and HLA-G02 showed the higher affinity in the FACS assay. The results confirmed that HLA-G03 had a lower affinity for HLA-G when expressed at the surface of cells naturally expressing HLA-G, notably as dimers, as compared to HLA-G01 and HLA-G02.

### 7.5. Binding to HLA-Is and JEG3 Wild Type (WT)/HLA-G Knock-Down

Binding to HLA-A/B/C/E/F consensus molecules expressed on HEK293 cells was measured to further explore specificity of binding of anti-HLA-G IgG. In the same assay, binding to HLA-G expressed on JEG3 cells was also measured and compared to JEG3 HLA-G knock-down (KD).

HEK293 cells transfected with either HLA-A, B, C, E or F consensus sequence/2m and JEG3 WT, and JEG3 KD were incubated with the anti-HLA-G IgG1 for two hours at 4 degrees in 384-well V-bottom plates (Greiner). IgG concentrations ranging from 100 nM to 0.05 nM—diluted in PBS, 1% FBS, 0.1% Sodium azide. After incubation period, cells were washed three times in assay buffer, then incubated with 20 μl of staining solution for 20 min at 4 degrees (R-Phycoerythrin AffiniPure F(ab′)₂ Fragment Goat Anti-Human IgG (H+L) (Jackson ImmunoResearch)—3.75 μg/ml and Viability Dye e780 (Life Technologies)). After washing step, cells were incubated for 10 min in 10% neutral buffered formalin solution (Sigma-Aldrich) at room temperature, protected from light. Cells were then washed and re-suspended in 40 μl PBS. Samples were run on a FACS Canto II instrument in HTS mode to determine the percentage of PE positive cells. EC50 and Emax were calculated from the median fluorescence intensity using FlowJo analysis software. The results are presented in Tables 14 and 15.

TABLE 14
Binding to HLA-A/B/C/E/F

| | HLA-A | HLA-A | HLA-B | HLA-B | HLA-C | HLA-C |
|---|---|---|---|---|---|---|
| | BINDING | BINDING | BINDING | BINDING | BINDING | BINDING |
| | EC50 | Emax | EC50 | Emax | EC50 | Emax |
| Antibody ID | (nM) | (MFI) | (nM) | (MFI) | (nM) | (MFI) |
| HLA-G01 | ND | 373.3 | ND | 221.6 | ND | 119 |
| HLA-G02 | ND | 57.3 | ND | 142.6 | ND | 65 |
| Afucosylated | ND | 191 | ND | 91 | ND | 117 |
| HLA-G02 | | | | | | |

| | HLA-E | HLA-E | HLA-F | |
|---|---|---|---|---|
| | BINDING | BINDING | BINDING | HLA-F |
| | EC50 | Emax | EC50 | Emax |
| Antibody ID | (nM) | (MFI) | (nM) | (MFI) |
| HLA-G01 | ND | 120.5 | ND | 54 |
| HLA-G02 | ND | 45 | ND | 67.6 |
| Afucosylated | ND | 54 | ND | 73 |
| HLA-G02 | | | | |

No binding to HLA-Is consensus was detected (“ND”). The results confirmed that the antibodies HLA-G01 and HLA-G02 were highly specific to HLA-G.

TABLE 15
Binding to JEG3 WT

| | JEG3 WT | JEG3 WT |
|---|---|---|
| | BINDING | BINDING |
| | EC50 | Emax |
| Antibody ID | (nM) | (MFI) |
| HLA-G01 | 0.38 | 58094 |
| HLA-G02 | 0.43 | 63979 |
| Afucosylated HLA-G02 | 0.2 | 44416 |
| HLA-G06 | 4.10 | 6966 |
| HLA-G03 | 0.41 | 38189 |
| HLA-G07 | 3.42 | 4900 |
| HLA-G04 | 3.65 | 31373 |
| HLA-G05 | 1.26 | 44413 |

In addition, no binding to JEG3 KD was detected. HLA-G03 had a similar EC50 but a much lower Emax as compared to HLA-G01 and HLA-G02 (or afucosylated HLA-G02), and was therefore not as good as HLA-G01 or HLA-G02.

### 7.6. Determination of the Binding Domain on HLA-G of the Anti-HLA G Antibodies

Binding to HLA-G Null3, HLA-G Null1,3 and HLA-G Null3 2AA expressed on Human embryonic kidney (HEK293) cells was measured to characterise the binding domain of anti-HLA-G IgG.

HEK293 cells transfected with either HLA-G Null3/β2m, HLA-G Null1,3/β2M and HLA-G Null3 2AA/β2M were incubated with the anti-HLA-G IgG1 for two hours at 4 degrees in 384-well V-bottom plates (Greiner). IgG concentrations ranging from 100 nM to 0.05 nM—diluted in PBS, 1% FBS, 0.1% Sodium azide. After incubation period, cells were washed three times in assay buffer, then incubated with 20 μl of staining solution for 20 min at 4 degrees (R-Phycoerythrin AffiniPure F(ab′)₂ Fragment Goat Anti-Human IgG (H+L) (Jackson ImmunoResearch)—3.75 μg/ml and Viability Dye e780 (Life Technologies)). After washing step, cells were incubated for 10 min in 10% neutral buffered formalin solution (Sigma-Aldrich) at room temperature, protected from light. Cells were then washed and re-suspended in 40 μl PBS. Samples were run on a FACS Canto II instrument in HTS mode to determine the percentage of PE positive cells. EC50 and Emax were calculated from the median fluorescence intensity using FlowJo analysis software. The results are presented in Table 16.

TABLE 16
Binding to HLA-G Null3, HLA-G Null 1,3
and HLA-G Null3 2AA expressed on HEK293

| | | | HLA-G | HLA-G | HLA-G | HLA-G |
|---|---|---|---|---|---|---|
| | HLA-G | HLA-G | Null | Null | Null3 | Null3 |
| | Null3 | Null3 | 1,3 | 1,3 | 2AA | 2AA |
| Antibody | EC50 | Emax | EC50 | Emax | EC50 | Emax |
| ID | (nM) | (MFI) | (nM) | (MFI) | (nM) | (MFI) |
| HLA-G01 | ND | 214 | ND | 182 | ND | 281 |
| HLA-G02 | ND | 97 | ND | 87 | ND | 44 |
| HLA-G06 | ND | 348 | ND | 318 | ND | 281 |
| HLA-G03 | ND | 166 | ND | 247 | ND | 186 |
| HLA-G07 | 0.09 | 124 | ND | 206 | ND | 97 |
| HLA-G04 | ND | 204 | ND | 278 | ND | 317 |
| HLA-G05 | ND | 17 | ND | 111 | ND | 178 |
| HLA-G08 | 13.38 | 4893 | 17.02 | 11716 | 15.50 | 2318 |

The data showed that the antibodies, including HLA-G02, were specific to HLA-G alpha 3 domain. The data confirmed the lowest specificity of HLA-G08.

### 7.7. Specificity of HLA-G02 Assessed in a PBMC Experiment

The specificity of HLA-G02 was further confirmed using PBMCs from 50 different donors, representing a variety of HLA-I alleles. The aim was to confirm that the anti-HLA-G antibody is specific and does not cross-react with other HLA-I molecules expressed on PBMCs and CD4+T lymphocytes in particular.

PBMCs were purified from peripheral venous blood and stored in Liquid nitrogen in Freezing medium (90% FBS+10% DMSO). PBMCs from 50 different donors were thawed and resuspended in 1 ml of complete RPMI medium (RPMI 1640 medium plus 10% Foetal Bovine Serum, 2 mM Glutamax and 1% Penicillin/Streptomycin). Cells were centrifuged at 300 rpm, 10 min and washed twice with PBS. Cell pellets were resuspended in 1 ml Facs buffer (PBS, 0.5% BSA and 2 mM EDTA) and cells were seeded in 96 well plate with 50 μl cell suspension/well.

The cells were stained with anti-Human CD4-APC (Biolegend, 2.5 μl/well) and with anti-HLA-G antibodies (HLA-G02 or control “pan-HLA”, an IgG1 which binds to HLA-Is and is not specific to HLA-G) or isotype control (50 μl of solution at 20 μg/ml per well). Cells were incubated at room temperature in the dark for 20 min and then washed twice with Facs buffer and resuspended in 50 μl of Facs buffer containing the secondary antibody Goat Anti-Human IgG-FITC (Jackson ImmunoResearch, dilution 1/100). The cells were incubated for a further 20 min at RT in the dark, then washed twice with Facs buffer. Cells were resuspended in 100 μl/well of Facs buffer and samples were acquired on the Canto II (HTS 1), 10,000 events collected per sample. Analysis was performed using FlowJo software v10.6.0 by measuring the Mean Fluorescence Intensity (MFI) of each CD4+ cell population for each donor. Graphs were performed using Graphpad Prism software.

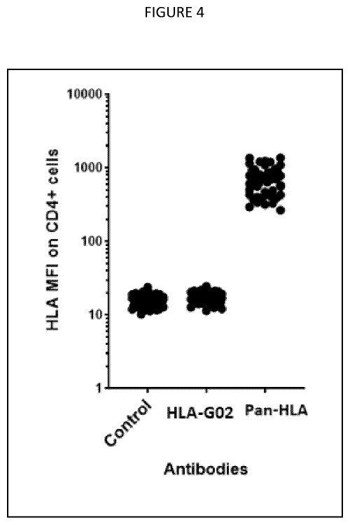

The results are presented in FIG. 4. FIG. 4 shows the lack of binding to CD4 T cells across 50 different donors of the specific anti-HLA-G antibody HLA-G02 in comparison to the pan-HLA antibody. Data are expressed in Mean Fluorescence Intensity (MFI) for each donor.

### 7.8. Binding of HLA-G02 to Cell-Membrane Bound HLA-G Isoforms: HLA-G1, HLA-G2, HLA-G3 or HLA-G4

The Expi293F human cells were transfected with plasmid encoding either HLA-G1, HLA-G2, HLA-G3 or HLA-G4 using the ExpiFectamine™ 293 Transfection Kit and following manufacturer's protocol (ThermoFisher Scientific, ref #A14524). 24 hrs post-transfection, cells were harvested, washed in PBS (300 rpm, 10 min) and resuspended in PBS. Cells seeded in 96 well/plate and stained with anti HLA-G antibodies HLA-G02 (human IgG1) or commercial antibody 4H84 (Abcam, Mouse IgG1) at 10 μg/ml final concentration and incubated in the dark for 20 min. Cells were then washed twice with Facs buffer (PBS, 0.5% BSA and 2 mM EDTA) and resuspended in 50 μl of Facs buffer containing the secondary antibody Goat Anti-Human IgG-PE (Jackson ImmunoResearch, dilution 1/100) or Goat Anti-Mouse IgG-PE (Jackson ImmunoResearch, dilution 1/100). The cells were incubated for a further 20 min at RT in the dark, then washed twice with Facs buffer. Cells were resuspended in 100 μl of Facs buffer and samples were acquired on the Canto II (HTS 1).

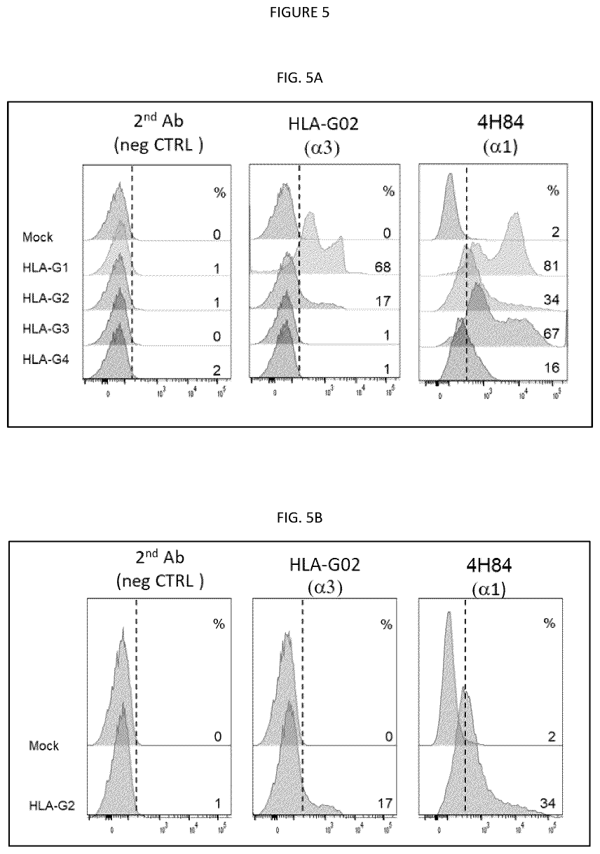

Results are presented in FIG. 5. FIG. 5A shows that the HLA-G02 antibody, specific for HLA-G alpha 3 domain, recognises both HLA-G1 and HLA-G2 (with respectively 68 and 17% positive cells) but not HLA-G3 nor HLA-G4 compared to the negative control (neg CTRL, irrelevant Ab). The commercial Mouse anti-HLA-G antibody 4H84, specific for α1 domain, was used as a positive control and recognises all isoforms HLA-G1, HLA-G2, HLA-G3 or HLA-G4 (respectively staining 81, 34, 67 and 16% of the cells).

FIG. 5B more particularly shows that the HLA-G02 antibody, specific for HLA-G alpha 3 domain, recognises HLA-G2 (17% positive cells) compared to the negative control (irrelevant Ab). The commercial Mouse anti-HLA-G antibody 4H84, specific for α1 domain, was used as a positive control for HLA-G2 (34% positive cells).

### Example 8: Assessment of the Ability of the HLA-G Antibodies to Block the Interaction Between HLA-G and ILT2 or ILT4

### 8.1. Blocking of the Interaction Between JEG3 and ILT2

Anti-HLA-G IgG were incubated with Human choriocarcinoma trophoblastic (JEG3) expressing HLA-G and with ILT2 rabbit Fe to measure their potency to block ILT2 binding to HLA-G.

JEG3 cells were incubated with the anti-HLA-G IgG1 for one hour at 4 degrees in 384-well V-bottom plates (Greiner). IgG concentrations ranging from 100 nM to 0.05 nM—diluted in PBS, 1% FBS, 0.1% Sodium azide. After incubation period, cells were washed in assay buffer, then incubated with 20 μl of ILT2rbFc solution at 3 μg/ml for one hour at 4 degrees. After incubation, 5 μl of staining solution (Fluorescein (FITC) AffiniPure F(ab′)₂ Fragment Goat Anti-Rabbit IgG, Fc fragment specific (Jackson ImmunoResearch)—7.5 μg/ml and Viability Dye e780 (Life Technologies)) were added to each well for 20 min at 4 degrees. After washing step, cells were incubated for 10 min in 10% neutral buffered formalin solution (Sigma-Aldrich) at room temperature, protected from light. Cells were then washed and re-suspended in 40 μl PBS. Samples were run on a FACS Canto II instrument in HTS mode to determine the percentage of FITC positive cells. IC50 and percentage inhibition were calculated from the median fluorescence intensity using FACSDiva analysis software. The results are presented in Table 17.

### Blocking of the Interaction Between JEG3 and ILT2: Large Volume of Reaction

Some antibodies have shown very low IC50 in the previous ILT2 blocking assay. At these low concentrations, ligand depletion is most likely to happen due to the small volume of reaction that may result in an over-estimation of the IC50 value. In order to overcome ligand depletion, large volume of anti-HLA-G IgG solutions (of the best blocking antibodies identified in the previous assay) were incubated with JEG3 expressing HLA-G and with ILT2 rabbit Fc to improve measurement of their potency to block ILT2 binding to HLA-G.

JEG3 cells were incubated with 1.5 ml anti-HLA-G IgG1 solution for two hours at 4 degrees in microcentrifuge tubes (Eppendorf). IgG concentrations ranging from 10 nM to 0.005 nM—diluted in PBS, 1% FBS, 0.1% Sodium azide. Cells were transferred into 384-well V-bottom plates (Greiner) and washed three times in assay buffer, then incubated with 20 μl of ILT2rbFc solution at 3 μg/ml for one hour at 4 degrees. After incubation period, cells were washed and incubated with 20 μl of staining solution (Fluorescein (FITC) AffiniPure F(ab′)₂ Fragment Goat Anti-Rabbit IgG, Fc fragment specific (Jackson ImmunoResearch)—7.5 μg/ml and Viability Dye e780 (Life Technologies)) for 20 min at 4 degrees. After washing step, cells were incubated for 10 min in 10% neutral buffered formalin solution (Sigma-Aldrich) at room temperature, protected from light. Cells were then washed and re-suspended in 20 μl PBS. Samples were run on a FACS Canto II instrument in HTS mode to determine the percentage of FITC positive cells. IC50 and percentage inhibition were calculated from the median fluorescence intensity using FlowJo analysis software. The results are presented in Table 17.

### 8.2. Blocking of the Interaction Between HLA-G Expressed on HEK and ILT2

Anti-HLA-G IgG were incubated with HEK293 cells transfected with HLA-G and with ILT2 rabbit Fc to measure their potency to block ILT2 binding to HLA-G.

HEK293 cells transfected with HLA-G/β2M were incubated with the anti-HLA-G IgG1 for one hour at 4 degrees in 384-well V-bottom plates (Greiner). IgG concentrations ranging from 100 nM to 0.05 nM—diluted in PBS, 1% FBS, 0.1% Sodium azide. After incubation period, cells were washed in assay buffer, then incubated with 20 μl of ILT2rbFc solution at 1 μg/ml for one hour at 4 degrees. After incubation, 5 μl of staining solution (Fluorescein (FITC) AffiniPure F(ab′)₂ Fragment Goat Anti-Rabbit IgG, Fc fragment specific (Jackson ImmunoResearch)—3 μg/ml and Viability Dye e780 (Life Technologies)) were added to each well for 20 min at 4 degrees. After washing step, cells were incubated for 10 min in 10% neutral buffered formalin solution (Sigma-Aldrich) at room temperature, protected from light. Cells were then washed and re-suspended in 40 μl PBS. Samples were run on a FACS Canto II instrument in HTS mode to determine the percentage of FITC positive cells. IC50 and percentage inhibition were calculated from the median fluorescence intensity using FACSDiva analysis software. The results are presented in Table 17.

### 8.3. Blocking of the Interaction Between HLA-G Expressed on HCT116 and ILT4

Anti-HLA-G IgG were incubated with Human colon cancer (HCT116) cells transfected with HLA-G and with ILT4 rabbit Fc to measure their potency to block ILT4 binding to HLA-G.

HCT116 cells transfected with HLA-G/β2M were incubated with the anti-HLA-G IgG for one hour at 4 degrees in 384-well V-bottom plates (Greiner). IgG1 concentrations ranging from 100 nM to 0.05 nM—diluted in PBS, 1% FBS, 0.1% Sodium azide. After incubation period, cells were washed in assay buffer, then incubated with 20 μl of ILT4rbFc solution at 0.4 μg/ml for one hour at 4 degrees. After incubation, 5 μl of staining solution (Fluorescein (FITC) AffiniPure F(ab′)₂ Fragment Goat Anti-Rabbit IgG, Fc fragment specific (Jackson ImmunoResearch)—1.5 μg/ml and Viability Dye e780 (Life Technologies)) were added to each well for 20 min at 4 degrees. After washing step, cells were incubated for 10 min in 10% neutral buffered formalin solution (Sigma-Aldrich) at room temperature, protected from light. Cells were then washed and re-suspended in 40 μl PBS. Samples were run on a FACS Canto II instrument in HTS mode to determine the percentage of FITC positive cells. IC50 and percentage inhibition were calculated from the median fluorescence intensity using FACSDiva analysis software. The results are presented in Table 17.

TABLE 17
Blocking activity of the HLA-G antibodies (IC50 values)

| | | ILT2rbFc | | |
|---|---|---|---|---|
| | | Large | | ILT4rbFc |
| | ILT2rbFc | Volume | ILT2rbFc | HCT116- |
| | JEG3 Flow | JEG3 | HEK-HLAG | HLAG |
| | Average | Average | Average IC50 | Average IC50 |
| Antibody ID | IC50 (nM) | IC50 (nM) | (nM) | (nM) |
| HLA-G01 | 0.59 | 0.039 | 1.58 | 1.78 |
| HLA-G02 | 0.53 | 0.017 | 1.37 | 1.31 |
| (VR12389 | | | | |
| gL2gH16) | | | | |
| Afucosylated | 0.51 | 0.021 | 0.83 | 1.38 |
| HLA-G02 | | | | |
| HLA-G06 | 7.40 | — | 10.54 | 4.58 |
| HLA-G03 | 0.47 | 0.038 | 1.59 | 1.86 |
| HLA-G07 | 1.82 | — | 2.33 | 2.82 |
| HLA-G04 | 1.10 | — | 2.05 | 3.32 |
| HLA-G05 | 0.74 | — | 1.51 | 2.59 |
| HLA-G08 | 1.58 | — | 9.84 | 2.75 |

HLA-G01 and HLA-G02 were identified as the best blockers of the association of HLA-G with ILT2 and of the association of HLA-G with ILT4. The blocking properties of the afucosylated HLA-G02 were similar to the properties of its conventional (i.e. fucosylated) counterpart.

### Example 9: Efficacy and Potency of HLA-G01-HLA-G08 Antibodies in ADCC of HLA-G Expressing Cells

Antibody dependent cellular cytotoxicity (ADCC) is an immune mechanism whereby cells expressing Fc receptors such as NK cells can recognise and kill antibody coated cells. It is a process crucial for anti-cancer responses and is a critical mechanism underlying the efficacy of many anti-cancer therapies. The ability of a panel of anti-HLA-G IgG1 antibodies to elicit ADCC in vitro was determined using two different types of target cells expressing HLA-G. These cells had either been transfected with HLA-G and human Beta-2-microglobulin (HCT116 colorectal cancer cells) or endogenously expressed the target on their cell surface (JEG3 cells).

### Methods

### Transfection of HCT116 Colorectal Cancer Cell Line

HCT116 cells were transfected with an HLA-G construct that also encoded a green fluorescent protein (GFP) tag. Cells successfully transfected with HLA-G therefore also expressed GFP and could be easily identified and accurately monitored by flow cytometry. A total of 40 μg DNA (20 μg each of HLA-G ECD GFP and human β2M plasmids) and 80 μL lipofectamine LTX was used to transfect 4×10⁶ HCT116 cells in a T75 tissue culture flask. After 24 hours the cells were detached from the flask and used as target cells as described below.

### In-Vitro ADCC Assay

HLA-G transfected HCT116 or JEG3 target cells were plated out (2×10⁴ cells/well in a volume of 50 μL) into a polypropylene round bottom plate in the appropriate culture medium. Anti-HLA-G or control antibodies were prepared as 4× concentrated stocks in the same medium and 50 μl/well was added to appropriate wells. All antibodies were tested in either duplicate or triplicate depending on the number of available donor NK cells. Some target cells were left without any antibody and were used as no treatment controls.

Primary human NK cells were isolated from whole blood by negative selection using a magnetic bead kit (Miltenyi Biotech). The purified NK cells were resuspended in RPMI+10% FBS, 2 mM L-Glutamine in the minimum volume needed for the assay. To appropriate wells of the assay plate 100 μl/well NK cells were added on top of the target cells and antibodies. The Effector: Target ratio was between 10:1 and 3:1 depending on donor NK cell number.

The assay plate was incubated at 37° C. 5% CO₂ for ˜3 hrs. After 3 hours the number of live target cells was measured by flow cytometry. The assay plate was centrifuged at 300 g for 3 minutes to pellet the cells and each well was stained for the epithelial cell marker Epcam and the NK cell marker CD56. The staining antibodies (anti-Epcam PE and anti-CD56 BV421) were diluted to 1/100 in cell staining buffer and 100 μL/well was added to each well. The plate was incubated at room temperature for 15 minutes. Following staining the cells were washed twice with 150 μl/well PBS and the plate was centrifuged at 300 g for 3 minutes in between each wash. At the end of the staining the cells in each well were resuspended in a final volume of 100 μp/well PBS containing 50 nM TO-PRO™-3 cell viability dye.

After 10 minutes, exactly 70 μL of sample from each well was acquired on a BD FACS Canto II Instrument and the data was analysed using FlowJoV10.60 software. The total number of live target cells was determined for each well. Live target cells were identified firstly as TO-PRO-™3 negative and then secondly as CD56 negative and SSC high. The cells were then gated on Epcam (JEG3) or Epcam and GFP expression (HCT 116). Percent depletion compared to either untreated cells or the isotype control was calculated for each test sample and the data was transferred to GraphPad Prism 8.1.1 Software for analysis.

### Results

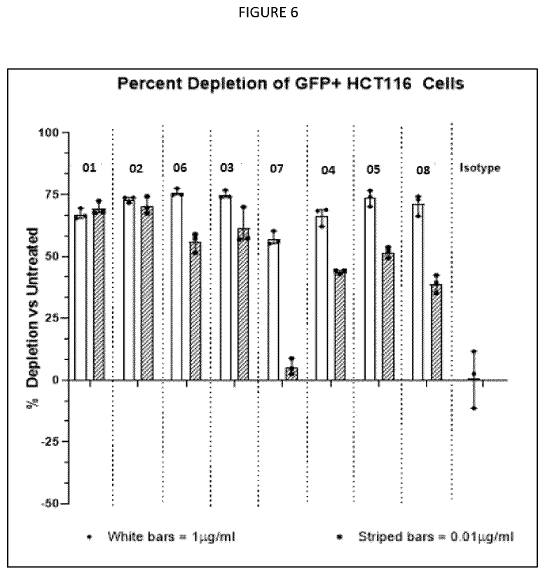

FIG. 6 shows the percentage of depleted Epcam+GFP+HCT116 target cells following treatment with different anti-HLA-G antibodies or an IgG1 isotype control antibody. Each antibody was tested at two different concentrations either 1 μg (white bars) or 0.01 μg/ml (striped bars). The E:T ratio was 3.5:1. Each bar represents the mean (and the range) of three data points and each dot/square is an individual replicate. Data is from one representative donor.

Table 18 lists the mean percentage depletion of Epcam+GFP+HCT116 cells by each antibody shown in FIG. 6 (N.D.: not detected)

TABLE 18
mean percentage depletion of Epcam+ GFP+ HCT116 cells

| | | Mean % depletion GFP+ |
|---|---|---|
| Antibody | Mean % depletion GFP+ | Cells |
| ID | Cells (1 μg/mL) | (0.01 μg/mL) |
| HLA-G01 | 67.15 | 69.41 |
| HLA-G02 | 73.11 | 70.51 |
| HLA-G06 | 75.79 | 55.90 |
| HLA-G03 | 75.01 | 61.45 |
| HLA-G07 | 57.20 | 2.46 |
| HLA-G04 | 66.48 | 43.83 |
| HLA-G05 | 73.68 | 51.78 |
| HLA-G08 | 71.20 | 39.00 |
| IgG1 Isotype | 0.93 | N.D |

Several antibodies showed a similar Mean % depletion GFP+ Cells at the highest concentration of antibodies (1 μg/mL). The highest Mean % depletion GFP+ Cells observed at a lowest concentration (0.01 μg/mL) was observed for antibodies HLA-G01 and HLA-G02. Those antibodies were taken forward for further characterization in ADCC assays.

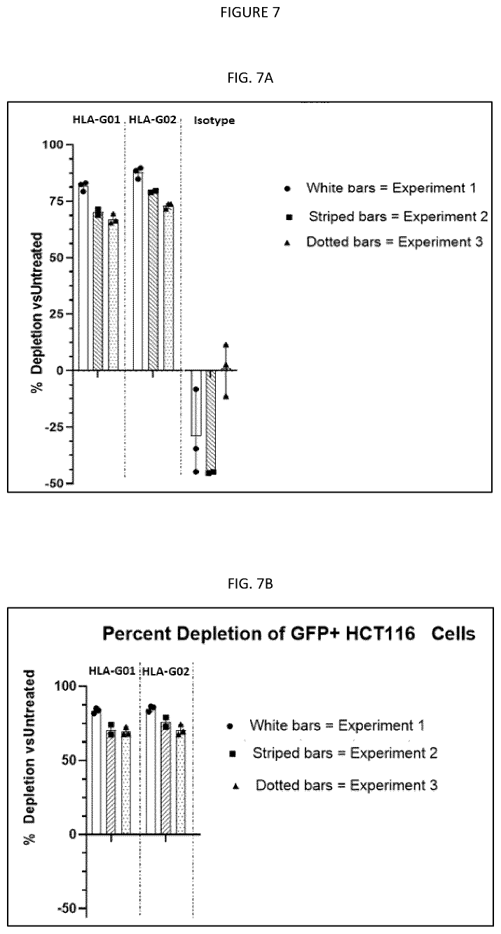

FIG. 7 shows the percentage of depleted Epcam+GFP+HCT116 target cells following treatment with anti-HLA-G antibodies HLA-G01 and HLA-G02 or an IgG1 isotype control antibody from three separate experiments (3 different donors). Antibodies were tested at 1 μg/ml (FIG. 7A) or 0.01 μg/ml (FIG. 7B). The E:T ratio was between 2.5 and 3:1. Each bar represents the mean (and range) of data from an individual experiment and each dot, square or triangle is an individual replicate.

Table 19 lists the mean percentage depletion of Epcam+GFP+HCT 116 cells by each antibody at 1 μg/mL shown in FIG. 7A.

TABLE 19
mean percentage depletion of Epcam+ GFP+
HCT116 (1 μg/mL antibody)

| | Mean % | Mean % | Mean % |
|---|---|---|---|
| Human IgG1 | Depletion | Depletion | Depletion |
| Antibody | Experiment 1 | Experiment 2 | Experiment 3 |
| HLA-G01 | 81.61 | 70.21 | 67.15 |
| HLA-G02 | 87.71 | 79.25 | 73.11 |
| Isotype | −34.56 | −44.76 | 0.93 |

Table 20 lists the mean percentage depletion of Epcam+GFP+HCT116 cells by each antibody at 0.01 μg/mL shown in FIG. 7B.

TABLE 20
mean percentage depletion of Epcam+ GFP+
HCT116 (0.01 μg/mL antibody)

| | Mean % | Mean % | Mean % |
|---|---|---|---|
| Human IgG1 | Depletion | Depletion | Depletion |
| Antibody | Experiment 1 | Experiment 2 | Experiment 3 |
| HLA-G01 | 83.83 | 70.79 | 69.41 |
| HLA-G02 | 85.09 | 75.92 | 69.42 |
| Isotype | N.D | N.D | N.D |

At both concentrations of antibodies, the antibody HLA-G02 had a better cell depleting activity than HLA-G01 as determined in the ADCC assay.

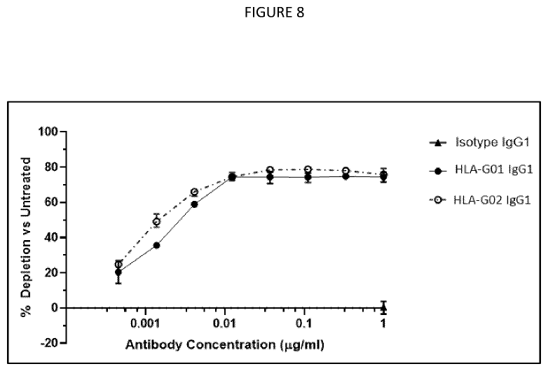

FIG. 8 shows the percentage depletion of Epcam+GFP+HCT116 cells following treatment with a titration of anti-HLA-G antibodies HLA-G01 and HLA-G02 compared to an isotype control. The E:T ratio was 4:1. Each point represents the mean (and range) of 3 replicates. Data shown is from a single representative donor.

Based on those results, the antibody HLA-G02 was selected for further characterization and development, notably an afucosylated version of HLA-G02 was prepared as described above for comparison with the conventional IgG1.

Afucosylation of antibodies has been shown to increase FcγRIII: Fc binding affinity and has been reported to increase the ADCC potential of IgG1 molecules. An afucosylated version of HLA-G02 was therefore tested for its ability to elicit ADCC of transfected HCT116 or JEG3 cells. The afucosylated antibodies were compared to the same antibody V regions made in conventional IgG1 format.

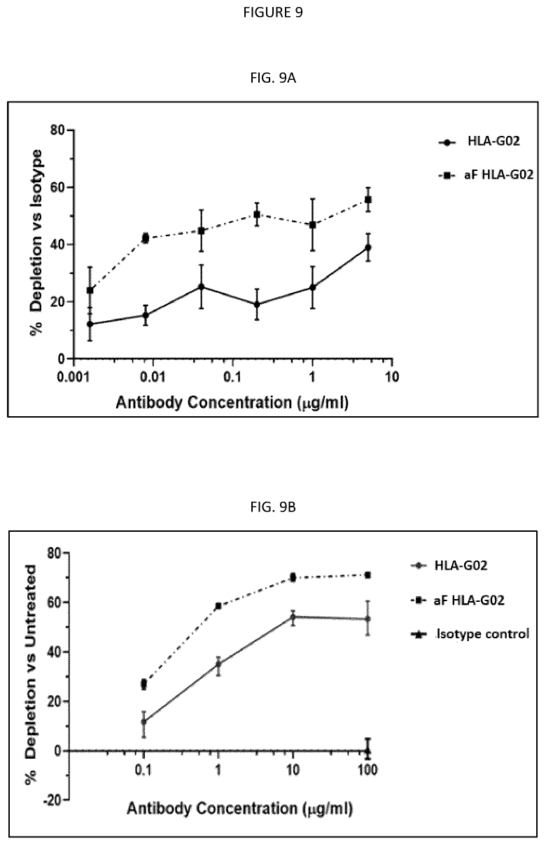

FIG. 9A shows the percentage depletion of JEG3 cells following treatment with conventional HLA-G02 IgG1 (solid line) or afucosylated HLA-G02 IgG1 (“aF HLA-G02”, dotted line). The E:T ratio was 10:1. Each point represents the mean (and range) of 2 replicates. Data is shown from a single representative donor.

FIG. 9B shows the percentage depletion of Epcam+GFP+HCT 116 cells following treatment with conventional HLA-G02 IgG1 (solid line) or afucosylated HLA-G02 IgG1 (“aF HLA-G02”, dotted line). The E:T ratio was 5:1. Each point represents the mean (and range) of 3 replicates. Data is shown from a single representative donor.

Antibody HLA-G02 showed a better potency and efficacy in cell killing assays, and was therefore selected for further development as a candidate for use in therapy. The afucosylated version of HLA-G02 had an improved ADCC as compared to its conventional (i.e. fucosylated) counterpart.

### Example 10: Efficacy and Potency of HLA-G Antibody HLA-G02 in Phagocytosis of HLA-G Expressing Cells

HLA negative K562 cells were used as target and transfected to express HLA-G. Prelabelled target cells (CTY+) were incubated with monocyte-derived Macrophages (CD11b+) with the HLA-G02 antibodies in hIgG1 format. Phagocytosis was analysed by measuring percentage of CTY+CD11b+ cells

### Methods

CD14+ monocytes were purified from peripheral venous blood using Pan Monocyte Isolation Kit, human (Miltenyi), an indirect magnetic labelling system for the isolation of untouched monocytes. Cells were differentiated into macrophages with 50 ng/ml recombinant MCSF in complete RPMI medium (RPMI 1640 medium plus 10% Foetal Bovine Serum, 2 mM Glutamax and 1% Penicillin/Streptomycin) for 7 days at 37 C, 5% CO2.

HLA-negative erythroleukemia K562 cells were either mock transfected or transfected with HLA-G and B2m using the 4D-Nucleofector System and the SF Cell Line 4D-Nucleofector™ X Kit L (Lonza, ref #V4XC-2024) and cultured in complete RPMI for 24 hrs at 37 C, 5% CO2. The next day, the cells were harvested, washed and labelled with Cell Trace Yellow (Thermofisher), washed again and plated at 25.000 cells per well in 100 μl complete RPMI in 96 well round bottom Ultra low attachment plate (Corning Costar). The cells were subsequently incubated with either anti-CD47 antibody or anti-HLA-G antibodies or isotype controls at 10 μg/ml for 1 hour at 37 C, 5% CO2. After a wash, the cells were combined with monocyte-derived macrophages (50.000 macrophages per well) at a ratio Macrophage:cell target=2:1. The mixed cells were incubated for 2 hours at 37° C., 5% CO2. Then, cells were washed and resuspended in PBS plus 10% Purified human Fc gammaR-binding inhibitor (Thermofisher) for 20 minutes at 4° C. and then stained with anti-CD11b-APC (Biolegend) for 20 minutes at 4° C. Cells were washed and resuspended in PBS plus 2 mM EDTA plus 0.5% BSA in presence of DAPI (500 ng/ml) dead cells exclusion. Samples were acquired by flow cytometry on the BD FACSCanto. Analysis was performed using FlowJo software v10.6.0 by measuring the percentage of CTY+CD11b+ double positive cells.

Anti-CD47 was used as a positive control; studies have shown that by inhibiting CD47 on target cells, this resulted in inhibiting the interaction of CD47 with SIRPa a receptor expressed on macrophages, leading to an increased phagocytosis activity. Expression of CD47 has been shown to be upregulated on tumor cells and anti-CD47 antibodies are currently tested in clinical trials. In the phagocytosis assay, it is a good control to evaluate the phagocytic activity of the monocyte-derived macrophages and it shows that both Mock and HLA-G transfected cells are both able to elicit phagocytosis.

### Results

Table 21 shows HLA-G-specific phagocytosis activity of the anti-HLA-G antibodies on Mock transfected K562 compared to HLA-G-expressing K562 target cells (HLA-G/B2m K562).

Data are expressed in percentage of CTY+CD11b+ double positive cells and are representative of 3 independent experiments, in duplicate.

TABLE 21
phagocytosis activity of the anti-HLA-G02

% CTY+CD11b+ cells (mean)

| IgG1 antibody | Mock K562 | HLA-G/B2m K562 |
|---|---|---|
| Isotype CTL | 26.8 | 24.6 |
| anti-CD47 | 53.6 | 49.6 |
| HLA-G02 | 25.6 | 39.6 |

Table 22 shows statistically significant HLA-G specific phagocytosis activity of HLA-G02, compared to IgG1 isotype control. Data represents pooled data from 6 donors (in duplicate). Data was exported to Excel and normalized to the mean of percentage of phagocytosis of mock transfected cells.

TABLE 22
phagocytosis activity of the anti-HLA-G02 (6 donors)

| | % CTY+CD11b+ cells relative to Mean Control | | | | | | | | | | | |
|---|---|---|---|---|---|---|---|---|---|---|---|---|
| | Mock K562 | | | | | | HLA-G/B2m K562 | | | | | |
| IgG1 | Donor | Donor | Donor | Donor | Donor | Donor | Donor | Donor | Donor | Donor | Donor | Donor |
| antibody | A | B | C | D | E | F | A | B | C | D | E | F |
| Isotype | 100.0 | 100.0 | 100.0 | 100.0 | 100.0 | 100.0 | 104.8 | 92.5 | 110.5 | 115.9 | 92.2 | 105.4 |
| CTL | | | | | | | | | | | | |
| anti- | 100.0 | 100.0 | 100.0 | 100.0 | 100.0 | 100.0 | 98.6 | 99.4 | 109.8 | 88.6 | 86.5 | 71.6 |
| CD47 | | | | | | | | | | | | |
| HLA-G02 | 100.0 | 100.0 | 100.0 | 100.0 | 100.0 | 100.0 | 123.6 | 109.8 | 215.9 | 256.1 | 202.4 | 184.8 |

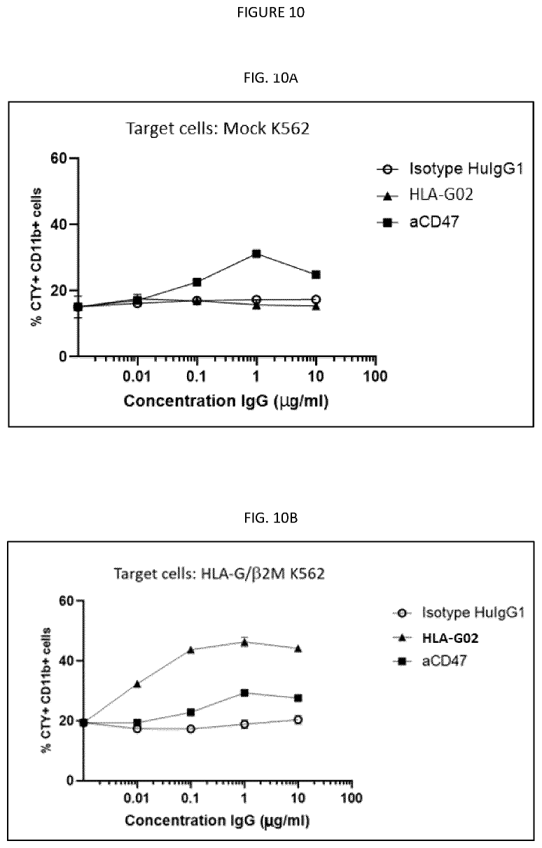

FIG. 10 shows titration of the HLA-G-specific phagocytosis activity of the Human anti-HLA-G IgG1 antibody HLA-G02 on Mock transfected (FIG. 10A) and HLA-G/B2m transfected K562 target cells (FIG. 10B) compared to anti-CD47 antibody and isotype control. Data are expressed in percentage of CTY+CD11b+ double positive cells and are representative of 1 out of 2 donors (Table 23).

TABLE 23
titration of the HLA-G-specific phagocytosis activity of HLA-G02

| | % CTY+CD11b+ cells (mean) | | | | | | | | | |
|---|---|---|---|---|---|---|---|---|---|---|
| | Mock K562 | | | | | HLA-G/B2m K562 | | | | |
| Concentration | Isotype | HLA- | Neg. | Neg. | | Isotype | HLA- | Neg. | Neg. | |
| (μg/ml) | IgG1 | G02 | CTL1 | CTL2 | aCD47 | IgG1 | G02 | CTL1 | CTL2 | aCD47 |
| 10 | 17.4 | 15.5 | 17.6 | 17.4 | 24.9 | 20.4 | 44.2 | 22.1 | 19.9 | 27.6 |
| 1 | 17.3 | 15.8 | 18.7 | 17.4 | 31.2 | 18.9 | 46.4 | 21.1 | 20.4 | 29.4 |
| 0.1 | 17.0 | 17.0 | 18.8 | 18.5 | 22.7 | 17.3 | 43.8 | 19.9 | 18.1 | 22.9 |
| 0.01 | 16.2 | 17.6 | 19.7 | 18.2 | 17.1 | 17.4 | 32.4 | 21.4 | 18.7 | 19.3 |
| 0.001 | 15.1 | 15.1 | 15.1 | 15.1 | 15.1 | 19.4 | 19.4 | 19.4 | 19.4 | 19.4 |

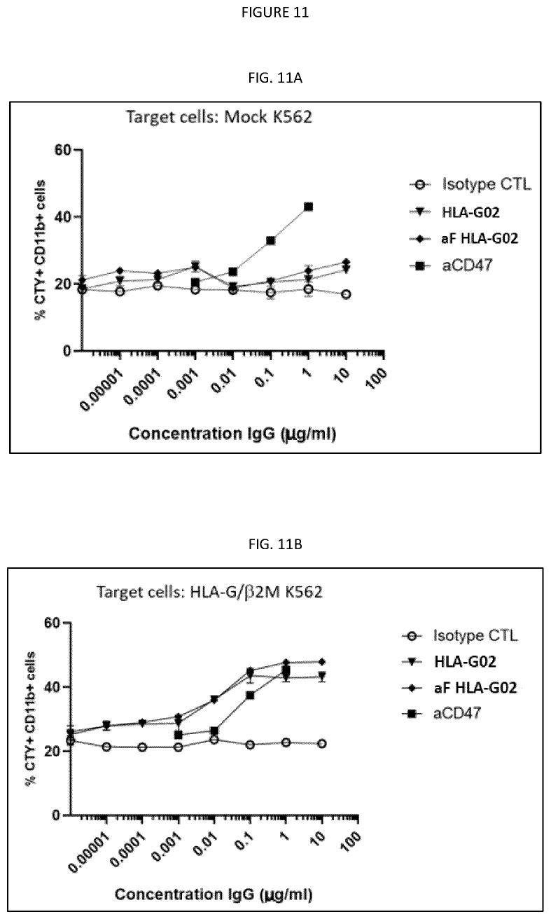

FIG. 11 shows titration of the HLA-G-specific phagocytosis activity of conventional and afucosylated (aF) formats of the HLA-G02 on Mock transfected (FIG. 11A) and HLA-G-expressing K562 target cells (FIG. 11B) compared to anti-CD47 antibody and isotype control. Data are expressed in percentage of CTY+CD11b+ double positive cells and are representative of 1 out of 3 donors in 2 independent experiments (Table 24).

TABLE 24
titration of the HLA-G-specific phagocytosis
activity of HLA-G02 and afucosylated HLA-G02

| | % CTY+CD11b+ cells (mean) | | | | | | | |
|---|---|---|---|---|---|---|---|---|
| | Mock K562 | | | | HLA-G/B2m K562 | | | |
| Antibodies | | | aF | | | | aF | |
| Concentration | Isotype | HLA- | HLA- | | Isotype | HLA- | HLA- | |
| (μg/ml) | HuIgG1 | G02 | G02 | aCD47 | HuIgG1 | 02 | G02 | aCD47 |
| 10 | 17.0 | 24.3 | 26.6 | | 22.5 | 43.3 | 48.0 | |
| 1 | 18.6 | 21.5 | 24.1 | 43.1 | 22.8 | 43.0 | 47.8 | 45.5 |
| 0.1 | 17.6 | 20.6 | 21.0 | 33.0 | 22.2 | 43.7 | 45.3 | 37.6 |
| 0.01 | 18.3 | 19.3 | 18.9 | 23.8 | 23.7 | 36.2 | 36.0 | 26.5 |
| 0.001 | 18.5 | 25.3 | 25.1 | 20.6 | 21.4 | 28.8 | 30.9 | 25.2 |
| 0.0001 | 19.6 | 21.4 | 23.3 | | 21.4 | 28.7 | 29.2 | |
| 0.00001 | 17.8 | 21.0 | 24.0 | | 21.5 | 28.0 | 28.0 | |
| 0.000001 | 18.5 | 18.6 | 21.3 | | 23.5 | 25.3 | 26.1 | |

Afucosylated HLA-G02 showed improved ADCP as compared to its conventional (i.e. fucosylated) counterpart HLA-G02.

### Example 11: Epitope Mapping of VR12389 Antibody by X-Ray Crystallography

### Protein Production of HLA-G Fusion Protein

PeptideB2mHLAG C42S mut tev10his-HLA-G (C42S) in which the cysteine required for homodimerization was mutated to serine (Signal peptide in bold (cleaved after expression), peptide underlined, GS linker in italic, B2m sequence, GS linker in italic, HLA-G sequence with C42S in gray shadow, tev cleavage site bold and italic, GS linker in italic, 10 histag),

(SEQ ID NO: 145)
MSVPTQVLGLLLLWLTDARCRIIPRHLQLGCGGSGGGGSGGGGS
IQRTPKIQVYSRHPAENGKSNFLNCYVSGFHPSDIEVDLLKNGE
RIEKVEHSDLSFSKDWSFYLLYYTEFTPTEKDEYACRVNHVTLS
QPKIVKWDRDMGGGGSGGGGSGGGGSGGGGSGSHSMRYFSAAVS
RPGRGEPRFIAMGYVDDTQFVRFDSDSA [symbol] PRMEPRAPWVEQEGP
EYWEEETRNTKAHAQTDRMNLQTLRGYYNQSEASSHTLQWMIGC
DLGSDGRLLRGYEQYAYDGKDYLALNEDLRSWTAADTAAQISKR
KCEAANVAEQRRAYLEGTCVEWLHRYLENGKEMLQRADPPKTHV
THHPVFDYEATLRCWALGFYPAEIILTWQRDGEDQTQDVELVET
RPAGDGTFQKWAAVVVPSGEEQRYTCHVQHEGLPEPLMLRWKQL
EENLYFQGSGGSHHHHHHHHHH

The protein PeptideB2mHLAG C42S mut tev10his was expressed by transient transfection using the Expi293™ Expression System (Life Technologies™) following manufacturers protocol. Cells were harvested 5 days post transfection and supernatants used immediately for purification. Supernatants comprising PeptideB2mHLAG C42S mut tev10his protein was applied to Histrap NiExcel column. Unbound protein and contaminants were washed with PBS, 500 mM NaCl, 20 mM Imidazole, pH 7.4 and the PeptideB2mHLAG C42S mut tev10his protein eluted with PBS, 500 mM NaCl, 500 mM Imidazole, pH 7.4. Fractions containing purified PeptideB2mHLAG C42S mut tev10his protein were pooled and the his tag removed by incubation of the protein with tev protease at a ratio of 1:100 for 2 hours at room temperature and 2 hours at 4° C. Protein was concentrated and purified further by size exclusion chromatography on S200 26/60 which had been equilibrated with 20 mM Tris, 50 mM NaCl, pH 7.4 buffer. Fractions containing purified PeptideB2mHLAG C42S mut protein were pooled, concentrated to 2.94 mg/ml and stored in 1 mg aliquots at −80° C.

PeptideB2mHLAG C42S mut protein was characterized by SDS-PAGE and migrated to a position on the gel consistent with the expected molecular weight (MW) of the glycosylated protein.

The amino acid sequence of the protein PeptideB2mHLAG C42S mut obtained, used for complexing and in crystal structure was as follows:

(SEQ ID NO: 146)
RIIPRHLQLGCGGSGGGGSGGGGSIQRTPKIQVYSRHPAENGKSNFL
NCYVSGFHPSDIEVDLLKNGERIEKVEHSDLSFSKDWSFYLLYYTEF
TPTEKDEYACRVNHVTLSQPKIVKWDRDMGGGGSGGGGSGGGGSGGG
GSGSHSMRYFSAAVSRPGRGEPRFIAMGYVDDTQFVRFDSDSASPRM
EPRAPWVEQEGPEYWEEETRNTKAHAQTDRMNLQTLRGYYNQSEASS
HTLQWMIGCDLGSDGRLLRGYEQYAYDGKDYLALNEDLRSWTAADTA
AQISKRKCEAANVAEQRRAYLEGTCVEWLHRYLENGKEMLQRADPPK
THVTHHPVFDYEATLRCWALGFYPAEIILTWQRDGEDQTQDVELVET
RPAGDGTFQKWAAVVVPSGEEQRYTCHVQHEGLPEPLMLRWKQLEEN
LYFQ

### Fab Purification VR12389

Rabbit Fab (VR12389) (Light chain sequence represented by SEQ ID NO:9 and heavy chain sequence represented by SEQ ID NO: 13) was expressed in ExpiCHO cells as secreted proteins. Expression constructs for light chains and heavy chains were co-transformed at a 1:1 molar ratio. The secreted proteins were purified by passing conditioned media over Protein G beads and eluted with 0.1M glycine, pH 2.7. Fractions were neutralized by the addition of 2M Tris-HCl, pH8.5. The protein was dialyzed into PBS, pH 7.2 then concentrated to 5.62 mg/ml and stored at 4° C.

PeptideB2mHLAG C42S Mut Protein with VR12389 RbFab

Fused peptide_β2m_HLA-G was incubated at a 1:1.1 molar ratio with VR12389 RbFab for 1 hour. The complex was then purified using a Superdex 200 16/600 column (GE Healthcare) using 10 mM Tris-HCl, 150 mM NaCl (pH 7.5) as the running buffer. Fractions were analysed by SDS-PAGE using NuPAGE 4-20% Tris-Glycine (Thermo), and purest complex fractions were then concentrated to 10.4 mg/ml using an Amicon® Ultra-15 Centrifugal Filter Unit (Millipore).

Crystallography PeptideB2mHLAG C42S Mut Protein with VR12389 RbFab

Crystallisation conditions for the complex were identified using several commercially available crystallisation screens. These were carried out in sitting drop format, using Swissci 96-well 2-drop MRC Crystallization plates (sourced from Molecular Dimensions, Cat No. MD11-00-100). First, the reservoirs were filled with 75 μL of each crystallisation condition in the screens using a Microlab STAR liquid handling system (Hamilton). Then, 300 nL of the HLA-G/VR12389 complex and 300 nL of the reservoir solutions were dispensed in the wells of the crystallisation plates using a Mosquito liquid handler (TTP LabTech). Crystals were identified in condition G4 of the ProPlex-HT96 screen (Molecular Dimensions) containing 2M Ammonium sulfate and 0.1 M Tris at pH 8.0. Crystals frozen using the reservoir solution containing 25% glycerol as cryoprotectant. Diffraction data were collected at the Diamond Light Source. The structure was solved using molecular replacement in Phaser. Phenix.refine and Coot were used in alternating cycles of automated and manual refinement.

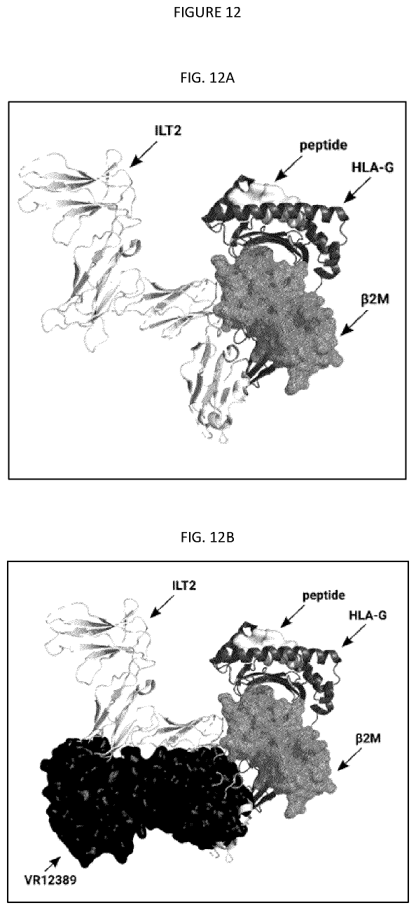

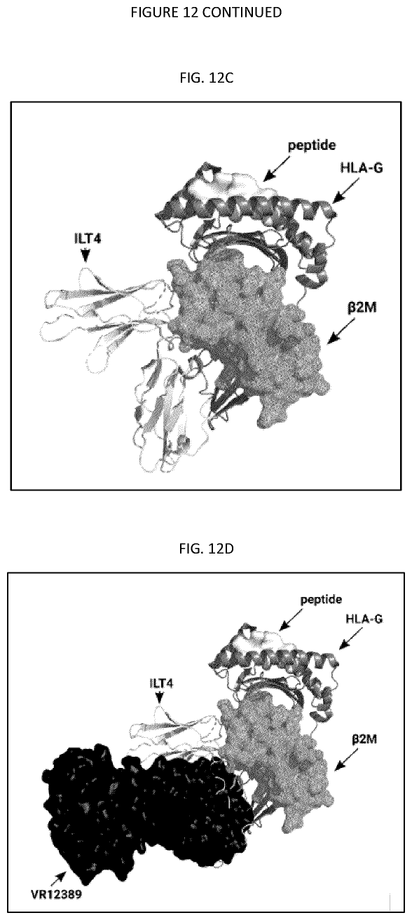

By superposing the crystal structure of HLA-G in complex with VR12389, with the crystal structures of HLA-G in complex with ILT2 and ILT4 (as reported in the literature, see e.g. Q Wang et al., Cellular & Molecular Immunology, 2019 and Shiroishi, PNAS vol 103, No 44, P 16412-16417, 2006), it was clear that VR12389 prevents HLA-G from interacting with the ILT2 and ILT4 receptors by steric hindrance (FIG. 12).

At <4 Å contact distance, the HLA-G epitope recognised by the VR12389 antibody comprises HLA-G residues V194, F195, Y197, E198, Q224, Q226, D227, V248, V249, P250 and Y257.

At <5 Å contact distance, the HLA-G epitope recognised by the VR12389 antibody comprises HLA-G residues V194, F195, Y197, E198, R219, Q224, Q226, D227, V248, V249, P250, E253 and Y257.

### Example 12: Epitope Mapping of 12389 Antibody by HDX-MS and NMR

In contrast to crystallography which is performed in static conditions, HDX-MS and NMR are techniques that analyse interactions in solution and allow to show allosteric or conformational changes that are not always apparent by crystallography.

### HDX-MS Materials and Methods

### Sample Preparation and Data Acquisition

For HDX-MS analysis, 12 μM of Human HLA-G ECD (SEQ ID NO: 110) was complexed with 36 μM of 12389 antibody (expressed either as an IgG1 or a Fab) and incubated for 1 hour at 4° C.

4 μl of HLA-G or the HLA-G complex were diluted into 57 μL of 10 mM phosphate in H₂O (pH 7.0), or into 10 mM phosphate in D20 (pD 7.0) at 25° C. The deuterated samples were then incubated for 0.5, 2, 15 and 60 min at 25° C. After the reaction, all samples were quenched by mixing at 1:1 with a quench buffer (4 M Guanadine Hydrochloride, 250 mM Tris(2-carboxyethyl) phosphine hydrochloride (TCEP), 100 mM phosphate) at 1° C. The mixed solution was at a final pH 2.5. The mixture was immediately injected into the nanoAcquity HDX module (Waters Corp.) for peptic digest. Peptide digestion was then performed on-line using a Enzymatic online digestion column (Waters) in 0.2% formic acid in water at 20° C. and with a flow rate of 100 μL/min. All deuterated time points and un-deuterated controls were carried out in triplicate with blanks run between each data-point.

Peptide fragments were then trapped using an Acquity BEH C18 1.7 μM VANGUARD chilled pre-column for 3 min. Peptides were then eluted into a chilled Acquity UPLC BEH C18 1.7 μM 1.0×100 using the following gradient: 0 min, 5% B; 6 min, 35% B; 7 min, 40% B; 8 min, 95% B, 11 min, 5% B; 12 min, 95% B; 13 min, 5% B; 14 min, 95% B; 15 min, 5% B (A: 0.2% HCOOH in H2O, B: 0.2% HCOOH in acetonitrile. Peptide fragments were ionized by positive electrospray into a Synapt G2-Si mass spectrometer (Waters). Data acquisition was run in ToF-only mode over an m/z range of 50-2000 Th, using an MSe method (low collision energy, 4V; high collision energy: ramp from 18V to 40V). Glu-1-Fibrinopeptide B peptide was used for internal lock mass correction.

### HDX-MS Data Processing

MS^{E} data from un-deuterated controls samples of HLA-G were used for sequence identification using the Waters Protein Lynx Global Server 2.5.1 (PLGS). Peptide search was performed against a database of the HLA-G sequence only, with precursor intensity threshold of 500 counts and 3 matched product ions required for assignment. Ion accounting files for the 3 control samples were combined into a peptide list imported into Dynamx v3.0 software. Peptides were subjected to further filtering in DynamX. Filtering parameters used were a minimum and maximum peptide sequence length of 4 and 25, respectively, minimum intensity of 1000, minimum MS/MS products of 2, minimum products per amino acid of 0.2, and a maximum MH +error threshold of 10 ppm. DynamX v3.0 was used to quantify the isotopic envelopes resulting from deuterium uptake for each peptide at each time-point. Furthermore, all the spectra were examined and checked visually to ensure correct assignment of m/z peaks and only peptides with a high signal to noise ratios were used for HDX-MS analysis.

Following manual filtration in Dynamx, statistical analysis and filtration were performed using Deuteros that uses statistical analysis published by Houde et al., 2011. Deuteros generates a woods plot that displays peptide length, start and end residues, global coverage and a y-axis metric which is the absolute uptake (in Daltons). It is the difference in uptake in the presence of a ligand (bound) and the apo form. Woods plots first apply confidence filtering to all peptides in each timepoint. Peptides with differential deuteration outside of the selected confidence limits are non-significant.

### Results

In the presence of VR12389, a total of twelve peptides showing statistically significant reduction in deuterium incorporation upon antibody binding were observed for HLA-G, nine of which showed major protection. The major protection covered residues 178-196 (MLQRADPPKTHVTHHPVFD) and 214-230 (ILTWQRDGEDQTQDVEL). Both regions are within the α3 domain (and the end five residues of α2). The region showing medium protection upon antibody binding covers residues 234-249 (RPAGDGTFQKWAAVVV) and is likely due to a conformational change. Peptides showing a similar exchange pattern in the presence and absence of the antibody have a non-significant deuterium incorporation.

TABLE 25
peptides showing reduction in
deuterium incorporation upon VR12389 binding
to HLA-G as determined by HDX-MS

| Start | End | Peptide sequence | Deuterium Uptake |
|---|---|---|---|
| 234 | 249 | RPAGDGTFQKWAAVVV | Medium protected |
| 184 | 195 | RADPPKTHVTHHPVF | Medium protected |
| 180 | 196 | QRADPPKTHVTHHPVFD | Medium protected |
| 181 | 195 | RADPPKTHVTHHPVF | Major protected |
| 217 | 229 | WQRDGEDQTQDVE | Major protected |
| 214 | 230 | ILTWQRDGEDQTQDVEL | Major protected |
| 178 | 196 | MLQRADPPKTHVTHHPVFD | Major protected |
| 216 | 229 | TWQRDGEDQTQDVE | Major protected |
| 180 | 195 | QRADPPKTHVTHHPVF | Major protected |
| 178 | 195 | MLQRADPPKTHVTHHPVF | Major protected |
| 181 | 196 | RADPPKTHVTHHPVFD | Major protected |
| 216 | 227 | TWQRDGEDQTQD | Major protected |

As a conclusion, from the HDX-MS at 30 seconds of deuterium incubation, potential binding domains for VR12389 are 178-MLQRADPPKTHVTHHPVFD-196 and 214-ILTWQRDGEDQTQDVEL-230.

### Nuclear Magnetic Resonance (NMR) Spectroscopy

Epitope mapping of the antibodies VR12389 was determined by NMR spectroscopy using the Fab fragments of the antibody.

### Materials

²H/¹³C/¹⁵N Labelled Expression of HLA-G α3 Domain

BL21(DE3) Competent E. coli (New England BioLabs #C2527H) were transformed via standard heat shock with 1 μg of HLA-G α3 short N-His ATUM #393044 (HLA-G residues: 207-300). Transformed cells were plated on LB agar plates containing 100 μg/ml carbenicillin and incubated overnight at 37° C. The following day a single colony was used to inoculate 10 ml LBroth containing 100 μg/ml carbenicillin (Merck #C1389) and grown at 37° C. shaking at 200 RMP for 5 h (New Brunswick Excella E25). 1 ml of starter culture was then used to inoculate 500 ml of 2H/¹³C/¹⁵N labelled minimal media (described below) and grown overnight in single use 2 L Erlenmeyer flasks (VWR #734-1904) at 37° C. shaking at 200 RPM. The following day the optical density (0D600) of the overnight culture was recorded (Amersham Biosciences Ultrospec 3100 pro). Expression cultures were then inoculated with the overnight culture to a final OD600 of 0.1.

²H/¹³C/¹⁵N labelled minimal media expression cultures were grown in 500 ml batches in single use 2 L Erlenmeyer flasks at 37° C. shaking at 200 RMP until an OD600 of 0.9 was reached. HLA-G α3 expression was then induced with 500 μM IPTG (Sigma #I6758). Induced cultures were then left at 37° C. for an additional 4 h before being harvested by centrifugation at 7,000 g for 30 mins (Beckman Coulter J6-MI). Harvested pellets were then frozen at −20° C. before cell lysis.

### Unlabelled Expression of Human β2m

BL21(DE3) Competent E. coli was transformed with 1 μg of Human β2m (residues: 21-119) ATUM #358573 and grown as above. The following day a single colony was used to inoculate 10 ml LBroth (10 g/L Tryptone, 5 g/L Yeast Extract, 5 g/L NaCl, 1 mM NaHO) containing 100 μg/ml carbenicillin and grown at 37° C. shaking at 200 RMP for 5 h. 1 ml of starter culture was then used to inoculate 500 ml LBroth containing 100 μg/ml carbenicillin and grown overnight in a single use 2 L Erlenmeyer flask at 37° C. shaking at 200 RPM. The following day the OD₆₀₀ of the overnight culture was recorded. Expression cultures were then inoculated with the overnight culture to a final OD600 of 0.1.

2×TY (Tryptone 16 g/L, Yeast Extract 10 g/L, NaCl 5 g/L) expression cultures were again grown in 500 ml batched in single use 2 L Erlenmeyer flasks at 37° C. shaking at 200 RMP until an OD₆₀₀ of 3.0 was reached. The incubator temperature was then dropped to 17° C. 30 minutes later cultures were fed with 20× feed solution (1 M MOPS pH 7.2, 20 mM MgCl2, 20 mM MgSO₄, 20% Glycerol) and expression was induced with 150 μM IPTG. Induced cultures were then left at 17° C. for 16 h before being harvested by centrifugation (7,000 g for 30 mins). Harvested pellets were then frozen at −20° C. before cell lysis.

### Bacterial Cell Lysis

Protocol for purification and refolding was adapted from: Craig S. Clements et al. The production, purification and crystallization of a soluble heterodimeric form of a highly selected T-cell receptor in its unliganded and liganded state. Biological Crystallography, 2002.

Cell pellets were lysed in Lysis Buffer: 50 mM Tris pH 8.0, 1% (v/v) Triton X-100, 1% (w/v) sodium deoxycholate, 100 mM NaCl, 10 mM DTT, 1 mg DNAse I (Biomedicals), 5 mM MgCl2, cOmplete protease inhibitors (Roche). After 10 min of continuous stirring at room temperature 10 mM EDTA was added. Resuspended cell pellet was then pass through a CF Cell Disrupter (Constant systems) 3× at 4° C. with a pressure of 20 psi. Lysate was then clarified via centrifugation at 48,000 g for 1 h at 4° C. (Beckman Coulter Avanti JXN-26). Insoluble pellet was then washed 2× with Wash Buffer 1:50 mM Tris pH 8.0, 0.5% (v/v) Triton X-100, 100 mM NaCl, 1 mM EDTA, 1 mM DTT, 0.2 mM, cOmplete protease inhibitors. Between each wash resuspended inclusion bodies were centrifuged at 48,000×g for 30 minutes. After the second wash inclusion bodies were washed a final time in Final Wash Buffer: 50 mM Tris pH 8.0, 1 mM EDTA, 1 mM DTT, cOmplete protease inhibitors. Purified inclusion bodies were then resolubilised in 20 mM Tris pH 8.0, 8 M urea (Sigma #U5378), 0.5 mM EDTA, 1 mM DTT. Purification fractions were analysed via SDS-PAGE using NuPAGE 4-12%, Bis-Tris (Thermo #NP0322) and NuPAGE MES SDS Running Buffer (Thermo #NP0002) stained with Quick Coomassie Stain.

²H/¹³C/¹⁵N HLA-G α3 and Unlabelled Human β2m Refolding

Resolubilised inclusion bodies were then diluted in 1.5 M guanidine HCl, 5 mM sodium acetate, 5 mM EDTA to roughly 1 mg/ml before refolding.

Human β2m was first added dropwise into Refolding Buffer: Tris pH 8.5, 0.4 M arginine, 0.5 mM oxidized glutathione, 5 mM reduced glutathione, 2 mM EDTA. Followed by ²H/¹³C/¹⁵N labelled HLA-G α3 domain in a 1:1 molar ratio. Refolding reaction was left at room temperature for 16 h with gentle stirring. 3,000 MWCO Spectra/Por dialysis membrane was then used to dialyse the refolding reaction in Dialysis Buffer: 5 mM Tris pH 8.5, at a 1:20 dilution factor. Dialysis buffer was changed once during a 24 h dialysis at room temperature.

### Purification of Folded Complex

Refolded HLA-G α3/β2m complex was then purified using a AKTA Pure (GE Healthcare) system and a HiTrap Q column (Cytiva Life Sciences) using the following buffers and purification sequence: Buffer A: 10 mM Tris, 10 mM NaCl pH 8.5 Buffer B: 10 mM Tris, 500 mM NaCl pH 8.5. Purification Sequence: Run at 5 ml/min, Equilibrate 5 CV Buffer A, Load dialysed refolding reaction, Wash 10 CV Buffer A, Elution: 0-40% B in 10 CV, Hold at 40% for 10 CV, 40-100% B in 20 CV, Hold 100% B for 10 CV.

Fractions were analysed via SDS-PAGE using NuPAGE 4-12%, Bis-Tris (Thermo) and NuPAGE MES SDS Running Buffer (Thermo) stained with Quick Coomassie Stain (VWR #SERA35081.01). Pure fractions were pooled before being concentrated using an 10,000 MWCO Amicon Ultra (Millipore) and loaded onto S75 300/10 increase gel filtration column (Cytiva Life Sciences) with 150 mM NaCl, 10 mM Tris pH 7.4, 0.02% NaN₃ as the running buffeR. Again, fractions were analysed via SDS-PAGE and pure fractions pooled. Final purified sample was analysed again via SDS-PAGE, before being concentrated to ˜350 μm. Protein concentration was determined using a Thermo Scientific Nanodrop 2000 specrophotometer.

### Purification of Fab Reagents:

Fab reagents were purified using an AKTA Pure system (GE Healthcare) and a packed Gammabind Plus Sepharose (Cytiva Life Sciences) column. Supernatants were concentrated using AKTA Flux system (GE Healthcare) to in excess of 300 mg/L before capture. The following buffers and purification sequence were used as follows: Buffer A: 10 mM PBS pH 7.4. Buffer B: 0.1 M glycine-HCl pH 2.7. Equilibrate 5 CV Buffer A. Load supernatant at a flowrate to ensure at least 20-minute contact time. Wash Buffer A 5 CV. Elute 100% Buffer B 5 CV. Elution fractions were neutralised with 2 M Tris pH 8.5.

Fractions were analysed via SDS-PAGE using NuPAGE 4-20% Tris-Glycine and NuPAGE MOPS SDS Running Buffer (stained with Quick Coomassie Stain. Pure fractions were pooled before being concentrated using an 10,000 MWCO Amicon Ultra (Millipore) and loaded onto S200 26/60 filtration column (Cytiva Life Sciences) with 10 mM PBS pH 7.4 as the running buffer. Again, fractions were analysed via SDS-PAGE and Acquity UPLC H-Class System (Waters) using a BEH200 SEC column (Waters). Pure fractions pooled before being concentrated to in excess of 5 mg/ml. Protein concentration was determined using a Thermo Scientific Nanodrop 2000 specrophotometer.

### Backbone Assignment of HLA-G α3

To obtain the backbone assignment of the HLA-G α3 domain a 500 μl ²H/¹³C/¹⁵N labelled sample of HLA-G α3 in complex with unlabelled β2M at a concentration of 320 μM in 150 mM NaCl, 10 mM Tris pH 7.4, 0.02% NaN₃ was prepared and transferred to a 5 mm NMR tube. All experiments were recorded at 35° C. on either a 600 MHz Bruker AVIII-HD or 600 MHz Bruker AVANCE NEO spectrometer fitted with cryogenically cooled probes. Sequential connections between the backbone NMR signals of residues in the protein were made using a 3D TROSY-HNCACB (Wittekind and Mueller, 1993 HNCACB, a High-Sensitivity 3D NMR Experiment to Correlate Amide-Proton and Nitrogen Resonances with the Alpha- and Beta-Carbon Resonances in Proteins. J. Magn. Reson. Ser. B 101, 201-205. doi:10.1006/jmrb.1993.1033; Salzmann et. al., 1999. TROSY-type Triple Resonance Experiments for Sequential NMR Assignment of Large Proteins. J. Am. Chem. Soc. 121, 844-848. doi: 10.1021/ja9834226) and a 3D TROSY-HNCOCACB (Salzmann et. al., 1999, TROSY-type Triple Resonance Experiments for Sequential NMR Assignment of Large Proteins. J. Am. Chem. Soc. 121, 844-848; Eletsky et. al., 2001. TROSY NMR with partially deuterated proteins. J. Biomol. NMR 120, 177-180). TROSY-HNCACB was recorded with spectral widths of 75, 36 and 16 ppm and acquisition times of 9 (F1), 21 (F2) and 70 (F3) ms in the ¹³C, ¹⁵N and ¹H dimensions respectively (16 scans per increment, 1.3 s relaxation delay, 5 days total acquisition time). TROSY-HNCOCACB was recorded with spectral widths of 75, 36 and 16 ppm and acquisition times of 9 (F1), 21 (F2) and 70 (F3) ms in the ¹³C, ¹⁵N and ¹H dimensions respectively (8 scans per increment, 1.3 s relaxation delay, 2 days 17 hours total acquisition time). NMR spectra were processed using NMRPipe (Delaglio et al., 1995 NMRPipe: a multidimensional spectral processing system based on UNIX pipes. J. Biomol. NMR 6, 277-93). Data analysis was carried out using Sparky (Goddard and Kneller, D. G. SPARKY 3. In., University of California, San Francisco), resulting in the assignment of the amide proton and nitrogen residues of 80 residues, corresponding to 93% of the residues of the native protein (excluding proline residues).

### Mapping the Binding Site of Fab Fragments

Mapping of the binding sites of the Fab fragments were carried out using 150 μM samples of ²H/¹³C/¹⁵N HLA-G α3 in complex with unlabelled β2M containing a 10% molar excess of the unlabelled Fabs. 200 μl samples were prepared in the same buffer as described above for the backbone assignment of the HLA-G α3 and transferred to 3 mm NMR tubes. ¹H and ¹⁵N chemical shift changes were determined by comparison of the TROSY (Pervushin et. al., 1998. Single Transition-to-single Transition Polarization Transfer (ST2-PT) in [¹⁵N,¹H]-TROSY. J. biomol. NMR 12, 345-348) spectrum recorded on the HLA-G α3/β2M/Fab complex with an equivalent control TROSY experiment of the HLA-G α3/β2M. The control TROSY experiment of the HLA-G α3/β2M was recorded with spectral widths of 36 and 16 ppm and acquisition times of 60 (F1) and 80 (F2) ms in the ¹⁵N and ¹H dimensions respectively (8 scans per increment, 1.5 s relaxation delay, 1 hour total acquisition time). The TROSY experiments of the HLA-G α3/β2M/Fabs were recorded with spectral widths of 36 and 16 ppm and acquisition times of 60 (F1) and 80 (F2) ms in the ¹⁵N and ¹H dimensions respectively (8 scans per increment, 1.5 s relaxation delay, 1 hour total acquisition time). Spectra were processed using NMRPipe (Delaglio et al., 1995 NMRPipe: a multidimensional spectral processing system based on UNIX pipes. J. Biomol. NMR 6, 277-93). Data analysis was carried out using Sparky.

Chemical shift changes were analysed using the minimal shift approach (Williamson et al., 1997, Mapping the binding site for matrix metalloproteinase on the N-terminal domain of the tissue inhibitor of metalloproteinases-2 by NMR chemical shift perturbation. Biochemistry 36, 13882-9) using the equation below to calculate the combined chemical shift change (Δδ):

$$Δ δ = \sqrt{{( {Δδ}_{HN} )}^{2} + {( Δ {δ}_{N} {α}_{N} )}^{2}}$$

where Δδ_{HN} and Δδ_{N} are the differences in the ¹H and ¹⁵N chemical shifts respectively. αN corresponds to the scaling factor of 0.2, used to account for difference in the chemical shift range of the nitrogen chemical shifts.

To identify the Fab binding sites on the HLA-G α3 a histogram of combined minimal shift versus protein sequence was used to identify regions of HLA-G α3 containing significantly perturbed signals. If the size of the combined minimal shift change for an amino acid exceeded a threshold value of the mean of the combined chemical shift change for all the amino acids, these residues were selected for further evaluation as possible contact residues in the Fab binding site.

Two thresholds were applied to identify residues bound by the Fab: those whose minimal shift exceeds the mean of all calculated shifts and those whose minimal shift exceeds the mean plus one standard deviation of all calculated minimal shifts. In these analyses Proline residues cannot be identified as they contain no amide proton.

### Results

The epitope as determined by NMR as defined with increasing stringency as exceeding the mean of all calculated shifts (>0.0764) comprises residues T200, L201, L215, W217, R219, D220, E229, A245, A246, V247, V249, S251, E253, Q255, T258, H260, V261 and W274. The epitope determined by NMR as defined with increasing stringency as exceeding the mean plus one standard deviation of all calculated shifts (>0.1597) comprises residues H191, Y197, E198, R202, L230, V248, G252, C259 and K275.

### Example 13: Characterization of Antibody Molecules by Liquid Chromatography-Mass Spectrometry (LC-MS)

The molecular weight (MW) of HLA-G02 (VR12389gL2gH16) (unmodified (fucosylated) and afucosylated), VR12389 gL2gH15 and HLA-G01 was measured by the separate heavy and light chains (reduced) by LC-MS using a Waters ACQUITY UPLC System with a Xevo G2 Q-ToF mass spectrometer. Samples (˜5 μg) were reduced with 5 mM tris(2-carboxyethyl) phosphine (TCEP) in 150 mM ammonium acetate at 37° C. for 40 minutes. The LC column was a Waters BioResolve™ RP mAb Polyphenyl, 450 Å, 2.7 μm held at 80° C., equilibrated with 95% solvent A (water/0.02% trifluoroacetic acid (TFA)/0.08% formic acid) and 5% Solvent B (95% acetonitrile/5% water/0.02% TFA/0.08% formic acid) at a flow rate of 0.6 mL/minute. Proteins were eluted with a gradient from 5% to 50% solvent B over 8.8 minutes followed by a 95% solvent B wash and re-equilibration. UV data were acquired at 280 nm. MS conditions were as follows: Ion mode: ESI positive ion, resolution mode, mass range: 400-5000m/z and external calibration with NaI.

Data were analyzed using Waters MassLynx™ and MaxEnt Software.

As shown in Table 26, the predicted MW from the sequences were consistent with the observed MW for the heavy and light chains by LC-MS for all antibodies. Also, for VR12389 gL2gH16, as expected there was a mass difference of ˜146 Da for the heavy chain of the corresponding afucosylated version.

TABLE 26
LC and HC molecular weight

| | Light Chain MW (Da) | | | Heavy Chain MW (Da) | | |
|---|---|---|---|---|---|---|
| Antibody | Expected | Observed | Δm | Expected | Observed | Δm |
| HLA-G01 | 23741.3 | 23741.4 | 0.1 | 49689.8 | 49691.8 | 2.0 |
| VR12389 | 23489.2 | 23489.0 | −0.2 | 49771.8 | 49775.0 | 3.2 |
| gL2gH15 | | | | | | |
| HLA-G02 | 23489.1 | 23488.4 | −0.7 | 49743.8 | 49744.0 | 0.2 |
| (VR12389 | | | | | | |
| gL2gH16) | | | | | | |
| Afucosylated | 23489.2 | 23487.0 | −2.2 | 49597.7 | 49598.0 | 0.3 |
| HLA-G02 | | | | | | |

### Example 14: Thermal Stability (Tm) Measurements

The melting temperature (Tm) or temperature at the midpoint of unfolding was determined using the thermal shift assay to assess the conformational stability of the molecules and hence robustness to manufacture and long term stability.

The fluorescent dye SYPRO® orange was used to monitor the protein unfolding process by binding to hydrophobic regions that become exposed as the temperature increases. The reaction mix contained 5 μL of 30×SYPRO® Orange Protein Gel Stain (Thermofisher scientific, S6651), diluted from 5000× concentrate with test buffer. 45 μL of sample at 0.2 mg/mL, in PBS pH 7.4, was added to the dye and mixed. 10 μL of this solution was dispensed in quadruplicate into a 384 PCR optical well plate and was run on a QuantStudio 7 Real-Time PCR System (Thermofisher™). The PCR system heating device was set at 20° C. and increased to 99° C. at a rate of 1.1° C./min. A charge-coupled device monitored fluorescence changes in the wells. Fluorescence intensity increases were plotted, the inflection point of the slope(s) was used to generate apparent midpoint temperatures (Tm). The data is shown in Table 27.

TABLE 27
Summary of Thermal shift assay data for samples in PBS pH 7.4.

| | CH2 domain | Fab domain |
|---|---|---|
| Antibody | (° C.) | (° C.) |
| HLA-G01 | 69.5 ± 0.2 | 86.8 ± 0.2 |
| HLA-G02 (VR12389gL2gH16) | 69.7 ± 0.1 | 85.4 ± 0.1 |
| Afucosylated HLA-G02 | 70.3 ± 0.2 | 84.2 ± 0.2 |
| VR12389 gL2gH15 | 69.6 ± 0.1 | 83.9 ± 0.1 |

The samples exhibited high and similar thermal stabilities suggesting no substantial structural differences between the grafts. The thermal stability of Fab domains can vary considerably (typical range is 70° C. to 84° C.), a high thermal stability is preferred due to greater machinal stability. As expected, no meaningful structural differences were observed between the conventional (i.e. fucosylated) and afucosylated HLA-G02.

### Example 15: Experimental Isoelectric Point (pI) Measurement

An iCE3™ whole-capillary imaged capillary isoelectric focusing (cIEF) system (ProteinSimple™) was used to experimentally determine pI.

The experimental pI of the main peak was found to be similar for HLA-G01 and HLA-G02. The pI was in a range that was expected to be good for manufacturing steps and formulation buffers. The presence of minor acidic and basic charged species was consistent with other IgG1 therapeutic molecules and could be attributed to common post-translation modifications.

### Example 16: Solubility Measurement Using Polyethylene Glycol (PEG) Aggregation Assay

The PEG aggregation assay was used as a mimic of high concentration solubility. PEG is a nonadsorbing, nondenaturing polymer and due to its inert nature, has been used to promote protein precipitation primarily via an excluded volume effect. Samples were exposed to increasing concentrations of PEG 3350; the amount of sample remaining in solution was determined by plotting absorbance at A280 nm. The determination of % PEG concentration at which half the sample had precipitated generated a PEG midpoint (PEGmdpnt) score. This score permitted the antibody molecules to be ranked on apparent native state aggregation propensity, a low PEGmdpnt score (for example <10) indicates a greater propensity for native state aggregation.

Stock 40% PEG 3350 solutions (w/v) were prepared in PBS pH 7.4 and in a pH 5.0 buffer (common pre-formulation storage buffers). A serial titration was performed by an ASSIST PLUS liquid handling robot (INTEGRA™ 4505), resulting in a range of 40% to 15.4% PEG 3350. To minimize non-equilibrium precipitation, sample preparation consisted of mixing antibody and PEG solutions at a 1:1 volume ratio. 35 μL of the PEG 3350 stock solutions was added to a 96 well v bottom PCR plate (A1 to H1) by a liquid handling robot. 35 μL of a 2 mg/mL sample solution was added to the PEG stock solutions resulting in a 1 mg/mL test concentration and a final PEG 3350 concentration of 20% to 7.7%. This solution was mixed by automated slow repeat pipetting and incubated at 37° C. for 0.5 h to re-dissolve any non-equilibrium aggregates. Samples were then incubated at 20° C. for 24 h. The sample plate was subsequently centrifuged at 4000×g for 1 h at 20° C. 50 μL of supernatant was dispensed into a UV-Star®, half area, 96 well, μClear®, microplate (Greiner, 675801). Protein concentrations were determined by UV spectrophotometry at 280 nm using a FLUOstar® Omega multi-detection microplate reader (BMG LABTECH™). The resulting values were plotted using Graphpad prism ver 7.04, PEG midpoint (PEG_{mdpnt}) score was derived from the midpoint of the sigmoidal dose-response (variable slope) fit.

The data is shown in Table 28 where the higher PEG mid-point (%) equates to greater high concentration solubility. NB * samples showed signs of aggregation at the lowest test concentration of PEG 3350 (7.7%) therefore accurate PEG midpoints could not be generated.

TABLE 28
PEG aggregation assay data in PBS pH 7.4
and a pH 5 buffer (PEG % midpoint values)

| | PBS | |
|---|---|---|
| Antibody | pH 7.4 | pH 5.0 Buffer |
| HLA-G01 | 9.5 | <7.7* |
| HLA-G02 (VR12389 | 9.4 | 15.8 |
| gL2gH16) | | |
| Afucosylated HLA-G02 | 9.5 | 15.2 |
| VR12389 gL2gH15 | 9.6 | 15.6 |

The PEG aggregation assay data indicated that all the tested samples showed moderate aggregation propensity in PBS. Notably HLA-G01 exhibited very high aggregation propensity at pH 5.0. In contrast, VR12389 grafts showed low aggregation propensity in the pH 5.0 buffer. No meaningful difference was observed between the conventional (i.e. fucosylated) HLA-G02 and its afucosylated counterpart.

### Example 17: kD Interaction Parameter Measurement (Colloidal Stability)

The kD interaction parameter was used to assess colloidal stability, where positive and negative values relate to repulsive and attractive intermolecular forces respectively.

Dynamic light scattering (DLS) was performed on a DynaPro III plate reader (Wyatt Technology Corp, Santa Barbara, CA, USA). Samples were diluted to 30 μL in PBS, pH7.4 or in a pH5 buffer and diluted from 7 mg/mL to 1 mg/mL in increments of 1 mg/mL. Wells containing buffer were selected as solvent offsets and the measurements performed at 25° C., with the laser power set to 20% and auto-attenuation enabled. Each measurement was the average of five, 5 s scans in triplicate (5×3). The Diffusion co-efficient was measured (Dm) and the interaction parameter (kD) calculated according to the equation below, where Do represents the diffusion coefficient at infinite dilution.

$$D m = {D}_{0} ( 1 + {k}_{D} C )$$

Equation: D₀ given by Debye plot at Y-intercept. The slope=kD*D₀.

The Diffusion coefficient was measured as a function of protein concentration and the kD used to assess colloidal stability, where positive and negative values suggest repulsive and attractive intermolecular forces respectively. For samples that show attractive forces/self-association, the diffusion coefficient gets larger as a function of protein concentration and this is reflected in a negative kD value. The data is shown in Table 29.

TABLE 29
kD Interaction parameter data for the anti-HLA-G antibodies

mL/g

| Sample | PBS | Ac pH 5 |
|---|---|---|
| HLA-G01 | −9.93 | −68.7 |
| VR12389 gL2gH15 | −9.30 | −4.04 |
| HLA-G02 | −7.92 | −2.98 |
| (VR12389 gL2H16) | | |

The kD interaction parameter was shown to be highly negative for HLA-G01 at pH5.0 and less negative in pH7.4, suggesting greater colloidal stability at physiological pH. VR12389 grafts had minor negative kD values suggesting good colloidal stability at either pH.

### Example 18: Effect of Mechanical Stress on Aggregation Stability (Aggregation Assay)

Proteins tend to unfold when exposed to an air-liquid interface, where hydrophobic surfaces are presented to the hydrophobic environment (air) and hydrophilic surfaces to the hydrophilic environment (water). Agitation of protein solutions achieves a large air-liquid interface that can drive aggregation. This assay serves to mimic stresses that the molecule would be subjected to during manufacture (for example ultra-filtration) and to provide stringent conditions in order to try to discriminate between different antibody molecules.

Samples in PBS pH 7.4 or in a pH 5 buffer were stressed by vortexing using an Eppendorf Thermomixer Comfort™. Prior to vortexing the concentration was adjusted to 1 mg/mL, and the absorbance at 595 nm obtained using a Varian Cary 50-Bio Spectrophotometer® to establish the time zero reading. Each sample was sub-aliquoted into 1.5 mL conical Eppendorf®-style capped tubes (3×250 μL) and subjected to vortexing at 1400 rpm at 25° C. for up to 24 hours. Aggregation (turbidity) was monitored by measurement of the samples at 595 nm at 3 hours and 24 hours post vortexing using a Varian Cary® 50-Bio spectrophotometer. The data is summarized in Table 30.

TABLE 30
Effect of Stress at an air-liquid interface (turbidity
at 595 nm) on anti HLA-G humanised graft molecules
in PBS pH 7.4 and in a pH 5 buffer.

| | OD 595 nm | | | |
|---|---|---|---|---|
| | PBS pH 7.4 | | pH 5 Buffer | |
| Antibody | 3 h | 24 h | 3 h | 24 h |
| HLA-G01 | 0.005 ± 0 | 0.029 ± 0.011 | 0.0030 | 0.20 ± 0.084 |
| VR12389 | 0.006 ± 0 | 0.016 ± 0 | 0 | 0.065 ± 0.010 |
| gL2gH15 | | | | |
| HLA-G02 | 0.004 ± 0 | 0.013 ± 0.001 | 0 | 0.041 ± 0.023 |
| (VR12389 | | | | |
| gL2gH16) | | | | |
| Afucosylated | ND | 0.050 ± 0.0043 | ND | ND |
| HLA-G02 | | | | |

At 3 h post vortexing, a low propensity to aggregate (low absorbance at 595 nm) was observed in both PBS pH 7.4 and pH 5 buffer for all antibody samples. It was only possible to discriminate between the samples and assess buffer dependency at the longer time point (24 h). At 24 h, there was a slightly greater propensity to aggregate in pH 5 buffer compared with PBS pH 7.4 and also HLA-G01 compared with VR12389 graft molecules. No meaningful difference was observed between the conventional (i.e. fucosylated) HLA-G02 and its afucosylated counterpart.

### Example 19: Hydrophobic Interaction Chromatography (HIC)

Hydrophobic Interaction chromatography (HIC) separates molecules in order of increasing hydrophobicity. Molecules bind to the hydrophobic stationary phase in the presence of high concentrations of polar salts and desorb into the mobile phase as the concentration of salt decreases. A longer retention time equates to a greater hydrophobicity.

The samples (2.0 mg/mL) were diluted 1:2 with 1.6 M ammonium sulphate, 100 mM phosphate pH 7.4 30 μg (30 μL) of sample was injected onto a Dionex ProPac™ HIC-10 column (100 mm×4.6 mm) connected in series to an Agilent 1200 binary HPLC with a fluorescence detector. The separation was monitored by intrinsic fluorescence (excitation and emission wavelengths, 280 nm and 340 nm respectively). Using Buffer A (0.8 M ammonium sulphate 50 mM Phosphate pH 7.4) and Buffer B (50 mM Phosphate pH 7.4) the sample was analysed using gradient elution as follows, (i) 2 minute hold at 0% B, (ii) linear gradient from 0 to 100% B in 30 minutes (0.8 mL/minute) (iii) the column was washed with 100% B for 2 minutes and re-equilibrated in 0% B for 10 minutes prior to next sample injection. The column temperature was maintained at 20° C. The retention time (in minutes) is shown in Table 31.

TABLE 31
Hydrophobic Interaction Chromatography
of anti-HLA-G humanised antibodies

| | HIC retention time |
|---|---|
| Antibody | (min) |
| HLA-G01 | 6.4 |
| HLA-G02 (VR12389 gL2gH16) | 5.6 |
| Afucosylated HLA-G02 | 5.6 |
| VR12389 gL2gH15 | 5.5 |

The molecules showed early elution times suggesting low apparent hydrophobicity. A low hydrophobic potential is reported to be a desirable characteristic and may indicate increased developability due to decreased aggregation propensity (Jarasch A et al 2015). No meaningful difference was observed between the conventional (i.e. fucosylated) HLA-G02 antibody and its afucosylated counterpart.

### Example 20: HLA-G Tissue Cross-Reactivity

The expression pattern of HLA-G in normal, non-tumoral tissues was investigated using an antibody optimised for staining of frozen tissues that recognises a similar epitope to HLA-G02 (“HLA-G Ab”) and surprisingly, it was found that the forms of HLA-G comprising the epitope bound by HLA-G Ab (and therefore the epitope bound by HLA-G02) were not expressed in healthy tissues, notably in pancreas and pituitary tissues.

This is in contrast to what has been previously reported in the literature where the expression of HLA-G protein in pancreatic islets was reported by Cirulli et al. (Cirulli et al, DIABETES, Vol. 55, May 2006); they observed a significant upregulation of HLA-G in islet cells cultured on an extracellular matrix supporting cell replication. Also for example, the gene expression of HLA-G in pituitary glands, as well as in pancreatic islets and testis has been reported by Boegel et al. (Boegel et al, BMC Medical Genomics (2018) 11:36).

### Methods

A Tissue Cross Reactivity study in Human Tissues was performed using the HLA-G Ab mentioned above, which specifically binds to HLA-G. The objective of this Tissue Cross Reactivity (TCR) study was to evaluate the potential cross reactivity of the HLA-G antibody using a FITC-conjugated HLA-G antibody in frozen human tissues and blood smears, using immunohistochemical (IHC) techniques.

A panel of 42 different frozen normal human tissues and blood smears (three donors per tissue) was evaluated. Two concentrations of HLA-G Ab-FITC set at 3 and 10 μg/mL were used, with negative control IgG1-FITC at the highest concentration of 10 μg/mL.

### Results

HLA-G Ab-FITC yielded membranous, variably cytoplasmic staining in extravillous trophoblast in placentas. As HLA-G is a major histocompatibility gene expressed almost exclusively in extravillous trophoblasts at the fetal-maternal interface (the extravillous trophoblasts invade the decidua and maternal spiral arteries), this pattern was considered to represent on-target binding of the HLA-G Ab (Goldman Whol, 2000).

In contrast, no positive staining was observed in the following tissues: adrenal gland, blood cells, bone marrow, breast, cecum, cerebellum, cerebral cortex, colon, duodenum, endothelium (vessels), eye, esophagus, fallopian tube (oviduct), gall bladder, heart, ileum, jejunum, kidney, liver, lung, lymph node, muscle, nerve, ovary, pancreas, parotid gland, parathyroid gland, pituitary gland, prostate, rectum, skin, spinal cord, spleen, stomach, testis, thymus, thyroid, tonsil, ureter, urinary bladder, uterus (cervix and endometrium).

In conclusion, it was found that the forms of HLA-G comprising the epitope bound by the HLA-G Ab are only expressed in extravillous trophoblast cells (as reported in the literature and used as a control in the present study) and that it was not expressed in any other normal tissue tested.

Therefore, the results are surprising, and show that contrary to what would have been expected from the teaching of the prior art, an antibody against HLA-G which is capable of killing cells expressing HLA-G, for example through Fc mediated effector functions, represents a potential candidate for the treatment of solid tumors, with no expectation of toxicity for the patients through binding to normal tissues. A posteriori, there are potential hypothesis that could explain the unexpected differences in results obtained in this assay compared to what has been reported in the literature: i) mRNA expression, as reported in pituitary, does not necessarily indicate membrane protein expression. For example, the mRNA may not be translated into protein, or the mRNA may encode soluble HLA-G isoform(s) that may not be detected in frozen tissue and which would not present a problem in terms of toxicity as they are not membrane bound. The mRNA may also be expressed by infiltrating immune cells rather than by pituitary cells, ii) the commercial antibody 4H84 was used for detecting HLA-G protein in pancreas and this antibody is known to be non-specific. 4H84 also recognizes an epitope in the α1 domain of HLA-G, whereas the antibody of the invention is highly specific to HLA-G and binds the α3 domain. It is therefore possible that the HLA-G isoforms expressed in pancreas do not contain the α3 domain but will still be detected by an α1 binder (e.g. HLA-G3, HLA-G4, HLA-G7). The positive detection of HLA-G in placental trophoblasts and lack of detection in normal tissues suggests that the antibody of the invention may be capable of binding to HLA-G proteins expressed in tumors containing the ILT2/4 binding α3 domain that is required for immune modulatory function of HLA-G, but may not bind cells in normal tissues.

The dual mechanism of such an antibody as described herein, capable of blocking the interaction between HLA-G and its inhibitory receptors, and capable of cell killing, represents a considerable advantage for the treatment of patients with upregulation of HLA-G, such as in solid cancers.

### Example 21: Functional Properties of HLA-G Antibodies in a 3D Tumoroid Model

HLA-G antibody activity was assessed in a primary ex-vivo human tumoroid platform using Nilogen Oncosystems (Tampa, Florida, US)' 3D tumoroid model technology. Nilogen's technology employs fresh patient derived tumor tissue and results in the generation of 3D tumor organoids that retain the intact tumor microenvironment including the infiltrating immune cells and capture the full tumor heterogeneity. The technology captures similar patient response rates to those seen in the clinic, and therefore provides a model useful to evaluate the potential of immunotherapy candidates for the treatment of patients.

Ten colorectal adenocarcinoma tumors (CRC) and ten renal clear cell carcinoma tumors (RCC) were obtained and each used to derive many thousands of tumoroids containing all tumor cellular (tumor cells, stromal cells, infiltrating immune cells) and matrix components using Nilogen's methodology.

Isolated tumoroids (100-400 per treatment well) were immediately, without prior culture, exposed to antibody for 72 h. Levels of tumor cell death were evaluated at 24 h and 72 h using Nilogen's 3D-Explore™ imaging platform. Culture media was collected at 24 h for assessment of cytokine levels. Tumoroids were disaggregated at 72 h and analysed by flow cytometry to assess effects on immune cell activation profiles. In addition, prior to treatment, tumoroids were analysed by flow cytometry to characterise their cell composition, and FFPE sections of each tumor were stained for HLA-G, ILT2 and ILT4.

Activity of the HLA-G02 antibody was analysed in an active, IgG1, format by comparison to an IgG1 isotype control. An anti-PD-L1 antibody with an active IgG1 format was used for comparison.

### Tumor Cell Death

Immediately after isolation, cultures containing ˜100 tumoroids per well were cultured in the presence of either isotype control IgG1, anti-PDL1 positive control or HLA-G02 IgG1 at 10 mg/ml. After 24 hours and 72 hours of culture tumoroids were stained with live/dead dyes, imaged and percent dead cells calculated using a proprietary algorithm.

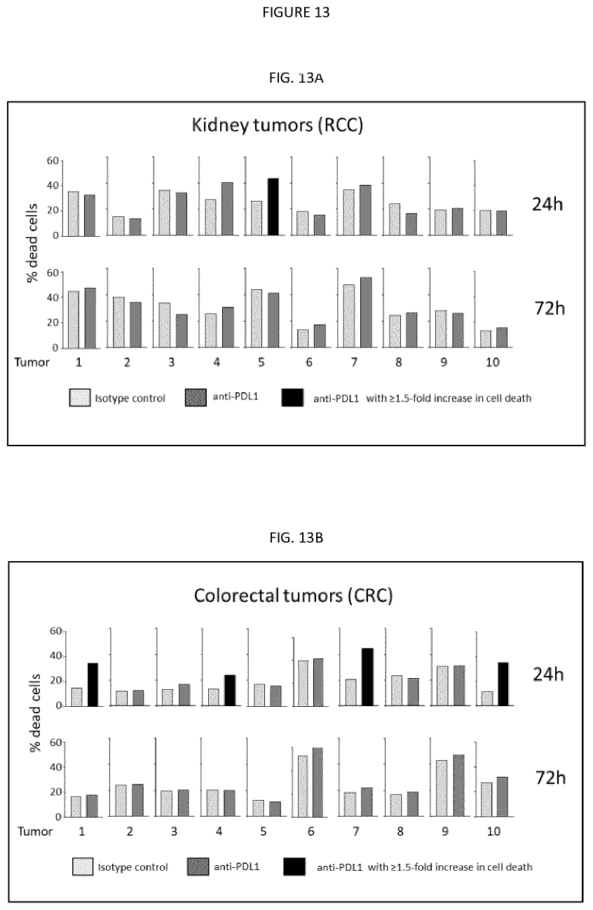

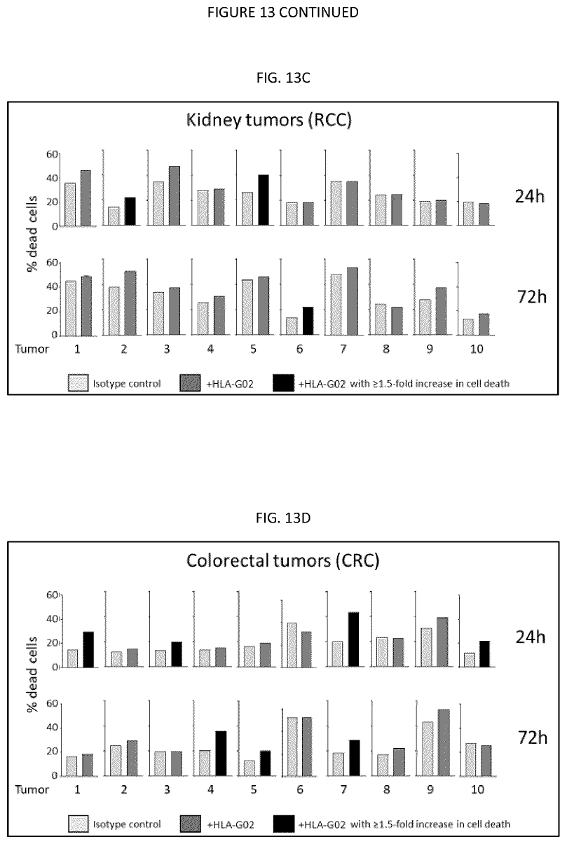

The results are presented in FIG. 13. FIG. 13A: data obtained with anti-PDL1 from RCC. FIG. 13B: data obtained with anti-PDL1 from CRC. FIG. 13C: data obtained with HLA-G02 from RCC. FIG. 13D: data obtained with HLA-G02 from CRC. Data is presented as % dead cells for isotype control in light grey, anti-PDL1 or HLA-G02 in dark grey and anti-PDL1 or HLA-G02 treated cultures where a 1.5-fold or greater increase in cell death was observed in black.

Each tumor may differ from one patient to another therefore the data represents well the potential of the antibody of the invention in the treatment of cancer, notably RCC and CRC. Overall, the data shows that the antibody of the invention is capable of killing tumor cells in a tumoral environment and under conditions where responses are dependent entirely on the infiltrated immune cells. Advantageously, the data provides evidence that the antibody may have an increased cell killing activity in certain tumors.

Of note, as dead cells are lost from the cultures over time, an increase in killing at 24 h may no longer be detected at 72 h. In addition, for the anti-PDL1 positive control a total of 5 tumors showed an increase in cell killing at 24 h (1×RCC and 4×CRC), with no increased killing observed at 72 h. For HLA-G02, 6 tumors showed increased killing at 24 h (2×RCC and 4×CRC) and 4 at 72 h (1×RCC and 3×CRC). In total only 5 of 20 tumors showed increased killing with anti-PDL1, but 9 of 20 tumors showed increased killing with HLA-G02.

Therefore, the data shows that the antibody of the invention may be advantageous in the treatment of solid tumors, as compared to an anti-immune checkpoint such as an anti-PD-L1.

### Example 22: Tissue Cross Reactivity Assay with Afucosylated HLA-G02

The expression pattern of HLA-G in normal, non-tumoral tissues was further investigated using afucosylated HLA-G02 using the same method as described in Example 20.

A panel of 37 different frozen normal human tissues and blood smears (three donors per tissue) was evaluated (except for pituitary and pancreas, where 8 donors were evaluated). Two concentrations of HLA-G Ab-FITC set at 1 and 10 μg/mL were used, with negative control IgG1-FITC at the highest concentration of 10 μg/mL.

Frozen sections of human placenta and wild type HLA-G expressing cells (HLA-G1) were used as the positive control samples. Frozen sections of human colon, HLA-G Null 1,2,3 cells (HLA-G unique amino acids in each of the 3 alpha domains mutated to MHC class I consensus amino acids) and untransfected cells were used as the negative control samples. The purpose of using the null protein is to test for potential cross-reactivity to other MHC class I molecules.

As a result, afucosylated HLA-G02 yielded membranous, variably cytoplasmic staining in extravillous trophoblast in placentas, consistent with what was previously reported, but no membrane staining on normal, healthy tissue was observed with afucosylated HLA-G02 at the optimal concentration, confirming that afucosylated HLA-G02 does not bind any epitope exposed on the membrane of normal, healthy cells.

### Example 23: Binding Affinity to JEG3 Cells as Determined by FACS Assay

Binding affinity of afucosylated HLA-G02 was measured in a flow cytometry cell-based assay using JEG3 naturally expressing HLA-G.

JEG3 cells were incubated with 1.5 ml afucosylated HLA-G02 solution for two hours at 4 degrees in microcentrifuge tubes (Eppendorf). IgG concentrations ranging from 10 nM to 0.00046 nM, diluted in PBS, 1% FBS, 0.1% Sodium azide. Cells were transferred into 384-well V-bottom plates (Greiner) and washed three times in assay buffer, incubated with 20 μl of staining solution for 20 min at 4 degrees (R-Phycoerythrin AffiniPure F(ab′)₂ Fragment Goat Anti-Human IgG (H+L) (Jackson ImmunoResearch)—7.5 μg/ml and Viability Dye e780 (Life Technologies)). After washing step, cells were incubated for 10 min in 10% neutral buffered formalin solution (Sigma-Aldrich) at room temperature, protected from light. Cells were then washed and re-suspended in 20 μl PBS. Samples were run on a FACS Canto II instrument in HTS mode to determine the percentage of PE positive cells. K_{D} were calculated from the median fluorescence intensity using FlowJo analysis software.

The results confirm that afucosylated HLA-G02 binds HLA-G on JEG-3 cells with high affinity/avidity in this assay. Afucosylated HLA-G02 had an EC₅₀ of 0.021±0.001 nM, as determined by the geometric mean of three independent assays, and the assay was highly reproducible with an EC₅₀ range of 0.020 to 0.021 nM.

### Example 24: Efficacy and Potency of HLA-G02 and Afucosylated HLA-G02 in ADCC of HLA-G Expressing Cells

### Methods

HLA-G transfected HCT116 (prepared as described previously in Example 9) target cells were plated out (2×10⁴ cells/well in a volume of 50 μL) into a polypropylene round bottom plate in the appropriate culture medium. Anti-HLA-G (conventional HLA-G02 or afucosylated HLA-G02) or control antibodies (isotype IgG1 or afucosylated IgG1 isotype) were prepared as 4× concentrated stocks in the same medium and 50 μl/well was added to appropriate wells. All antibodies were tested in either duplicate or triplicate depending on the number of available donor NK cells. Some target cells were left without any antibody and were used as no treatment controls.

Primary human NK cells were isolated from whole blood by negative selection using a magnetic bead kit (Miltenyi Biotech). The purified NK cells were resuspended in RPMI+10% FBS, 2 mM L-Glutamine in the minimum volume needed for the assay. To appropriate wells of the assay plate 100 μl/well NK cells were added on top of the target cells and antibodies.

For effector:target titration experiments, the NK cells were diluted and plated out (100 μL/well) into the 96 well assay plate containing the target cells and antibodies to give a final effector:target ratio range of 20:1 to 0.313:1.

The assay plate was incubated at 37° C. 5% CO₂ for 2.5 to 3 hours. After 2.5 to 3 hours, the number of live target cells was measured by flow cytometry. The assay plate was centrifuged at 300 g for 2 minutes to pellet the cells and each well was stained for the epithelial cell marker Epcam and the NK cell marker CD56. The staining antibodies (anti-Epcam PE and anti-CD56 BV421) were diluted to 1/100 in cell staining buffer and 100 μL/well was added to each well. The plate was incubated at room temperature for 15 minutes. Following staining the cells were washed twice with 150 μl/well PBS and the plate was centrifuged at 300 g for 3 minutes in between each wash. At the end of the staining the cells in each well were resuspended in a final volume of 125 μL/well PBS containing 50 nM TO-PRO™-3 cell viability dye.

After 30 seconds, exactly 100 μL of sample from each well was acquired on a BD FACS Canto II Instrument and the data was analysed using FlowJoV10.60 software. The total number of live target cells was determined for each well. Live target cells were identified firstly as TO-PRO-™3 negative and then secondly as CD56 negative and SSC high. The cells were then gated on or Epcam and GFP expression (HCT116). Percent depletion compared to either untreated cells or the isotype control was calculated for each test sample and the data was transferred to GraphPad Prism 8.1.1 Software for analysis.

### Results

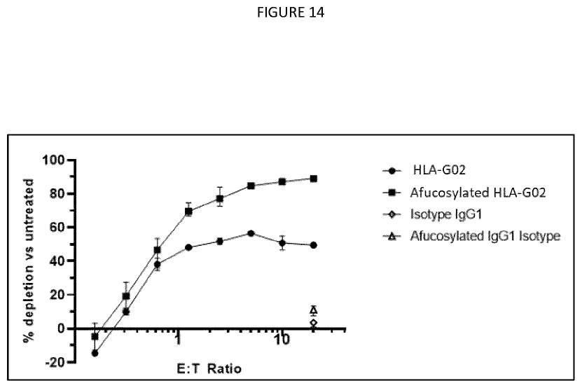

Data are shown in Table 32 below and in FIG. 14. A higher maximum mean depletion was observed when cells were treated with afucosylated HLA-G02 compared to its conventional (i.e. fucosylated) counterpart, at all effector:target ratios, for a given donor. Importantly, HLA-G positive HCT 116 cells were still depleted at low effector:target ratios of below 1:1. The mean depletion observed at an effector target ratio of 0.625:1 was still 46.63% and 38.15% for afucosylated HLA-G02 and its conventional (i.e. fucosylated) counterpart, respectively. This could be important in the tumor microenvironment where the number of immune cells such as NK cells is often limited. These experiments confirm that afucosylation of HLA-G02 has increased the depletion activity of the molecule compared to a conventional IgG1 format of the antibody.

TABLE 32
mean depletion of HLA-G + GFP + transfected
HCT116 cells at different E:T ratio

| | HLA-G02 | | | Afucosylated HLA-G02 | | |
|---|---|---|---|---|---|---|
| E:T Ratio | Donor 1 | Donor 2 | Donor 3 | Donor 1 | Donor 2 | Donor 3 |
| 20:1 | 49.58 | 58.46 | 23.26 | 89.07 | 87.19 | 51.67 |
| 10:1 | 50.80 | 51.95 | 15.33 | 87.05 | 80.85 | 40.75 |
| 5:1 | 56.48 | 45.42 | 14.88 | 84.72 | 73.50 | 34.03 |
| 2.5:1 | 51.78 | 41.11 | 3.49 | 77.15 | 66.73 | 18.99 |
| 1.25:1 | 48.16 | 34.11 | −4.09 | 69.57 | 64.42 | 2.17 |
| 0.625:1 | 38.15 | 32.88 | −17.56 | 46.63 | 46.11 | −7.15 |
| 0.313:1 | 9.94 | 32.75 | −16.85 | 19.30 | 52.57 | −8.06 |

### Example 25: Specificity of Afucosylated HLA-G02 Assessed in a PBMC Experiment

The specificity of afucosylated HLA-G02 was further confirmed using PBMCs from 10 different donors, representing a variety of HLA-I alleles. The aim was to confirm that the anti-HLA-G antibody is specific and does not cross-react with other HLA-I molecules expressed on PBMCs and CD4+T lymphocytes in particular.

PBMCs were purified from peripheral venous blood and stored in Liquid nitrogen in Freezing medium (90% FBS+10% DMSO). PBMCs from 10 different donors were thawed and resuspended in 1 ml of complete RPMI medium (RPMI 1640 medium plus 10% Foetal Bovine Serum, 2 mM Glutamax and 1% Penicillin/Streptomycin). Cells were centrifuged at 300 rpm, 10 min and washed twice with PBS. Cell pellets were resuspended in 1 ml Facs buffer (PBS, 0.5% BSA and 2 mM EDTA) and cells were seeded in 96 well plate with 50 μl cell suspension/well.

The cells were stained with anti-Human CD4-APC (Biolegend, 2.5 μl/well) and with anti-HLA-G antibodies (afucosylated HLA-G02 or the two controls “pan-HLA”, two IgG1 which bind to HLA-Is and are not specific to HLA-G) or isotype control (50 μl of solution at 20 μg/ml per well). Cells were incubated at room temperature in the dark for 20 min and then washed twice with Facs buffer and resuspended in 50 μl of Facs buffer containing the secondary antibody Goat Anti-Human IgG-FITC (Jackson ImmunoResearch, dilution 1/100). The cells were incubated for a further 20 min at RT in the dark, then washed twice with Facs buffer. Cells were resuspended in 100 μl/well of Facs buffer and samples were acquired on the Canto II (HTS 1), 10,000 events collected per sample. Analysis was performed using FlowJo software v10.6.0 by measuring the Mean Fluorescence Intensity (MFI) of each CD4+ cell population for each donor. Graphs were performed using Graphpad Prism software.

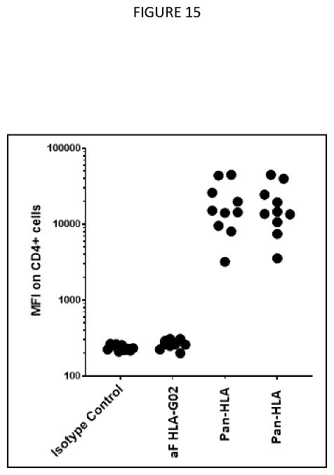

The results are presented in FIG. 15. FIG. 15 shows the lack of binding to CD4 T cells across 10 different donors of the specific anti-HLA-G antibody afucosylated HLA-G02 in comparison to two pan-HLA antibodies. Data are expressed in Mean Fluorescence Intensity (MFI) for each donor.

### Example 26: Assessment of Complement Dependent Cytotoxicity (CDC) Mediated by Afucosylated HLA-G02

### Methods

The following method was used to assess CDC mediated by afucosylated HLA-G02 (“aF HLA-G02”): HLAG-β2m-Reh cells were loaded with fluorescent calcein-AM dye and incubated in antibody solution for 2 h at 37° C. in the presence of pooled human serum. Cells were pelleted and supernatants were collected. Fluorescence within supernatants was quantified using a spectrophotometer to quantify cell lysis. Data was processed in Excel and exported to Prism where curves were plotted, allowing calculation of the EC₅₀ (half maximal effective concentration) and Emax (maximal effective concentration) for each test sample. Details of the method are provided below.

### Cell Line

The Reh cell line (ATCC CRL-8286) exhibits lymphoblastic morphology and was isolated from human tissue from an acute lymphocytic leukemia patient. The human lymphoblastic HLAG-β2m-Reh cell line is a polyclonal pool of cells which stably express human HLA-G and β2 microglobulin. To create the HLAG-β2m-Reh cell line, HLA-G and β2 microglobulin codon optimised sequences were cloned into a mammalian gene expression lentiviral vector and packaged into lentivirus (pLV-Neo-EF1A-HumanHLA-G:IRES:HumanB2m; Vectorbuilder). Reh cells were spinoculated with HLAG-β2m lentivirus at a multiplicity of infection of 30, after which transfected cells were maintained in medium containing gentamicin (1 mg/ml). After 7 days, expression of HLA-G on cells was quantified by flow cytometry. Briefly, untransfected Reh and HLAG-β2m-Reh cells were bound by PE-labelled HLA-G antibody (MEM-G/9; Invitrogen) and binding of antibody to the cell surface was quantified. In parallel the fluorescence of Quantibrite beads (BD) was quantified. Using these beads the average number of HLA-G receptors on the cell surface of HLAG-β2m-Reh cells was determined to be 76665. The HLAG-β2m-Reh cell line was maintained in RPMI-1640 supplemented with Foetal Bovine Serum (FBS, 10%) and glutamax (1%) and gentamicin (1 mg/ml).

### CDC Assay

Pooled human serum was thawed and aliquoted after receipt from the vendor. It was stored at 80° C. until use. Immediately prior to use, serum was allowed to thaw on the bench at RT. Serum was inactivated by heating at 56° C. for 30 min, as required. HLAG-β2m-Reh cells were centrifuged for 3 min at 300×g, supernatant was aspirated and cells were re-suspended in PBS at 1×10⁷ cells/ml. Calcein-AM was added (10 μM) and HLAG-β2m-Reh cells were incubated for 1 hour at 37° C. Cells were centrifuged for 3 min at 300×g, supernatant was aspirated and cells were resuspended in assay buffer. This was repeated for a total of two washes. After the final wash, cells were re-suspended in assay medium (RPMI-1640, 2% FBS, 1% glutamax) at 0.2×10⁶ cells/ml. Cells were dispensed across a 384-well v-bottom plate (50 μl/well). Active or inactive pooled human complement serum solution was prepared (1 part serum, 4 parts assay medium) and dispensed across the assay plates (25 μl/well) as required. A serial dilution of afucosylated HLA-G02 was created in a 96-well v-bottom plate, in assay medium. Initially, a 400 nM top concentration was prepared in assay medium. This solution was diluted 1:3 across the plate using the Assist (Integra), to form a 10-point serial dilution. Additional wells were prepared containing assay medium, for assessing background fluorescence (MIN) and maximum lysis (MAX).

Test reagent (afucosylated HLA-G02 at 3.7 mg/ml or Human IgG₁ isotype control antibody at 1 mg/ml; 25 μl) and MIN/MAX controls (25 μl) were transferred from the 96-well plate to the 384-well assay plate, in duplicate using the Viaflo (Integra).

The assay plate was sealed with a breathable membrane, vortexed briefly and centrifuged for 10 sec at 300×g. The assay plate was incubated for 2 h at 37° C. and 5% CO₂.

After the 2 h incubation period, 10 μl lysis buffer (10% Triton-X, assay medium) was added to all MAX control wells and mixed well by pipetting. The assay plate was incubated for 10 min at 37° C. and 5% CO₂. The assay plate was centrifuged at 300×g for 3 min.

Using a Viaflo (Integra), 40 μl supernatant was removed from each well and transferred to a 384-well black walled, flat, clear bottom plate. Care was taken not to disturb the cell pellet.

The new assay plate was centrifuged at 1000×g for 5 min. The new assay plate was analysed on a spectrophotometer with an excitation of 488 nm and an emission of 520 nm.

### Statistical Analysis

Fluorescence emission data was exported to Excel. The fluorescent signal was normalized against an average of treatment wells containing assay buffer (MIN) and 1% Triton X-100 (MAX) to generate the percentage lysis achieved at a given concentration of test reagent. 4-parameter logistic fit (4-PL) curve fitting and calculation of EC₅₀ values was performed using Graphpad Prism® 8.0 software.

### Results

### Impact of Serum Inactivation on Lysis of HLAG-β2m-Reh Cell Line

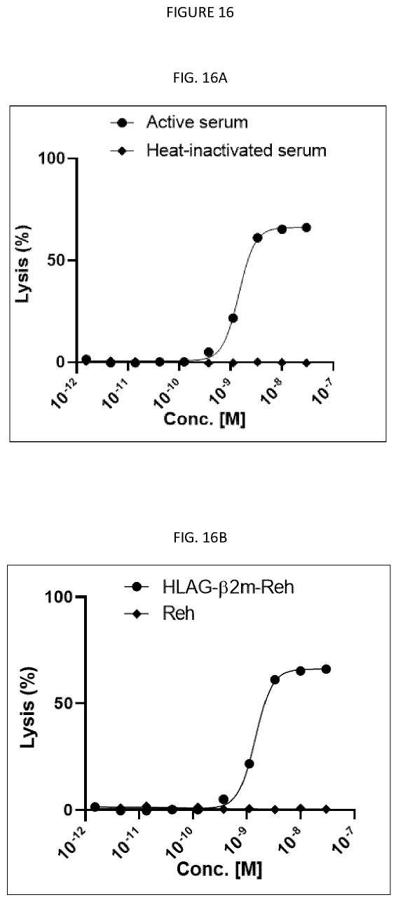

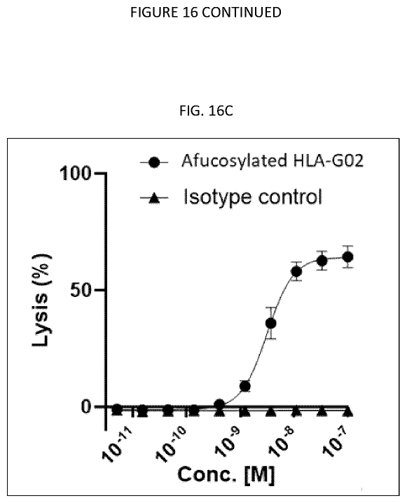

The results confirm that active human serum was essential for aF HLA-G02 mediated CDC of HLAG-β2m-Reh cells. When serum was inactivated by heating, aF HLA-G02 was unable to mediate CDC. Data are visualized in FIG. 16 A.

### Dependence on HLA-G for Depletion

In the presence of active human serum, lysis of the HLAG-β2m-Reh cell line was mediated by aF HLA-G02 in a concentration dependent manner. Lysis of the parental Reh cell line was not observed up to a maximal aF HLA-G02 concentration of 100 nM. Data are visualized in FIG. 16 B.

### CDC of HLAG-β2m-Reh Cells

The results confirm that in the presence of active serum, aF HLA-G02 mediated CDC of HLAG-β2m-Reh cells. aF HLA-G02 was tested at concentrations ranging from 100 nM to 0.0051 nM. aF HLA-G02 had an EC₅₀ of 3.17±0.60 nM, as determined by the mean of three independent assays. Data are summarised in Tables 33 and 34 below and visualized in FIG. 16 C.

TABLE 33
Lysis (%) mediated by aF HLA-G02 at different concentrations, EC50 and
Emax. Values were calculated from three independent experiments.

| aF HLA- | Lysis (%) at concentration (nM) | | | | | | | | | | EC50 | Emax |
|---|---|---|---|---|---|---|---|---|---|---|---|---|
| G02 lot | 100.0 | 33.3 | 11.1 | 3.70 | 1.23 | 0.41 | 0.14 | 0.046 | 0.015 | 0.0051 | (nM) | (%) |
| 1 | 68.5 | 67.9 | 64.3 | 46.2 | 11.5 | 1.8 | −0.4 | −0.7 | −0.6 | 0.3 | 2.51 | 68.5 |
| 2 | 66.2 | 63.1 | 57.4 | 35.6 | 3.7 | −0.4 | −1.8 | −1.3 | −1.6 | −1.5 | 3.29 | 66.2 |
| 3 | 54.9 | 54.8 | 50.9 | 26.5 | 7.8 | 2.0 | −2.0 | −2.0 | −1.9 | −1.9 | 3.70 | 54.9 |

TABLE 34
Summary of the mean EC50 and Emax
(SEM (standard error of the mean))

| | |
|---|---|
| EC₅₀ (nM) | Eₘₐₓ (%) |

| | Mean | SEM | Mean | SEM |
|---|---|---|---|---|
| aF HLA-G02 (N = 3) | 3.17 | 0.60 | 63.2 | 7.23 |

### Conclusion

Afucosylated HLA-G02 binding to HLAG-β2m-Reh triggered potent and efficacious complement dependent lysis (EC₅₀=3.17±0.60 nM; Eₘₐₓ=63.2±7.23%).

To ensure the observed lysis was CDC mediated, an exploratory study using heat-inactivated serum was performed. It was shown that afucosylated HLA-G02 triggered lysis of HLAG-β2m-Reh cells only when in the presence of active pooled human serum. When pooled human serum was inactivated by prolonged exposure to high temperature, the ability of afucosylated HLA-G02 to mediate CDC was annulled.

To ensure the observed lysis was HLA-G dependent, the parental, untransfected Reh cell line was exposed to afucosylated HLA-G02 in the presence of active pooled human serum. It was shown that afucosylated HLA-G02 was unable to mediate CDC of Reh cells within the concentration range tested. However, afucosylated HLA-G02 was both potent and efficacious at mediating CDC of HLAG-β2m-Reh cells within the same concentration range.

In conclusion, these results have demonstrated the selectivity, potency, and efficaciousness of afucosylated HLA-G02 at mediating CDC of an HLA-G expressing cell.

### Example 27: Phagocytosis Mediated by HLA-G02 in Different Active and Inactive Fc Formats, and Combination Treatment of HLA-G02 with Anti-CD47 Antibody

The aim of this study was to evaluate the ability of afucosylated HLA-G02 to promote macrophage mediated phagocytosis of HLA-G expressing tumor cells using an in vitro macrophage-dependent phagocytosis assay.

We investigated the ability of afucosylated HLA-G02 to direct phagocytosis of HLA-G expressing K562 target cells when expressed in different Fc formats: ‘active’ Fc formats capable of interaction with Fc receptors expressed on macrophages (afucosylated HLA-G02 and its conventional (i.e. fucosylated) counterpart to assess the impact of the antibody Fc optimization), and an ‘inactive’ Fc format (HLA-G02 IgG4P FALA) that does not interact with Fc receptors, to evaluate the possibility of an FcR independent mechanism of action.

In these studies, anti-CD47 antibody was used as a positive control. CD47, widely expressed on human cells, interacts with the receptor SIRPa on myeloid cells to prevent phagocytosis. CD47 has been shown to be overexpressed in tumor cells and anti-CD47 blocking antibodies that promote tumor cell phagocytosis are currently being tested in clinical trials.

In addition, we tested the potential effect of combined treatment with anti-CD47 and afucosylated HLA-G02.

### Methods

CD14+ monocytes were purified from peripheral venous blood using Pan Monocyte Isolation Kit, human (Miltenyi), an indirect magnetic labelling system for the isolation of untouched monocytes. Cells were differentiated into macrophages with 50 ng/ml recombinant MCSF in complete RPMI medium (RPMI 1640 medium plus 10% Foetal Bovine Serum, 2 mM Glutamax and 1% Penicillin/Streptomycin) for 7 days at 37 C, 5% CO2.

HLA-negative erythroleukemia K562 cells were either mock transfected or transfected with HLA-G and B2m using the 4D-Nucleofector System and the SF Cell Line 4D-Nucleofector™ X Kit L (Lonza, ref #V4XC-2024) and cultured in complete RPMI for 24 hrs at 37 C, 5% CO2. The next day, the cells were harvested, washed and labelled with Cell Trace Yellow (Thermofisher), washed again and plated at 25.000 cells per well in 100 μl complete RPMI in 96 well round bottom Ultra low attachment plate (Corning Costar). The cells were subsequently incubated with either anti-CD47 antibody or anti-HLA-G antibodies or isotype controls at 10 μg/ml for 1 hour at 37 C, 5% CO2. After a wash, the cells were combined with monocyte-derived macrophages (50.000 macrophages per well) at a ratio Macrophage:cell target=2:1. The mixed cells were incubated for 2 hours at 37° C., 5% CO2. Then, cells were washed and resuspended in PBS plus 10% Purified human Fc gammaR-binding inhibitor (Thermofisher) for 20 minutes at 4° C. and then stained with anti-CD11b-APC (Biolegend) for 20 minutes at 4° C. Cells were washed and resuspended in PBS plus 2 mM EDTA plus 0.5% BSA in presence of DAPI (500 ng/ml) dead cells exclusion. Samples were acquired by flow cytometry on the BD FACSCanto. Analysis was performed using FlowJo software v10.6.0 by measuring the percentage of phagocytosis corresponding to the percentage of macrophages that are CTY+CD11b+ double positive cells.

The Data from duplicates have been normalized to the corresponding isotype control and expressed as % depletion versus control (% depletion=100−[(Mean target cell aF HLA-G02)×100/(Mean Target cells isotype control)].

Data were exported to excel files and graphs were generated using Graphpad Prism software.

### Results

### Phagocytosis Mediated by HLA-G02 and Afucosylated HLA-G02

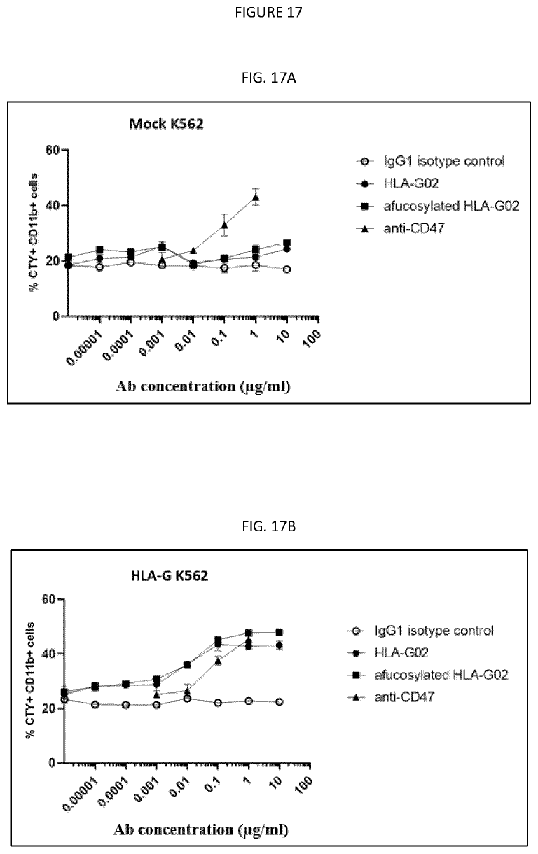

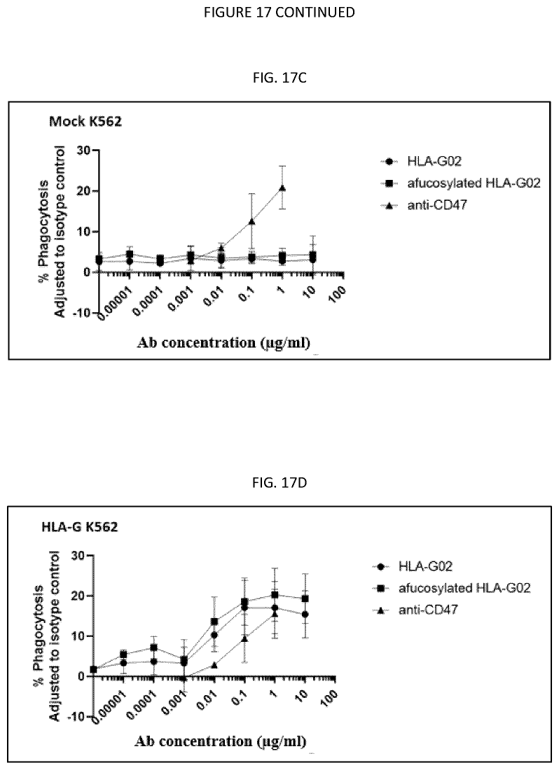

Comparison of phagocytosis of mock-transfected and HLA-G transfected cells (mock transfected cells were subjected to the transfection protocol in the absence of HLA-G DNA) show the specificity of afucosylated HLA-G02 (and of its fucosylated counterpart) mediated ADCP for HLA-G expressing cells compared to the positive control anti-CD47 antibody that induces phagocytosis of both cell types. A concentration-dependent phagocytosis of HLA-G expressing target cells is observed with both conventional HLA-G02 and afucosylated HLA-G02 compared to isotype control. An increasing phagocytosis is observed with afucosylated HLA-G02 at concentrations starting from 0.001 μg/mL. Afucosylated HLA-G02 shows a minimal enhancement of the macrophage-mediated phagocytosis compared to its conventional, fucosylated, counterpart. The results are illustrated in FIG. 17.

### Target Cell Killing Mediated by Afucosylated HLA-G02

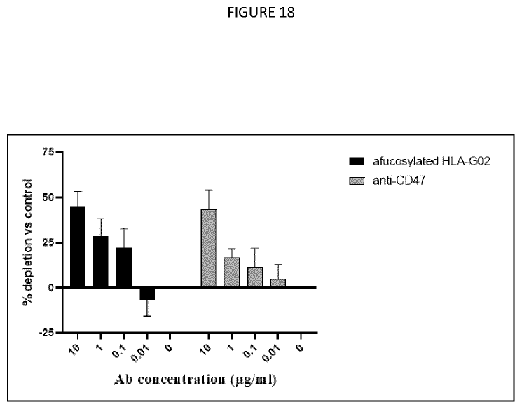

The killing of HLA-G expressing target cells was assessed by comparing the number of target cells remaining after a defined time of phagocytosis (overnight) with afucosylated HLA-G02 or anti-CD47 antibody compared to isotype control. The data show that both antibodies induce similar concentration-dependent depletion of target cells. Afucosylated HLA-G02 treatment results in the depletion of 22.0±10.8% of target cells at 0.1 μg/mL and 44.8±8.6% at 10 μg/mL, and anti-CD47 treatment to 11.3±10.4% and 43.3±10.8%, respectively. The results are illustrated in FIG. 18.

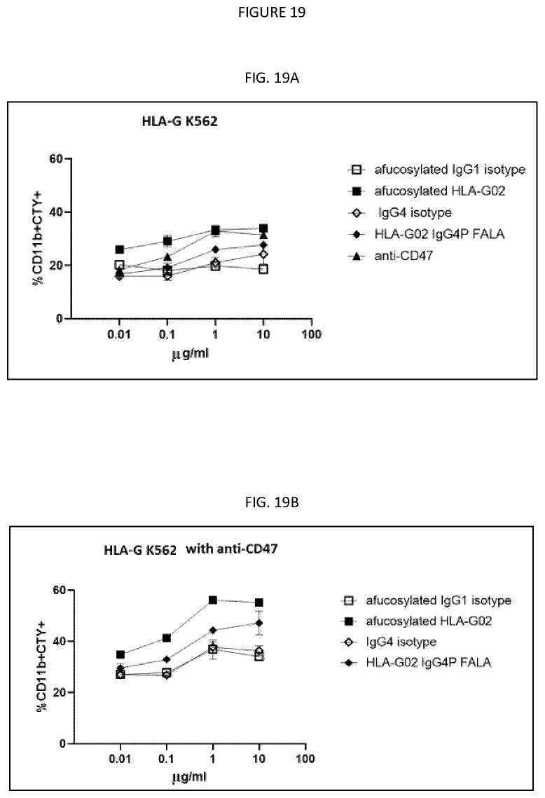

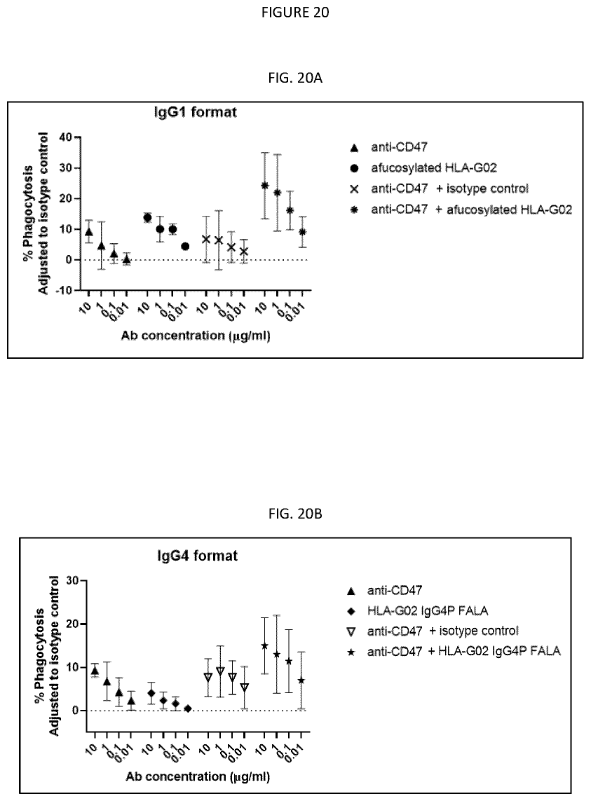

Anti-CD47 antibodies are currently tested in clinical trials as cancer therapeutics. We subsequently investigated whether combined treatment with afucosylated HLA-G02 and anti-CD47 antibody would increase phagocytosis of HLA-G expressing cells to levels greater than observed with either agent alone. In this experiment, HLA-G expressing cells were treated with anti-CD47 antibody (1 μg/mL) in combination with increasing concentrations of afucosylated HLA-G02 or HLA-G02 IgG4P FALA. Representative data are shown in FIG. 19 and combined data from several donors shown in FIG. 20.

The IgG4P FALA format was included to determine if the increased phagocytosis observed with afucosylated HLA-G02 was dependent upon its active Fc format. Afucosylated HLA-G02 alone induced a concentration-dependent phagocytosis in its afucosylated IgG1 format. HLA-G02 IgG4P FALA also increased phagocytosis, although to a lesser extent. Unlike IgG1, IgG4P FALA cannot induce ADCP through Fc receptors, clearly suggesting that the phagocytosis observed with HLA-G02 IgG4P FALA is mediated by blocking HLA-G from engaging its receptors ILT2/4 on macrophages. In addition, the data show that combination of anti-CD47 and afucosylated HLA-G02, in either format, results in an enhanced phagocytosis of HLA-G expressing cells.

As a summary, the study showed that afucosylated HLA-G02 and its conventional (i.e. fucosylated) counterpart both induced specific antibody-dependent cellular phagocytosis (ADCP) of HLA-G expressing cells. The IgG4P FALA format of HLA-G02 also increased phagocytosis but to a lesser extent. The data indicate that afucosylated HLA-G02 and its fucosylated counterpart promote phagocytosis through two mechanisms of action: (1) through Fc receptor mediated antibody-dependent cellular phagocytosis and (2) by blocking the interaction of HLA-G with its receptors.

In addition, we have found that combination of afucosylated HLA-G02 and an antibody targeting the phagocytic checkpoint CD47 together increase the phagocytosis of HLA-G expressing cells.

### Example 28: Cytokine Release Mediated by Afucosylated HLA-G02

Cytokine release assays (CRAs) provide a method for evaluating the potential for novel therapeutics to induce cytokine release from immune cells. Typically, these assays use peripheral blood mononuclear cells (PBMCs) or whole blood, treated with a therapeutic of interest before pro-inflammatory cytokines are measured.

This study aims to evaluate the effect of soluble afucosylated HLA-G02 on pro-inflammatory cytokine release from sixteen human PBMC donors and compare the cytokine level to known positive controls. Cytokine release was measured using an MSD multiplex sandwich immunoassay. The level of cytokine release was also measured following a co-culture of afucosylated HLA-G02-treated PBMCs with JEG3 cells (which express high levels of HLA-G). This co-culture aimed to mimic the potential level of cytokine release in an HLA-G positive tumor microenvironment following active Fc depletion mechanisms.

### Methods

PBMC were isolated from human whole blood according to standard methods. JEG3 cells were cultured and maintained at a density of 4×10⁵ cells/mL in RPMI complete media. To appropriate wells of a 96-well flat bottom tissue culture plate JEG3 cells were added (50 μL/well). For the PBMC only wells, 50 μL of RPMI complete media (RPMI 1640 (500 mL)+10% FBS (50 mL)+2 mM Glutamax™ (5 mL)) was plated out in place of the JEG3 cells.

Afucosylated HLA-G02 was diluted to a starting concentration of 200 μg/mL (4× the maximum final concentration of 50 μg/mL) then serially diluted to 100, 40, 4, 0.4, 0.04, 0.004 and 0.0004 μg/ml (to give final concentrations of 25, 10, 1, 0.1, 0.01, 0.001, and 0.0001 μg/ml). An isotype control antibody and anti-CD3 antibody were prepared at the highest antibody concentration only (200 μg/mL) for use as negative and positive controls respectively. As an alternative positive control, LPS was also diluted to 400 ng/mL in RPMI complete media (4× the final concentration of 100 ng/mL). Once diluted, the test antibodies/substances were plated out (50 μL/well) in triplicate on top of the JEG3 or media in the 96 well flat-bottom assay plate. PBMCs were then plated out into the flat bottom plate on top of the antibodies −100 μL (1×10⁵) cells/well. The total volume in each well was 200 μL. The assay plates were then transferred to a 37° C. 5% CO₂ 100% humidity incubator for 24 hours.

After 24 hours the 96-well culture plates were centrifuged at 400 g for 3 min to ensure no cells remained in the supernatant in each well. From each well 150 μL supernatant was transferred to a new 96-well plate and the samples were stored at −20° C. until the multiplex assay was performed.

The level of cytokine was measured in the supernatant using MSD Proinflammatory Panel 1 kit according to the manufacturer's instructions.

### Results

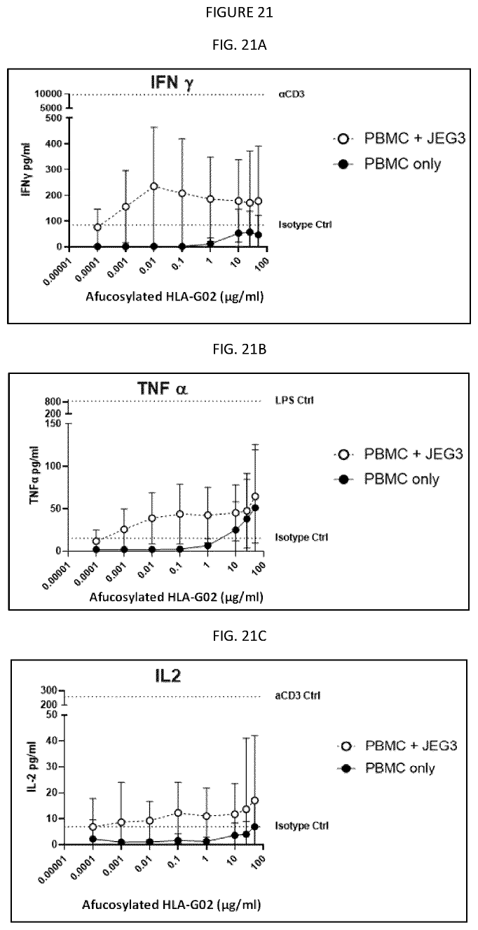

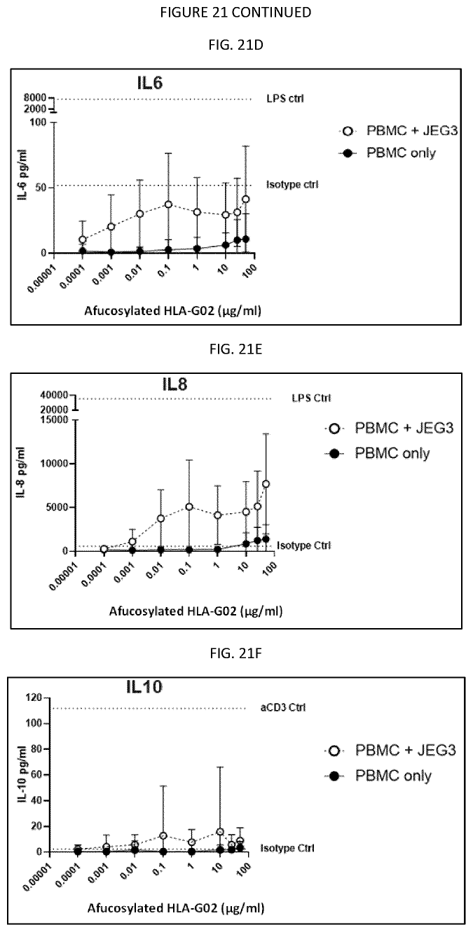

The mean level of 10 pro-inflammatory cytokines released from 16 PBMC donors treated with either 50 μg/mL afucosylated HLA-G02, 50 μg/mL isotype control, 50 μg/mL anti-CD3, or 100 ng/mL LPS in the presence or absence of HLA-G expressing JEG3 cells is summarized in Table 35 below. Mean values generated across the concentration range of HLA-G02 from 0.0001 to 50 μg/ml in the presence and absence of JEG3 cells for the key cytokines IFNγ, TNFα, IL2, IL6, IL8 and IL10 are summarised in FIG. 21 (A-F).

TABLE 35
mean level of 10 pro-inflammatory cytokines released

| Mean value for | PBMC alone | | | | PBMC + JEG3 cells | | | |
|---|---|---|---|---|---|---|---|---|
| 16 donors | | | | aF HLA- | | | | aF HLA- |
| (three replicates) | LPS | CD3 | Isotype | G02 | LPS | CD3 | Isotype | G02 |
| IFN-γ (pg/mL) | 8260.6 | 7399.0 | 302.0 | 56.8 | 26772.7 | 8792.0 | 81.0 | 177.9 |
| IL-10(pg/mL) | 148.0 | 64.4 | 4.5 | 3.1 | 522.5 | 112.0 | 2.3 | 26.9 |
| IL-12p70 (pg/mL) | 30.8 | 5.9 | 0.7 | 0.7 | 24.2 | 9.2 | 3.2 | 6.5 |
| IL-13 (pg/mL) | 95.4 | 58.6 | 11.5 | 26.1 | 168.0 | 95.4 | 27.5 | 120.1 |
| IL-1β (pg/mL) | 1401.9 | 34.7 | 5.9 | 3.1 | 1065.1 | 30.3 | 12.6 | 10.4 |
| IL-2 (pg/mL) | 85.5 | 212.0 | 26.5 | 6.6 | 274.8 | 247.2 | 7.0 | 17.1 |
| IL-4 (pg/mL) | 13.0 | 3.8 | 0.5 | 0.2 | 21.2 | 6.6 | 0.3 | 1.3 |
| IL-6 (pg/mL) | 7070.3 | 55.0 | 15.7 | 10.5 | 7392.0 | 114.7 | 52.8 | 41.5 |
| IL-8 (pg/mL) | 10119.9 | 8234.4 | 681.5 | 1531.1 | 35308.6 | 8618.2 | 565.1 | 7706.5 |
| TNF-α (pg/mL) | 1712.5 | 865.9 | 68.0 | 55.5 | 844.0 | 415.2 | 14.4 | 64.6 |

A low level of ten pro-inflammatory cytokines (IFN-7, IL-10, IL-12p70, IL-13, IL-1β, IL-2, IL4, IL-6, IL-8, and TNF-α) was produced from PBMCs treated with 50 μg/mL afucosylated HLA-G02. This level of cytokine release was comparable to the level observed when PBMCs were treated with an isotype control antibody. Depending on the cytokine tested, the amount produced following treatment with 50 μg/mL afucosylated HLA-G02 was between 2 to 130-fold lower or 3.5 to 700-fold lower than the anti-CD3 and lipopolysaccharide (LPS) positive controls, respectively.

A co-culture of PBMCs with JEG3 cells, resulted in increased pro-inflammatory cytokine release following treatment with afucosylated HLA-G02. In comparison to PBMC alone, the level of 9/10 cytokines (all except TNF-α) were increased in supernatants from PBMC-JEG3 co-cultures at 50 mg/ml. Depending on the cytokine measured, this increase ranged from between a 3-fold (IFN-γ, IL1-β, IL-2) to a 9 fold (IL-10) change compared to afucosylated HLA-G02-treated PBMCs alone. In addition, the level of cytokines increased in the presence of HLA-G expressing JEG3 cells across the concentration range of HLA-G02.

The increased cytokine release observed in the presence of HLA-G expressing cells provides an indication of the level of pro-inflammatory cytokine release which may occur as a result of Fc mediated activity in HLA-G positive tumors treated with afucosylated HLA-G02.

The 12389 antibody sequences and human frameworks included in the present invention are shown in Table 36 below (“(nt.)”: nucleic sequence):

TABLE 36

| | | SEQ |
|---|---|---|
| | | ID |
| Name | Sequence | NO: |
| CDR-L1 | QASQSIYSYLS | 1 |
| CDR-L2 | KASTLAS | 2 |
| CDR-L3 | QNHWNVGGNGWP | 3 |
| CDR-H1 | GIDLSSNAMS | 4 |
| CDR-H2 | TISSGGRTYYASWAKG | 5 |
| CDR-H3 | GDGATGFNI | 6 |
| Rabbit VL | ALVMTQTPASVSEPVGGTVTIKCQASQSIYSYLSWYQQKPG | 7 |
| | QPPKLLIYKASTLASGVSSRFKGSGSGTQFTLTISDLECGDAA | |
| | TYYCQNHWNVGGNGWPFGGGTEVVVK | |
| Rabbit VL | gcccttgtgatgacccagactccagcctccgtgtctgaacctgtgggaggcacagtcaccatca | 8 |
| (nt.) | agtgccaggccagtcagagcatttacagctacttatcctggtatcagcagaaaccagggcagcc | |
| | tcccaagctcctaatctacaaggcatccactctggcatctggggtctcatcgcggttcaaaggca | |
| | gtggatctgggacacagttcactctcaccatcagcgacctggagtgtggcgatgctgccacttac | |
| | tactgtcaaaatcattggaatgttggtggtaatggttggcctttcggcggagggaccgaggtggt | |
| | ggtcaaa | |
| Rabbit | ALVMTQTPASVSEPVGGTVTIKCQASQSIYSYLSWYQQKPG | 9 |
| Light chain | QPPKLLIYKASTLASGVSSRFKGSGSGTQFTLTISDLECGDAA | |
| | TYYCQNHWNVGGNGWPFGGGTEVVVKRTPVAPTVLIFPPA | |
| | ADQVATGTVTIVCVANKYFPDVTVTWEVDGTTQTTGIENSK | |
| | TPQNSADCTYNLSSTLTLTSTQYNSHKEYTCKVTQGTTSVV | |
| | QSFNRGDC | |
| Rabbit | gcccttgtgatgacccagactccagcctccgtgtctgaacctgtgggaggcacagtcaccatca | 10 |
| Light chain | agtgccaggccagtcagagcatttacagctacttatcctggtatcagcagaaaccagggcagcc | |
| (nt.) | tcccaagctcctaatctacaaggcatccactctggcatctggggtctcatcgcggttcaaaggca | |
| | gtggatctgggacacagttcactctcaccatcagcgacctggagtgtggcgatgctgccacttac | |
| | tactgtcaaaatcattggaatgttggtggtaatggttggcctttcggcggagggaccgaggtggt | |
| | ggtcaaacgtacgccagttgcacctactgtcctcatcttcccaccagctgctgatcaggtggcaa | |
| | ctggaacagtcaccatcgtgtgtgtggcgaataaatactttcccgatgtcaccgtcacctgggag | |
| | gtggatggcaccacccaaacaactggcatcgagaacagtaaaacaccgcagaattctgcagat | |
| | tgtacctacaacctcagcagcactctgacactgaccagcacacagtacaacagccacaaagag | |
| | tacacctgcaaggtgacccagggcacgacctcagtcgtccagagcttcaataggggtgactgt | |
| Rabbit VH | QSVEESGGRLVTPGTPLTLTCTVSGIDLSSNAMSWVRQAPGE | 11 |
| | GLEWIGTISSGGRTYYASWAKGRFTISKTSTTVDLKIPSPTTE | |
| | DTATYFCGRGDGATGFNIWGPGTLVTVSS | |
| Rabbit VH | cagtcggtggaggagtccgggggtcgcctggtcacgcctgggacacccctgacactcacctg | 12 |
| (nt.) | cacagtctctggaatcgacctcagtagcaatgcaatgagctgggtccgccaggctccagggga | |
| | ggggctggaatggatcggaaccattagtagtggtggtaggacatactacgcgagctgggcaaa | |
| | aggccgattcaccatctccaaaacctcgaccacggtggatctgaaaatccccagtccgacaacc | |
| | gaggacacggccacctatttctgtggcagaggagatggtgctactggctttaacatctggggcc | |
| | caggcaccctggtcaccgtctcgagt | |
| Rabbit | QSVEESGGRLVTPGTPLTLTCTVSGIDLSSNAMSWVRQAPGE | 13 |
| Heavy | GLEWIGTISSGGRTYYASWAKGRFTISKTSTTVDLKIPSPTTE | |
| chain (Fab) | DTATYFCGRGDGATGFNIWGPGTLVTVSSGQPKAPSVFPLAP | |
| | CCGDTPSSTVTLGCLVKGYLPEPVTVTWNSGTLTNGVRTFPS | |
| | VRQSSGLYSLSSVVSVTSSSQPVTCNVAHPATNTKVDKTVAP | |
| | STCSKP | |
| Rabbit | cagtcggtggaggagtccgggggtcgcctggtcacgcctgggacacccctgacactcacctg | 14 |
| Heavy | cacagtctctggaatcgacctcagtagcaatgcaatgagctgggtccgccaggctccagggga | |
| chain (Fab) | ggggctggaatggatcggaaccattagtagtggtggtaggacatactacgcgagctgggcaaa | |
| (nt.) | aggccgattcaccatctccaaaacctcgaccacggtggatctgaaaatccccagtccgacaacc | |
| | gaggacacggccacctatttctgtggcagaggagatggtgctactggctttaacatctggggcc | |
| | caggcaccctggtcaccgtctcgagtgggcaacctaaggctccatcagtcttcccactggcccc | |
| | ctgctgcggggacacacccagctccacggtgaccctgggctgcctggtcaaaggctacctccc | |
| | ggagccagtgaccgtgacctggaactcgggcaccctcaccaatggggtacgcaccttcccgtc | |
| | cgtccggcagtcctcaggcctctactcgctgagcagcgtggtgagcgtgacctcaagcagcca | |
| | gcccgtcacctgcaacgtggcccacccagccaccaacaccaaagtggacaagaccgttgcgc | |
| | cctcgacatgcagcaagccc | |
| 12389gL1 | AIVLTQSPSSLSASVGDRVTITCQASQSIYSYLSWYQQKPGK | 15 |
| VL | APKLLIYKASTLASGVPSRFSGSGSGTQFTLTISSLQPEDFATY | |
| | YCQNHWNVGGNGWPFGGGTKVEIK | |
| 12389gL1 | gccattgtgttgactcagagcccgtcgtcactgagcgcttccgtgggcgacagagtgaccatca | 16 |
| VL (nt.) | cctgtcaagccagccagtccatctactcctacctgtcatggtaccagcagaagccagggaaagc | |
| | cccgaagctgctgatctacaaggcctctacccttgcgtccggagtgccttcgcggttttcgggttc | |
| | cggttccggaactcagttcacgctcaccattagctccctccaacccgaggatttcgcaacctacta | |
| | ttgccaaaaccactggaacgtcgggggcaatggctggcccttcggaggaggcactaaggtcg | |
| | aaatcaag | |
| 12389gL1 | AIVLTQSPSSLSASVGDRVTITCQASQSIYSYLSWYQQKPGK | 17 |
| Light chain | APKLLIYKASTLASGVPSRFSGSGSGTQFTLTISSLQPEDFATY | |
| | YCQNHWNVGGNGWPFGGGTKVEIKRTVAAPSVFIFPPSDEQ | |
| | LKSGTASVVCLLNNFYPREAKVQWKVDNALQSGNSQESVT | |
| | EQDSKDSTYSLSSTLTLSKADYEKHKVYACEVTHQGLSSPVT | |
| | KSFNRGEC | |
| 12389gL1 | gccattgtgttgactcagagcccgtcgtcactgagcgcttccgtgggcgacagagtgaccatca | 18 |
| Light chain | cctgtcaagccagccagtccatctactcctacctgtcatggtaccagcagaagccagggaaagc | |
| (nt.) | cccgaagctgctgatctacaaggcctctacccttgcgtccggagtgccttcgcggttttcgggttc | |
| | cggttccggaactcagttcacgctcaccattagctccctccaacccgaggatttcgcaacctacta | |
| | ttgccaaaaccactggaacgtcgggggcaatggctggcccttcggaggaggcactaaggtcg | |
| | aaatcaagcgtacggtagcggccccatctgtcttcatcttcccgccatctgatgagcagttgaaat | |
| | ctggaactgcctctgttgtgtgcctgctgaataacttctatcccagagaggccaaagtacagtgga | |
| | aggtggataacgccctccaatcgggtaactcccaggagagtgtcacagagcaggacagcaag | |
| | gacagcacctacagcctcagcagcaccctgacgctgagcaaagcagactacgagaaacacaa | |
| | agtctacgcctgcgaagtcacccatcagggcctgagctcgcccgtcacaaagagcttcaacag | |
| | gggagagtgt | |
| 12389gL2 | AIVLTQSPSSLSASVGDRVTITCQASQSIYSYLSWYQQKPGK | 19 |
| VL | APKLLIYKASTLASGVPSRFSGSGSGTDFTLTISSLQPEDFATY | |
| | YCQNHWNVGGNGWPFGGGTKVEIK | |
| 12389gL2 | gccattgtgctgactcagtcaccgagctcacttagcgcctccgtgggggaccgggtcacaatca | 20 |
| VL (nt.) | cttgccaagcgtcgcagtcaatctactcgtacctctcgtggtatcagcagaagcctgggaaggc | |
| | acctaaactcctgatctacaaggcttcaactttggcatctggagtgccgagcagattcagcggat | |
| | cgggaagcggaactgattttaccctcactatctcgtcgctccaaccggaagatttcgcgacctact | |
| | actgtcaaaaccattggaatgtcggtggaaacggttggcctttcggcgggggaaccaaagtgg | |
| | agattaag | |
| 12389gL2 | AIVLTQSPSSLSASVGDRVTITCQASQSIYSYLSWYQQKPGK | 21 |
| Light chain | APKLLIYKASTLASGVPSRFSGSGSGTDFTLTISSLQPEDFATY | |
| | YCQNHWNVGGNGWPFGGGTKVEIKRTVAAPSVFIFPPSDEQ | |
| | LKSGTASVVCLLNNFYPREAKVQWKVDNALQSGNSQESVT | |
| | EQDSKDSTYSLSSTLTLSKADYEKHKVYACEVTHQGLSSPVT | |
| | KSFNRGEC | |
| 12389gL2 | gccattgtgctgactcagtcaccgagctcacttagcgcctccgtgggggaccgggtcacaatca | 22 |
| Light chain | cttgccaagcgtcgcagtcaatctactcgtacctctcgtggtatcagcagaagcctgggaaggc | |
| (nt.) | acctaaactcctgatctacaaggcttcaactttggcatctggagtgccgagcagattcagcggat | |
| | cgggaagcggaactgattttaccctcactatctcgtcgctccaaccggaagatttcgcgacctact | |
| | actgtcaaaaccattggaatgtcggtggaaacggttggcctttcggcgggggaaccaaagtgg | |
| | agattaagcgtacggtggccgctccctccgtgttcatcttcccaccctccgacgagcagctgaag | |
| | tccggcaccgcctccgtcgtgtgcctgctgaacaacttctacccccgcgaggccaaggtgcagt | |
| | ggaaggtggacaacgccctgcagtccggcaactcccaggaatccgtcaccgagcaggactcc | |
| | aaggacagcacctactccctgtcctccaccctgaccctgtccaaggccgactacgagaagcac | |
| | aaggtgtacgcctgcgaagtgacccaccagggcctgtccagccccgtgaccaagtccttcaac | |
| | cggggcgagtgc | |
| 12389gL3 | AIQLTQSPSSLSASVGDRVTITCQASQSIYSYLSWYQQKPGK | 23 |
| VL | APKLLIYKASTLASGVPSRFSGSGSGTDFTLTISSLQPEDFATY | |
| | YCQNHWNVGGNGWPFGGGTKVEIK | |
| 12389gL3 | gccattcagttgactcagagcccgtcgtcactgagcgcttccgtgggcgacagagtgaccatca | 24 |
| VL (nt.) | cctgtcaagccagccagtccatctactcctacctgtcatggtaccagcagaagccagggaaagc | |
| | cccgaagctgctgatctacaaggcctctacccttgcgtccggagtgccttcgcggttttcgggttc | |
| | cggttccggaactgacttcacgctcaccattagctccctccaacccgaggatttcgcaacctacta | |
| | ttgccaaaaccactggaacgtcgggggcaatggctggcccttcggaggaggcactaaggtcg | |
| | aaatcaag | |
| 12389gL3 | AIQLTQSPSSLSASVGDRVTITCQASQSIYSYLSWYQQKPGK | 25 |
| Light chain | APKLLIYKASTLASGVPSRFSGSGSGTDFTLTISSLQPEDFATY | |
| | YCQNHWNVGGNGWPFGGGTKVEIKRTVAAPSVFIFPPSDEQ | |
| | LKSGTASVVCLLNNFYPREAKVQWKVDNALQSGNSQESVT | |
| | EQDSKDSTYSLSSTLTLSKADYEKHKVYACEVTHQGLSSPVT | |
| | KSFNRGEC | |
| 12389gL3 | gccattcagttgactcagagcccgtcgtcactgagcgcttccgtgggcgacagagtgaccatca | 26 |
| Light chain | cctgtcaagccagccagtccatctactcctacctgtcatggtaccagcagaagccagggaaagc | |
| (nt.) | cccgaagctgctgatctacaaggcctctacccttgcgtccggagtgccttcgcggttttcgggttc | |
| | cggttccggaactgacttcacgctcaccattagctccctccaacccgaggatttcgcaacctacta | |
| | ttgccaaaaccactggaacgtcgggggcaatggctggcccttcggaggaggcactaaggtcg | |
| | aaatcaagcgtacggtagcggccccatctgtcttcatcttcccgccatctgatgagcagttgaaat | |
| | ctggaactgcctctgttgtgtgcctgctgaataacttctatcccagagaggccaaagtacagtgga | |
| | aggtggataacgccctccaatcgggtaactcccaggagagtgtcacagagcaggacagcaag | |
| | gacagcacctacagcctcagcagcaccctgacgctgagcaaagcagactacgagaaacacaa | |
| | agtctacgcctgcgaagtcacccatcagggcctgagctcgcccgtcacaaagagcttcaacag | |
| | gggagagtgt | |
| 12389gH1 | EVQLVESGGGLVQPGGSLRLSCAVSGIDLSSNAMSWVRQAP | 27 |
| VH | GKGLEWIGTISSGGRTYYASWAKGRFTISKDSSKNTVYLQM | |
| | NSLRAEDTAVYYCGRGDGATGFNIWGQGTLVTVSS | |
| 12389gH1 | gaagtgcagctggtcgaatccgggggtggtctggtgcagccgggaggttccctgcgcttgtcat | 28 |
| VH (nt.) | gcgcggtgtccggcattgaccttagctccaacgccatgagctgggtcagacaggcccctggca | |
| | aagggctggagtggattggcaccatctcaagcggagggcggacttactatgcctcctgggcca | |
| | agggacgcttcaccatctcgaaggactcctcgaagaacaccgtgtacctccaaatgaactcgct | |
| | gagggcagaggacactgctgtgtactactgtggacggggagatggagccaccggcttcaatat | |
| | ctggggccagggaaccctcgtgactgtctcgagc | |
| 12389gH1 | EVQLVESGGGLVQPGGSLRLSCAVSGIDLSSNAMSWVRQAP | 29 |
| Heavy | GKGLEWIGTISSGGRTYYASWAKGRFTISKDSSKNTVYLQM | |
| chain | NSLRAEDTAVYYCGRGDGATGFNIWGQGTLVTVSSASTKGP | |
| (IgG1) | SVFPLAPSSKSTSGGTAALGCLVKDYFPEPVTVSWNSGALTS | |
| | GVHTFPAVLQSSGLYSLSSVVTVPSSSLGTQTYICNVNHKPS | |
| | NTKVDKKVEPKSCDKTHTCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLSLSPGK | |
| 12389gH1 | gaagtgcagctggtcgaatccgggggtggtctggtgcagccgggaggttccctgcgcttgtcat | 30 |
| Heavy | gcgcggtgtccggcattgaccttagctccaacgccatgagctgggtcagacaggcccctggca | |
| chain | aagggctggagtggattggcaccatctcaagcggagggcggacttactatgcctcctgggcca | |
| (IgG1)(nt.) | agggacgcttcaccatctcgaaggactcctcgaagaacaccgtgtacctccaaatgaactcgct | |
| | gagggcagaggacactgctgtgtactactgtggacggggagatggagccaccggcttcaatat | |
| | ctggggccagggaaccctcgtgactgtctcgagcgcttctacaaagggcccatcggtcttcccc | |
| | ctggcaccctcctccaagagcacctctgggggcacagcggccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacccagacctacatctgcaacgtgaatcacaagcccagcaacaccaaggtg | |
| | gacaagaaagttgagcccaaatcttgtgacaaaactcacacatgcccaccgtgcccagcacctg | |
| | aactcctggggggaccgtcagtcttcctcttccccccaaaacccaaggacaccctcatgatctcc | |
| | cggacccctgaggtcacatgcgtggtggtggacgtgagccacgaagaccctgaggtcaagttc | |
| | aactggtacgtggacggcgtggaggtgcataatgccaagacaaagccgcgggaggagcagt | |
| | acaacagcacgtaccgtgtggtcagcgtcctcaccgtcctgcaccaggactggctgaatggca | |
| | aggagtacaagtgcaaggtctccaacaaagccctcccagcccccatcgagaaaaccatctcca | |
| | aagccaaagggcagccccgagaaccacaggtgtacaccctgcccccatcccgggatgagct | |
| | gaccaagaaccaggtcagcctgacctgcctggtcaaaggcttctatcccagcgacatcgccgt | |
| | ggagtgggagagcaatgggcagccggagaacaactacaagaccacgcctcccgtgctggac | |
| | tccgacggctccttcttcctctacagcaagctcaccgtggacaagagcaggtggcagcagggg | |
| | aacgtcttctcatgctccgtgatgcatgaggctctgcacaaccactacacgcagaagagcctctc | |
| | cctgtctccgggtaaa | |
| 12389gH1 | EVQLVESGGGLVQPGGSLRLSCAVSGIDLSSNAMSWVRQAP | 31 |
| Heavy | GKGLEWIGTISSGGRTYYASWAKGRFTISKDSSKNTVYLQM | |
| chain | NSLRAEDTAVYYCGRGDGATGFNIWGQGTLVTVSSASTKGP | |
| (IgG4P) | SVFPLAPCSRSTSESTAALGCLVKDYFPEPVTVSWNSGALTS | |
| | GVHTFPAVLQSSGLYSLSSVVTVPSSSLGTKTYTCNVDHKPS | |
| | NTKVDKRVESKYGPPCPPCPAPEFLGGPSVFLFPPKPKDTLMI | |
| | SRTPEVTCVVVDVSQEDPEVQFNWYVDGVEVHNAKTKPRE | |
| | EQFNSTYRVVSVLTVLHQDWLNGKEYKCKVSNKGLPSSIEK | |
| | TISKAKGQPREPQVYTLPPSQEEMTKNQVSLTCLVKGFYPSD | |
| | IAVEWESNGQPENNYKTTPPVLDSDGSFFLYSRLTVDKSRW | |
| | QEGNVFSCSVMHEALHNHYTQKSLSLSLGK | |
| 12389gH1 | gaagtgcagctggtcgaatccgggggtggtctggtgcagccgggaggttccctgcgcttgtcat | 32 |
| Heavy | gcgcggtgtccggcattgaccttagctccaacgccatgagctgggtcagacaggcccctggca | |
| chain | aagggctggagtggattggcaccatctcaagcggagggcggacttactatgcctcctgggcca | |
| (IgG4P) | agggacgcttcaccatctcgaaggactcctcgaagaacaccgtgtacctccaaatgaactcgct | |
| (nt.) | gagggcagaggacactgctgtgtactactgtggacggggagatggagccaccggcttcaatat | |
| | ctggggccagggaaccctcgtgactgtctcgagcgcttctacaaagggcccatccgtcttcccc | |
| | ctggcgccctgctccaggagcacctccgagagcacagccgccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacgaagacctacacctgcaacgtagatcacaagcccagcaacaccaaggtg | |
| | gacaagagagttgagtccaaatatggtcccccatgcccaccatgcccagcacctgagttcctgg | |
| | ggggaccatcagtcttcctgttccccccaaaacccaaggacactctcatgatctcccggacccct | |
| | gaggtcacgtgcgtggtggtggacgtgagccaggaagaccccgaggtccagttcaactggta | |
| | cgtggatggcgtggaggtgcataatgccaagacaaagccgcgggaggagcagttcaacagc | |
| | acgtaccgtgtggtcagcgtcctcaccgtcctgcaccaggactggctgaacggcaaggagtac | |
| | aagtgcaaggtctccaacaaaggcctcccgtcctccatcgagaaaaccatctccaaagccaaa | |
| | gggcagccccgagagccacaggtgtacaccctgcccccatcccaggaggagatgaccaaga | |
| | accaggtcagcctgacctgcctggtcaaaggcttctaccccagcgacatcgccgtggagtggg | |
| | agagcaatgggcagccggagaacaactacaagaccacgcctcccgtgctggactccgacgg | |
| | ctccttcttcctctacagcaggctaaccgtggacaagagcaggtggcaggaggggaatgtcttct | |
| | catgctccgtgatgcatgaggctctgcacaaccactacacacagaagagcctctccctgtctctg | |
| | ggtaaa | |
| 12389gH4 | EVQLVESGGGLVQPGGSLRLSCAASGIDLSSNAMSWVRQAP | 33 |
| VH | GKGLEWIGTISSGGRTYYASWAKGRFTISKDSSKNTVYLQM | |
| | NSLRAEDTAVYYCGRGDGATGFNIWGQGTLVTVSS | |
| 12389gH4 | gaagtgcagctggtcgaatccgggggtggtctggtgcagccgggaggttccctgcgcttgtcat | 34 |
| VH (nt.) | gcgcggcttccggcattgaccttagctccaacgccatgagctgggtcagacaggcccctggca | |
| | aagggctggagtggattggcaccatctcaagcggagggcggacttactatgcctcctgggcca | |
| | agggacgcttcaccatctcgaaggactcctcgaagaacaccgtgtacctccaaatgaactcgct | |
| | gagggcagaggacactgctgtgtactactgtggacggggagatggagccaccggcttcaatat | |
| | ctggggccagggaaccctcgtgactgtctcgagc | |
| 12389gH4 | EVQLVESGGGLVQPGGSLRLSCAASGIDLSSNAMSWVRQAP | 35 |
| Heavy | GKGLEWIGTISSGGRTYYASWAKGRFTISKDSSKNTVYLQM | |
| chain | NSLRAEDTAVYYCGRGDGATGFNIWGQGTLVTVSSASTKGP | |
| (IgG1) | SVFPLAPSSKSTSGGTAALGCLVKDYFPEPVTVSWNSGALTS | |
| | GVHTFPAVLQSSGLYSLSSVVTVPSSSLGTQTYICNVNHKPS | |
| | NTKVDKKVEPKSCDKTHTCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLSLSPGK | |
| 12389gH4 | gaagtgcagctggtcgaatccgggggtggtctggtgcagccgggaggttccctgcgcttgtcat | 36 |
| Heavy | gcgcggcttccggcattgaccttagctccaacgccatgagctgggtcagacaggcccctggca | |
| chain | aagggctggagtggattggcaccatctcaagcggagggcggacttactatgcctcctgggcca | |
| (IgG1)(nt.) | agggacgcttcaccatctcgaaggactcctcgaagaacaccgtgtacctccaaatgaactcgct | |
| | gagggcagaggacactgctgtgtactactgtggacggggagatggagccaccggcttcaatat | |
| | ctggggccagggaaccctcgtgactgtctcgagcgcttctacaaagggcccatcggtcttcccc | |
| | ctggcaccctcctccaagagcacctctgggggcacagcggccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacccagacctacatctgcaacgtgaatcacaagcccagcaacaccaaggtg | |
| | gacaagaaagttgagcccaaatcttgtgacaaaactcacacatgcccaccgtgcccagcacctg | |
| | aactcctggggggaccgtcagtcttcctcttccccccaaaacccaaggacaccctcatgatctcc | |
| | cggacccctgaggtcacatgcgtggtggtggacgtgagccacgaagaccctgaggtcaagttc | |
| | aactggtacgtggacggcgtggaggtgcataatgccaagacaaagccgcgggaggagcagt | |
| | acaacagcacgtaccgtgtggtcagcgtcctcaccgtcctgcaccaggactggctgaatggca | |
| | aggagtacaagtgcaaggtctccaacaaagccctcccagcccccatcgagaaaaccatctcca | |
| | aagccaaagggcagccccgagaaccacaggtgtacaccctgcccccatcccgggatgagct | |
| | gaccaagaaccaggtcagcctgacctgcctggtcaaaggcttctatcccagcgacatcgccgt | |
| | ggagtgggagagcaatgggcagccggagaacaactacaagaccacgcctcccgtgctggac | |
| | tccgacggctccttcttcctctacagcaagctcaccgtggacaagagcaggtggcagcagggg | |
| | aacgtcttctcatgctccgtgatgcatgaggctctgcacaaccactacacgcagaagagcctctc | |
| | cctgtctccgggtaaa | |
| 12389gH4 | EVQLVESGGGLVQPGGSLRLSCAASGIDLSSNAMSWVRQAP | 37 |
| Heavy | GKGLEWIGTISSGGRTYYASWAKGRFTISKDSSKNTVYLQM | |
| chain | NSLRAEDTAVYYCGRGDGATGFNIWGQGTLVTVSSASTKGP | |
| (IgG4P) | SVFPLAPCSRSTSESTAALGCLVKDYFPEPVTVSWNSGALTS | |
| | GVHTFPAVLQSSGLYSLSSVVTVPSSSLGTKTYTCNVDHKPS | |
| | NTKVDKRVESKYGPPCPPCPAPEFLGGPSVFLFPPKPKDTLMI | |
| | SRTPEVTCVVVDVSQEDPEVQFNWYVDGVEVHNAKTKPRE | |
| | EQFNSTYRVVSVLTVLHQDWLNGKEYKCKVSNKGLPSSIEK | |
| | TISKAKGQPREPQVYTLPPSQEEMTKNQVSLTCLVKGFYPSD | |
| | IAVEWESNGQPENNYKTTPPVLDSDGSFFLYSRLTVDKSRW | |
| | QEGNVFSCSVMHEALHNHYTQKSLSLSLGK | |
| 12389gH4 | gaagtgcagctggtcgaatccgggggtggtctggtgcagccgggaggttccctgcgcttgtcat | 38 |
| Heavy | gcgcggcttccggcattgaccttagctccaacgccatgagctgggtcagacaggcccctggca | |
| chain | aagggctggagtggattggcaccatctcaagcggagggcggacttactatgcctcctgggcca | |
| (IgG4P) | agggacgcttcaccatctcgaaggactcctcgaagaacaccgtgtacctccaaatgaactcgct | |
| (nt.) | gagggcagaggacactgctgtgtactactgtggacggggagatggagccaccggcttcaatat | |
| | ctggggccagggaaccctcgtgactgtctcgagcgcttctacaaagggcccatccgtcttcccc | |
| | ctggcgccctgctccaggagcacctccgagagcacagccgccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacgaagacctacacctgcaacgtagatcacaagcccagcaacaccaaggtg | |
| | gacaagagagttgagtccaaatatggtcccccatgcccaccatgcccagcacctgagttcctgg | |
| | ggggaccatcagtcttcctgttccccccaaaacccaaggacactctcatgatctcccggacccct | |
| | gaggtcacgtgcgtggtggtggacgtgagccaggaagaccccgaggtccagttcaactggta | |
| | cgtggatggcgtggaggtgcataatgccaagacaaagccgcgggaggagcagttcaacagc | |
| | acgtaccgtgtggtcagcgtcctcaccgtcctgcaccaggactggctgaacggcaaggagtac | |
| | aagtgcaaggtctccaacaaaggcctcccgtcctccatcgagaaaaccatctccaaagccaaa | |
| | gggcagccccgagagccacaggtgtacaccctgcccccatcccaggaggagatgaccaaga | |
| | accaggtcagcctgacctgcctggtcaaaggcttctaccccagcgacatcgccgtggagtggg | |
| | agagcaatgggcagccggagaacaactacaagaccacgcctcccgtgctggactccgacgg | |
| | ctccttcttcctctacagcaggctaaccgtggacaagagcaggtggcaggaggggaatgtcttct | |
| | catgctccgtgatgcatgaggctctgcacaaccactacacacagaagagcctctccctgtctctg | |
| | ggtaaa | |
| 12389gH5 | EVQLVESGGGLVQPGGSLRLSCAVSGIDLSSNAMSWVRQAP | 39 |
| VH | GKGLEWVGTISSGGRTYYASWAKGRFTISKDSSKNTVYLQM | |
| | NSLRAEDTAVYYCGRGDGATGFNIWGQGTLVTVSS | |
| 12389gH5 | gaagtgcagctggtcgaatccgggggtggtctggtgcagccgggaggttccctgcgcttgtcat | 40 |
| VH (nt.) | gcgcggtgtccggcattgaccttagctccaacgccatgagctgggtcagacaggcccctggca | |
| | aagggctggagtgggtcggcaccatctcaagcggagggcggacttactatgcctcctgggcca | |
| | agggacgcttcaccatctcgaaggactcctcgaagaacaccgtgtacctccaaatgaactcgct | |
| | gagggcagaggacactgctgtgtactactgtggacggggagatggagccaccggcttcaatat | |
| | ctggggccagggaaccctcgtgactgtctcgagc | |
| 12389gH5 | EVQLVESGGGLVQPGGSLRLSCAVSGIDLSSNAMSWVRQAP | 41 |
| Heavy | GKGLEWVGTISSGGRTYYASWAKGRFTISKDSSKNTVYLQM | |
| chain | NSLRAEDTAVYYCGRGDGATGFNIWGQGTLVTVSSASTKGP | |
| (IgG1) | SVFPLAPSSKSTSGGTAALGCLVKDYFPEPVTVSWNSGALTS | |
| | GVHTFPAVLQSSGLYSLSSVVTVPSSSLGTQTYICNVNHKPS | |
| | NTKVDKKVEPKSCDKTHTCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLSLSPGK | |
| 12389gH5 | gaagtgcagctggtcgaatccgggggtggtctggtgcagccgggaggttccctgcgcttgtcat | 42 |
| Heavy | gcgcggtgtccggcattgaccttagctccaacgccatgagctgggtcagacaggcccctggca | |
| chain | aagggctggagtgggtcggcaccatctcaagcggagggcggacttactatgcctcctgggcca | |
| (IgG1)(nt.) | agggacgcttcaccatctcgaaggactcctcgaagaacaccgtgtacctccaaatgaactcgct | |
| | gagggcagaggacactgctgtgtactactgtggacggggagatggagccaccggcttcaatat | |
| | ctggggccagggaaccctcgtgactgtctcgagcgcttctacaaagggcccatcggtcttcccc | |
| | ctggcaccctcctccaagagcacctctgggggcacagcggccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacccagacctacatctgcaacgtgaatcacaagcccagcaacaccaaggtg | |
| | gacaagaaagttgagcccaaatcttgtgacaaaactcacacatgcccaccgtgcccagcacctg | |
| | aactcctggggggaccgtcagtcttcctcttccccccaaaacccaaggacaccctcatgatctcc | |
| | cggacccctgaggtcacatgcgtggtggtggacgtgagccacgaagaccctgaggtcaagttc | |
| | aactggtacgtggacggcgtggaggtgcataatgccaagacaaagccgcgggaggagcagt | |
| | acaacagcacgtaccgtgtggtcagcgtcctcaccgtcctgcaccaggactggctgaatggca | |
| | aggagtacaagtgcaaggtctccaacaaagccctcccagcccccatcgagaaaaccatctcca | |
| | aagccaaagggcagccccgagaaccacaggtgtacaccctgcccccatcccgggatgagct | |
| | gaccaagaaccaggtcagcctgacctgcctggtcaaaggcttctatcccagcgacatcgccgt | |
| | ggagtgggagagcaatgggcagccggagaacaactacaagaccacgcctcccgtgctggac | |
| | tccgacggctccttcttcctctacagcaagctcaccgtggacaagagcaggtggcagcagggg | |
| | aacgtcttctcatgctccgtgatgcatgaggctctgcacaaccactacacgcagaagagcctctc | |
| | cctgtctccgggtaaa | |
| 12389gH5 | EVQLVESGGGLVQPGGSLRLSCAVSGIDLSSNAMSWVRQAP | 43 |
| Heavy | GKGLEWVGTISSGGRTYYASWAKGRFTISKDSSKNTVYLQM | |
| chain | NSLRAEDTAVYYCGRGDGATGFNIWGQGTLVTVSSASTKGP | |
| (IgG4P) | SVFPLAPCSRSTSESTAALGCLVKDYFPEPVTVSWNSGALTS | |
| | GVHTFPAVLQSSGLYSLSSVVTVPSSSLGTKTYTCNVDHKPS | |
| | NTKVDKRVESKYGPPCPPCPAPEFLGGPSVFLFPPKPKDTLMI | |
| | SRTPEVTCVVVDVSQEDPEVQFNWYVDGVEVHNAKTKPRE | |
| | EQFNSTYRVVSVLTVLHQDWLNGKEYKCKVSNKGLPSSIEK | |
| | TISKAKGQPREPQVYTLPPSQEEMTKNQVSLTCLVKGFYPSD | |
| | IAVEWESNGQPENNYKTTPPVLDSDGSFFLYSRLTVDKSRW | |
| | QEGNVFSCSVMHEALHNHYTQKSLSLSLGK | |
| 12389gH5 | gaagtgcagctggtcgaatccgggggtggtctggtgcagccgggaggttccctgcgcttgtcat | 44 |
| Heavy | gcgcggtgtccggcattgaccttagctccaacgccatgagctgggtcagacaggcccctggca | |
| chain | aagggctggagtgggtcggcaccatctcaagcggagggcggacttactatgcctcctgggcca | |
| (IgG4P) | agggacgcttcaccatctcgaaggactcctcgaagaacaccgtgtacctccaaatgaactcgct | |
| (nt.) | gagggcagaggacactgctgtgtactactgtggacggggagatggagccaccggcttcaatat | |
| | ctggggccagggaaccctcgtgactgtctcgagcgcttctacaaagggcccatccgtcttcccc | |
| | ctggcgccctgctccaggagcacctccgagagcacagccgccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacgaagacctacacctgcaacgtagatcacaagcccagcaacaccaaggtg | |
| | gacaagagagttgagtccaaatatggtcccccatgcccaccatgcccagcacctgagttcctgg | |
| | ggggaccatcagtcttcctgttccccccaaaacccaaggacactctcatgatctcccggacccct | |
| | gaggtcacgtgcgtggtggtggacgtgagccaggaagaccccgaggtccagttcaactggta | |
| | cgtggatggcgtggaggtgcataatgccaagacaaagccgcgggaggagcagttcaacagc | |
| | acgtaccgtgtggtcagcgtcctcaccgtcctgcaccaggactggctgaacggcaaggagtac | |
| | aagtgcaaggtctccaacaaaggcctcccgtcctccatcgagaaaaccatctccaaagccaaa | |
| | gggcagccccgagagccacaggtgtacaccctgcccccatcccaggaggagatgaccaaga | |
| | accaggtcagcctgacctgcctggtcaaaggcttctaccccagcgacatcgccgtggagtggg | |
| | agagcaatgggcagccggagaacaactacaagaccacgcctcccgtgctggactccgacgg | |
| | ctccttcttcctctacagcaggctaaccgtggacaagagcaggtggcaggaggggaatgtcttct | |
| | catgctccgtgatgcatgaggctctgcacaaccactacacacagaagagcctctccctgtctctg | |
| | ggtaaa | |
| 12389gH6 | EVQLVESGGGLVQPGGSLRLSCAVSGIDLSSNAMSWVRQAP | 45 |
| VH | GKGLEWISTISSGGRTYYASWAKGRFTISKDSSKNTVYLQM | |
| | NSLRAEDTAVYYCGRGDGATGFNIWGQGTLVTVSS | |
| 12389gH6 | gaagtgcagctggtcgaatccgggggtggtctggtgcagccgggaggttccctgcgcttgtcat | 46 |
| VH (nt.) | gcgcggtgtccggcattgaccttagctccaacgccatgagctgggtcagacaggcccctggca | |
| | aagggctggagtggatttccaccatctcaagcggagggcggacttactatgcctcctgggccaa | |
| | gggacgcttcaccatctcgaaggactcctcgaagaacaccgtgtacctccaaatgaactcgctg | |
| | agggcagaggacactgctgtgtactactgtggacggggagatggagccaccggcttcaatatc | |
| | tggggccagggaaccctcgtgactgtctcgagc | |
| 12389gH6 | EVQLVESGGGLVQPGGSLRLSCAVSGIDLSSNAMSWVRQAP | 47 |
| Heavy | GKGLEWISTISSGGRTYYASWAKGRFTISKDSSKNTVYLQM | |
| chain | NSLRAEDTAVYYCGRGDGATGFNIWGQGTLVTVSSASTKGP | |
| (IgG1) | SVFPLAPSSKSTSGGTAALGCLVKDYFPEPVTVSWNSGALTS | |
| | GVHTFPAVLQSSGLYSLSSVVTVPSSSLGTQTYICNVNHKPS | |
| | NTKVDKKVEPKSCDKTHTCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLSLSPGK | |
| 12389gH6 | gaagtgcagctggtcgaatccgggggtggtctggtgcagccgggaggttccctgcgcttgtcat | 48 |
| Heavy | gcgcggtgtccggcattgaccttagctccaacgccatgagctgggtcagacaggcccctggca | |
| chain | aagggctggagtggatttccaccatctcaagcggagggcggacttactatgcctcctgggccaa | |
| (IgG1)(nt.) | gggacgcttcaccatctcgaaggactcctcgaagaacaccgtgtacctccaaatgaactcgctg | |
| | agggcagaggacactgctgtgtactactgtggacggggagatggagccaccggcttcaatatc | |
| | tggggccagggaaccctcgtgactgtctcgagcgcttctacaaagggcccatcggtcttccccc | |
| | tggcaccctcctccaagagcacctctgggggcacagcggccctgggctgcctggtcaaggact | |
| | acttccccgaaccggtgacggtgtcgtggaactcaggcgccctgaccagcggcgtgcacacct | |
| | tcccggctgtcctacagtcctcaggactctactccctcagcagcgtggtgaccgtgccctccagc | |
| | agcttgggcacccagacctacatctgcaacgtgaatcacaagcccagcaacaccaaggtggac | |
| | aagaaagttgagcccaaatcttgtgacaaaactcacacatgcccaccgtgcccagcacctgaac | |
| | tcctggggggaccgtcagtcttcctcttccccccaaaacccaaggacaccctcatgatctcccgg | |
| | acccctgaggtcacatgcgtggtggtggacgtgagccacgaagaccctgaggtcaagttcaac | |
| | tggtacgtggacggcgtggaggtgcataatgccaagacaaagccgcgggaggagcagtaca | |
| | acagcacgtaccgtgtggtcagcgtcctcaccgtcctgcaccaggactggctgaatggcaagg | |
| | agtacaagtgcaaggtctccaacaaagccctcccagcccccatcgagaaaaccatctccaaag | |
| | ccaaagggcagccccgagaaccacaggtgtacaccctgcccccatcccgggatgagctgacc | |
| | aagaaccaggtcagcctgacctgcctggtcaaaggcttctatcccagcgacatcgccgtggagt | |
| | gggagagcaatgggcagccggagaacaactacaagaccacgcctcccgtgctggactccga | |
| | cggctccttcttcctctacagcaagctcaccgtggacaagagcaggtggcagcaggggaacgt | |
| | cttctcatgctccgtgatgcatgaggctctgcacaaccactacacgcagaagagcctctccctgt | |
| | ctccgggtaaa | |
| 12389gH6 | EVQLVESGGGLVQPGGSLRLSCAVSGIDLSSNAMSWVRQAP | 49 |
| Heavy | GKGLEWISTISSGGRTYYASWAKGRFTISKDSSKNTVYLQM | |
| chain | NSLRAEDTAVYYCGRGDGATGFNIWGQGTLVTVSSASTKGP | |
| (IgG4P) | SVFPLAPCSRSTSESTAALGCLVKDYFPEPVTVSWNSGALTS | |
| | GVHTFPAVLQSSGLYSLSSVVTVPSSSLGTKTYTCNVDHKPS | |
| | NTKVDKRVESKYGPPCPPCPAPEFLGGPSVFLFPPKPKDTLMI | |
| | SRTPEVTCVVVDVSQEDPEVQFNWYVDGVEVHNAKTKPRE | |
| | EQFNSTYRVVSVLTVLHQDWLNGKEYKCKVSNKGLPSSIEK | |
| | TISKAKGQPREPQVYTLPPSQEEMTKNQVSLTCLVKGFYPSD | |
| | IAVEWESNGQPENNYKTTPPVLDSDGSFFLYSRLTVDKSRW | |
| | QEGNVFSCSVMHEALHNHYTQKSLSLSLGK | |
| 12389gH6 | gaagtgcagctggtcgaatccgggggtggtctggtgcagccgggaggttccctgcgcttgtcat | 50 |
| Heavy | gcgcggtgtccggcattgaccttagctccaacgccatgagctgggtcagacaggcccctggca | |
| chain | aagggctggagtggatttccaccatctcaagcggagggcggacttactatgcctcctgggccaa | |
| (IgG4P) | gggacgcttcaccatctcgaaggactcctcgaagaacaccgtgtacctccaaatgaactcgctg | |
| (nt.) | agggcagaggacactgctgtgtactactgtggacggggagatggagccaccggcttcaatatc | |
| | tggggccagggaaccctcgtgactgtctcgagcgcttctacaaagggcccatccgtcttccccc | |
| | tggcgccctgctccaggagcacctccgagagcacagccgccctgggctgcctggtcaaggac | |
| | tacttccccgaaccggtgacggtgtcgtggaactcaggcgccctgaccagcggcgtgcacacc | |
| | ttcccggctgtcctacagtcctcaggactctactccctcagcagcgtggtgaccgtgccctccag | |
| | cagcttgggcacgaagacctacacctgcaacgtagatcacaagcccagcaacaccaaggtgg | |
| | acaagagagttgagtccaaatatggtcccccatgcccaccatgcccagcacctgagttcctggg | |
| | gggaccatcagtcttcctgttccccccaaaacccaaggacactctcatgatctcccggacccctg | |
| | aggtcacgtgcgtggtggtggacgtgagccaggaagaccccgaggtccagttcaactggtac | |
| | gtggatggcgtggaggtgcataatgccaagacaaagccgcgggaggagcagttcaacagca | |
| | cgtaccgtgtggtcagcgtcctcaccgtcctgcaccaggactggctgaacggcaaggagtaca | |
| | agtgcaaggtctccaacaaaggcctcccgtcctccatcgagaaaaccatctccaaagccaaag | |
| | ggcagccccgagagccacaggtgtacaccctgcccccatcccaggaggagatgaccaagaa | |
| | ccaggtcagcctgacctgcctggtcaaaggcttctaccccagcgacatcgccgtggagtggga | |
| | gagcaatgggcagccggagaacaactacaagaccacgcctcccgtgctggactccgacggct | |
| | ccttcttcctctacagcaggctaaccgtggacaagagcaggtggcaggaggggaatgtcttctc | |
| | atgctccgtgatgcatgaggctctgcacaaccactacacacagaagagcctctccctgtctctgg | |
| | gtaaa | |
| 12389gH8 | EVQLVESGGGLVQPGGSLRLSCAVSGIDLSSNAMSWVRQAP | 51 |
| VH | GKGLEWIGTISSGGRTYYASWAKGRFTISRDSSKNTVYLQM | |
| | NSLRAEDTAVYYCGRGDGATGFNIWGQGTLVTVSS | |
| 12389gH8 | gaagtgcagctggtcgaatccgggggtggtctggtgcagccgggaggttccctgcgcttgtcat | 52 |
| VH (nt.) | gcgcggtgtccggcattgaccttagctccaacgccatgagctgggtcagacaggcccctggca | |
| | aagggctggagtggattggcaccatctcaagcggagggcggacttactatgcctcctgggcca | |
| | agggacgcttcaccatctcgcgggactcctcgaagaacaccgtgtacctccaaatgaactcgct | |
| | gagggcagaggacactgctgtgtactactgtggacggggagatggagccaccggcttcaatat | |
| | ctggggccagggaaccctcgtgactgtctcgagc | |
| 12389gH8 | EVQLVESGGGLVQPGGSLRLSCAVSGIDLSSNAMSWVRQAP | 53 |
| Heavy | GKGLEWIGTISSGGRTYYASWAKGRFTISRDSSKNTVYLQM | |
| chain | NSLRAEDTAVYYCGRGDGATGFNIWGQGTLVTVSSASTKGP | |
| (IgG1) | SVFPLAPSSKSTSGGTAALGCLVKDYFPEPVTVSWNSGALTS | |
| | GVHTFPAVLQSSGLYSLSSVVTVPSSSLGTQTYICNVNHKPS | |
| | NTKVDKKVEPKSCDKTHTCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLSLSPGK | |
| 12389gH8 | gaagtgcagctggtcgaatccgggggtggtctggtgcagccgggaggttccctgcgcttgtcat | 54 |
| Heavy | gcgcggtgtccggcattgaccttagctccaacgccatgagctgggtcagacaggcccctggca | |
| chain | aagggctggagtggattggcaccatctcaagcggagggcggacttactatgcctcctgggcca | |
| (IgG1)(nt.) | agggacgcttcaccatctcgcgggactcctcgaagaacaccgtgtacctccaaatgaactcgct | |
| | gagggcagaggacactgctgtgtactactgtggacggggagatggagccaccggcttcaatat | |
| | ctggggccagggaaccctcgtgactgtctcgagcgcttctacaaagggcccatcggtcttcccc | |
| | ctggcaccctcctccaagagcacctctgggggcacagcggccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacccagacctacatctgcaacgtgaatcacaagcccagcaacaccaaggtg | |
| | gacaagaaagttgagcccaaatcttgtgacaaaactcacacatgcccaccgtgcccagcacctg | |
| | aactcctggggggaccgtcagtcttcctcttccccccaaaacccaaggacaccctcatgatctcc | |
| | cggacccctgaggtcacatgcgtggtggtggacgtgagccacgaagaccctgaggtcaagttc | |
| | aactggtacgtggacggcgtggaggtgcataatgccaagacaaagccgcgggaggagcagt | |
| | acaacagcacgtaccgtgtggtcagcgtcctcaccgtcctgcaccaggactggctgaatggca | |
| | aggagtacaagtgcaaggtctccaacaaagccctcccagcccccatcgagaaaaccatctcca | |
| | aagccaaagggcagccccgagaaccacaggtgtacaccctgcccccatcccgggatgagct | |
| | gaccaagaaccaggtcagcctgacctgcctggtcaaaggcttctatcccagcgacatcgccgt | |
| | ggagtgggagagcaatgggcagccggagaacaactacaagaccacgcctcccgtgctggac | |
| | tccgacggctccttcttcctctacagcaagctcaccgtggacaagagcaggtggcagcagggg | |
| | aacgtcttctcatgctccgtgatgcatgaggctctgcacaaccactacacgcagaagagcctctc | |
| | cctgtctccgggtaaa | |
| 12389gH8 | EVQLVESGGGLVQPGGSLRLSCAVSGIDLSSNAMSWVRQAP | 55 |
| Heavy | GKGLEWIGTISSGGRTYYASWAKGRFTISRDSSKNTVYLQM | |
| chain | NSLRAEDTAVYYCGRGDGATGFNIWGQGTLVTVSSASTKGP | |
| (IgG4P) | SVFPLAPCSRSTSESTAALGCLVKDYFPEPVTVSWNSGALTS | |
| | GVHTFPAVLQSSGLYSLSSVVTVPSSSLGTKTYTCNVDHKPS | |
| | NTKVDKRVESKYGPPCPPCPAPEFLGGPSVFLFPPKPKDTLMI | |
| | SRTPEVTCVVVDVSQEDPEVQFNWYVDGVEVHNAKTKPRE | |
| | EQFNSTYRVVSVLTVLHQDWLNGKEYKCKVSNKGLPSSIEK | |
| | TISKAKGQPREPQVYTLPPSQEEMTKNQVSLTCLVKGFYPSD | |
| | IAVEWESNGQPENNYKTTPPVLDSDGSFFLYSRLTVDKSRW | |
| | QEGNVFSCSVMHEALHNHYTQKSLSLSLGK | |
| 12389gH8 | gaagtgcagctggtcgaatccgggggtggtctggtgcagccgggaggttccctgcgcttgtcat | 56 |
| Heavy | gcgcggtgtccggcattgaccttagctccaacgccatgagctgggtcagacaggcccctggca | |
| chain | aagggctggagtggattggcaccatctcaagcggagggcggacttactatgcctcctgggcca | |
| (IgG4P) | agggacgcttcaccatctcgcgggactcctcgaagaacaccgtgtacctccaaatgaactcgct | |
| (nt.) | gagggcagaggacactgctgtgtactactgtggacggggagatggagccaccggcttcaatat | |
| | ctggggccagggaaccctcgtgactgtctcgagcgcttctacaaagggcccatccgtcttcccc | |
| | ctggcgccctgctccaggagcacctccgagagcacagccgccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacgaagacctacacctgcaacgtagatcacaagcccagcaacaccaaggtg | |
| | gacaagagagttgagtccaaatatggtcccccatgcccaccatgcccagcacctgagttcctgg | |
| | ggggaccatcagtcttcctgttccccccaaaacccaaggacactctcatgatctcccggacccct | |
| | gaggtcacgtgcgtggtggtggacgtgagccaggaagaccccgaggtccagttcaactggta | |
| | cgtggatggcgtggaggtgcataatgccaagacaaagccgcgggaggagcagttcaacagc | |
| | acgtaccgtgtggtcagcgtcctcaccgtcctgcaccaggactggctgaacggcaaggagtac | |
| | aagtgcaaggtctccaacaaaggcctcccgtcctccatcgagaaaaccatctccaaagccaaa | |
| | gggcagccccgagagccacaggtgtacaccctgcccccatcccaggaggagatgaccaaga | |
| | accaggtcagcctgacctgcctggtcaaaggcttctaccccagcgacatcgccgtggagtggg | |
| | agagcaatgggcagccggagaacaactacaagaccacgcctcccgtgctggactccgacgg | |
| | ctccttcttcctctacagcaggctaaccgtggacaagagcaggtggcaggaggggaatgtcttct | |
| | catgctccgtgatgcatgaggctctgcacaaccactacacacagaagagcctctccctgtctctg | |
| | ggtaaa | |
| 12389gH9 | EVQLVESGGGLVQPGGSLRLSCAVSGIDLSSNAMSWVRQAP | 57 |
| VH | GKGLEWIGTISSGGRTYYASWAKGRFTISKDNSKNTVYLQM | |
| | NSLRAEDTAVYYCGRGDGATGFNIWGQGTLVTVSS | |
| 12389gH9 | gaagtgcagctggtcgaatccgggggtggtctggtgcagccgggaggttccctgcgcttgtcat | 58 |
| VH (nt.) | gcgcggtgtccggcattgaccttagctccaacgccatgagctgggtcagacaggcccctggca | |
| | aagggctggagtggattggcaccatctcaagcggagggcggacttactatgcctcctgggcca | |
| | agggacgcttcaccatctcgaaggacaactcgaagaacaccgtgtacctccaaatgaactcgct | |
| | gagggcagaggacactgctgtgtactactgtggacggggagatggagccaccggcttcaatat | |
| | ctggggccagggaaccctcgtgactgtctcgagc | |
| 12389gH9 | EVQLVESGGGLVQPGGSLRLSCAVSGIDLSSNAMSWVRQAP | 59 |
| Heavy | GKGLEWIGTISSGGRTYYASWAKGRFTISKDNSKNTVYLQM | |
| chain | NSLRAEDTAVYYCGRGDGATGFNIWGQGTLVTVSSASTKGP | |
| (IgG1) | SVFPLAPSSKSTSGGTAALGCLVKDYFPEPVTVSWNSGALTS | |
| | GVHTFPAVLQSSGLYSLSSVVTVPSSSLGTQTYICNVNHKPS | |
| | NTKVDKKVEPKSCDKTHTCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLSLSPGK | |
| 12389gH9 | gaagtgcagctggtcgaatccgggggtggtctggtgcagccgggaggttccctgcgcttgtcat | 60 |
| Heavy | gcgcggtgtccggcattgaccttagctccaacgccatgagctgggtcagacaggcccctggca | |
| chain | aagggctggagtggattggcaccatctcaagcggagggcggacttactatgcctcctgggcca | |
| (IgG1)(nt.) | agggacgcttcaccatctcgaaggacaactcgaagaacaccgtgtacctccaaatgaactcgct | |
| | gagggcagaggacactgctgtgtactactgtggacggggagatggagccaccggcttcaatat | |
| | ctggggccagggaaccctcgtgactgtctcgagcgcttctacaaagggcccatcggtcttcccc | |
| | ctggcaccctcctccaagagcacctctgggggcacagcggccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacccagacctacatctgcaacgtgaatcacaagcccagcaacaccaaggtg | |
| | gacaagaaagttgagcccaaatcttgtgacaaaactcacacatgcccaccgtgcccagcacctg | |
| | aactcctggggggaccgtcagtcttcctcttccccccaaaacccaaggacaccctcatgatctcc | |
| | cggacccctgaggtcacatgcgtggtggtggacgtgagccacgaagaccctgaggtcaagttc | |
| | aactggtacgtggacggcgtggaggtgcataatgccaagacaaagccgcgggaggagcagt | |
| | acaacagcacgtaccgtgtggtcagcgtcctcaccgtcctgcaccaggactggctgaatggca | |
| | aggagtacaagtgcaaggtctccaacaaagccctcccagcccccatcgagaaaaccatctcca | |
| | aagccaaagggcagccccgagaaccacaggtgtacaccctgcccccatcccgggatgagct | |
| | gaccaagaaccaggtcagcctgacctgcctggtcaaaggcttctatcccagcgacatcgccgt | |
| | ggagtgggagagcaatgggcagccggagaacaactacaagaccacgcctcccgtgctggac | |
| | tccgacggctccttcttcctctacagcaagctcaccgtggacaagagcaggtggcagcagggg | |
| | aacgtcttctcatgctccgtgatgcatgaggctctgcacaaccactacacgcagaagagcctctc | |
| | cctgtctccgggtaaa | |
| 12389gH9 | EVQLVESGGGLVQPGGSLRLSCAVSGIDLSSNAMSWVRQAP | 61 |
| Heavy | GKGLEWIGTISSGGRTYYASWAKGRFTISKDNSKNTVYLQM | |
| chain | NSLRAEDTAVYYCGRGDGATGFNIWGQGTLVTVSSASTKGP | |
| (IgG4P) | SVFPLAPCSRSTSESTAALGCLVKDYFPEPVTVSWNSGALTS | |
| | GVHTFPAVLQSSGLYSLSSVVTVPSSSLGTKTYTCNVDHKPS | |
| | NTKVDKRVESKYGPPCPPCPAPEFLGGPSVFLFPPKPKDTLMI | |
| | SRTPEVTCVVVDVSQEDPEVQFNWYVDGVEVHNAKTKPRE | |
| | EQFNSTYRVVSVLTVLHQDWLNGKEYKCKVSNKGLPSSIEK | |
| | TISKAKGQPREPQVYTLPPSQEEMTKNQVSLTCLVKGFYPSD | |
| | IAVEWESNGQPENNYKTTPPVLDSDGSFFLYSRLTVDKSRW | |
| | QEGNVFSCSVMHEALHNHYTQKSLSLSLGK | |
| 12389gH9 | gaagtgcagctggtcgaatccgggggtggtctggtgcagccgggaggttccctgcgcttgtcat | 62 |
| Heavy | gcgcggtgtccggcattgaccttagctccaacgccatgagctgggtcagacaggcccctggca | |
| chain | aagggctggagtggattggcaccatctcaagcggagggcggacttactatgcctcctgggcca | |
| (IgG4P) | agggacgcttcaccatctcgaaggacaactcgaagaacaccgtgtacctccaaatgaactcgct | |
| (nt.) | gagggcagaggacactgctgtgtactactgtggacggggagatggagccaccggcttcaatat | |
| | ctggggccagggaaccctcgtgactgtctcgagcgcttctacaaagggcccatccgtcttcccc | |
| | ctggcgccctgctccaggagcacctccgagagcacagccgccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacgaagacctacacctgcaacgtagatcacaagcccagcaacaccaaggtg | |
| | gacaagagagttgagtccaaatatggtcccccatgcccaccatgcccagcacctgagttcctgg | |
| | ggggaccatcagtcttcctgttccccccaaaacccaaggacactctcatgatctcccggacccct | |
| | gaggtcacgtgcgtggtggtggacgtgagccaggaagaccccgaggtccagttcaactggta | |
| | cgtggatggcgtggaggtgcataatgccaagacaaagccgcgggaggagcagttcaacagc | |
| | acgtaccgtgtggtcagcgtcctcaccgtcctgcaccaggactggctgaacggcaaggagtac | |
| | aagtgcaaggtctccaacaaaggcctcccgtcctccatcgagaaaaccatctccaaagccaaa | |
| | gggcagccccgagagccacaggtgtacaccctgcccccatcccaggaggagatgaccaaga | |
| | accaggtcagcctgacctgcctggtcaaaggcttctaccccagcgacatcgccgtggagtggg | |
| | agagcaatgggcagccggagaacaactacaagaccacgcctcccgtgctggactccgacgg | |
| | ctccttcttcctctacagcaggctaaccgtggacaagagcaggtggcaggaggggaatgtcttct | |
| | catgctccgtgatgcatgaggctctgcacaaccactacacacagaagagcctctccctgtctctg | |
| | ggtaaa | |
| 12389gH11 | EVQLVESGGGLVQPGGSLRLSCAVSGIDLSSNAMSWVRQAP | 63 |
| VH | GKGLEWIGTISSGGRTYYASWAKGRFTISKDSSKNTLYLQM | |
| | NSLRAEDTAVYYCGRGDGATGFNIWGQGTLVTVSS | |
| 12389gH11 | gaagtgcagctggtcgaatccgggggtggtctggtgcagccgggaggttccctgcgcttgtcat | 64 |
| VH (nt.) | gcgcggtgtccggcattgaccttagctccaacgccatgagctgggtcagacaggcccctggca | |
| | aagggctggagtggattggcaccatctcaagcggagggcggacttactatgcctcctgggcca | |
| | agggacgcttcaccatctcgaaggactcctcgaagaacaccctgtacctccaaatgaactcgct | |
| | gagggcagaggacactgctgtgtactactgtggacggggagatggagccaccggcttcaatat | |
| | ctggggccagggaaccctcgtgactgtctcgagc | |
| 12389gH11 | EVQLVESGGGLVQPGGSLRLSCAVSGIDLSSNAMSWVRQAP | 65 |
| Heavy | GKGLEWIGTISSGGRTYYASWAKGRFTISKDSSKNTLYLQM | |
| chain | NSLRAEDTAVYYCGRGDGATGFNIWGQGTLVTVSSASTKGP | |
| (IgG1) | SVFPLAPSSKSTSGGTAALGCLVKDYFPEPVTVSWNSGALTS | |
| | GVHTFPAVLQSSGLYSLSSVVTVPSSSLGTQTYICNVNHKPS | |
| | NTKVDKKVEPKSCDKTHTCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLSLSPGK | |
| 12389gH11 | gaagtgcagctggtcgaatccgggggtggtctggtgcagccgggaggttccctgcgcttgtcat | 66 |
| Heavy | gcgcggtgtccggcattgaccttagctccaacgccatgagctgggtcagacaggcccctggca | |
| chain | aagggctggagtggattggcaccatctcaagcggagggcggacttactatgcctcctgggcca | |
| (IgG1)(nt.) | agggacgcttcaccatctcgaaggactcctcgaagaacaccctgtacctccaaatgaactcgct | |
| | gagggcagaggacactgctgtgtactactgtggacggggagatggagccaccggcttcaatat | |
| | ctggggccagggaaccctcgtgactgtctcgagcgcttctacaaagggcccatcggtcttcccc | |
| | ctggcaccctcctccaagagcacctctgggggcacagcggccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacccagacctacatctgcaacgtgaatcacaagcccagcaacaccaaggtg | |
| | gacaagaaagttgagcccaaatcttgtgacaaaactcacacatgcccaccgtgcccagcacctg | |
| | aactcctggggggaccgtcagtcttcctcttccccccaaaacccaaggacaccctcatgatctcc | |
| | cggacccctgaggtcacatgcgtggtggtggacgtgagccacgaagaccctgaggtcaagttc | |
| | aactggtacgtggacggcgtggaggtgcataatgccaagacaaagccgcgggaggagcagt | |
| | acaacagcacgtaccgtgtggtcagcgtcctcaccgtcctgcaccaggactggctgaatggca | |
| | aggagtacaagtgcaaggtctccaacaaagccctcccagcccccatcgagaaaaccatctcca | |
| | aagccaaagggcagccccgagaaccacaggtgtacaccctgcccccatcccgggatgagct | |
| | gaccaagaaccaggtcagcctgacctgcctggtcaaaggcttctatcccagcgacatcgccgt | |
| | ggagtgggagagcaatgggcagccggagaacaactacaagaccacgcctcccgtgctggac | |
| | tccgacggctccttcttcctctacagcaagctcaccgtggacaagagcaggtggcagcagggg | |
| | aacgtcttctcatgctccgtgatgcatgaggctctgcacaaccactacacgcagaagagcctctc | |
| | cctgtctccgggtaaa | |
| 12389gH11 | EVQLVESGGGLVQPGGSLRLSCAVSGIDLSSNAMSWVRQAP | 67 |
| Heavy | GKGLEWIGTISSGGRTYYASWAKGRFTISKDSSKNTLYLQM | |
| chain | NSLRAEDTAVYYCGRGDGATGFNIWGQGTLVTVSSASTKGP | |
| (IgG4P) | SVFPLAPCSRSTSESTAALGCLVKDYFPEPVTVSWNSGALTS | |
| | GVHTFPAVLQSSGLYSLSSVVTVPSSSLGTKTYTCNVDHKPS | |
| | NTKVDKRVESKYGPPCPPCPAPEFLGGPSVFLFPPKPKDTLMI | |
| | SRTPEVTCVVVDVSQEDPEVQFNWYVDGVEVHNAKTKPRE | |
| | EQFNSTYRVVSVLTVLHQDWLNGKEYKCKVSNKGLPSSIEK | |
| | TISKAKGQPREPQVYTLPPSQEEMTKNQVSLTCLVKGFYPSD | |
| | IAVEWESNGQPENNYKTTPPVLDSDGSFFLYSRLTVDKSRW | |
| | QEGNVFSCSVMHEALHNHYTQKSLSLSLGK | |
| 12389gH11 | gaagtgcagctggtcgaatccgggggtggtctggtgcagccgggaggttccctgcgcttgtcat | 68 |
| Heavy | gcgcggtgtccggcattgaccttagctccaacgccatgagctgggtcagacaggcccctggca | |
| chain | aagggctggagtggattggcaccatctcaagcggagggcggacttactatgcctcctgggcca | |
| (IgG4P) | agggacgcttcaccatctcgaaggactcctcgaagaacaccctgtacctccaaatgaactcgct | |
| (nt.) | gagggcagaggacactgctgtgtactactgtggacggggagatggagccaccggcttcaatat | |
| | ctggggccagggaaccctcgtgactgtctcgagcgcttctacaaagggcccatccgtcttcccc | |
| | ctggcgccctgctccaggagcacctccgagagcacagccgccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacgaagacctacacctgcaacgtagatcacaagcccagcaacaccaaggtg | |
| | gacaagagagttgagtccaaatatggtcccccatgcccaccatgcccagcacctgagttcctgg | |
| | ggggaccatcagtcttcctgttccccccaaaacccaaggacactctcatgatctcccggacccct | |
| | gaggtcacgtgcgtggtggtggacgtgagccaggaagaccccgaggtccagttcaactggta | |
| | cgtggatggcgtggaggtgcataatgccaagacaaagccgcgggaggagcagttcaacagc | |
| | acgtaccgtgtggtcagcgtcctcaccgtcctgcaccaggactggctgaacggcaaggagtac | |
| | aagtgcaaggtctccaacaaaggcctcccgtcctccatcgagaaaaccatctccaaagccaaa | |
| | gggcagccccgagagccacaggtgtacaccctgcccccatcccaggaggagatgaccaaga | |
| | accaggtcagcctgacctgcctggtcaaaggcttctaccccagcgacatcgccgtggagtggg | |
| | agagcaatgggcagccggagaacaactacaagaccacgcctcccgtgctggactccgacgg | |
| | ctccttcttcctctacagcaggctaaccgtggacaagagcaggtggcaggaggggaatgtcttct | |
| | catgctccgtgatgcatgaggctctgcacaaccactacacacagaagagcctctccctgtctctg | |
| | ggtaaa | |
| 12389gH12 | EVQLVESGGGLVQPGGSLRLSCAVSGIDLSSNAMSWVRQAP | 69 |
| VH | GKGLEWIGTISSGGRTYYASWAKGRFTISKDSSKNTVYLQM | |
| | NSLRAEDTAVYYCARGDGATGFNIWGQGTLVTVSS | |
| 12389gH12 | gaagtgcagctggtcgaatccgggggtggtctggtgcagccgggaggttccctgcgcttgtcat | 70 |
| VH (nt.) | gcgcggtgtccggcattgaccttagctccaacgccatgagctgggtcagacaggcccctggca | |
| | aagggctggagtggattggcaccatctcaagcggagggcggacttactatgcctcctgggcca | |
| | agggacgcttcaccatctcgaaggactcctcgaagaacaccgtgtacctccaaatgaactcgct | |
| | gagggcagaggacactgctgtgtactactgtgcgcggggagatggagccaccggcttcaatat | |
| | ctggggccagggaaccctcgtgactgtctcgagc | |
| 12389gH12 | EVQLVESGGGLVQPGGSLRLSCAVSGIDLSSNAMSWVRQAP | 71 |
| Heavy | GKGLEWIGTISSGGRTYYASWAKGRFTISKDSSKNTVYLQM | |
| chain | NSLRAEDTAVYYCARGDGATGFNIWGQGTLVTVSSASTKGP | |
| (IgG1) | SVFPLAPSSKSTSGGTAALGCLVKDYFPEPVTVSWNSGALTS | |
| | GVHTFPAVLQSSGLYSLSSVVTVPSSSLGTQTYICNVNHKPS | |
| | NTKVDKKVEPKSCDKTHTCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLSLSPGK | |
| 12389gH12 | gaagtgcagctggtcgaatccgggggtggtctggtgcagccgggaggttccctgcgcttgtcat | 72 |
| Heavy | gcgcggtgtccggcattgaccttagctccaacgccatgagctgggtcagacaggcccctggca | |
| chain | aagggctggagtggattggcaccatctcaagcggagggcggacttactatgcctcctgggcca | |
| (IgG1)(nt.) | agggacgcttcaccatctcgaaggactcctcgaagaacaccgtgtacctccaaatgaactcgct | |
| | gagggcagaggacactgctgtgtactactgtgcgcggggagatggagccaccggcttcaatat | |
| | ctggggccagggaaccctcgtgactgtctcgagcgcttctacaaagggcccatcggtcttcccc | |
| | ctggcaccctcctccaagagcacctctgggggcacagcggccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacccagacctacatctgcaacgtgaatcacaagcccagcaacaccaaggtg | |
| | gacaagaaagttgagcccaaatcttgtgacaaaactcacacatgcccaccgtgcccagcacctg | |
| | aactcctggggggaccgtcagtcttcctcttccccccaaaacccaaggacaccctcatgatctcc | |
| | cggacccctgaggtcacatgcgtggtggtggacgtgagccacgaagaccctgaggtcaagttc | |
| | aactggtacgtggacggcgtggaggtgcataatgccaagacaaagccgcgggaggagcagt | |
| | acaacagcacgtaccgtgtggtcagcgtcctcaccgtcctgcaccaggactggctgaatggca | |
| | aggagtacaagtgcaaggtctccaacaaagccctcccagcccccatcgagaaaaccatctcca | |
| | aagccaaagggcagccccgagaaccacaggtgtacaccctgcccccatcccgggatgagct | |
| | gaccaagaaccaggtcagcctgacctgcctggtcaaaggcttctatcccagcgacatcgccgt | |
| | ggagtgggagagcaatgggcagccggagaacaactacaagaccacgcctcccgtgctggac | |
| | tccgacggctccttcttcctctacagcaagctcaccgtggacaagagcaggtggcagcagggg | |
| | aacgtcttctcatgctccgtgatgcatgaggctctgcacaaccactacacgcagaagagcctctc | |
| | cctgtctccgggtaaa | |
| 12389gH12 | EVQLVESGGGLVQPGGSLRLSCAVSGIDLSSNAMSWVRQAP | 73 |
| Heavy | GKGLEWIGTISSGGRTYYASWAKGRFTISKDSSKNTVYLQM | |
| chain | NSLRAEDTAVYYCARGDGATGFNIWGQGTLVTVSSASTKGP | |
| (IgG4P) | SVFPLAPCSRSTSESTAALGCLVKDYFPEPVTVSWNSGALTS | |
| | GVHTFPAVLQSSGLYSLSSVVTVPSSSLGTKTYTCNVDHKPS | |
| | NTKVDKRVESKYGPPCPPCPAPEFLGGPSVFLFPPKPKDTLMI | |
| | SRTPEVTCVVVDVSQEDPEVQFNWYVDGVEVHNAKTKPRE | |
| | EQFNSTYRVVSVLTVLHQDWLNGKEYKCKVSNKGLPSSIEK | |
| | TISKAKGQPREPQVYTLPPSQEEMTKNQVSLTCLVKGFYPSD | |
| | IAVEWESNGQPENNYKTTPPVLDSDGSFFLYSRLTVDKSRW | |
| | QEGNVFSCSVMHEALHNHYTQKSLSLSLGK | |
| 12389gH12 | gaagtgcagctggtcgaatccgggggtggtctggtgcagccgggaggttccctgcgcttgtcat | 74 |
| Heavy | gcgcggtgtccggcattgaccttagctccaacgccatgagctgggtcagacaggcccctggca | |
| chain | aagggctggagtggattggcaccatctcaagcggagggcggacttactatgcctcctgggcca | |
| (IgG4P) | agggacgcttcaccatctcgaaggactcctcgaagaacaccgtgtacctccaaatgaactcgct | |
| (nt.) | gagggcagaggacactgctgtgtactactgtgcgcggggagatggagccaccggcttcaatat | |
| | ctggggccagggaaccctcgtgactgtctcgagcgcttctacaaagggcccatccgtcttcccc | |
| | ctggcgccctgctccaggagcacctccgagagcacagccgccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacgaagacctacacctgcaacgtagatcacaagcccagcaacaccaaggtg | |
| | gacaagagagttgagtccaaatatggtcccccatgcccaccatgcccagcacctgagttcctgg | |
| | ggggaccatcagtcttcctgttccccccaaaacccaaggacactctcatgatctcccggacccct | |
| | gaggtcacgtgcgtggtggtggacgtgagccaggaagaccccgaggtccagttcaactggta | |
| | cgtggatggcgtggaggtgcataatgccaagacaaagccgcgggaggagcagttcaacagc | |
| | acgtaccgtgtggtcagcgtcctcaccgtcctgcaccaggactggctgaacggcaaggagtac | |
| | aagtgcaaggtctccaacaaaggcctcccgtcctccatcgagaaaaccatctccaaagccaaa | |
| | gggcagccccgagagccacaggtgtacaccctgcccccatcccaggaggagatgaccaaga | |
| | accaggtcagcctgacctgcctggtcaaaggcttctaccccagcgacatcgccgtggagtggg | |
| | agagcaatgggcagccggagaacaactacaagaccacgcctcccgtgctggactccgacgg | |
| | ctccttcttcctctacagcaggctaaccgtggacaagagcaggtggcaggaggggaatgtcttct | |
| | catgctccgtgatgcatgaggctctgcacaaccactacacacagaagagcctctccctgtctctg | |
| | ggtaaa | |
| 12389gH13 | EVQLVESGGGLVQPGGSLRLSCAASGIDLSSNAMSWVRQAP | 75 |
| VH | GKGLEWIGTISSGGRTYYASWAKGRFTISKDNSKNTVYLQM | |
| | NSLRAEDTAVYYCGRGDGATGFNIWGQGTLVTVSS | |
| 12389gH13 | gaagtgcagctggtcgaatccgggggtggtctggtgcagccgggaggttccctgcgcttgtcat | 76 |
| VH (nt.) | gcgcggcttccggcattgaccttagctccaacgccatgagctgggtcagacaggcccctggca | |
| | aagggctggagtggattggcaccatctcaagcggagggggacttactatgcctcctgggcca | |
| | agggacgcttcaccatctcgaaggacaactcgaagaacaccgtgtacctccaaatgaactcgct | |
| | gagggcagaggacactgctgtgtactactgtggacggggagatggagccaccggcttcaatat | |
| | ctggggccagggaaccctcgtgactgtctcgagc | |
| 12389gH13 | EVQLVESGGGLVQPGGSLRLSCAASGIDLSSNAMSWVRQAP | 77 |
| Heavy | GKGLEWIGTISSGGRTYYASWAKGRFTISKDNSKNTVYLQM | |
| chain | NSLRAEDTAVYYCGRGDGATGFNIWGQGTLVTVSSASTKGP | |
| (IgG1) | SVFPLAPSSKSTSGGTAALGCLVKDYFPEPVTVSWNSGALTS | |
| | GVHTFPAVLQSSGLYSLSSVVTVPSSSLGTQTYICNVNHKPS | |
| | NTKVDKKVEPKSCDKTHTCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLSLSPGK | |
| 12389gH13 | gaagtgcagctggtcgaatccgggggtggtctggtgcagccgggaggttccctgcgcttgtcat | 78 |
| Heavy | gcgcggcttccggcattgaccttagctccaacgccatgagctgggtcagacaggcccctggca | |
| chain | aagggctggagtggattggcaccatctcaagcggagggcggacttactatgcctcctgggcca | |
| (IgG1)(nt.) | agggacgcttcaccatctcgaaggacaactcgaagaacaccgtgtacctccaaatgaactcgct | |
| | gagggcagaggacactgctgtgtactactgtggacggggagatggagccaccggcttcaatat | |
| | ctggggccagggaaccctcgtgactgtctcgagcgcttctacaaagggcccatcggtcttcccc | |
| | ctggcaccctcctccaagagcacctctgggggcacagcggccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacccagacctacatctgcaacgtgaatcacaagcccagcaacaccaaggtg | |
| | gacaagaaagttgagcccaaatcttgtgacaaaactcacacatgcccaccgtgcccagcacctg | |
| | aactcctggggggaccgtcagtcttcctcttccccccaaaacccaaggacaccctcatgatctcc | |
| | cggacccctgaggtcacatgcgtggtggtggacgtgagccacgaagaccctgaggtcaagttc | |
| | aactggtacgtggacggcgtggaggtgcataatgccaagacaaagccgcgggaggagcagt | |
| | acaacagcacgtaccgtgtggtcagcgtcctcaccgtcctgcaccaggactggctgaatggca | |
| | aggagtacaagtgcaaggtctccaacaaagccctcccagcccccatcgagaaaaccatctcca | |
| | aagccaaagggcagccccgagaaccacaggtgtacaccctgcccccatcccgggatgagct | |
| | gaccaagaaccaggtcagcctgacctgcctggtcaaaggcttctatcccagcgacatcgccgt | |
| | ggagtgggagagcaatgggcagccggagaacaactacaagaccacgcctcccgtgctggac | |
| | tccgacggctccttcttcctctacagcaagctcaccgtggacaagagcaggtggcagcagggg | |
| | aacgtcttctcatgctccgtgatgcatgaggctctgcacaaccactacacgcagaagagcctctc | |
| | cctgtctccgggtaaa | |
| 12389gH13 | EVQLVESGGGLVQPGGSLRLSCAASGIDLSSNAMSWVRQAP | 79 |
| Heavy | GKGLEWIGTISSGGRTYYASWAKGRFTISKDNSKNTVYLQM | |
| chain | NSLRAEDTAVYYCGRGDGATGFNIWGQGTLVTVSSASTKGP | |
| (IgG4P) | SVFPLAPCSRSTSESTAALGCLVKDYFPEPVTVSWNSGALTS | |
| | GVHTFPAVLQSSGLYSLSSVVTVPSSSLGTKTYTCNVDHKPS | |
| | NTKVDKRVESKYGPPCPPCPAPEFLGGPSVFLFPPKPKDTLMI | |
| | SRTPEVTCVVVDVSQEDPEVQFNWYVDGVEVHNAKTKPRE | |
| | EQFNSTYRVVSVLTVLHQDWLNGKEYKCKVSNKGLPSSIEK | |
| | TISKAKGQPREPQVYTLPPSQEEMTKNQVSLTCLVKGFYPSD | |
| | IAVEWESNGQPENNYKTTPPVLDSDGSFFLYSRLTVDKSRW | |
| | QEGNVFSCSVMHEALHNHYTQKSLSLSLGK | |
| 12389gH13 | gaagtgcagctggtcgaatccgggggtggtctggtgcagccgggaggttccctgcgcttgtcat | 80 |
| Heavy | gcgcggcttccggcattgaccttagctccaacgccatgagctgggtcagacaggcccctggca | |
| chain | aagggctggagtggattggcaccatctcaagcggagggcggacttactatgcctcctgggcca | |
| (IgG4P) | agggacgcttcaccatctcgaaggacaactcgaagaacaccgtgtacctccaaatgaactcgct | |
| (nt.) | gagggcagaggacactgctgtgtactactgtggacggggagatggagccaccggcttcaatat | |
| | ctggggccagggaaccctcgtgactgtctcgagcgcttctacaaagggcccatccgtcttcccc | |
| | ctggcgccctgctccaggagcacctccgagagcacagccgccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacgaagacctacacctgcaacgtagatcacaagcccagcaacaccaaggtg | |
| | gacaagagagttgagtccaaatatggtcccccatgcccaccatgcccagcacctgagttcctgg | |
| | ggggaccatcagtcttcctgttccccccaaaacccaaggacactctcatgatctcccggacccct | |
| | gaggtcacgtgcgtggtggtggacgtgagccaggaagaccccgaggtccagttcaactggta | |
| | cgtggatggcgtggaggtgcataatgccaagacaaagccgcgggaggagcagttcaacagc | |
| | acgtaccgtgtggtcagcgtcctcaccgtcctgcaccaggactggctgaacggcaaggagtac | |
| | aagtgcaaggtctccaacaaaggcctcccgtcctccatcgagaaaaccatctccaaagccaaa | |
| | gggcagccccgagagccacaggtgtacaccctgcccccatcccaggaggagatgaccaaga | |
| | accaggtcagcctgacctgcctggtcaaaggcttctaccccagcgacatcgccgtggagtggg | |
| | agagcaatgggcagccggagaacaactacaagaccacgcctcccgtgctggactccgacgg | |
| | ctccttcttcctctacagcaggctaaccgtggacaagagcaggtggcaggaggggaatgtcttct | |
| | catgctccgtgatgcatgaggctctgcacaaccactacacacagaagagcctctccctgtctctg | |
| | ggtaaa | |
| 12389gH14 | EVQLVESGGGLVQPGGSLRLSCAASGIDLSSNAMSWVRQAP | 81 |
| VH | GKGLEWIGTISSGGRTYYASWAKGRFTISKDSSKNTVYLQM | |
| | NSLRAEDTAVYYCARGDGATGFNIWGQGTLVTVSS | |
| 12389gH14 | gaagtgcagctggtcgaatccgggggtggtctggtgcagccgggaggttccctgcgcttgtcat | 82 |
| VH (nt.) | gcgcggcttccggcattgaccttagctccaacgccatgagctgggtcagacaggcccctggca | |
| | aagggctggagtggattggcaccatctcaagcggagggcggacttactatgcctcctgggcca | |
| | agggacgcttcaccatctcgaaggactcctcgaagaacaccgtgtacctccaaatgaactcgct | |
| | gagggcagaggacactgctgtgtactactgtgcgcggggagatggagccaccggcttcaatat | |
| | ctggggccagggaaccctcgtgactgtctcgagc | |
| 12389gH14 | EVQLVESGGGLVQPGGSLRLSCAASGIDLSSNAMSWVRQAP | 83 |
| Heavy | GKGLEWIGTISSGGRTYYASWAKGRFTISKDSSKNTVYLQM | |
| chain | NSLRAEDTAVYYCARGDGATGFNIWGQGTLVTVSSASTKGP | |
| (IgG1) | SVFPLAPSSKSTSGGTAALGCLVKDYFPEPVTVSWNSGALTS | |
| | GVHTFPAVLQSSGLYSLSSVVTVPSSSLGTQTYICNVNHKPS | |
| | NTKVDKKVEPKSCDKTHTCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLSLSPGK | |
| 12389gH14 | gaagtgcagctggtcgaatccgggggtggtctggtgcagccgggaggttccctgcgcttgtcat | 84 |
| Heavy | gcgcggcttccggcattgaccttagctccaacgccatgagctgggtcagacaggcccctggca | |
| chain | aagggctggagtggattggcaccatctcaagcggagggcggacttactatgcctcctgggcca | |
| (IgG1)(nt.) | agggacgcttcaccatctcgaaggactcctcgaagaacaccgtgtacctccaaatgaactcgct | |
| | gagggcagaggacactgctgtgtactactgtgcgcggggagatggagccaccggcttcaatat | |
| | ctggggccagggaaccctcgtgactgtctcgagcgcttctacaaagggcccatcggtcttcccc | |
| | ctggcaccctcctccaagagcacctctgggggcacagcggccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacccagacctacatctgcaacgtgaatcacaagcccagcaacaccaaggtg | |
| | gacaagaaagttgagcccaaatcttgtgacaaaactcacacatgcccaccgtgcccagcacctg | |
| | aactcctggggggaccgtcagtcttcctcttccccccaaaacccaaggacaccctcatgatctcc | |
| | cggacccctgaggtcacatgcgtggtggtggacgtgagccacgaagaccctgaggtcaagttc | |
| | aactggtacgtggacggcgtggaggtgcataatgccaagacaaagccgcgggaggagcagt | |
| | acaacagcacgtaccgtgtggtcagcgtcctcaccgtcctgcaccaggactggctgaatggca | |
| | aggagtacaagtgcaaggtctccaacaaagccctcccagcccccatcgagaaaaccatctcca | |
| | aagccaaagggcagccccgagaaccacaggtgtacaccctgcccccatcccgggatgagct | |
| | gaccaagaaccaggtcagcctgacctgcctggtcaaaggcttctatcccagcgacatcgccgt | |
| | ggagtgggagagcaatgggcagccggagaacaactacaagaccacgcctcccgtgctggac | |
| | tccgacggctccttcttcctctacagcaagctcaccgtggacaagagcaggtggcagcagggg | |
| | aacgtcttctcatgctccgtgatgcatgaggctctgcacaaccactacacgcagaagagcctctc | |
| | cctgtctccgggtaaa | |
| 12389gH14 | EVQLVESGGGLVQPGGSLRLSCAASGIDLSSNAMSWVRQAP | 85 |
| Heavy | GKGLEWIGTISSGGRTYYASWAKGRFTISKDSSKNTVYLQM | |
| chain | NSLRAEDTAVYYCARGDGATGFNIWGQGTLVTVSSASTKGP | |
| (IgG4P) | SVFPLAPCSRSTSESTAALGCLVKDYFPEPVTVSWNSGALTS | |
| | GVHTFPAVLQSSGLYSLSSVVTVPSSSLGTKTYTCNVDHKPS | |
| | NTKVDKRVESKYGPPCPPCPAPEFLGGPSVFLFPPKPKDTLMI | |
| | SRTPEVTCVVVDVSQEDPEVQFNWYVDGVEVHNAKTKPRE | |
| | EQFNSTYRVVSVLTVLHQDWLNGKEYKCKVSNKGLPSSIEK | |
| | TISKAKGQPREPQVYTLPPSQEEMTKNQVSLTCLVKGFYPSD | |
| | IAVEWESNGQPENNYKTTPPVLDSDGSFFLYSRLTVDKSRW | |
| | QEGNVFSCSVMHEALHNHYTQKSLSLSLGK | |
| 12389gH14 | gaagtgcagctggtcgaatccgggggtggtctggtgcagccgggaggttccctgcgcttgtcat | 86 |
| Heavy | gcgcggcttccggcattgaccttagctccaacgccatgagctgggtcagacaggcccctggca | |
| chain | aagggctggagtggattggcaccatctcaagcggagggggacttactatgcctcctgggcca | |
| (IgG4P) | agggacgcttcaccatctcgaaggactcctcgaagaacaccgtgtacctccaaatgaactcgct | |
| (nt.) | gagggcagaggacactgctgtgtactactgtgcgcggggagatggagccaccggcttcaatat | |
| | ctggggccagggaaccctcgtgactgtctcgagcgcttctacaaagggcccatccgtcttcccc | |
| | ctggcgccctgctccaggagcacctccgagagcacagccgccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacgaagacctacacctgcaacgtagatcacaagcccagcaacaccaaggtg | |
| | gacaagagagttgagtccaaatatggtcccccatgcccaccatgcccagcacctgagttcctgg | |
| | ggggaccatcagtcttcctgttccccccaaaacccaaggacactctcatgatctcccggacccct | |
| | gaggtcacgtgcgtggtggtggacgtgagccaggaagaccccgaggtccagttcaactggta | |
| | cgtggatggcgtggaggtgcataatgccaagacaaagccgcgggaggagcagttcaacagc | |
| | acgtaccgtgtggtcagcgtcctcaccgtcctgcaccaggactggctgaacggcaaggagtac | |
| | aagtgcaaggtctccaacaaaggcctcccgtcctccatcgagaaaaccatctccaaagccaaa | |
| | gggcagccccgagagccacaggtgtacaccctgcccccatcccaggaggagatgaccaaga | |
| | accaggtcagcctgacctgcctggtcaaaggcttctaccccagcgacatcgccgtggagtggg | |
| | agagcaatgggcagccggagaacaactacaagaccacgcctcccgtgctggactccgacgg | |
| | ctccttcttcctctacagcaggctaaccgtggacaagagcaggtggcaggaggggaatgtcttct | |
| | catgctccgtgatgcatgaggctctgcacaaccactacacacagaagagcctctccctgtctctg | |
| | ggtaaa | |
| 12389gH15 | EVQLVESGGGLVQPGGSLRLSCAVSGIDLSSNAMSWVRQAP | 87 |
| VH | GKGLEWIGTISSGGRTYYASWAKGRFTISKDNSKNTVYLQM | |
| | NSLRAEDTAVYYCARGDGATGFNIWGQGTLVTVSS | |
| 12389gH15 | gaagtgcagctggtcgagtccggtggaggcctggtgcagcccggcggctccctgcggctgtc | 88 |
| VH (nt.) | ctgcgccgtctccgggatcgacctgtcctccaacgctatgagctgggtcagacaggcacccgg | |
| | gaagggtctggagtggatcgggacaatctccagcggcggccgcacctactacgcatcctggg | |
| | ccaagggaaggttcaccatttcgaaggacaactccaagaacaccgtgtaccttcagatgaactc | |
| | cctgcgggccgaggatactgccgtgtattactgtgcgaggggagatggcgcgactgggttcaa | |
| | catctggggacagggaacgctggtgactgtgtcctcg | |
| 12389gH15 | EVQLVESGGGLVQPGGSLRLSCAVSGIDLSSNAMSWVRQAP | 89 |
| Heavy | GKGLEWIGTISSGGRTYYASWAKGRFTISKDNSKNTVYLQM | |
| chain | NSLRAEDTAVYYCARGDGATGFNIWGQGTLVTVSSASTKGP | |
| (IgG1) | SVFPLAPSSKSTSGGTAALGCLVKDYFPEPVTVSWNSGALTS | |
| | GVHTFPAVLQSSGLYSLSSVVTVPSSSLGTQTYICNVNHKPS | |
| | NTKVDKKVEPKSCDKTHTCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLSLSPGK | |
| 12389gH15 | gaagtgcagctggtcgagtccggtggaggcctggtgcagcccggcggctccctgcggctgtc | 90 |
| Heavy | ctgcgccgtctccgggatcgacctgtcctccaacgctatgagctgggtcagacaggcacccgg | |
| chain | gaagggtctggagtggatcgggacaatctccagcggcggccgcacctactacgcatcctggg | |
| (IgG1)(nt.) | ccaagggaaggttcaccatttcgaaggacaactccaagaacaccgtgtaccttcagatgaactc | |
| | cctgcgggccgaggatactgccgtgtattactgtgcgaggggagatggcgcgactgggttcaa | |
| | catctggggacagggaacgctggtgactgtgtcctcggcttcgaccaaggggccctcggtgttc | |
| | cctctggcgcctagctccaagagcacttcaggcggaacggctgccctcggatgcctggtcaag | |
| | gactacttcccagagcccgtgaccgtgtcatggaacagcggagctctgactagcggagtgcac | |
| | acctttccggcggtgctgcaaagctcaggcctgtactcgctctcctcagtggtcactgtcccgtcc | |
| | tcctcgctggggactcaaacgtacatctgcaacgtcaatcacaaaccgtcaaataccaaagtcg | |
| | acaagaaggtcgagcctaagtcgtgcgataagacccacacctgtccgccatgcccagcccctg | |
| | aactgctgggaggaccaagcgtgttcctcttcccgccaaaaccaaaagacaccttgatgatttca | |
| | cggactcctgaggtcacgtgcgtcgtggtggacgtgagccatgaggacccagaagtgaaattc | |
| | aattggtacgtggacggagtcgaagtgcataacgccaagaccaagcccagagaggaacagta | |
| | taacagcacctaccgggtcgtgagcgtgctcactgtgttgcaccaggactggctcaatggaaag | |
| | gagtacaagtgcaaagtgtccaataaggccctccctgctcccatcgaaaagaccatcagcaag | |
| | gcaaagggccagccacgcgaaccgcaagtgtacacccttccgccgtcgcgcgatgaactgac | |
| | taaaaaccaagtctcgctgacctgtctcgtgaagggcttttacccgtccgacattgcggtggagt | |
| | gggagtcgaacggtcagccggaaaacaattacaagaccactccgccggtgctcgactcggat | |
| | gggtccttcttcctctactcgaagttgaccgtcgataagtccagatggcagcagggtaacgtgttc | |
| | tcgtgcagcgtgatgcacgaggccctgcacaaccattacactcagaagtcgctttccttgtcacc | |
| | gggcaaa | |
| 12389gH15 | EVQLVESGGGLVQPGGSLRLSCAVSGIDLSSNAMSWVRQAP | 91 |
| Heavy | GKGLEWIGTISSGGRTYYASWAKGRFTISKDNSKNTVYLQM | |
| chain | NSLRAEDTAVYYCARGDGATGFNIWGQGTLVTVSSASTKGP | |
| (IgG4P) | SVFPLAPCSRSTSESTAALGCLVKDYFPEPVTVSWNSGALTS | |
| | GVHTFPAVLQSSGLYSLSSVVTVPSSSLGTKTYTCNVDHKPS | |
| | NTKVDKRVESKYGPPCPPCPAPEFLGGPSVFLFPPKPKDTLMI | |
| | SRTPEVTCVVVDVSQEDPEVQFNWYVDGVEVHNAKTKPRE | |
| | EQFNSTYRVVSVLTVLHQDWLNGKEYKCKVSNKGLPSSIEK | |
| | TISKAKGQPREPQVYTLPPSQEEMTKNQVSLTCLVKGFYPSD | |
| | IAVEWESNGQPENNYKTTPPVLDSDGSFFLYSRLTVDKSRW | |
| | QEGNVFSCSVMHEALHNHYTQKSLSLSLGK | |
| 12389gH15 | gaagtgcagctggtcgagtccggtggaggcctggtgcagcccggcggctccctgcggctgtc | 92 |
| Heavy | ctgcgccgtctccgggatcgacctgtcctccaacgctatgagctgggtcagacaggcacccgg | |
| chain | gaagggtctggagtggatcgggacaatctccagcggcggccgcacctactacgcatcctggg | |
| (IgG4P) | ccaagggaaggttcaccatttcgaaggacaactccaagaacaccgtgtaccttcagatgaactc | |
| (nt.) | cctgcgggccgaggatactgccgtgtattactgtgcgaggggagatggcgcgactgggttcaa | |
| | catctggggacagggaacgctggtgactgtgtcctcggcttctacaaagggcccatccgtcttcc | |
| | ccctggcgccctgctccaggagcacctccgagagcacagccgccctgggctgcctggtcaag | |
| | gactacttccccgaaccggtgacggtgtcgtggaactcaggcgccctgaccagcggcgtgcac | |
| | accttcccggctgtcctacagtcctcaggactctactccctcagcagcgtggtgaccgtgccctc | |
| | cagcagcttgggcacgaagacctacacctgcaacgtagatcacaagcccagcaacaccaagg | |
| | tggacaagagagttgagtccaaatatggtcccccatgcccaccatgcccagcacctgagttcct | |
| | ggggggaccatcagtcttcctgttccccccaaaacccaaggacactctcatgatctcccggacc | |
| | cctgaggtcacgtgcgtggtggtggacgtgagccaggaagaccccgaggtccagttcaactg | |
| | gtacgtggatggcgtggaggtgcataatgccaagacaaagccgcgggaggagcagttcaaca | |
| | gcacgtaccgtgtggtcagcgtcctcaccgtcctgcaccaggactggctgaacggcaaggagt | |
| | acaagtgcaaggtctccaacaaaggcctcccgtcctccatcgagaaaaccatctccaaagcca | |
| | aagggcagccccgagagccacaggtgtacaccctgcccccatcccaggaggagatgaccaa | |
| | gaaccaggtcagcctgacctgcctggtcaaaggcttctaccccagcgacatcgccgtggagtg | |
| | ggagagcaatgggcagccggagaacaactacaagaccacgcctcccgtgctggactccgac | |
| | ggctccttcttcctctacagcaggctaaccgtggacaagagcaggtggcaggaggggaatgtct | |
| | tctcatgctccgtgatgcatgaggctctgcacaaccactacacacagaagagcctctccctgtct | |
| | ctgggtaaa | |
| 12389gH16 | EVQLVESGGGLVQPGGSLRLSCAASGIDLSSNAMSWVRQAP | 93 |
| VH | GKGLEWIGTISSGGRTYYASWAKGRFTISKDNSKNTVYLQM | |
| | NSLRAEDTAVYYCARGDGATGFNIWGQGTLVTVSS | |
| 12389gH16 | gaagtgcagctggtcgagtccggtggaggcctggtgcagcccggcggctccctgcggctgtc | 94 |
| VH (nt.) | ctgcgccgcctccgggatcgacctgtcctccaacgctatgagctgggtcagacaggcacccgg | |
| | gaagggtctggagtggatcgggacaatctccagcggcggccgcacctactacgcatcctggg | |
| | ccaagggaaggttcaccatttcgaaggacaactccaagaacaccgtgtaccttcagatgaactc | |
| | cctgcgggccgaggatactgccgtgtattactgtgcgaggggagatggcgcgactgggttcaa | |
| | catctggggacagggaacgctggtgactgtgtcctcg | |
| 12389gH16 | EVQLVESGGGLVQPGGSLRLSCAASGIDLSSNAMSWVRQAP | 95 |
| Heavy | GKGLEWIGTISSGGRTYYASWAKGRFTISKDNSKNTVYLQM | |
| chain | NSLRAEDTAVYYCARGDGATGFNIWGQGTLVTVSSASTKGP | |
| (IgG1) | SVFPLAPSSKSTSGGTAALGCLVKDYFPEPVTVSWNSGALTS | |
| | GVHTFPAVLQSSGLYSLSSVVTVPSSSLGTQTYICNVNHKPS | |
| | NTKVDKKVEPKSCDKTHTCPPCPAPELLGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLSLSPGK | |
| 12389gH16 | gaagtgcagctggtcgagtccggtggaggcctggtgcagcccggcggctccctgcggctgtc | 96 |
| Heavy | ctgcgccgcctccgggatcgacctgtcctccaacgctatgagctgggtcagacaggcacccgg | |
| chain | gaagggtctggagtggatcgggacaatctccagcggcggccgcacctactacgcatcctggg | |
| (IgG1)(nt.) | ccaagggaaggttcaccatttcgaaggacaactccaagaacaccgtgtaccttcagatgaactc | |
| | cctgcgggccgaggatactgccgtgtattactgtgcgaggggagatggcgcgactgggttcaa | |
| | catctggggacagggaacgctggtgactgtgtcctcggcttcgaccaaggggccctcggtgttc | |
| | cctctggcgcctagctccaagagcacttcaggcggaacggctgccctcggatgcctggtcaag | |
| | gactacttcccagagcccgtgaccgtgtcatggaacagcggagctctgactagcggagtgcac | |
| | acctttccggcggtgctgcaaagctcaggcctgtactcgctctcctcagtggtcactgtcccgtcc | |
| | tcctcgctggggactcaaacgtacatctgcaacgtcaatcacaaaccgtcaaataccaaagtcg | |
| | acaagaaggtcgagcctaagtcgtgcgataagacccacacctgtccgccatgcccagcccctg | |
| | aactgctgggaggaccaagcgtgttcctcttcccgccaaaaccaaaagacaccttgatgatttca | |
| | cggactcctgaggtcacgtgcgtcgtggtggacgtgagccatgaggacccagaagtgaaattc | |
| | aattggtacgtggacggagtcgaagtgcataacgccaagaccaagcccagagaggaacagta | |
| | taacagcacctaccgggtcgtgagcgtgctcactgtgttgcaccaggactggctcaatggaaag | |
| | gagtacaagtgcaaagtgtccaataaggccctccctgctcccatcgaaaagaccatcagcaag | |
| | gcaaagggccagccacgcgaaccgcaagtgtacacccttccgccgtcgcgcgatgaactgac | |
| | taaaaaccaagtctcgctgacctgtctcgtgaagggcttttacccgtccgacattgcggtggagt | |
| | gggagtcgaacggtcagccggaaaacaattacaagaccactccgccggtgctcgactcggat | |
| | gggtccttcttcctctactcgaagttgaccgtcgataagtccagatggcagcagggtaacgtgttc | |
| | tcgtgcagcgtgatgcacgaggccctgcacaaccattacactcagaagtcgctttccttgtcacc | |
| | gggcaaa | |
| 12389gH16 | EVQLVESGGGLVQPGGSLRLSCAASGIDLSSNAMSWVRQAP | 97 |
| Heavy | GKGLEWIGTISSGGRTYYASWAKGRFTISKDNSKNTVYLQM | |
| chain | NSLRAEDTAVYYCARGDGATGFNIWGQGTLVTVSSASTKGP | |
| (IgG1 | SVFPLAPSSKSTSGGTAALGCLVKDYFPEPVTVSWNSGALTS | |
| LALA) | GVHTFPAVLQSSGLYSLSSVVTVPSSSLGTQTYICNVNHKPS | |
| | NTKVDKKVEPKSCDKTHTCPPCPAPEAAGGPSVFLFPPKPKD | |
| | TLMISRTPEVTCVVVDVSHEDPEVKFNWYVDGVEVHNAKT | |
| | KPREEQYNSTYRVVSVLTVLHQDWLNGKEYKCKVSNKALP | |
| | APIEKTISKAKGQPREPQVYTLPPSRDELTKNQVSLTCLVKGF | |
| | YPSDIAVEWESNGQPENNYKTTPPVLDSDGSFFLYSKLTVDK | |
| | SRWQQGNVFSCSVMHEALHNHYTQKSLSLSPGK | |
| 12389gH16 | gaagtgcagctggtcgaatccgggggtggtctggtgcagccgggaggttccctgcgcttgtcat | 98 |
| Heavy | gcgcggcttccggcattgaccttagctccaacgccatgagctgggtcagacaggcccctggca | |
| chain | aagggctggagtggattggcaccatctcaagcggagggcggacttactatgcctcctgggcca | |
| (IgG1 | agggacgcttcaccatctcgaaggacaactcgaagaacaccgtgtacctccaaatgaactcgct | |
| LALA) | gagggcagaggacactgctgtgtactactgtgcgcggggagatggagccaccggcttcaatat | |
| (nt.) | ctggggccagggaaccctcgtgactgtctcgagcgcttctacaaagggcccatcggtcttcccc | |
| | ctggcaccctcctccaagagcacctctgggggcacagcggccctgggctgcctggtcaagga | |
| | ctacttccccgaacctgtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacccagacctacatctgcaacgtgaatcacaagcccagcaacaccaaggtcg | |
| | ataagaaagttgagcccaaatcttgtgacaaaactcacacatgcccaccgtgcccagcacctga | |
| | agctgctgggggaccgtcagtcttcctcttccccccaaaacccaaggacaccctcatgatctccc | |
| | ggacccctgaggtcacatgcgtggtggtggacgtgagccacgaagaccctgaggtcaagttca | |
| | actggtacgtggacggcgtggaggtgcataatgccaagacaaagccgcgggaggagcagta | |
| | caacagcacgtaccgtgtggtcagcgtcctcaccgtcctgcaccaggactggctgaatggcaa | |
| | ggagtacaagtgcaaggtatccaacaaagccctcccagcccccatcgagaaaaccatctccaa | |
| | agccaaagggcagccccgagaaccacaggtgtacaccctgcccccatcccgggatgagctg | |
| | accaagaaccaggtcagcctgacctgcctggtcaaaggcttctatcccagcgacatcgccgtg | |
| | gagtgggagagcaatgggcagccggagaacaactacaagaccacgcctcccgtgctggact | |
| | ccgacggctccttcttcctctacagcaagctcaccgtggacaagagcaggtggcagcagggga | |
| | acgtcttctcatgctccgtgatgcatgaggctctgcacaaccactacacgcagaagagcctctcc | |
| | ctgtctccgggtaaa | |
| 12389gH16 | EVQLVESGGGLVQPGGSLRLSCAASGIDLSSNAMSWVRQAP | 99 |
| Heavy | GKGLEWIGTISSGGRTYYASWAKGRFTISKDNSKNTVYLQM | |
| chain | NSLRAEDTAVYYCARGDGATGFNIWGQGTLVTVSSASTKGP | |
| (IgG4P) | SVFPLAPCSRSTSESTAALGCLVKDYFPEPVTVSWNSGALTS | |
| | GVHTFPAVLQSSGLYSLSSVVTVPSSSLGTKTYTCNVDHKPS | |
| | NTKVDKRVESKYGPPCPPCPAPEFLGGPSVFLFPPKPKDTLMI | |
| | SRTPEVTCVVVDVSQEDPEVQFNWYVDGVEVHNAKTKPRE | |
| | EQFNSTYRVVSVLTVLHQDWLNGKEYKCKVSNKGLPSSIEK | |
| | TISKAKGQPREPQVYTLPPSQEEMTKNQVSLTCLVKGFYPSD | |
| | IAVEWESNGQPENNYKTTPPVLDSDGSFFLYSRLTVDKSRW | |
| | QEGNVFSCSVMHEALHNHYTQKSLSLSLGK | |
| 12389gH16 | gaagtgcagctggtcgaatccgggggtggtctggtgcagccgggaggttccctgcgcttgtcat | 100 |
| Heavy | gcgcggcttccggcattgaccttagctccaacgccatgagctgggtcagacaggcccctggca | |
| chain | aagggctggagtggattggcaccatctcaagcggagggcggacttactatgcctcctgggcca | |
| (IgG4P) | agggacgcttcaccatctcgaaggacaactcgaagaacaccgtgtacctccaaatgaactcgct | |
| (nt.) | gagggcagaggacactgctgtgtactactgtgcgcggggagatggagccaccggcttcaatat | |
| | ctggggccagggaaccctcgtgactgtctcgagcgcttctacaaagggcccatccgtcttcccc | |
| | ctggcgccctgctccaggagcacctccgagagcacagccgccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacgaagacctacacctgcaacgtagatcacaagcccagcaacaccaaggtg | |
| | gacaagagagttgagtccaaatatggtcccccatgcccaccatgcccagcacctgagttcctgg | |
| | ggggaccatcagtcttcctgttccccccaaaacccaaggacactctcatgatctcccggacccct | |
| | gaggtcacgtgcgtggtggtggacgtgagccaggaagaccccgaggtccagttcaactggta | |
| | cgtggatggcgtggaggtgcataatgccaagacaaagccgcgggaggagcagttcaacagc | |
| | acgtaccgtgtggtcagcgtcctcaccgtcctgcaccaggactggctgaacggcaaggagtac | |
| | aagtgcaaggtctccaacaaaggcctcccgtcctccatcgagaaaaccatctccaaagccaaa | |
| | gggcagccccgagagccacaggtgtacaccctgcccccatcccaggaggagatgaccaaga | |
| | accaggtcagcctgacctgcctggtcaaaggcttctaccccagcgacatcgccgtggagtggg | |
| | agagcaatgggcagccggagaacaactacaagaccacgcctcccgtgctggactccgacgg | |
| | ctccttcttcctctacagcaggctaaccgtggacaagagcaggtggcaggaggggaatgtcttct | |
| | catgctccgtgatgcatgaggctctgcacaaccactacacacagaagagcctctccctgtctctg | |
| | ggtaaa | |
| 12389gH16 | EVQLVESGGGLVQPGGSLRLSCAASGIDLSSNAMSWVRQAP | 101 |
| Heavy | GKGLEWIGTISSGGRTYYASWAKGRFTISKDNSKNTVYLQM | |
| chain | NSLRAEDTAVYYCARGDGATGFNIWGQGTLVTVSSASTKGP | |
| (IgG4P | SVFPLAPCSRSTSESTAALGCLVKDYFPEPVTVSWNSGALTS | |
| FALA) | GVHTFPAVLQSSGLYSLSSVVTVPSSSLGTKTYTCNVDHKPS | |
| | NTKVDKRVESKYGPPCPPCPAPEAAGGPSVFLFPPKPKDTLM | |
| | ISRTPEVTCVVVDVSQEDPEVQFNWYVDGVEVHNAKTKPRE | |
| | EQFNSTYRVVSVLTVLHQDWLNGKEYKCKVSNKGLPSSIEK | |
| | TISKAKGQPREPQVYTLPPSQEEMTKNQVSLTCLVKGFYPSD | |
| | IAVEWESNGQPENNYKTTPPVLDSDGSFFLYSRLTVDKSRW | |
| | QEGNVFSCSVMHEALHNHYTQKSLSLSLGK | |
| 12389gH16 | gaagtgcagctggtcgaatccgggggtggtctggtgcagccgggaggttccctgcgcttgtcat | 102 |
| Heavy | gcgcggcttccggcattgaccttagctccaacgccatgagctgggtcagacaggcccctggca | |
| chain | aagggctggagtggattggcaccatctcaagcggagggcggacttactatgcctcctgggcca | |
| (IgG4P | agggacgcttcaccatctcgaaggacaactcgaagaacaccgtgtacctccaaatgaactcgct | |
| FALA) | gagggcagaggacactgctgtgtactactgtgcgcggggagatggagccaccggcttcaatat | |
| (nt.) | ctggggccagggaaccctcgtgactgtctcgagcgcttctacaaagggcccatccgtcttcccc | |
| | ctggcgccctgctccaggagcacctccgagagcacagccgccctgggctgcctggtcaagga | |
| | ctacttccccgaaccggtgacggtgtcgtggaactcaggcgccctgaccagcggcgtgcacac | |
| | cttcccggctgtcctacagtcctcaggactctactccctcagcagcgtggtgaccgtgccctcca | |
| | gcagcttgggcacgaagacctacacctgcaacgtagatcacaagcccagcaacaccaaggtg | |
| | gacaagagagttgagtccaaatatggtcccccatgcccaccatgcccagcacctgaagccgcg | |
| | gggggaccgtcagtcttcctgttccccccaaaacccaaggacactctcatgatctcccggaccc | |
| | ctgaggtcacgtgcgtggtggtggacgtgagccaggaagaccccgaggtccagttcaactggt | |
| | acgtggatggcgtggaggtgcataatgccaagacaaagccgcgggaggagcagttcaacag | |
| | cacgtaccgtgtggtcagcgtcctcaccgtcctgcaccaggactggctgaacggcaaggagta | |
| | caagtgcaaggtctccaacaaaggcctcccgtcctccatcgagaaaaccatctccaaagccaa | |
| | agggcagccccgagagccacaggtgtacaccctgcccccatcccaggaggagatgaccaag | |
| | aaccaggtcagcctgacctgcctggtcaaaggcttctaccccagcgacatcgccgtggagtgg | |
| | gagagcaatgggcagccggagaacaactacaagaccacgcctcccgtgctggactccgacg | |
| | gctccttcttcctctacagcaggctaaccgtggacaagagcaggtggcaggaggggaatgtctt | |
| | ctcatgctccgtgatgcatgaggctctgcacaaccactacacacagaagagcctctccctgtctc | |
| | tgggtaaa | |
| Human | AIQLTQSPSSLSASVGDRVTITCRASQGISSALAWYQQKPGK | 103 |
| IGKV1D- | APKLLIYDASSLESGVPSRFSGSGSGTDFTLTISSLQPEDFATY | |
| 13 IGKJ4 | YCQQFNSYPLTFGGGTKVEIK | |
| acceptor | | |
| framework | | |
| Human | gccatccagttgacccagtctccatcctccctgtctgcatctgtaggagacagagtcaccatcact | 104 |
| IGKV1D- | tgccgggcaagtcagggcattagcagtgctttagcctggtatcagcagaaaccagggaaagct | |
| 13 IGKJ4 | cctaagctcctgatctatgatgcctccagtttggaaagtggggtcccatcaaggttcagcggcag | |
| acceptor | tggatctgggacagatttcactctcaccatcagcagcctgcagcctgaagattttgcaacttattac | |
| framework | tgtcaacagtttaatagttaccctctcactttcggcggagggaccaaggtggagatcaaa | |
| (nt.) | | |
| Human | EVQLVESGGGLVQPGGSLRLSCAASGFTVSSNYMSWVRQAP | 105 |
| IGHV3-66 | GKGLEWVSVIYSGGSTYYADSVKGRFTISRDNSKNTLYLQM | |
| IGHJ4 | NSLRAEDTAVYYCARYFDYWGQGTLVTVSS | |
| acceptor | | |
| framework | | |
| Human | gaggtgcagctggtggagtctgggggaggcttggtccagcctggggggtccctgagactctcc | 106 |
| IGHV3-66 | tgtgcagcctctggattcaccgtcagtagcaactacatgagctgggtccgccaggctccaggga | |
| IGHJ4 | aggggctggagtgggtctcagttatttatagcggtggtagcacatactacgcagactccgtgaag | |
| acceptor | ggcagattcaccatctccagagacaattccaagaacacgctgtatcttcaaatgaacagcctgag | |
| framework | agccgaggacacggctgtgtattactgtgcgagatactttgactactggggccaaggaaccctg | |
| (nt.) | gtcaccgtctcctca | |

1-41. (canceled)

42. An antibody that specifically binds to HLA-G, wherein the antibody comprises:
a) a light chain variable region comprising:
i) a CDR-L1 comprising SEQ ID NO: 1;
ii) a CDR-L2 comprising SEQ ID NO: 2 and
iii) a CDR-L3 comprising SEQ ID NO: 3; and
b) a heavy chain variable region comprising:
i) a CDR-H1 comprising SEQ ID NO: 4;
ii) a CDR-H2 comprising SEQ ID NO: 5 and
iii) a CDR-H3 comprising SEQ ID NO: 6.

43. The antibody according to claim 42, wherein the antibody is chimeric or humanized.

44. The antibody according to claim 42, wherein the antibody comprises:
a) a light chain variable region comprising SEQ ID NO: 19 or 15 or 23; and
b) a heavy chain variable region comprising SEQ ID NO: 93, 27, 33, 57, 69, 75, 81 or 87; or
c) a light chain variable region comprising SEQ ID NO: 19; and
d) a heavy chain variable region comprising SEQ ID NO: 93.

45. The antibody according to claim 42, wherein the antibody is a full-length antibody.

46. The antibody according to claim 45, wherein the full-length antibody is an IgG1, IgG1 LALA, IgG1LALAGA, IgG4, IgG4P, or IgG4P FALA.

47. The antibody according to claim 46, wherein the antibody is an IgG1 or an afucosylated IgG1.

48. The antibody according to claim 47, wherein the antibody comprises:
a) a light chain comprising SEQ ID NO: 21 or 17 or 25; and
b) a heavy chain comprising SEQ ID NO: 95, 29, 35, 59, 71, 77, 83, or 89; or
c) a light chain comprising at least 90% identity or similarity to SEQ ID NO: 21 or 17 or 25; and
d) a heavy chain comprising at least 90% identity or similarity to SEQ ID NO: 95, 29, 35, 59, 71, 77, 83, or 89.

49. The antibody according to claim 47, wherein the antibody comprises a light chain variable region comprising SEQ ID NO: 19 and a heavy chain variable region comprising SEQ ID NO: 93, and wherein the reminder of the light chain and heavy chain has at least 90% identity or similarity to SEQ ID NOs: 21 and 95 respectively.

50. The antibody according to claim 47, wherein the antibody comprises a light chain comprising SEQ ID NO: 21 and a heavy chain comprising SEQ ID NO: 95.

51. The antibody according to claim 47, wherein the antibody inhibits HLA-G mediated immune suppressive function and inhibits tumor growth by depleting tumor cells expressing HLA-G.

52. The antibody according to claim 50, wherein the antibody comprises a light chain comprising SEQ ID NO: 21 and a heavy chain comprising SEQ ID NO: 95, and wherein the antibody has an improved ADCC and/or ADCP and/or CDC function, and/or has an improved ability to deplete tumor cells expressing HLA-G.

53. The antibody according to claim 42, wherein the antibody is a Fab, a Fab′, a F(ab′)₂, a dsFv, a scFv, or a dsscFv.

54. An antibody that cross-competes with the antibody of claim 42 for binding to HLA-G or binds to an epitope of HLA-G comprising V194, F195, Y197, E198, Q224, Q226, D227, V248, V249, P250 and Y257 of HLA-G (SEQ ID NO: 107).

55. An isolated polynucleotide encoding the antibody according to claim 42.

56. The isolated polynucleotide according to claim 55, wherein the polynucleotide encodes:
a) a light chain variable region, wherein the polynucleotide:
i) is at least 90% identical to SEQ ID NO: 20, 16 or 24; or
ii) comprises or consists of SEQ ID NO: 20, 16 or 24; or
b) a heavy chain variable region, wherein the polynucleotide:
i) is at least 90% identical to SEQ ID NO: 94, 28, 34, 58, 70, 76, 82 or 88; or
ii) comprises or consists of SEQ ID NO: 94, 28, 34, 58, 70, 76, 82 or 88; or
c) a light chain, wherein the polynucleotide:
i) is at least 90% identical to SEQ ID NO: 22, 18, or 26; or
ii) comprises or consists of SEQ ID NO: 22, 18, or 26; or
d) a heavy chain, wherein the polynucleotide:
i) is at least 90% identical to SEQ ID NO: 96, 30, 36, 60, 72, 78, 84, or 90; or
ii) comprises or consists of SEQ ID NO: 96, 30, 36, 60, 72, 78, 84, or 90.

57. A cloning or expression vector comprising one or more polynucleotides according to claim 55.

58. A host cell comprising one or more polynucleotides according to claim 55 or one or more expression vectors comprising one or more of said polynucleotides.

59. A host cell comprising one or more polynucleotides according to claim 55 or one or more expression vectors comprising said polynucleotides, wherein the host cell has been genetically modified to decrease or abolish the function of the alpha1,6 fucosyltransferase.

60. A process for the production of an antibody comprising culturing the host cell according to claim 58 under suitable conditions for producing the antibody and isolating the antibody, the host cell having optionally been genetically modified to decrease or abolish the function of the alpha1,6 fucosyltransferase.

61. A process for the production of a pharmaceutical composition comprising formulating the antibody according to claim 42 and one or more pharmaceutically acceptable carriers, excipients or diluents into a pharmaceutical composition.

62. A pharmaceutical composition comprising the antibody according to claim 42 and one or more pharmaceutically acceptable carriers, excipients or diluents.

63. A method of treating a solid tumor in a patient comprising administering to said patient a therapeutically effective amount of an antibody according to claim 42 or a pharmaceutical composition comprising said antibody.

64. The method according to claim 63, wherein the solid tumor is selected from renal clear cell carcinoma (RCC), colorectal carcinoma (CRC), pancreatic cancer, ovarian cancer, head and neck carcinoma, stomach cancer and hepatocellular carcinoma.

65. A method for diagnosing renal clear cell carcinoma (RCC), colorectal carcinoma (CRC), pancreatic cancer, ovarian cancer, head and neck carcinoma, stomach cancer or hepatocellular carcinoma by using an antibody according to claim 42 or a pharmaceutical composition comprising said antibody.


SEQUENCE LISTING (146 sequences)

SEQ ID NO: 1 - protein, 11 aa, synthetic construct
QASQSIYSYLS

SEQ ID NO: 2 - protein, 7 aa, synthetic construct
KASTLAS

SEQ ID NO: 3 - protein, 12 aa, synthetic construct
QNHWNVGGNGWP

SEQ ID NO: 4 - protein, 10 aa, synthetic construct
GIDLSSNAMS

SEQ ID NO: 5 - protein, 16 aa, synthetic construct
TISSGGRTYYASWAKG

SEQ ID NO: 6 - protein, 9 aa, synthetic construct
GDGATGFNI

SEQ ID NO: 7 - protein, 110 aa, synthetic construct
ALVMTQTPASVSEPVGGTVTIKCQASQSIYSYLSWYQQKPGQPPKLLIYKASTLASGVSSRFKGSGSGTQFTLTISDLECGDAATYYCQNHWNVGGNGWPFGGGTEVVVK

SEQ ID NO: 8 - DNA, 330 bp, synthetic construct
  source 1..330: mol_type = other DNA
gcccttgtgatgacccagactccagcctccgtgtctgaacctgtgggaggcacagtcaccatcaagtgccaggccagtcagagcatttacagctacttatcctggtatcagcagaaaccagggcagcctcccaagctcctaatctacaaggcatccactctggcatctggggtctcatcgcggttcaaaggcagtggatctgggacacagttcactctcaccatcagcgacctggagtgtggcgatgctgccacttactactgtcaaaatcattggaatgttggtggtaatggttggcctttcggcggagggaccgaggtggtggtcaaa

SEQ ID NO: 9 - protein, 214 aa, synthetic construct
ALVMTQTPASVSEPVGGTVTIKCQASQSIYSYLSWYQQKPGQPPKLLIYKASTLASGVSSRFKGSGSGTQFTLTISDLECGDAATYYCQNHWNVGGNGWPFGGGTEVVVKRTPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 10 - DNA, 642 bp, synthetic construct
  source 1..642: mol_type = other DNA
gcccttgtgatgacccagactccagcctccgtgtctgaacctgtgggaggcacagtcaccatcaagtgccaggccagtcagagcatttacagctacttatcctggtatcagcagaaaccagggcagcctcccaagctcctaatctacaaggcatccactctggcatctggggtctcatcgcggttcaaaggcagtggatctgggacacagttcactctcaccatcagcgacctggagtgtggcgatgctgccacttactactgtcaaaatcattggaatgttggtggtaatggttggcctttcggcggagggaccgaggtggtggtcaaacgtacgccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgt

SEQ ID NO: 11 - protein, 114 aa, synthetic construct
QSVEESGGRLVTPGTPLTLTCTVSGIDLSSNAMSWVRQAPGEGLEWIGTISSGGRTYYASWAKGRFTISKTSTTVDLKIPSPTTEDTATYFCGRGDGATGFNIWGPGTLVTVSS

SEQ ID NO: 12 - DNA, 342 bp, synthetic construct
  source 1..342: mol_type = other DNA
cagtcggtggaggagtccgggggtcgcctggtcacgcctgggacacccctgacactcacctgcacagtctctggaatcgacctcagtagcaatgcaatgagctgggtccgccaggctccaggggaggggctggaatggatcggaaccattagtagtggtggtaggacatactacgcgagctgggcaaaaggccgattcaccatctccaaaacctcgaccacggtggatctgaaaatccccagtccgacaaccgaggacacggccacctatttctgtggcagaggagatggtgctactggctttaacatctggggcccaggcaccctggtcaccgtctcgagt

SEQ ID NO: 13 - protein, 217 aa, synthetic construct
QSVEESGGRLVTPGTPLTLTCTVSGIDLSSNAMSWVRQAPGEGLEWIGTISSGGRTYYASWAKGRFTISKTSTTVDLKIPSPTTEDTATYFCGRGDGATGFNIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKP

SEQ ID NO: 14 - DNA, 651 bp, synthetic construct
  source 1..651: mol_type = other DNA
cagtcggtggaggagtccgggggtcgcctggtcacgcctgggacacccctgacactcacctgcacagtctctggaatcgacctcagtagcaatgcaatgagctgggtccgccaggctccaggggaggggctggaatggatcggaaccattagtagtggtggtaggacatactacgcgagctgggcaaaaggccgattcaccatctccaaaacctcgaccacggtggatctgaaaatccccagtccgacaaccgaggacacggccacctatttctgtggcagaggagatggtgctactggctttaacatctggggcccaggcaccctggtcaccgtctcgagtgggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagccc

SEQ ID NO: 15 - protein, 110 aa, synthetic construct
AIVLTQSPSSLSASVGDRVTITCQASQSIYSYLSWYQQKPGKAPKLLIYKASTLASGVPSRFSGSGSGTQFTLTISSLQPEDFATYYCQNHWNVGGNGWPFGGGTKVEIK

SEQ ID NO: 16 - DNA, 330 bp, synthetic construct
  source 1..330: mol_type = other DNA
gccattgtgttgactcagagcccgtcgtcactgagcgcttccgtgggcgacagagtgaccatcacctgtcaagccagccagtccatctactcctacctgtcatggtaccagcagaagccagggaaagccccgaagctgctgatctacaaggcctctacccttgcgtccggagtgccttcgcggttttcgggttccggttccggaactcagttcacgctcaccattagctccctccaacccgaggatttcgcaacctactattgccaaaaccactggaacgtcgggggcaatggctggcccttcggaggaggcactaaggtcgaaatcaag

SEQ ID NO: 17 - protein, 217 aa, synthetic construct
AIVLTQSPSSLSASVGDRVTITCQASQSIYSYLSWYQQKPGKAPKLLIYKASTLASGVPSRFSGSGSGTQFTLTISSLQPEDFATYYCQNHWNVGGNGWPFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - DNA, 651 bp, synthetic construct
  source 1..651: mol_type = other DNA
gccattgtgttgactcagagcccgtcgtcactgagcgcttccgtgggcgacagagtgaccatcacctgtcaagccagccagtccatctactcctacctgtcatggtaccagcagaagccagggaaagccccgaagctgctgatctacaaggcctctacccttgcgtccggagtgccttcgcggttttcgggttccggttccggaactcagttcacgctcaccattagctccctccaacccgaggatttcgcaacctactattgccaaaaccactggaacgtcgggggcaatggctggcccttcggaggaggcactaaggtcgaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 19 - protein, 110 aa, synthetic construct
AIVLTQSPSSLSASVGDRVTITCQASQSIYSYLSWYQQKPGKAPKLLIYKASTLASGVPSRFSGSGSGTDFTLTISSLQPEDFATYYCQNHWNVGGNGWPFGGGTKVEIK

SEQ ID NO: 20 - DNA, 330 bp, synthetic construct
  source 1..330: mol_type = other DNA
gccattgtgctgactcagtcaccgagctcacttagcgcctccgtgggggaccgggtcacaatcacttgccaagcgtcgcagtcaatctactcgtacctctcgtggtatcagcagaagcctgggaaggcacctaaactcctgatctacaaggcttcaactttggcatctggagtgccgagcagattcagcggatcgggaagcggaactgattttaccctcactatctcgtcgctccaaccggaagatttcgcgacctactactgtcaaaaccattggaatgtcggtggaaacggttggcctttcggcgggggaaccaaagtggagattaag

SEQ ID NO: 21 - protein, 217 aa, synthetic construct
AIVLTQSPSSLSASVGDRVTITCQASQSIYSYLSWYQQKPGKAPKLLIYKASTLASGVPSRFSGSGSGTDFTLTISSLQPEDFATYYCQNHWNVGGNGWPFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - DNA, 651 bp, synthetic construct
  source 1..651: mol_type = other DNA
gccattgtgctgactcagtcaccgagctcacttagcgcctccgtgggggaccgggtcacaatcacttgccaagcgtcgcagtcaatctactcgtacctctcgtggtatcagcagaagcctgggaaggcacctaaactcctgatctacaaggcttcaactttggcatctggagtgccgagcagattcagcggatcgggaagcggaactgattttaccctcactatctcgtcgctccaaccggaagatttcgcgacctactactgtcaaaaccattggaatgtcggtggaaacggttggcctttcggcgggggaaccaaagtggagattaagcgtacggtggccgctccctccgtgttcatcttcccaccctccgacgagcagctgaagtccggcaccgcctccgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 23 - protein, 110 aa, synthetic construct
AIQLTQSPSSLSASVGDRVTITCQASQSIYSYLSWYQQKPGKAPKLLIYKASTLASGVPSRFSGSGSGTDFTLTISSLQPEDFATYYCQNHWNVGGNGWPFGGGTKVEIK

SEQ ID NO: 24 - DNA, 330 bp, synthetic construct
  source 1..330: mol_type = other DNA
gccattcagttgactcagagcccgtcgtcactgagcgcttccgtgggcgacagagtgaccatcacctgtcaagccagccagtccatctactcctacctgtcatggtaccagcagaagccagggaaagccccgaagctgctgatctacaaggcctctacccttgcgtccggagtgccttcgcggttttcgggttccggttccggaactgacttcacgctcaccattagctccctccaacccgaggatttcgcaacctactattgccaaaaccactggaacgtcgggggcaatggctggcccttcggaggaggcactaaggtcgaaatcaag

SEQ ID NO: 25 - protein, 217 aa, synthetic construct
AIQLTQSPSSLSASVGDRVTITCQASQSIYSYLSWYQQKPGKAPKLLIYKASTLASGVPSRFSGSGSGTDFTLTISSLQPEDFATYYCQNHWNVGGNGWPFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 651 bp, synthetic construct
  source 1..651: mol_type = other DNA
gccattcagttgactcagagcccgtcgtcactgagcgcttccgtgggcgacagagtgaccatcacctgtcaagccagccagtccatctactcctacctgtcatggtaccagcagaagccagggaaagccccgaagctgctgatctacaaggcctctacccttgcgtccggagtgccttcgcggttttcgggttccggttccggaactgacttcacgctcaccattagctccctccaacccgaggatttcgcaacctactattgccaaaaccactggaacgtcgggggcaatggctggcccttcggaggaggcactaaggtcgaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 27 - protein, 117 aa, synthetic construct
EVQLVESGGGLVQPGGSLRLSCAVSGIDLSSNAMSWVRQAPGKGLEWIGTISSGGRTYYASWAKGRFTISKDSSKNTVYLQMNSLRAEDTAVYYCGRGDGATGFNIWGQGTLVTVSS

SEQ ID NO: 28 - DNA, 351 bp, synthetic construct
  source 1..351: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggattggcaccatctcaagcggagggcggacttactatgcctcctgggccaagggacgcttcaccatctcgaaggactcctcgaagaacaccgtgtacctccaaatgaactcgctgagggcagaggacactgctgtgtactactgtggacggggagatggagccaccggcttcaatatctggggccagggaaccctcgtgactgtctcgagc

SEQ ID NO: 29 - protein, 447 aa, synthetic construct
EVQLVESGGGLVQPGGSLRLSCAVSGIDLSSNAMSWVRQAPGKGLEWIGTISSGGRTYYASWAKGRFTISKDSSKNTVYLQMNSLRAEDTAVYYCGRGDGATGFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - DNA, 1341 bp, synthetic construct
  source 1..1341: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggattggcaccatctcaagcggagggcggacttactatgcctcctgggccaagggacgcttcaccatctcgaaggactcctcgaagaacaccgtgtacctccaaatgaactcgctgagggcagaggacactgctgtgtactactgtggacggggagatggagccaccggcttcaatatctggggccagggaaccctcgtgactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 31 - protein, 444 aa, synthetic construct
EVQLVESGGGLVQPGGSLRLSCAVSGIDLSSNAMSWVRQAPGKGLEWIGTISSGGRTYYASWAKGRFTISKDSSKNTVYLQMNSLRAEDTAVYYCGRGDGATGFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - DNA, 1332 bp, synthetic construct
  source 1..1332: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggattggcaccatctcaagcggagggcggacttactatgcctcctgggccaagggacgcttcaccatctcgaaggactcctcgaagaacaccgtgtacctccaaatgaactcgctgagggcagaggacactgctgtgtactactgtggacggggagatggagccaccggcttcaatatctggggccagggaaccctcgtgactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 33 - protein, 117 aa, synthetic construct
EVQLVESGGGLVQPGGSLRLSCAASGIDLSSNAMSWVRQAPGKGLEWIGTISSGGRTYYASWAKGRFTISKDSSKNTVYLQMNSLRAEDTAVYYCGRGDGATGFNIWGQGTLVTVSS

SEQ ID NO: 34 - DNA, 351 bp, synthetic construct
  source 1..351: mol_type = other DNA
gaagtgcagctggtcgaatccgggggtggtctggtgcagccgggaggttccctgcgcttgtcatgcgcggcttccggcattgaccttagctccaacgccatgagctgggtcagacaggcccctggcaaagggctggagtggattggcaccatctcaagcggagggcggacttactatgcctcctgggccaagggacgcttcaccatctcgaaggactcctcgaagaacaccgtgtacctccaaatgaactcgctgagggcagaggacactgctgtgtactactgtggacggggagatggagccaccggcttcaatatctggggccagggaaccctcgtgactgtctcgagc

SEQ ID NO: 35 - protein, 447 aa, synthetic construct
EVQLVESGGGLVQPGGSLRLSCAASGIDLSSNAMSWVRQAPGKGLEWIGTISSGGRTYYASWAKGRFTISKDSSKNTVYLQMNSLRAEDTAVYYCGRGDGATGFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - DNA, 1341 bp, synthetic construct
  source 1..1341: mol_type = other DNA
gaagtgcagctggtcgaatccgggggtggtctggtgcagccgggaggttccctgcgcttgtcatgcgcggcttccggcattgaccttagctccaacgccatgagctgggtcagacaggcccctggcaaagggctggagtggattggcaccatctcaagcggagggcggacttactatgcctcctgggccaagggacgcttcaccatctcgaaggactcctcgaagaacaccgtgtacctccaaatgaactcgctgagggcagaggacactgctgtgtactactgtggacggggagatggagccaccggcttcaatatctggggccagggaaccctcgtgactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 37 - protein, 444 aa, synthetic construct
EVQLVESGGGLVQPGGSLRLSCAASGIDLSSNAMSWVRQAPGKGLEWIGTISSGGRTYYASWAKGRFTISKDSSKNTVYLQMNSLRAEDTAVYYCGRGDGATGFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - DNA, 1332 bp, synthetic construct
  source 1..1332: mol_type = other DNA
gaagtgcagctggtcgaatccgggggtggtctggtgcagccgggaggttccctgcgcttgtcatgcgcggcttccggcattgaccttagctccaacgccatgagctgggtcagacaggcccctggcaaagggctggagtggattggcaccatctcaagcggagggcggacttactatgcctcctgggccaagggacgcttcaccatctcgaaggactcctcgaagaacaccgtgtacctccaaatgaactcgctgagggcagaggacactgctgtgtactactgtggacggggagatggagccaccggcttcaatatctggggccagggaaccctcgtgactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 39 - protein, 117 aa, synthetic construct
EVQLVESGGGLVQPGGSLRLSCAVSGIDLSSNAMSWVRQAPGKGLEWVGTISSGGRTYYASWAKGRFTISKDSSKNTVYLQMNSLRAEDTAVYYCGRGDGATGFNIWGQGTLVTVSS

SEQ ID NO: 40 - DNA, 351 bp, synthetic construct
  source 1..351: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtgggtcggcaccatctcaagcggagggcggacttactatgcctcctgggccaagggacgcttcaccatctcgaaggactcctcgaagaacaccgtgtacctccaaatgaactcgctgagggcagaggacactgctgtgtactactgtggacggggagatggagccaccggcttcaatatctggggccagggaaccctcgtgactgtctcgagc

SEQ ID NO: 41 - protein, 447 aa, synthetic construct
EVQLVESGGGLVQPGGSLRLSCAVSGIDLSSNAMSWVRQAPGKGLEWVGTISSGGRTYYASWAKGRFTISKDSSKNTVYLQMNSLRAEDTAVYYCGRGDGATGFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - DNA, 1341 bp, synthetic construct
  source 1..1341: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtgggtcggcaccatctcaagcggagggcggacttactatgcctcctgggccaagggacgcttcaccatctcgaaggactcctcgaagaacaccgtgtacctccaaatgaactcgctgagggcagaggacactgctgtgtactactgtggacggggagatggagccaccggcttcaatatctggggccagggaaccctcgtgactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 43 - protein, 444 aa, synthetic construct
EVQLVESGGGLVQPGGSLRLSCAVSGIDLSSNAMSWVRQAPGKGLEWVGTISSGGRTYYASWAKGRFTISKDSSKNTVYLQMNSLRAEDTAVYYCGRGDGATGFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - DNA, 1332 bp, synthetic construct
  source 1..1332: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtgggtcggcaccatctcaagcggagggcggacttactatgcctcctgggccaagggacgcttcaccatctcgaaggactcctcgaagaacaccgtgtacctccaaatgaactcgctgagggcagaggacactgctgtgtactactgtggacggggagatggagccaccggcttcaatatctggggccagggaaccctcgtgactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 45 - protein, 117 aa, synthetic construct
EVQLVESGGGLVQPGGSLRLSCAVSGIDLSSNAMSWVRQAPGKGLEWISTISSGGRTYYASWAKGRFTISKDSSKNTVYLQMNSLRAEDTAVYYCGRGDGATGFNIWGQGTLVTVSS

SEQ ID NO: 46 - DNA, 351 bp, synthetic construct
  source 1..351: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggatttccaccatctcaagcggagggcggacttactatgcctcctgggccaagggacgcttcaccatctcgaaggactcctcgaagaacaccgtgtacctccaaatgaactcgctgagggcagaggacactgctgtgtactactgtggacggggagatggagccaccggcttcaatatctggggccagggaaccctcgtgactgtctcgagc

SEQ ID NO: 47 - protein, 447 aa, synthetic construct
EVQLVESGGGLVQPGGSLRLSCAVSGIDLSSNAMSWVRQAPGKGLEWISTISSGGRTYYASWAKGRFTISKDSSKNTVYLQMNSLRAEDTAVYYCGRGDGATGFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - DNA, 1341 bp, synthetic construct
  source 1..1341: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggatttccaccatctcaagcggagggcggacttactatgcctcctgggccaagggacgcttcaccatctcgaaggactcctcgaagaacaccgtgtacctccaaatgaactcgctgagggcagaggacactgctgtgtactactgtggacggggagatggagccaccggcttcaatatctggggccagggaaccctcgtgactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 49 - protein, 444 aa, synthetic construct
EVQLVESGGGLVQPGGSLRLSCAVSGIDLSSNAMSWVRQAPGKGLEWISTISSGGRTYYASWAKGRFTISKDSSKNTVYLQMNSLRAEDTAVYYCGRGDGATGFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - DNA, 1332 bp, synthetic construct
  source 1..1332: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggatttccaccatctcaagcggagggcggacttactatgcctcctgggccaagggacgcttcaccatctcgaaggactcctcgaagaacaccgtgtacctccaaatgaactcgctgagggcagaggacactgctgtgtactactgtggacggggagatggagccaccggcttcaatatctggggccagggaaccctcgtgactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 51 - protein, 117 aa, synthetic construct
EVQLVESGGGLVQPGGSLRLSCAVSGIDLSSNAMSWVRQAPGKGLEWIGTISSGGRTYYASWAKGRFTISRDSSKNTVYLQMNSLRAEDTAVYYCGRGDGATGFNIWGQGTLVTVSS

SEQ ID NO: 52 - DNA, 351 bp, synthetic construct
  source 1..351: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggattggcaccatctcaagcggagggcggacttactatgcctcctgggccaagggacgcttcaccatctcgcgggactcctcgaagaacaccgtgtacctccaaatgaactcgctgagggcagaggacactgctgtgtactactgtggacggggagatggagccaccggcttcaatatctggggccagggaaccctcgtgactgtctcgagc

SEQ ID NO: 53 - protein, 447 aa, synthetic construct
EVQLVESGGGLVQPGGSLRLSCAVSGIDLSSNAMSWVRQAPGKGLEWIGTISSGGRTYYASWAKGRFTISRDSSKNTVYLQMNSLRAEDTAVYYCGRGDGATGFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - DNA, 1341 bp, synthetic construct
  source 1..1341: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggattggcaccatctcaagcggagggcggacttactatgcctcctgggccaagggacgcttcaccatctcgcgggactcctcgaagaacaccgtgtacctccaaatgaactcgctgagggcagaggacactgctgtgtactactgtggacggggagatggagccaccggcttcaatatctggggccagggaaccctcgtgactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 55 - protein, 444 aa, synthetic construct
EVQLVESGGGLVQPGGSLRLSCAVSGIDLSSNAMSWVRQAPGKGLEWIGTISSGGRTYYASWAKGRFTISRDSSKNTVYLQMNSLRAEDTAVYYCGRGDGATGFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - DNA, 1332 bp, synthetic construct
  source 1..1332: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggattggcaccatctcaagcggagggcggacttactatgcctcctgggccaagggacgcttcaccatctcgcgggactcctcgaagaacaccgtgtacctccaaatgaactcgctgagggcagaggacactgctgtgtactactgtggacggggagatggagccaccggcttcaatatctggggccagggaaccctcgtgactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 57 - protein, 117 aa, synthetic construct
EVQLVESGGGLVQPGGSLRLSCAVSGIDLSSNAMSWVRQAPGKGLEWIGTISSGGRTYYASWAKGRFTISKDNSKNTVYLQMNSLRAEDTAVYYCGRGDGATGFNIWGQGTLVTVSS

SEQ ID NO: 58 - DNA, 351 bp, synthetic construct
  source 1..351: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggattggcaccatctcaagcggagggcggacttactatgcctcctgggccaagggacgcttcaccatctcgaaggacaactcgaagaacaccgtgtacctccaaatgaactcgctgagggcagaggacactgctgtgtactactgtggacggggagatggagccaccggcttcaatatctggggccagggaaccctcgtgactgtctcgagc

SEQ ID NO: 59 - protein, 447 aa, synthetic construct
EVQLVESGGGLVQPGGSLRLSCAVSGIDLSSNAMSWVRQAPGKGLEWIGTISSGGRTYYASWAKGRFTISKDNSKNTVYLQMNSLRAEDTAVYYCGRGDGATGFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - DNA, 1341 bp, synthetic construct
  source 1..1341: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggattggcaccatctcaagcggagggcggacttactatgcctcctgggccaagggacgcttcaccatctcgaaggacaactcgaagaacaccgtgtacctccaaatgaactcgctgagggcagaggacactgctgtgtactactgtggacggggagatggagccaccggcttcaatatctggggccagggaaccctcgtgactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 61 - protein, 444 aa, synthetic construct
EVQLVESGGGLVQPGGSLRLSCAVSGIDLSSNAMSWVRQAPGKGLEWIGTISSGGRTYYASWAKGRFTISKDNSKNTVYLQMNSLRAEDTAVYYCGRGDGATGFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - DNA, 1332 bp, synthetic construct
  source 1..1332: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggattggcaccatctcaagcggagggcggacttactatgcctcctgggccaagggacgcttcaccatctcgaaggacaactcgaagaacaccgtgtacctccaaatgaactcgctgagggcagaggacactgctgtgtactactgtggacggggagatggagccaccggcttcaatatctggggccagggaaccctcgtgactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 63 - protein, 117 aa, synthetic construct
EVQLVESGGGLVQPGGSLRLSCAVSGIDLSSNAMSWVRQAPGKGLEWIGTISSGGRTYYASWAKGRFTISKDSSKNTLYLQMNSLRAEDTAVYYCGRGDGATGFNIWGQGTLVTVSS

SEQ ID NO: 64 - DNA, 351 bp, synthetic construct
  source 1..351: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggattggcaccatctcaagcggagggcggacttactatgcctcctgggccaagggacgcttcaccatctcgaaggactcctcgaagaacaccctgtacctccaaatgaactcgctgagggcagaggacactgctgtgtactactgtggacggggagatggagccaccggcttcaatatctggggccagggaaccctcgtgactgtctcgagc

SEQ ID NO: 65 - protein, 447 aa, synthetic construct
EVQLVESGGGLVQPGGSLRLSCAVSGIDLSSNAMSWVRQAPGKGLEWIGTISSGGRTYYASWAKGRFTISKDSSKNTLYLQMNSLRAEDTAVYYCGRGDGATGFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - DNA, 1341 bp, synthetic construct
  source 1..1341: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggattggcaccatctcaagcggagggcggacttactatgcctcctgggccaagggacgcttcaccatctcgaaggactcctcgaagaacaccctgtacctccaaatgaactcgctgagggcagaggacactgctgtgtactactgtggacggggagatggagccaccggcttcaatatctggggccagggaaccctcgtgactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 67 - protein, 444 aa, synthetic construct
EVQLVESGGGLVQPGGSLRLSCAVSGIDLSSNAMSWVRQAPGKGLEWIGTISSGGRTYYASWAKGRFTISKDSSKNTLYLQMNSLRAEDTAVYYCGRGDGATGFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 68 - DNA, 1332 bp, synthetic construct
  source 1..1332: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggattggcaccatctcaagcggagggcggacttactatgcctcctgggccaagggacgcttcaccatctcgaaggactcctcgaagaacaccctgtacctccaaatgaactcgctgagggcagaggacactgctgtgtactactgtggacggggagatggagccaccggcttcaatatctggggccagggaaccctcgtgactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 69 - protein, 117 aa, synthetic construct
EVQLVESGGGLVQPGGSLRLSCAVSGIDLSSNAMSWVRQAPGKGLEWIGTISSGGRTYYASWAKGRFTISKDSSKNTVYLQMNSLRAEDTAVYYCARGDGATGFNIWGQGTLVTVSS

SEQ ID NO: 70 - DNA, 351 bp, synthetic construct
  source 1..351: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggattggcaccatctcaagcggagggcggacttactatgcctcctgggccaagggacgcttcaccatctcgaaggactcctcgaagaacaccgtgtacctccaaatgaactcgctgagggcagaggacactgctgtgtactactgtgcgcggggagatggagccaccggcttcaatatctggggccagggaaccctcgtgactgtctcgagc

SEQ ID NO: 71 - protein, 447 aa, synthetic construct
EVQLVESGGGLVQPGGSLRLSCAVSGIDLSSNAMSWVRQAPGKGLEWIGTISSGGRTYYASWAKGRFTISKDSSKNTVYLQMNSLRAEDTAVYYCARGDGATGFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - DNA, 1341 bp, synthetic construct
  source 1..1341: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggattggcaccatctcaagcggagggcggacttactatgcctcctgggccaagggacgcttcaccatctcgaaggactcctcgaagaacaccgtgtacctccaaatgaactcgctgagggcagaggacactgctgtgtactactgtgcgcggggagatggagccaccggcttcaatatctggggccagggaaccctcgtgactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 73 - protein, 444 aa, synthetic construct
EVQLVESGGGLVQPGGSLRLSCAVSGIDLSSNAMSWVRQAPGKGLEWIGTISSGGRTYYASWAKGRFTISKDSSKNTVYLQMNSLRAEDTAVYYCARGDGATGFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - DNA, 1332 bp, synthetic construct
  source 1..1332: mol_type = other DNA
gaagtgcagctggtcgaatccgggggtggtctggtgcagccgggaggttccctgcgcttgtcatgcgcggtgtccggcattgaccttagctccaacgccatgagctgggtcagacaggcccctggcaaagggctggagtggattggcaccatctcaagcggagggcggacttactatgcctcctgggccaagggacgcttcaccatctcgaaggactcctcgaagaacaccgtgtacctccaaatgaactcgctgagggcagaggacactgctgtgtactactgtgcgcggggagatggagccaccggcttcaatatctggggccagggaaccctcgtgactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 75 - protein, 117 aa, synthetic construct
EVQLVESGGGLVQPGGSLRLSCAASGIDLSSNAMSWVRQAPGKGLEWIGTISSGGRTYYASWAKGRFTISKDNSKNTVYLQMNSLRAEDTAVYYCGRGDGATGFNIWGQGTLVTVSS

SEQ ID NO: 76 - DNA, 351 bp, synthetic construct
  source 1..351: mol_type = other DNA
gaagtgcagctggtcgaatccgggggtggtctggtgcagccgggaggttccctgcgcttgtcatgcgcggcttccggcattgaccttagctccaacgccatgagctgggtcagacaggcccctggcaaagggctggagtggattggcaccatctcaagcggagggcggacttactatgcctcctgggccaagggacgcttcaccatctcgaaggacaactcgaagaacaccgtgtacctccaaatgaactcgctgagggcagaggacactgctgtgtactactgtggacggggagatggagccaccggcttcaatatctggggccagggaaccctcgtgactgtctcgagc

SEQ ID NO: 77 - protein, 447 aa, synthetic construct
EVQLVESGGGLVQPGGSLRLSCAASGIDLSSNAMSWVRQAPGKGLEWIGTISSGGRTYYASWAKGRFTISKDNSKNTVYLQMNSLRAEDTAVYYCGRGDGATGFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - DNA, 1341 bp, synthetic construct
  source 1..1341: mol_type = other DNA
gaagtgcagctggtcgaatccgggggtggtctggtgcagccgggaggttccctgcgcttgtcatgcgcggcttccggcattgaccttagctccaacgccatgagctgggtcagacaggcccctggcaaagggctggagtggattggcaccatctcaagcggagggcggacttactatgcctcctgggccaagggacgcttcaccatctcgaaggacaactcgaagaacaccgtgtacctccaaatgaactcgctgagggcagaggacactgctgtgtactactgtggacggggagatggagccaccggcttcaatatctggggccagggaaccctcgtgactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 79 - protein, 444 aa, synthetic construct
EVQLVESGGGLVQPGGSLRLSCAASGIDLSSNAMSWVRQAPGKGLEWIGTISSGGRTYYASWAKGRFTISKDNSKNTVYLQMNSLRAEDTAVYYCGRGDGATGFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - DNA, 1332 bp, synthetic construct
  source 1..1332: mol_type = other DNA
gaagtgcagctggtcgaatccgggggtggtctggtgcagccgggaggttccctgcgcttgtcatgcgcggcttccggcattgaccttagctccaacgccatgagctgggtcagacaggcccctggcaaagggctggagtggattggcaccatctcaagcggagggcggacttactatgcctcctgggccaagggacgcttcaccatctcgaaggacaactcgaagaacaccgtgtacctccaaatgaactcgctgagggcagaggacactgctgtgtactactgtggacggggagatggagccaccggcttcaatatctggggccagggaaccctcgtgactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 81 - protein, 117 aa, synthetic construct
EVQLVESGGGLVQPGGSLRLSCAASGIDLSSNAMSWVRQAPGKGLEWIGTISSGGRTYYASWAKGRFTISKDSSKNTVYLQMNSLRAEDTAVYYCARGDGATGFNIWGQGTLVTVSS

SEQ ID NO: 82 - DNA, 351 bp, synthetic construct
  source 1..351: mol_type = other DNA
gaagtgcagctggtcgaatccgggggtggtctggtgcagccgggaggttccctgcgcttgtcatgcgcggcttccggcattgaccttagctccaacgccatgagctgggtcagacaggcccctggcaaagggctggagtggattggcaccatctcaagcggagggcggacttactatgcctcctgggccaagggacgcttcaccatctcgaaggactcctcgaagaacaccgtgtacctccaaatgaactcgctgagggcagaggacactgctgtgtactactgtgcgcggggagatggagccaccggcttcaatatctggggccagggaaccctcgtgactgtctcgagc

SEQ ID NO: 83 - protein, 447 aa, synthetic construct
EVQLVESGGGLVQPGGSLRLSCAASGIDLSSNAMSWVRQAPGKGLEWIGTISSGGRTYYASWAKGRFTISKDSSKNTVYLQMNSLRAEDTAVYYCARGDGATGFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - DNA, 1341 bp, synthetic construct
  source 1..1341: mol_type = other DNA
gaagtgcagctggtcgaatccgggggtggtctggtgcagccgggaggttccctgcgcttgtcatgcgcggcttccggcattgaccttagctccaacgccatgagctgggtcagacaggcccctggcaaagggctggagtggattggcaccatctcaagcggagggcggacttactatgcctcctgggccaagggacgcttcaccatctcgaaggactcctcgaagaacaccgtgtacctccaaatgaactcgctgagggcagaggacactgctgtgtactactgtgcgcggggagatggagccaccggcttcaatatctggggccagggaaccctcgtgactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 85 - protein, 444 aa, synthetic construct
EVQLVESGGGLVQPGGSLRLSCAASGIDLSSNAMSWVRQAPGKGLEWIGTISSGGRTYYASWAKGRFTISKDSSKNTVYLQMNSLRAEDTAVYYCARGDGATGFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - DNA, 1332 bp, synthetic construct
  source 1..1332: mol_type = other DNA
gaagtgcagctggtcgaatccgggggtggtctggtgcagccgggaggttccctgcgcttgtcatgcgcggcttccggcattgaccttagctccaacgccatgagctgggtcagacaggcccctggcaaagggctggagtggattggcaccatctcaagcggagggcggacttactatgcctcctgggccaagggacgcttcaccatctcgaaggactcctcgaagaacaccgtgtacctccaaatgaactcgctgagggcagaggacactgctgtgtactactgtgcgcggggagatggagccaccggcttcaatatctggggccagggaaccctcgtgactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 87 - protein, 117 aa, synthetic construct
EVQLVESGGGLVQPGGSLRLSCAVSGIDLSSNAMSWVRQAPGKGLEWIGTISSGGRTYYASWAKGRFTISKDNSKNTVYLQMNSLRAEDTAVYYCARGDGATGFNIWGQGTLVTVSS

SEQ ID NO: 88 - DNA, 351 bp, synthetic construct
  source 1..351: mol_type = other DNA
gaagtgcagctggtcgagtccggtggaggcctggtgcagcccggcggctccctgcggctgtcctgcgccgtctccgggatcgacctgtcctccaacgctatgagctgggtcagacaggcacccgggaagggtctggagtggatcgggacaatctccagcggcggccgcacctactacgcatcctgggccaagggaaggttcaccatttcgaaggacaactccaagaacaccgtgtaccttcagatgaactccctgcgggccgaggatactgccgtgtattactgtgcgaggggagatggcgcgactgggttcaacatctggggacagggaacgctggtgactgtgtcctcg

SEQ ID NO: 89 - protein, 447 aa, synthetic construct
EVQLVESGGGLVQPGGSLRLSCAVSGIDLSSNAMSWVRQAPGKGLEWIGTISSGGRTYYASWAKGRFTISKDNSKNTVYLQMNSLRAEDTAVYYCARGDGATGFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - DNA, 1341 bp, synthetic construct
  source 1..1341: mol_type = other DNA
gaagtgcagctggtcgagtccggtggaggcctggtgcagcccggcggctccctgcggctgtcctgcgccgtctccgggatcgacctgtcctccaacgctatgagctgggtcagacaggcacccgggaagggtctggagtggatcgggacaatctccagcggcggccgcacctactacgcatcctgggccaagggaaggttcaccatttcgaaggacaactccaagaacaccgtgtaccttcagatgaactccctgcgggccgaggatactgccgtgtattactgtgcgaggggagatggcgcgactgggttcaacatctggggacagggaacgctggtgactgtgtcctcggcttcgaccaaggggccctcggtgttccctctggcgcctagctccaagagcacttcaggcggaacggctgccctcggatgcctggtcaaggactacttcccagagcccgtgaccgtgtcatggaacagcggagctctgactagcggagtgcacacctttccggcggtgctgcaaagctcaggcctgtactcgctctcctcagtggtcactgtcccgtcctcctcgctggggactcaaacgtacatctgcaacgtcaatcacaaaccgtcaaataccaaagtcgacaagaaggtcgagcctaagtcgtgcgataagacccacacctgtccgccatgcccagcccctgaactgctgggaggaccaagcgtgttcctcttcccgccaaaaccaaaagacaccttgatgatttcacggactcctgaggtcacgtgcgtcgtggtggacgtgagccatgaggacccagaagtgaaattcaattggtacgtggacggagtcgaagtgcataacgccaagaccaagcccagagaggaacagtataacagcacctaccgggtcgtgagcgtgctcactgtgttgcaccaggactggctcaatggaaaggagtacaagtgcaaagtgtccaataaggccctccctgctcccatcgaaaagaccatcagcaaggcaaagggccagccacgcgaaccgcaagtgtacacccttccgccgtcgcgcgatgaactgactaaaaaccaagtctcgctgacctgtctcgtgaagggcttttacccgtccgacattgcggtggagtgggagtcgaacggtcagccggaaaacaattacaagaccactccgccggtgctcgactcggatgggtccttcttcctctactcgaagttgaccgtcgataagtccagatggcagcagggtaacgtgttctcgtgcagcgtgatgcacgaggccctgcacaaccattacactcagaagtcgctttccttgtcaccgggcaaa

SEQ ID NO: 91 - protein, 444 aa, synthetic construct
EVQLVESGGGLVQPGGSLRLSCAVSGIDLSSNAMSWVRQAPGKGLEWIGTISSGGRTYYASWAKGRFTISKDNSKNTVYLQMNSLRAEDTAVYYCARGDGATGFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 92 - DNA, 1332 bp, synthetic construct
  source 1..1332: mol_type = other DNA
gaagtgcagctggtcgagtccggtggaggcctggtgcagcccggcggctccctgcggctgtcctgcgccgtctccgggatcgacctgtcctccaacgctatgagctgggtcagacaggcacccgggaagggtctggagtggatcgggacaatctccagcggcggccgcacctactacgcatcctgggccaagggaaggttcaccatttcgaaggacaactccaagaacaccgtgtaccttcagatgaactccctgcgggccgaggatactgccgtgtattactgtgcgaggggagatggcgcgactgggttcaacatctggggacagggaacgctggtgactgtgtcctcggcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 93 - protein, 117 aa, synthetic construct
EVQLVESGGGLVQPGGSLRLSCAASGIDLSSNAMSWVRQAPGKGLEWIGTISSGGRTYYASWAKGRFTISKDNSKNTVYLQMNSLRAEDTAVYYCARGDGATGFNIWGQGTLVTVSS

SEQ ID NO: 94 - DNA, 351 bp, synthetic construct
  source 1..351: mol_type = other DNA
gaagtgcagctggtcgagtccggtggaggcctggtgcagcccggcggctccctgcggctgtcctgcgccgcctccgggatcgacctgtcctccaacgctatgagctgggtcagacaggcacccgggaagggtctggagtggatcgggacaatctccagcggcggccgcacctactacgcatcctgggccaagggaaggttcaccatttcgaaggacaactccaagaacaccgtgtaccttcagatgaactccctgcgggccgaggatactgccgtgtattactgtgcgaggggagatggcgcgactgggttcaacatctggggacagggaacgctggtgactgtgtcctcg

SEQ ID NO: 95 - protein, 447 aa, synthetic construct
EVQLVESGGGLVQPGGSLRLSCAASGIDLSSNAMSWVRQAPGKGLEWIGTISSGGRTYYASWAKGRFTISKDNSKNTVYLQMNSLRAEDTAVYYCARGDGATGFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - DNA, 1341 bp, synthetic construct
  source 1..1341: mol_type = other DNA
gaagtgcagctggtcgagtccggtggaggcctggtgcagcccggcggctccctgcggctgtcctgcgccgcctccgggatcgacctgtcctccaacgctatgagctgggtcagacaggcacccgggaagggtctggagtggatcgggacaatctccagcggcggccgcacctactacgcatcctgggccaagggaaggttcaccatttcgaaggacaactccaagaacaccgtgtaccttcagatgaactccctgcgggccgaggatactgccgtgtattactgtgcgaggggagatggcgcgactgggttcaacatctggggacagggaacgctggtgactgtgtcctcggcttcgaccaaggggccctcggtgttccctctggcgcctagctccaagagcacttcaggcggaacggctgccctcggatgcctggtcaaggactacttcccagagcccgtgaccgtgtcatggaacagcggagctctgactagcggagtgcacacctttccggcggtgctgcaaagctcaggcctgtactcgctctcctcagtggtcactgtcccgtcctcctcgctggggactcaaacgtacatctgcaacgtcaatcacaaaccgtcaaataccaaagtcgacaagaaggtcgagcctaagtcgtgcgataagacccacacctgtccgccatgcccagcccctgaactgctgggaggaccaagcgtgttcctcttcccgccaaaaccaaaagacaccttgatgatttcacggactcctgaggtcacgtgcgtcgtggtggacgtgagccatgaggacccagaagtgaaattcaattggtacgtggacggagtcgaagtgcataacgccaagaccaagcccagagaggaacagtataacagcacctaccgggtcgtgagcgtgctcactgtgttgcaccaggactggctcaatggaaaggagtacaagtgcaaagtgtccaataaggccctccctgctcccatcgaaaagaccatcagcaaggcaaagggccagccacgcgaaccgcaagtgtacacccttccgccgtcgcgcgatgaactgactaaaaaccaagtctcgctgacctgtctcgtgaagggcttttacccgtccgacattgcggtggagtgggagtcgaacggtcagccggaaaacaattacaagaccactccgccggtgctcgactcggatgggtccttcttcctctactcgaagttgaccgtcgataagtccagatggcagcagggtaacgtgttctcgtgcagcgtgatgcacgaggccctgcacaaccattacactcagaagtcgctttccttgtcaccgggcaaa

SEQ ID NO: 97 - protein, 447 aa, synthetic construct
EVQLVESGGGLVQPGGSLRLSCAASGIDLSSNAMSWVRQAPGKGLEWIGTISSGGRTYYASWAKGRFTISKDNSKNTVYLQMNSLRAEDTAVYYCARGDGATGFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - DNA, 1341 bp, synthetic construct
  source 1..1341: mol_type = other DNA
gaagtgcagctggtcgaatccgggggtggtctggtgcagccgggaggttccctgcgcttgtcatgcgcggcttccggcattgaccttagctccaacgccatgagctgggtcagacaggcccctggcaaagggctggagtggattggcaccatctcaagcggagggcggacttactatgcctcctgggccaagggacgcttcaccatctcgaaggacaactcgaagaacaccgtgtacctccaaatgaactcgctgagggcagaggacactgctgtgtactactgtgcgcggggagatggagccaccggcttcaatatctggggccagggaaccctcgtgactgtctcgagcgcttctacaaagggcccatcggtcttccccctggcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgataagaaagttgagcccaaatcttgtgacaaaactcacacatgcccaccgtgcccagcacctgaagctgctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtatccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 99 - protein, 444 aa, synthetic construct
EVQLVESGGGLVQPGGSLRLSCAASGIDLSSNAMSWVRQAPGKGLEWIGTISSGGRTYYASWAKGRFTISKDNSKNTVYLQMNSLRAEDTAVYYCARGDGATGFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - DNA, 1332 bp, synthetic construct
  source 1..1332: mol_type = other DNA
gaagtgcagctggtcgaatccgggggtggtctggtgcagccgggaggttccctgcgcttgtcatgcgcggcttccggcattgaccttagctccaacgccatgagctgggtcagacaggcccctggcaaagggctggagtggattggcaccatctcaagcggagggcggacttactatgcctcctgggccaagggacgcttcaccatctcgaaggacaactcgaagaacaccgtgtacctccaaatgaactcgctgagggcagaggacactgctgtgtactactgtgcgcggggagatggagccaccggcttcaatatctggggccagggaaccctcgtgactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 101 - protein, 444 aa, synthetic construct
EVQLVESGGGLVQPGGSLRLSCAASGIDLSSNAMSWVRQAPGKGLEWIGTISSGGRTYYASWAKGRFTISKDNSKNTVYLQMNSLRAEDTAVYYCARGDGATGFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - DNA, 1332 bp, synthetic construct
  source 1..1332: mol_type = other DNA
gaagtgcagctggtcgaatccgggggtggtctggtgcagccgggaggttccctgcgcttgtcatgcgcggcttccggcattgaccttagctccaacgccatgagctgggtcagacaggcccctggcaaagggctggagtggattggcaccatctcaagcggagggcggacttactatgcctcctgggccaagggacgcttcaccatctcgaaggacaactcgaagaacaccgtgtacctccaaatgaactcgctgagggcagaggacactgctgtgtactactgtgcgcggggagatggagccaccggcttcaatatctggggccagggaaccctcgtgactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgaagccgcggggggaccgtcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 103 - protein, 107 aa, Homo sapiens [sequence omitted]

SEQ ID NO: 104 - DNA, 321 bp, Homo sapiens
  source 1..321: mol_type = genomic DNA
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttaccctctcactttcggcggagggaccaaggtggagatcaaa

SEQ ID NO: 105 - protein, 112 aa, Homo sapiens
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCARYFDYWGQGTLVTVSS

SEQ ID NO: 106 - DNA, 336 bp, Homo sapiens
  source 1..336: mol_type = genomic DNA
gaggtgcagctggtggagtctgggggaggcttggtccagcctggggggtccctgagactctcctgtgcagcctctggattcaccgtcagtagcaactacatgagctgggtccgccaggctccagggaaggggctggagtgggtctcagttatttatagcggtggtagcacatactacgcagactccgtgaagggcagattcaccatctccagagacaattccaagaacacgctgtatcttcaaatgaacagcctgagagccgaggacacggctgtgtattactgtgcgagatactttgactactggggccaaggaaccctggtcaccgtctcctca

SEQ ID NO: 107 - protein, 314 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 108 - protein, 276 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQ

SEQ ID NO: 109 - protein, 337 aa, synthetic construct
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQGLNDIFEAQKIEWHELEENLYFQGSGGSHHHHHHHHH

SEQ ID NO: 110 - protein, 313 aa, synthetic construct
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQGLNDIFEAQKIEWHELEENLYFQGSGGSHHHHHHHHH

SEQ ID NO: 111 - DNA, 1014 bp, synthetic construct
  source 1..1014: mol_type = other DNA
atggtcgtcatggcgcccaggactctgtttctgcttctgtccggcgccttgaccttgaccgagacttgggccggaagccactcgatgcggtacttctccgcggccgtgtctagaccgggtcggggagaaccccggttcatcgccatgggctacgtggatgacacccagttcgtgcggttcgacagcgattcagcctgccctcgcatggagccgagagccccttgggtggaacaggaagggccggagtactgggaagaggaaacacgcaacaccaaggcccacgctcaaaccgaccggatgaacttgcagacgctgcggggatactataaccagtccgaggcgtcgagccatacccttcaatggatgattggctgtgacctgggttccgacgggagactgctgcggggctacgagcagtacgcctatgacggaaaggactacctggccctcaacgaagatctccgctcctggaccgctgccgatactgcggcccagatctccaagcgcaaatgcgaagcggctaatgtcgccgaacagcgaagggcctacctggaaggcacttgcgtggagtggctgcaccgctacctggagaacggaaaggaaatgctgcagagggcagacccccctaagacccatgtcacccaccatcccgtgttcgactacgaagccaccctgagatgctgggcgctgggcttttaccctgccgagatcatcctgacctggcaacgcgacggggaagatcagacccaagacgtggaacttgtggagactagaccagccggcgatgggactttccagaaatgggcagccgtggtcgtgccgtcgggagaggaacaacgctacacctgtcacgtgcagcacgagggtctgccagagcccctgatgctgcggtggaagcagagctccctccccaccattccgatcatgggaattgtggcgggactcgtggtgctcgccgctgtcgtgactggagccgcagtggcagctgtgctctggcggaagaagtcctcagac

SEQ ID NO: 112 - protein, 338 aa, synthetic construct
MVVMAPRTLFLLLSGALTLTETWAGSHSMRYFSTAVSRPGRGEPRFIAVGYVDDTQFVRFDSDAASPRMEPRAPWVEQEGPEYWERETRNAKANAQTDRVNLRTLRGYYNQSEAGSHTLQWMYGCDLGSDGRLLRGYEQYAYDGKDYLALNEDLRSWTAADTAAQISKRKCEAAREAEQLRAYLEGTCVEWLHRYLENGKETLQRADPPKTHVTHHPVSDHEATLRCWALGFYPAEITLTWQRDGEDQTQDTELVETRPAGDGTFQKWAAVVVPSGEEQRYTCHVQHEGLPEPLTLRWKQGLNDIFEAQKIEWHELEENLYFQGSGGSHHHHHHHHHH

SEQ ID NO: 113 - protein, 314 aa, synthetic construct
GSHSMRYFSTAVSRPGRGEPRFIAVGYVDDTQFVRFDSDAASPRMEPRAPWVEQEGPEYWERETRNAKANAQTDRVNLRTLRGYYNQSEAGSHTLQWMYGCDLGSDGRLLRGYEQYAYDGKDYLALNEDLRSWTAADTAAQISKRKCEAAREAEQLRAYLEGTCVEWLHRYLENGKETLQRADPPKTHVTHHPVSDHEATLRCWALGFYPAEITLTWQRDGEDQTQDTELVETRPAGDGTFQKWAAVVVPSGEEQRYTCHVQHEGLPEPLTLRWKQGLNDIFEAQKIEWHELEENLYFQGSGGSHHHHHHHHHH

SEQ ID NO: 114 - DNA, 1014 bp, synthetic construct
  source 1..1014: mol_type = other DNA
atggtcgtcatggcgcccaggactctgtttctgcttctgtccggcgccttgaccttgaccgagacttgggccggaagccactcgatgcggtacttctccaccgccgtgtctagaccgggtcggggagaaccccggttcatcgccgtgggctacgtggatgacacccagttcgtgcggttcgacagcgatgccgcctcacctcgcatggagccgagagccccttgggtggaacaggaagggccggagtactgggaacgcgaaacacgcaacgccaaggccaacgctcaaaccgaccgggtcaacttgagaacgctgcggggatactataaccagtccgaggcgggaagccatacccttcaatggatgtacggctgtgacctgggttccgacgggagactgctgcggggctacgagcagtacgcctatgacggaaaggactacctggccctcaacgaagatctccgctcctggaccgctgccgatactgcggcccagatctccaagcgcaaatgcgaagcggctagagaagccgaacagctgagggcctacctggaaggcacttgcgtggagtggctgcaccgctacctggagaacggaaaggaaacgctgcagagggcagacccccctaagacccatgtcactcaccacccggtgtccgatcacgaggccaccctgaggtgctgggcactgggattctacccggcggagatcaccctgacctggcaacgggacggcgaagatcagacccaagacaccgagctcgtggaaaccaggcctgcgggtgatggaaccttccagaagtgggctgccgtggtggtgccatccggggaggagcaacggtacacttgtcacgtgcagcacgagggactgcctgaacccctgactctgcggtggaagcagagctccctccccaccattccgatcatgggaattgtggcgggactcgtggtgctcgccgctgtcgtgactggagccgcagtggcagctgtgctctggcggaagaagtcctcagac

SEQ ID NO: 115 - protein, 314 aa, synthetic construct
GSHSMRYFSTAVSRPGRGEPRFIAVGYVDDTQFVRFDSDAASPRMEPRAPWVEQEGPEYWERETRNAKANAQTDRVNLRTLRGYYNQSEAGSHTLQWMYGCDLGSDGRLLRGYEQYAYDGKDYLALNEDLRSWTAADTAAQISKRKCEAAREAEQLRAYLEGTCVEWLHRYLENGKETLQRADPPKTHVTHHPVSDHEATLRCWALGFYPAEITLTWQRDGEDQTQDTELVETRPAGDGTFQKWAAVVVPSGEEQRYTCHVQHEGLPEPLTLRWKQSSLPTIPIMGIVAGLVVLAAVVTGAAVAAVLWRKKSSD

SEQ ID NO: 116 - DNA, 1011 bp, synthetic construct
  source 1..1011: mol_type = other DNA
atggtcgtcatggcgcccaggactctgtttctgcttctgtccggcgccttgaccttgaccgagacttgggccggaagccactcgatgcggtacttctccaccgccgtgtctagaccgggtcggggagaaccccggttcatcgccgtgggctacgtggatgacacccagttcgtgcggttcgacagcgatgccgcctcacctcgcatggagccgagagccccttgggtggaacaggaagggccggagtactgggaacgcgaaacacgcaacgccaaggccaacgctcaaaccgaccgggtcaacttgagaacgctgcggggatactataaccagtccgaggcgtcgagccatacccttcaatggatgattggctgtgacctgggttccgacgggagactgctgcggggctacgagcagtacgcctatgacggaaaggactacctggccctcaacgaagatctccgctcctggaccgctgccgatactgcggcccagatctccaagcgcaaatgcgaagcggctaatgtcgccgaacagcgaagggcctacctggaaggcacttgcgtggagtggctgcaccgctacctggagaacggaaaggaaatgctgcagagggcagacccccctaagacccatgtcactcaccacccggtgtccgatcacgaggccaccctgaggtgctgggcactgggattctacccggcggagatcaccctgacctggcaacgggacggcgaagatcagacccaagacaccgagctcgtggaaaccaggcctgcgggtgatggaaccttccagaagtgggctgccgtggtggtgccatccggggaggagcaacggtacacttgtcacgtgcagcacgagggactgcctgaacccctgactctgcggtggaagcagagctccctccccaccattccgatcatgggaattgtggcgggactcgtggtgctcgccgctgtcgtgactggagccgcagtggcagctgtgctctggcggaagaagtcctca

SEQ ID NO: 117 - protein, 314 aa, synthetic construct
GSHSMRYFSTAVSRPGRGEPRFIAVGYVDDTQFVRFDSDAASPRMEPRAPWVEQEGPEYWERETRNAKANAQTDRVNLRTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVSDHEATLRCWALGFYPAEITLTWQRDGEDQTQDTELVETRPAGDGTFQKWAAVVVPSGEEQRYTCHVQHEGLPEPLTLRWKQSSLPTIPIMGIVAGLVVLAAVVTGAAVAAVLWRKKSSD

SEQ ID NO: 118 - DNA, 1014 bp, synthetic construct
  source 1..1014: mol_type = other DNA
atggtcgtcatggcgcccaggactctgtttctgcttctgtccggcgccttgaccttgaccgagacttgggccggaagccactcgatgcggtacttctccgcggccgtgtctagaccgggtcggggagaaccccggttcatcgccatgggctacgtggatgacacccagttcgtgcggttcgacagcgattcagcctgccctcgcatggagccgagagccccttgggtggaacaggaagggccggagtactgggaagaggaaacacgcaacaccaaggcccacgctcaaaccgaccggatgaacttgcagacgctgcggggatactataaccagtccgaggcgtcgagccatacccttcaatggatgattggctgtgacctgggttccgacgggagactgctgcggggctacgagcagtacgcctatgacggaaaggactacctggccctcaacgaagatctccgctcctggaccgctgccgatactgcggcccagatctccaagcgcaaatgcgaagcggctaatgtcgccgaacagcgaagggcctacctggaaggcacttgcgtggagtggctgcaccgctacctggagaacggaaaggaaatgctgcagagggcagacccccctaagacccatgtcactcaccacccggtgtccgatcacgaggccaccctgaggtgctgggcactgggattctacccggcggagatcaccctgacctggcaacgggacggcgaagatcagacccaagacaccgagctcgtggaaaccaggcctgcgggtgatggaaccttccagaagtgggctgccgtggtggtgccatccggggaggagcaacggtacacttgtcacgtgcagcacgagggactgcctgaacccctgactctgcggtggaagcagagctccctccccaccattccgatcatgggaattgtggcgggactcgtggtgctcgccgctgtcgtgactggagccgcagtggcagctgtgctctggcggaagaagtcctcagac

SEQ ID NO: 119 - protein, 314 aa, synthetic construct
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVSDHEATLRCWALGFYPAEITLTWQRDGEDQTQDTELVETRPAGDGTFQKWAAVVVPSGEEQRYTCHVQHEGLPEPLTLRWKQSSLPTIPIMGIVAGLVVLAAVVTGAAVAAVLWRKKSSD

SEQ ID NO: 120 - DNA, 1014 bp, synthetic construct
  source 1..1014: mol_type = other DNA
atggtcgtcatggcgcccaggactctgtttctgcttctgtccggcgccttgaccttgaccgagacttgggccggaagccactcgatgcggtacttctccgcggccgtgtctagaccgggtcggggagaaccccggttcatcgccatgggctacgtggatgacacccagttcgtgcggttcgacagcgattcagcctgccctcgcatggagccgagagccccttgggtggaacaggaagggccggagtactgggaagaggaaacacgcaacaccaaggcccacgctcaaaccgaccggatgaacttgcagacgctgcggggatactataaccagtccgaggcgtcgagccatacccttcaatggatgattggctgtgacctgggttccgacgggagactgctgcggggctacgagcagtacgcctatgacggaaaggactacctggccctcaacgaagatctccgctcctggaccgctgccgatactgcggcccagatctccaagcgcaaatgcgaagcggctaatgtcgccgaacagcgaagggcctacctggaaggcacttgcgtggagtggctgcaccgctacctggagaacggaaaggaaatgctgcagagggcagacccccctaagacccatgtcacccaccatcccgtgtcagaccacgaagccaccctgagatgctgggcgctgggcttttaccctgccgagatcatcctgacctggcaacgcgacggggaagatcagacccaagacgtggaacttgtggagactagaccagccggcgatgggactttccagaaatgggcagccgtggtcgtgccgtcgggagaggaacaacgctacacctgtcacgtgcagcacgagggtctgccagagcccctgatgctgcggtggaagcagagctccctccccaccattccgatcatgggaattgtggcgggactcgtggtgctcgccgctgtcgtgactggagccgcagtggcagctgtgctctggcggaagaagtcctcagac

SEQ ID NO: 121 - protein, 314 aa, synthetic construct
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVSDHEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 122 - protein, 368 aa, synthetic construct
MSVPTQVLGLLLLWLTDARCDPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPILEENLYFQGVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 110 aa, synthetic construct
DPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPILEENLYFQ

SEQ ID NO: 124 - DNA, 456 bp, synthetic construct
  source 1..456: mol_type = other DNA
atgtccgtgccgacccaagtgctgggactgctcctgctctggctgactgacgctcgctgtgacccccctaagacccacgtcactcatcaccctgtgtccgaccatgaagctaccctgagatgctgggccctgggtttctaccccgccgagattaccttgacctggcaaagggacggcgaagatcagacgcaagacaccgagctcgtggagactcggccagcgggggatggaacattccagaaatgggccgcagtggtcgtgccgtccggagaagaacagcggtacacttgccacgtgcagcacgaaggcctgccggagcctctgacccttcgctggaagcagtcgagcctccccaccatcccgatcatggggattgtggccggccttgtggtgctggccgcagtcgtgaccggagcagctgtggcggctgtcctgtggcggaagaagtcaagcgat

SEQ ID NO: 125 - protein, 132 aa, Homo sapiens
DPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 126 - protein, 165 aa, synthetic construct
MSVPTQVLGLLLLWLTDARCGGSHHHHHHHHHHGSGSENLYFQGLNDIFEAQKIEWHGGGSGSDPPKTHVTHHPVSDHEATLRCWALGFYPAEITLTWQRDGEDQTQDTELVETRPAGDGTFQKWAAVVVPSGEEQRYTCHVQHEGLPEPLTLRWKQSSLPTIPI

SEQ ID NO: 127 - protein, 122 aa, synthetic construct
GLNDIFEAQKIEWHGGGSGSDPPKTHVTHHPVSDHEATLRCWALGFYPAEITLTWQRDGEDQTQDTELVETRPAGDGTFQKWAAVVVPSGEEQRYTCHVQHEGLPEPLTLRWKQSSLPTIPI

SEQ ID NO: 128 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 129 - protein, 99 aa, Homo sapiens [sequence omitted]

SEQ ID NO: 130 - DNA, 357 bp, synthetic construct
  source 1..357: mol_type = other DNA
atgtcacgctccgtggcactggctgtgctggccctgctctccctgtcgggtcttgaggccatccagaggactccgaagattcaagtctactcccgccatcctgccgaaaacggaaagtccaattttctgaactgctatgtgtcgggcttccacccctccgacatcgaagtggacctcctgaagaacggggagcggattgaaaaggtcgagcacagcgacctgagcttctcgaaggactggtcattctacctcctgtactacactgaattcaccccaaccgaaaaggatgagtacgcgtgcagagtgaaccacgtgaccttgagccagccgaagatcgtgaaatgggaccgggatatg

SEQ ID NO: 131 - DNA, 1095 bp, synthetic construct
  source 1..1095: mol_type = other DNA
atggccgtgatggccccaaggacccttctgctcctcctgtcgggagcgctcgcactgactcagacctgggctggctcacactccatgagatacttcttcacttctgtgtcccggcctggaagaggggagcccaggttcatcgcggtcggctacgtggacgacacccagttcgtgcgcttcgactccgatgccgcctcgcaacgcatggagccgagagctccgtggatcgaacaagagggcccggagtactgggaccaggaaactagaaacgtgaaggcccacagccagaccgaccgcgtggatctgggaaccctccgcggttactacaatcagtcggaagctggatcccacacgattcagatgatgtacggttgcgacgtgggctccgatggacggtttctgcgggggtatcggcaggatgcctatgacgggaaggactacatcgccttgaacgaggacctccggtcatggactgccgcagacatggcggcccaaatcaccaagcgcaaatgggaagccgcgcatgtggcagagcagctgcgggcctaccttgagggcacttgcgtggaatggctgcgccgatacctggaaaacgggaaggaaaccctgcagcggactgacccacctaagacccacatgacccaccacgccgtgtccgaccatgaggccacactgcggtgctgggccttgtccttctaccctgccgaaatcaccctcacttggcaacgcgacggagaggatcagacccaagacaccgaactggtcgaaactcggcctgcgggagatggaaccttccagaaatgggccgctgtcgtggtgccgagcggacaggaacagaggtacacctgtcatgtgcagcacgagggtctgccgaagcccctgacgctgagatgggagctgtcaagccagcccactattcccattgtgggcattatcgccggactggtgctgcttggcgccgtcatcaccggtgctgtggtggcagccgtcatgtggcgccggaagtccagcgacaggaaggggggctcctacacccaagcggcgtcgagcgatagcgcccagggatccgacgtgtccctcaccgcctgcaaggtc

SEQ ID NO: 132 - protein, 341 aa, synthetic construct
GSHSMRYFFTSVSRPGRGEPRFIAVGYVDDTQFVRFDSDAASQRMEPRAPWIEQEGPEYWDQETRNVKAHSQTDRVDLGTLRGYYNQSEAGSHTIQMMYGCDVGSDGRFLRGYRQDAYDGKDYIALNEDLRSWTAADMAAQITKRKWEAAHVAEQLRAYLEGTCVEWLRRYLENGKETLQRTDPPKTHMTHHAVSDHEATLRCWALSFYPAEITLTWQRDGEDQTQDTELVETRPAGDGTFQKWAAVVVPSGQEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 133 - DNA, 1086 bp, synthetic construct
  source 1..1086: mol_type = other DNA
atgcgcgtgactgcccctcgaaccgtgctcctgctgctctggggagctgtggcactcaccgaaacttgggccggatcccacagcatgcggtacttttacactgcgatgtcgcgccctggaagaggggagccacgcttcattgccgtgggctacgtcgacgatacccagttcgtgcggttcgacagcgatgcagcctcgcctagagaggaacccagagccccgtggatcgaacaggaaggcccggagtactgggatcggaacacccagatctccaagaccaacacgcagacctatcgggaatccctgaggaacctcaggggttactacaaccagtccgaggccggaagccacaccctgcaaaggatgtacggatgcgacgtgggacccgatgggagactcctgcgcggtcacaaccagtacgcctacgacgggaaggactacatcgccctgaatgaggacctgtcatcctggaccgcggctgatacagcagcccagatcacccagcggaaatgggaggccgccagagtggcagaacagctgcgcgcgtatctggagggtttgtgcgtggaatggctgcggcggtacctcgaaaacggaaaggaaaccctgcagagagcagacccccccaagactcacgtcacccatcacccgatctctgaccatgaagccaccctgcggtgttgggccctcggcttctacccggcggaaattactctgacatggcagcgggacggagaggaccagacccaggacaccgagctggtcgaaactcgccctgccggagacaggactttccagaaatgggctgccgtggtggtgccgagcggagaggaacagcggtacacctgtcacgtgcaacatgagggccttccgaagcccctgactctgcgctgggagccttcctcccaatcgacgatcccaattgtcggcatcgtggccggtctggctgtgcttgcggtggtggtcattggcgcggtggtggctactgtgatgtgccgccgcaagagctcaggagggaagggcggctcctactcgcaagccgcctcctcggactccgcccaaggatccgatgtctcattgaccgcc

SEQ ID NO: 134 - protein, 338 aa, synthetic construct
GSHSMRYFYTAMSRPGRGEPRFIAVGYVDDTQFVRFDSDAASPREEPRAPWIEQEGPEYWDRNTQISKTNTQTYRESLRNLRGYYNQSEAGSHTLQRMYGCDVGPDGRLLRGHNQYAYDGKDYIALNEDLSSWTAADTAAQITQRKWEAARVAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTIPIVGIVAGLAVLAVVVIGAVVATVMCRRKSSGGKGGSYSQAASSDSAQGSDVSLTA

SEQ ID NO: 135 - DNA, 1098 bp, synthetic construct
  source 1..1098: mol_type = other DNA
atgcgcgtgatggcccctcgcaccttgattctgctcctgtccggtgcccttgctctcaccgagacttgggcctgctcgcactccatgcggtacttctacaccgccgtgtctaggccgggaaggggagagccgcggttcatcgccgtgggctacgtcgacgatacccagtttgtgcggttcgacagcgatgccgcaagccccagaggagagcctagagctccgtgggtcgaacaggagggccccgagtactgggaccgggaaacccagaagtacaagcgccaggcacaagcggatagagtgtccctgcggaacctccggggttactacaaccagtcagaagccggttcccatactctgcaacggatgtacggttgcgaccttggacccgacggcagactgctccggggctacgatcagtccgcctacgacgggaaggactacattgccctgaacgaggacctccggtcctggactgcggcagataccgcggcgcagatcacccagcgcaaatgggaagcggcccgagaagcagagcagctgagagcctatctggagggcacctgtgtcgagtggctgaggcgctatttggaaaacgggaaggagactctgcagagagcggaacaccccaagactcacgtgacccaccaccctgtgtccgaccatgaagccaccctgcgctgttgggccctggggttctaccctgccgaaatcaccctgacctggcaaagggacggagaggaccagacccaggacaccgaactggtggagactcggcccgctggagatgggaccttccaaaaatgggccgctgtggtcgtgccatccggggaggaacaacggtacacttgccacgtgcagcatgagggactcccggaaccgctgacacttcgctgggaaccgagcagccagccaacgatccccattgtgggcatcgtggccggactcgcagtgctggccgtgctggctgtcctgggagccgtgatggccgtcgtgatgtgccggcgcaagtcgtccggcggaaagggaggcagctgctcccaagccgcgtcctcgaattcggcccagggctcagacgaatcactgatcgcctgcaaggcg

SEQ ID NO: 136 - protein, 342 aa, synthetic construct
CSHSMRYFYTAVSRPGRGEPRFIAVGYVDDTQFVRFDSDAASPRGEPRAPWVEQEGPEYWDRETQKYKRQAQADRVSLRNLRGYYNQSEAGSHTLQRMYGCDLGPDGRLLRGYDQSAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEPSSQPTIPIVGIVAGLAVLAVLAVLGAVMAVVMCRRKSSGGKGGSCSQAASSNSAQGSDESLIACKA

SEQ ID NO: 137 - DNA, 1074 bp, synthetic construct
  source 1..1074: mol_type = other DNA
atggtcgacgggacgttgctcctcctgttgtccgaagccctggcgcttactcaaacctgggccggatcgcactccctgaagtatttccacacctccgtgtcaagacccggtagaggcgaacctcggttcatctccgtgggctacgtggacgacactcagttcgtgcgcttcgacaatgatgccgcatcgcctcggatggtgcccagagccccttggatggaacaggagggatcggagtactgggaccgcgagactcggtcagcccgcgatactgcccagattttccgcgtcaacctgaggaccctgaggggctactataaccagagcgaagcgggttcccataccctccaatggatgcatggctgcgaactgggtccagacaggcgcttcttgcggggctacgaacagtttgcctacgatggaaaggattacctgaccctcaacgaggacctccgctcttggaccgccgtggacactgcggcccaaatctccgaacagaagtcgaacgacgcctcagaggcggagcaccagcgagcttaccttgaggacacttgcgtggagtggctgcacaagtacctggaaaagggaaaggaaaccctcctgcacctggagcctcccaagacccatgtcacccaccacccgatctccgaccacgaagctaccctccggtgttgggccctgggcttttacccggcggaaatcaccctgacgtggcagcaggacggggagggacacacccaggacaccgaactggtggagactagaccagccggcgatgggactttccagaaatgggccgctgtggtagtgcccagcggcgaagaacagcggtacacatgccatgtgcaacacgagggactgcccgagcctgtgacccttcggtggaagccggcctcacaaccgaccatcccgattgtcggaattatcgccggactggtgctgctcgggtccgtcgtgtccggagcagtggtggccgctgtgatctggcggaagaagtccagcggaggaaaggggggttcctactcgaaagcggagtggagcgatagcgcacagggatccgagagccactcgctg

SEQ ID NO: 138 - protein, 337 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 139 - DNA, 1038 bp, synthetic construct
  source 1..1038: mol_type = other DNA
atggcaccacggtcgttgctcctgctcctgtcgggcgctcttgccctcaccgacacttgggccggcagccattctcttcggtacttctccaccgccgtcagcagaccgggaaggggagaaccgcggtatatcgcggtggaatatgtggacgatacccagttcctgcggttcgactccgatgccgcgattccaaggatggagcccagagaaccctgggtggagcaggaaggcccgcagtactgggaatggaccaccggctacgccaaggccaacgctcagaccgatagggtggcgctgcgcaacctcttgcggcggtacaatcagtcagaagcgggttcccacacgctgcaagggatgaacggctgcgacatgggacctgacggtagactgctccgaggctaccaccaacacgcgtacgatggaaaggactacattagcctgaacgaggatctgcggtcctggactgcggccgacactgtggcccaaatcacccagcgcttttacgaggcagaagaatacgccgaagaattccgcacctacctggagggcgaatgcttggagcttctgcggcgctacctggaaaacgggaaggagactctgcagagagccgaccctcccaaggcccatgtggcccaccaccctatctcggaccatgaggcgaccctgcggtgttgggccctggggttctacccggctgagattaccctgacctggcaacgcgacggagaggagcagacccaggacaccgaactcgtggaaaccagaccggccggagatggaacattccagaaatgggccgctgtcgtggtgccctccggagaggaacagcgctacacttgccacgtgcagcacgagggactgccacagcccctgatcctgcgctgggagcagtcccctcaaccgactatccctatcgtcggtatcgtggctggtctggtggtgctcggagccgtcgtgactggggcagtggtggcagccgtgatgtggcgcaagaagtcctcagaccggaacaggggcagctactcccaagccgccgtc

SEQ ID NO: 140 - protein, 325 aa, synthetic construct
GSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPSGEEQRYTCHVQHEGLPQPLILRWEQSPQPTIPIVGIVAGLVVLGAVVTGAVVAAVMWRKKSSDRNRGSYSQAAV

SEQ ID NO: 141 - protein, 691 aa, synthetic construct
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLEKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 142 - protein, 668 aa, synthetic construct
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLEKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 143 - protein, 693 aa, synthetic construct
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLELEKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 144 - protein, 672 aa, synthetic construct
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLELEKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 145 - protein, 462 aa, synthetic construct
MSVPTQVLGLLLLWLTDARCRIIPRHLQLGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSASPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQLEENLYFQGSGGSHHHHHHHHHH

SEQ ID NO: 146 - protein, 427 aa, synthetic construct
RIIPRHLQLGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSASPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQLEENLYFQ